(19) United States
(12) Patent Application Publication
(10) Pub. No.: US 2022/0407028 A1
(43) Pub. Date: Dec. 22, 2022

(54) LIGHT EMITTING DIODE AND DISPLAY DEVICE INCLUDING THE SAME

(71) Applicant: Samsung Display Co., Ltd., Yongin-si (KR)

(72) Inventors: BORA LEE, Hwaseong-si (KR); HYOMIN KO, Suwon-si (KR); ILLHUN CHO, Seoul (KR); EUNJAE JEONG, Hwaseong-si (KR); MINJI KIM, Hwaseong-si (KR); SOHEE JO, Cheonan-si (KR); DONGJUN KIM, Suwon-si (KR); HANKYU PAK, Suwon-si (KR); SANGHYUN HAN, Hwaseong-si (KR)

(73) Assignee: Samsung Display Co., Ltd., Yongin-si (KR)

(21) Appl. No.: 17/874,578

(22) Filed: Jul. 27, 2022

Related U.S. Application Data
(63) Continuation-in-part of application No. 17/153,310, filed on Jan. 20, 2021.

(30) Foreign Application Priority Data
Jan. 21, 2020 (KR) 10-2020-0007949
Dec. 16, 2020 (KR) 10-2020-0176483
Apr. 18, 2022 (KR) 10-2022-0047678

(51) Int. Cl. H01L 51/50; C09K 11/06; C07C 211/54; C07D 307/91; C07C 211/61; C07D 209/88; C07D 333/76

(52) U.S. Cl. CPC H01L 51/5064; H01L 51/5044; C09K 11/06; C07C 211/54; C07D 307/91; C07C 211/61; C07D 209/88; C07D 333/76; H01L 2251/558; C07C 2601/14; C07C 2603/74; H01L 51/006

(57) ABSTRACT

A light emitting diode of an embodiment includes a first electrode, a hole transport region disposed on the first electrode, an emission layer disposed on the hole transport region, an electron transport region disposed on the emission layer, and a second electrode disposed on the electron transport region. The hole transport region includes a first hole transport layer disposed adjacent to the first electrode and having a first refractive index, a second hole transport layer disposed adjacent to the emission layer and having a second refractive index, and a third hole transport layer disposed between the first hole transport layer and the second hole transport layer and having a third refractive index which is greater than each of the first refractive index and the second refractive index, thereby showing high light extraction efficiency and high emission efficiency properties.

### CROSS-REFERENCE TO RELATED APPLICATION(S)

This is a continuation-in-part application of U.S. patent application Ser. No. 17/153,310, filed Jan. 20, 2021 (now pending), the disclosure of which is incorporated herein by reference in its entirety. U.S. patent application Ser. No. 17/153,310 claims priority to and benefits of Korean Patent Application Nos. 10-2020-0007949 and 10-2020-0176483 under 35 U.S.C. § 119, filed on Jan. 21, 2020 and Dec. 16, 2020, respectively, in the Korean Intellectual Property Office, the entire contents of which are incorporated herein by reference. This application claims priority to and benefits of Korean Patent Application No. 10-2022-0047678 under 35 U.S.C. § 119, filed on Apr. 18, 2022 in the Korean Intellectual Property Office, the entire contents of which are incorporated herein by reference.

### BACKGROUND

### 1. Technical Field

The disclosure relates to a light emitting diode including multiple hole transport layers having different refractive indexes and a display device including the same.

### 2. Description of the Related Art

Various display devices used in multimedia apparatuses such as televisions, cellular phones, tablet computers, navigations, and game consoles are being developed. In such a display devices, a so-called self-luminescent display device in which a light emitting material including an organic compound or quantum dots in an emission layer disposed between oppositely disposed electrodes emits light to achieve display, is used.

In the application of a light emitting diode to a display device, the increase of emission efficiency and life of the light emitting diode is required, and development on materials and structures for a light emitting diode stably achieving the requirement is being continuously required.

### SUMMARY

The disclosure provides a light emitting diode having excellent light emission efficiency.

The disclosure also provides a display device including a light emitting diode having high emission efficiency.

An embodiment provides a light emitting diode that may include a first electrode, a hole transport region disposed on the first electrode, an emission layer disposed on the hole transport region, an electron transport region disposed on the emission layer, and a second electrode disposed on the electron transport region. The hole transport region may include a first hole transport layer disposed adjacent to the first electrode, the first hole transport layer having a first refractive index; a second hole transport layer disposed adjacent to the emission layer, the second hole transport layer having a second refractive index; and a third hole transport layer disposed between the first hole transport layer and the second hole transport layer, the third hole transport layer having a third refractive index which is greater than each of the first refractive index and the second refractive index.

In an embodiment, a difference between the third refractive index and the first refractive index may be greater than about 0.1, and a difference between the third refractive index and the second refractive index may be greater than about 0.1.

In an embodiment, the first refractive index and the second refractive index may each be in a range of about 1.30 to about 1.80 at a wavelength of about 460 nm, and the third refractive index may be in a range of about 1.85 to about 2.40 at a wavelength of about 460 nm.

In an embodiment, the first refractive index and the second refractive index may be the same.

In an embodiment, the second hole transport layer may be disposed directly below the emission layer.

In an embodiment, a refractive index of the emission layer may be greater than the second refractive index of the second hole transport layer, and a difference between the refractive index of the emission layer and the second refractive index may be greater than about 0.1 at a wavelength of about 460 nm.

In an embodiment, the refractive index of the emission layer may be in a range of about 1.80 to about 2.40 at a wavelength of about 460 nm.

In an embodiment, the first hole transport layer may be disposed directly above the first electrode.

In an embodiment, a refractive index of the first electrode may be greater than the first refractive index of the first hole transport layer, and a difference between the refractive index of the first electrode and the first refractive index may be greater than about 0.1 at a wavelength of about 460 nm.

In an embodiment, the refractive index of the first electrode may be in a range of about 1.80 to about 2.40 at a wavelength of about 460 nm.

In an embodiment, a thickness ratio of the first hole transport layer, the third hole transport layer, and the second hole transport layer may be in a range of about 0.1:0.8:0.1 to about 0.45:0.1:0.45.

In an embodiment, the first electrode may be a reflective electrode, and the second electrode may be a transmissive electrode or a transflective electrode.

In an embodiment, the emission layer may emit light having a central wavelength in a range of about 430 nm to about 470 nm.

In an embodiment, a thickness of the first hole transport layer may be in a range of about 100 Å to about 1,000 Å, a thickness of the second hole transport layer may be in a range of about 100 Å to about 1,000 Å, and the third hole transport layer may be in a range of about 100 Å to about 1,000 Å.

In an embodiment, the first hole transport layer and the second hole transport layer may each independently include an amine compound represented by Formula 1 below.

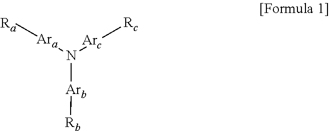

In Formula 1, Arₐ to Ar_{c} may each independently be a substituted or unsubstituted aryl group of 6 to 30 ring-forming carbon atoms, or a substituted or unsubstituted heteroaryl group of 3 to 30 ring-forming carbon atoms, at least two of Rₐ to R_{c} may each independently be an adamantyl group or a cyclohexyl group, and the remainder of Rₐ to R_{c} may be a hydrogen atom, a deuterium atom, a halogen atom, a cyano group, a substituted or unsubstituted oxy group, a substituted or unsubstituted thio group, a substituted or unsubstituted amine group, or a substituted or unsubstituted alkyl group of 1 to 20 carbon atoms.

In an embodiment, Arₐ to Ar_{c} may each independently be a substituted or unsubstituted phenyl group, a substituted or unsubstituted naphthyl group, a substituted or unsubstituted biphenyl group, a substituted or unsubstituted terphenyl group, a substituted or unsubstituted fluorenyl group, a substituted or unsubstituted carbazole group, a substituted or unsubstituted dibenzofuranyl group, or a substituted or unsubstituted dibenzothiophenyl group.

In an embodiment, the third hole transport layer may include a compound represented by Formula 2 below.

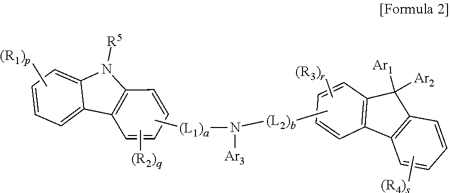

In Formula 2, Ar₁ and Ar₂ may each independently be a hydrogen atom, a deuterium atom, a halogen atom, a substituted or unsubstituted alkyl group of 1 to 30 carbon atoms, a substituted or unsubstituted aryl group of 6 to 30 ring-forming carbon atoms, or a substituted or unsubstituted heteroaryl group of 2 to 30 ring-forming carbon atoms, or combined with an adjacent group to form a ring, and Arₙ may be a substituted or unsubstituted aryl group of 6 to 30 ring-forming carbon atoms, or a substituted or unsubstituted heteroaryl group of 2 to 30 ring-forming carbon atoms. In Formula 2, a and b may each independently be 0 or 1, and Li and L₂ may each independently be a substituted or unsubstituted cycloalkylene group of 3 to 10 ring-forming carbon atoms, a substituted or unsubstituted heterocycloalkylene group of 2 to 10 ring-forming carbon atoms, a substituted or unsubstituted cycloalkenylene group of 3 to 10 ring-forming carbon atoms, a substituted or unsubstituted arylene group of 6 to 30 ring-forming carbon atoms, or a substituted or unsubstituted heteroarylene group of 2 to 60 ring-forming carbon atoms. In Formula 2, p and s may each independently be an integer from 0 to 4, q and r may each independently be an integer from 0 to 3, and R₁ to R₅ may each independently be a hydrogen atom, a deuterium atom, a halogen atom, a hydroxyl group, a cyano group, a nitro group, an amino group, a substituted or unsubstituted silyl group, a substituted or unsubstituted oxy group, a substituted or unsubstituted alkyl group of 1 to 60 carbon atoms, a substituted or unsubstituted heterocycloalkyl group of 3 to 60 ring-forming carbon atoms, a substituted or unsubstituted aryl group of 6 to 60 ring-forming carbon atoms, or a substituted or unsubstituted heteroaryl group of 2 to 60 ring-forming carbon atoms.

In an embodiment, the hole transport region may further include a fourth hole transport layer disposed between the first hole transport layer and the third hole transport layer, the fourth hole transport layer having a refractive index greater than the first refractive index and less than the third refractive index; and a fifth hole transport layer disposed between the second hole transport layer and the third hole transport layer, the fifth hole transport layer having a refractive index greater than the second refractive index and less than the third refractive index.

In an embodiment, the first hole transport layer and the second hole transport layer may each independently include an amine compound represented by Formula 1 above, the third hole transport layer may include a compound represented by Formula 2 above, and the fourth hole transport layer and the fifth hole transport layer may each independently include an amine compound represented by Formula 1 above and a compound represented by Formula 2 above.

In an embodiment, the first to fifth hole transport layers may each have a thickness in a range of about 100 Å to about 1,000 Å.

According to an embodiment, there is provided a display device including light emitting diodes, each of the light emitting diodes including a first electrode; a hole transport region disposed on the first electrode; an emission layer disposed on the hole transport region; an electron transport region disposed on the emission layer; and a second electrode disposed on the electron transport region. The hole transport region of at least one of the light emitting diodes may include a first hole transport layer disposed adjacent to the first electrode, the first hole transport layer having a first refractive index; a second hole transport layer disposed adjacent to the emission layer, the second hole transport layer having a second refractive index; and a third hole transport layer disposed between the first hole transport layer and the second hole transport layer, the third hole transport layer having a third refractive index which is greater than each of the first refractive index and the second refractive index.

In an embodiment, a difference between the third refractive index and the first refractive index may be greater than about 0.1, and a difference between the third refractive index and the second refractive index may be greater than about 0.1.

In an embodiment, the first electrode may be a reflective electrode, and the second electrode may be a transmissive electrode or a transflective electrode.

An embodiment provides a light emitting diode which may include a first electrode, a second electrode oppositely disposed to the first electrode, and light emitting units disposed between the first electrode and the second electrode, wherein each of the light emitting units may include a hole transport region, an emission layer, and an electron transport region stacked in order, and the hole transport region of at least one of the light emitting units may include an amine compound represented by Formula 1.

In an embodiment, the hole transport region of a light emitting unit adjacent to the second electrode may include the amine compound.

In an embodiment, the light emitting units may include at least one blue light emitting unit that emits blue light.

In an embodiment, the light emitting diode may further include a charge generating layer disposed between the light emitting units.

In an embodiment, the charge generating layer may include a p-type charge generating layer including a p-dopant, and an n-type charge generating layer including an n-dopant.

In an embodiment, the light emitting diode may further include a capping layer disposed on the second electrode, wherein the capping layer may have a refractive index equal to or greater than about 1.6.

In an embodiment, Arₐ to Ar_{c} may each independently be a substituted or unsubstituted phenyl group, a substituted or unsubstituted naphthyl group, a substituted or unsubstituted biphenyl group, a substituted or unsubstituted terphenyl group, a substituted or unsubstituted fluorenyl group, a substituted or unsubstituted carbazole group, a substituted or unsubstituted dibenzofuranyl group, or a substituted or unsubstituted dibenzothiophenyl group.

In an embodiment, the hole transport region including the amine compound may have a refractive index in a range of about 1.30 to about 1.80, with respect to light at a wavelength of about 460 nm.

In an embodiment, the hole transport region of at least one of the light emitting units including the amine compound may include a first hole transport layer having a first refractive index, a second hole transport layer disposed adjacent to the emission layer and having a second refractive index, and a third hole transport layer disposed between the first hole transport layer and the second hole transport layer and having a third refractive index greater than each of the first refractive index and the second refractive index.

In an embodiment, the first hole transport layer and the second hole transport layer may each include the amine compound, each independently represented by Formula 1.

In an embodiment, the third hole transport layer may include a compound represented by Formula 2.

An embodiment provides a light emitting diode which may include a first electrode; first to fourth light emitting units each including a hole transport region, an emission layer, and an electron transport region, and disposed in order on the first electrode; a charge generating layer disposed between the first to fourth light emitting units; and a second electrode disposed on the fourth light emitting unit, wherein the fourth light emitting unit may emit light having a longer wavelength than the first to third light emitting units, and the hole transport region of the fourth light emitting unit may include an amine compound represented by Formula 1.

In an embodiment, the hole transport region of at least one of the first to third light emitting units may include the amine compound.

In an embodiment, the hole transport region including the amine compound may have a refractive index in a range of about 1.30 to about 1.80, with respect to light at a wavelength of about 460 nm.

In an embodiment, each of the first light emitting unit, the second light emitting unit, and the third light emitting unit may emit light having a first wavelength in a range of about 430 nm to about 470 nm, and the fourth light emitting unit may emit light having a second wavelength in a range of about 520 nm to about 600 nm.

In an embodiment, each of the first light emitting unit, the second light emitting unit, and the third light emitting unit may include: a first sub-emission layer including a first host, and a first dopant emitting the light having a first wavelength; and a second sub-emission layer including a second host that is different from the first host, and a second dopant emitting the light having a first wavelength.

In an embodiment, Arₐ to Ar_{c} may each independently be a substituted or unsubstituted phenyl group, a substituted or unsubstituted naphthyl group, a substituted or unsubstituted biphenyl group, a substituted or unsubstituted terphenyl group, a substituted or unsubstituted fluorenyl group, a substituted or unsubstituted carbazole group, a substituted or unsubstituted dibenzofuranyl group, or a substituted or unsubstituted dibenzothiophenyl group.

An embodiment provides a display device, which may be divided into: a first luminous area emitting light with a first wavelength, a second luminous area emitting light with a second wavelength that is different from the first wavelength, and a third luminous area emitting light with a third wavelength that is different from the first wavelength and the second wavelength.

The display device may include a display device layer disposed on a base substrate and including the light emitting diode, and a light controlling layer disposed on the display device layer and including a quantum dot. The light emitting diode may include a first electrode, a second electrode oppositely disposed to the first electrode, and light emitting units disposed between the first electrode and the second electrode, wherein each of the light emitting units may include a hole transport region, an emission layer, and an electron transport region stacked in order, and the hole transport region of at least one of the light emitting units may include an amine compound represented by Formula 1.

In an embodiment, the hole transport region of a light emitting unit adjacent to the second electrode may include the amine compound.

In an embodiment, the light emitting units may include at least one blue light emitting unit that emits blue light.

In an embodiment, the display device may further include a charge generating layer disposed between the light emitting units.

In an embodiment, Arₐ to Ar_{c} may each independently be a substituted or unsubstituted phenyl group, a substituted or unsubstituted naphthyl group, a substituted or unsubstituted biphenyl group, a substituted or unsubstituted terphenyl group, a substituted or unsubstituted fluorenyl group, a substituted or unsubstituted carbazole group, a substituted or unsubstituted dibenzofuranyl group, or a substituted or unsubstituted dibenzothiophenyl group.

In an embodiment, the hole transport region including the amine compound may have a refractive index in a range of about 1.30 to about 1.80, with respect to light at a wavelength of about 460 nm.

In an embodiment, the light emitting units may include first to fourth light emitting units disposed in order, each of the first light emitting unit, the second light emitting unit, and the third light emitting unit may emit light having a first wavelength in a range of about 430 nm to about 470 nm, and the fourth light emitting unit may emit light having a second wavelength in a range of about 520 nm to about 600 nm.

It is to be understood that the embodiments above are described in a generic and explanatory sense only and not for the purpose of limitation, and the disclosure is not limited to the embodiments described above.

### BRIEF DESCRIPTION OF THE DRAWINGS

The accompanying drawings are included to provide a further understanding of the disclosure and are incorporated in and constitute a part of this specification. The drawings illustrate embodiments of disclosure and, together with the description, serve to explain principles thereof. In the drawings:

FIG. 1 is a perspective view showing an electronic device according to an embodiment;

FIG. 2 is a plan view of a display device according to an embodiment;

FIG. 3 is a schematic cross-sectional view of a display device of an embodiment, corresponding to line I-I′ in FIG. 2;

FIG. 4 is a schematic cross-sectional view of a light emitting diode of an embodiment;

FIG. 5 is a schematic cross-sectional view of a portion of a light emitting diode according to an embodiment;

FIG. 6 is a schematic cross-sectional view of a portion of a light emitting diode according to an embodiment;

FIG. 7 is a graph comparing and showing efficiency properties of light emitting diodes of the Comparative Examples and the Example;

FIG. 8 is a schematic cross-sectional view of a display device of an embodiment corresponding to line I-I′ in FIG. 2;

FIG. 9 is a schematic cross-sectional view showing a light emitting diode according to an embodiment;

FIG. 10A is a schematic cross-sectional view showing a light emitting unit according to an embodiment;

FIG. 10B is a schematic cross-sectional view showing a light emitting unit according to an embodiment;

FIG. 10C is a schematic cross-sectional view showing a light emitting unit according to an embodiment;

FIG. 11 is a schematic cross-sectional view showing a light emitting diode according to an embodiment; and

FIG. 12 is a schematic cross-sectional view showing a light emitting diode according to an embodiment.

### DETAILED DESCRIPTION OF THE EMBODIMENTS

The disclosure will now be described more fully hereinafter with reference to the accompanying drawings, in which embodiments are shown. This disclosure may, however, be embodied in different forms and should not be construed as limited to the embodiments set forth herein. Rather, these embodiments are provided so that this disclosure will be thorough and complete, and will fully convey the scope of the disclosure to those skilled in the art.

In the description, it will be understood that when an element (or region, layer, part, etc.) is referred to as being “on”, “above”, “connected to”, “coupled to”, or “adjacent to” another element, it can be directly on, above, connected to, coupled to, or adjacent to the other element, or one or more intervening elements may be present therebetween.

Like reference numerals refer to like elements throughout the specification. In the drawings, the thickness, the ratio, and the dimensions of constituent elements may be exaggerated for an effective explanation of its technical contents. Therefore, as the sizes and thicknesses of components in the drawings may be arbitrarily illustrated for convenience of explanation, the following embodiments of the disclosure are not limited thereto.

As used herein, the expressions used in the singular such as “a,” “an,” and “the” are intended to include the plural forms as well, unless the context clearly indicates otherwise.

As used herein, the term “and/or” includes any and all combinations of one or more of the associated listed items. For example, “A and/or B” may be understood to mean “A, B, or A and B.” The terms “and” and “or” may be used in the conjunctive or disjunctive sense and may be understood to be equivalent to “and/or”.

The term “at least one of” is intended to include the meaning of “at least one selected from” for the purpose of its meaning and interpretation. For example, “at least one of A and B” may be understood to mean “A, B, or A and B.” When preceding a list of elements, the term, “at least one of,” modifies the entire list of elements and does not modify the individual elements of the list.

It will be understood that, although the terms first, second, etc. may be used herein to describe various elements, these elements should not be limited by these terms. These terms are only used to distinguish one element from another element. For example, a first element could be termed a second element without departing from the teachings of the invention.

Similarly, a second element could be termed a first element without departing from the teachings of the invention.

The terms “below”, “beneath”, “on” and “above” are used for explaining the relation of elements shown in the drawings. The terms are a relative concept and are explained based on the direction shown in the drawings.

The terms “about” or “approximately” as used herein is inclusive of the stated value and means within an acceptable range of deviation for the recited value as determined by one of ordinary skill in the art, considering the measurement in question and the error associated with measurement of the recited quantity (i.e., the limitations of the measurement system). For example, “about” may mean within one or more standard deviations, or within +20%, 10%, or 5% of the stated value.

It will be further understood that the terms “comprises,” “comprising,” “includes,” “including,” “have,” “having,” “contains,” “containing,” and the like when used in this specification, specify the presence of stated features, numerals, steps, operations, elements, parts, or combinations thereof, but do not preclude the presence or addition of one or more other features, numerals, steps, operations, elements, parts, or combinations thereof.

Unless otherwise defined or implied herein, all terms (including technical and scientific terms) used have the same meaning as commonly understood by those skilled in the art to which this disclosure pertains. It will be further understood that terms, such as those defined in commonly used dictionaries, should be interpreted as having a meaning that is consistent with their meaning in the context of the relevant art and should not be interpreted in an ideal or excessively formal sense unless clearly defined in the specification.

Hereinafter, a light emitting diode and a display device including the same according to an embodiment will be explained with reference to the accompanying drawings.

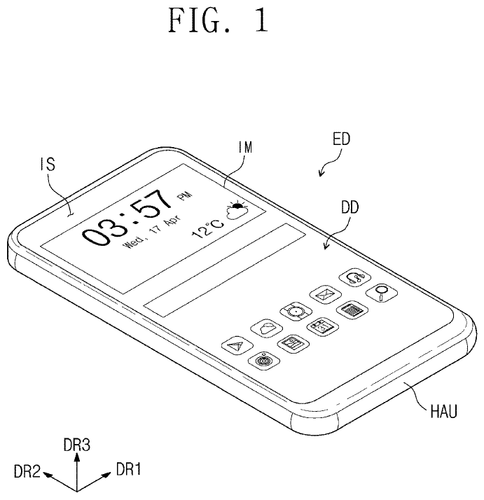

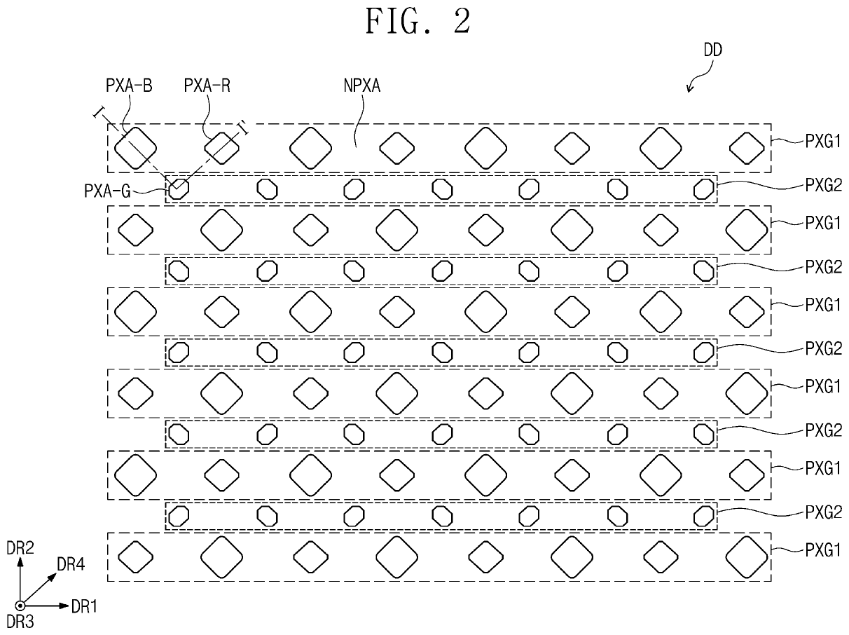

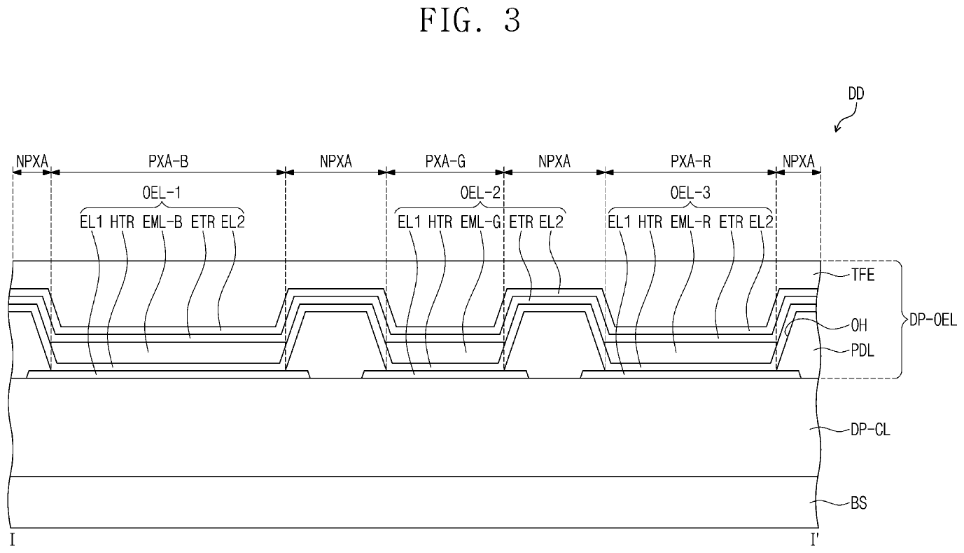

FIG. 1 is a perspective view showing an embodiment of an electronic device ED. FIG. 2 is a plan view of a display device DD according to an embodiment. FIG. 3 is a schematic cross-sectional view of a display device DD according to an embodiment. FIG. 3 is a schematic cross-sectional view showing a portion corresponding to line I-I′ in FIG. 2.

In an embodiment, the electronic device ED may be a small- or medium-sized electronic device such as smart phones, tablets, personal computers, laptop computers, personal digital terminals, car navigation units, game consoles, and cameras. The electronic device ED may be a large-sized electronic device such as televisions, monitors, and external billboards. However, these are only embodiments, and other electronic devices may be employed as long as they do not deviate from the inventive concept.

The electronic device ED may include a display device DD and a housing HAU. The display device DD may display images IM through a display surface IS. In FIG. 1, the display surface IS is shown parallel to a plane defined by a first directional axis DR1 and a second directional axis DR2 crossing the first directional axis DR1. However, this is an illustration, and in other embodiments, the display surface IS of the display device DD may have a bent shape.

Among the directions of the normal line of the display surface IS, i.e., the thickness directions of the display device DD, a direction displaying the images IM is indicated by a third directional axis DR3. The front (or top) and rear (or bottom) of each member may be divided by the third directional axis DR3. The directions indicated by the first to third directional axes DR1, DR2, and DR3 are a relative concept and may be changed into other directions.

The housing HAU may receive the display device DD. The housing HAU may be disposed so as to cover the display device DD and expose the top surface which is the display surface IS of the display device DD. The housing HAU may cover the side and bottom surface of the display device DD while exposing the entire top surface. However, embodiments are not limited thereto, and the housing HAU may cover a portion of the top as well as the side and bottom surface of the display device DD.

The display device DD may include abase substrate BS, a circuit layer DP-CL provided on the base substrate BS, and a display device layer DP-OEL. The display device layer DP-OEL may include a pixel defining layer PDL, light emitting diodes OEL-1, OEL-2, and OEL-3 disposed in the pixel defining layer PDL, and an encapsulating layer TFE disposed on the light emitting diodes OEL-1, OEL-2, and OEL-3.

The base substrate BS may be a member providing a base surface where the display device layer DP-OEL is disposed. The base substrate BS may be a glass substrate, a metal substrate, a plastic substrate, etc. However, embodiments are not limited thereto, and the base substrate BS may be an inorganic layer, an organic layer, or a composite material layer.

In an embodiment, the circuit layer DP-CL is disposed on the base substrate BS, and the circuit layer DP-CL may include multiple transistors (not shown). Each of the transistors (not shown) may include a control electrode, an input electrode, and an output electrode. For example, the circuit layer DP-CL may include switching transistors and driving transistors for driving the light emitting diodes OEL-1, OEL-2, and OEL-3 of the display device layer DP-OEL.

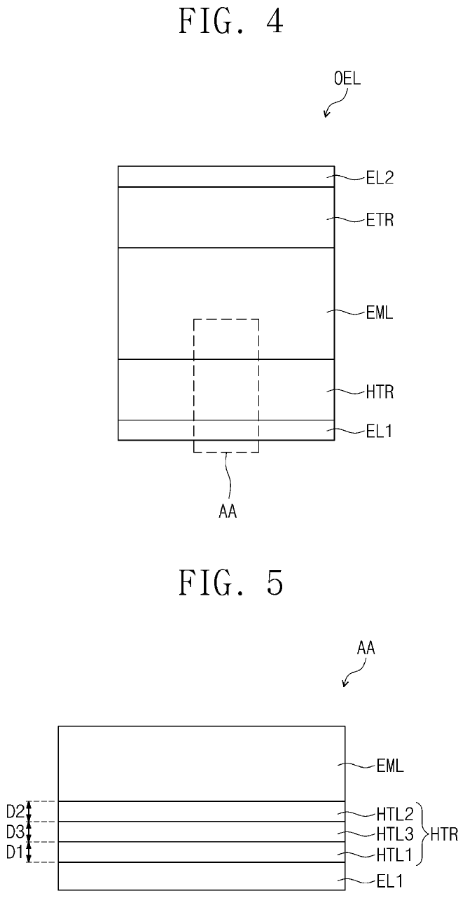

Each of the light emitting diodes OEL-1, OEL-2, and OEL-3 may include a first electrode EL1, a hole transport region HTR, emission layers EML-B, EML-G, and EML-R, an electron transport region ETR and a second electrode EL2. Each of the light emitting diodes OEL-1, OEL-2, and OEL-3 included in the display device DD of an embodiment may have the structure of a light emitting diode OEL of an embodiment (FIG. 4), which will be explained later. The hole transport region HTR included in each of the light emitting diodes OEL-1, OEL-2, and OEL-3 included in the display device DD of an embodiment may include hole transport layers having refractive index values different from each other.

In FIG. 3, an embodiment is shown where the emission layers EML-B, EML-G, and EML-R of light emitting diodes OEL-1, OEL-2, and OEL-3, which are in opening portions OH defined in a pixel defining layer PDL, are disposed, and a hole transport region HTR, an electron transport region ETR and a second electrode EL2 are provided as common layers in all light emitting diodes OEL-1, OEL-2, and OEL-3. However, embodiments are not limited thereto. In contrast to FIG. 3, in an embodiment, the hole transport region HTR or the electron transport region ETR may be divided by the pixel defining layer PDL and may be patterned and provided in the opening portions OH defined in the pixel defining layer PDL.

In an embodiment, the hole transport region HTR, the emission layers EML-B, EML-G, and EML-R, and the electron transport region ETR of the light emitting diodes OEL-1, OEL-2, and OEL-3 may be provided by using various methods such as a vacuum deposition method, a spin coating method, a cast method, a Langmuir-Blodgett (LB) method, an ink jet printing method, a laser printing method, and a laser induced thermal imaging (LITI) method.

The encapsulating layer TFE may cover the light emitting diodes OEL-1, OEL-2, and OEL-3. The encapsulating layer TFE may encapsulate the display device layer DP-OEL. The encapsulating layer TFE may be disposed on the second electrode EL2 and may be disposed while filling up the opening portion OH.

The encapsulating layer TFE may be a thin film encapsulating layer. The encapsulating layer TFE may be one layer or a stack of multiple layers. The encapsulating layer TFE may include at least one insulating layer. The encapsulating layer TFE according to an embodiment may include at least one inorganic layer (hereinafter, encapsulating inorganic layer). The encapsulating layer TFE according to an embodiment may include at least one organic layer (hereinafter, encapsulating organic layer) and at least one encapsulating inorganic layer.

The encapsulating inorganic layer protects the display device layer DP-OEL from moisture and/or oxygen, and the encapsulating organic layer protects the display device layer DP-OEL from foreign materials such as dust particles. The encapsulating inorganic layer may include a silicon nitride layer, a silicon oxy nitride layer, a silicon oxide layer, a titanium oxide layer, or an aluminum oxide layer, without specific limitation. The encapsulating organic layer may include an acrylic organic layer, without specific limitation.

Although not shown in the drawings, a capping layer (not shown) may be further disposed on the second electrode EL2. For example, the capping layer (not shown) may be disposed between the second electrode EL2 and the encapsulating layer TFE.

Referring to FIG. 2 and FIG. 3, the display device DD may include a non-light emitting region NPXA and light emitting regions PXA-B, PXA-G, and PXA-R. The light emitting regions PXA-B, PXA-G, and PXA-R may be areas emitting light produced from the light emitting diodes OEL-1, OEL-2, and OEL-3, respectively. The light emitting regions PXA-B, PXA-G, and PXA-R may be separated from each other on a plane.

The light emitting regions PXA-B, PXA-G, and PXA-R may be areas separated by the pixel defining layer PDL. The non-light emitting regions NPXA may be areas between neighboring light emitting regions PXA-B, PXA-G, and PXA-R and may be areas corresponding to the pixel defining layer PDL. In the disclosure, each of the light emitting regions PXA-B, PXA-G, and PXA-R may correspond to each pixel. The pixel defining layer PDL may divide the light emitting diodes OEL-1, OEL-2, and OEL-3. The emission layers EML-B, EML-G, and EML-R of the light emitting diodes OEL-1, OEL-2, and OEL-3 may be disposed and divided in the opening portions OH defined in the pixel defining layer PDL. The emission layers EML-B, EML-G, and EML-R defined by the pixel defining layer PDL may be formed by an ink jet printing method, etc.

The pixel defining layer PDL may be formed using a polymer resin. For example, the pixel defining layer PDL may be formed by including a polyacrylate-based resin or a polyimide-based resin. The pixel defining layer PDL may be formed by further including an inorganic material in addition to the polymer resin. The pixel defining layer PDL may be formed by including a light-absorbing material, or by including a black pigment or a black dye. The pixel defining layer PDL formed by including the black pigment or the black dye may form a black pixel defining layer. During forming the pixel defining layer PDL, carbon black may be used as the black pigment or the black dye, but embodiments are not limited thereto.

The pixel defining layer PDL may be formed using an inorganic material. For example, the pixel defining layer PDL may be formed by including silicon nitride (SiNₓ), silicon oxide (SiOₓ), silicon oxynitride (SiOₓN_{y}), etc. The pixel defining layer PDL may define the light emitting regions PXA-B, PXA-G, and PXA-R. The light emitting regions PXA-B, PXA-G, and PXA-R and the non-light emitting region NPXA may be defined by the pixel defining layer PDL.

The light emitting regions PXA-B, PXA-G, and PXA-R may be divided into multiple groups according to the color of light produced from the light emitting diodes OEL-1, OEL-2, and OEL-3. In the display device DD of an embodiment, shown in FIG. 2 and FIG. 3, three light emitting regions PXA-B, PXA-G, and PXA-R emitting blue light, green light, and red light are illustrated as an embodiment. For example, the display device DD of an embodiment may include a red light emitting region PXA-R, a green light emitting region PXA-G, and a blue light emitting region PXA-B, which are separated from each other.

The display device DD according to an embodiment includes multiple light emitting diodes OEL-1, OEL-2, and OEL-3, and the multiple light emitting diodes OEL-1, OEL-2, and OEL-3 may emit light having different wavelength regions. For example, in an embodiment, the display device DD may include a first light emitting diode OEL-1 emitting blue light, a second light emitting diode OEL-2 emitting green light, and a third light emitting diode OEL-3 emitting red light. However, embodiments are not limited thereto, and the first to third light emitting diodes OEL-1, OEL-2 and OEL-3 may emit light in the same wavelength region, or at least one thereof may emit light in a different wavelength region. For example, the blue light emitting region PXA-B, the green light emitting region PXA-G, and the red light emitting region PXA-R may correspond to the first light emitting diode OEL-1, the second light emitting diode OEL-2, and the third light emitting diode OEL-3, respectively.

In an embodiment, all the first to third light emitting diodes OEL-1, OEL-2, and OEL-3 may emit light in a blue wavelength region. The display device DD may further include a color controlling layer on the display device layer DP-OEL. The color controlling layer may be a part transmitting light or converting the wavelength of light provided from the first to third light emitting diodes OEL-1, OEL-2, and OEL-3.

Referring to FIG. 2, the blue light emitting region PXA-B and the red light emitting region PXA-R may be alternately arranged along the first directional axis DR1 to from a first group PXG1. The green light emitting regions PXA-G may be arranged along the first directional axis DR1 to form a second group PXG2. The first group PXG1 may be separately disposed from the second group PXG2 in the second directional axis DR2. Each of the first group PXG1 and the second group PXG2 may be provided in numbers. The first groups PXG1 and the second groups PXG2 may be alternately arranged along the second directional axis DR2.

One green light emitting region PXA-G may be separately disposed from one blue light emitting region PXA-B or one red light emitting region PXA-R in a fourth directional axis DR4. The fourth directional axis DR4 may be a direction between the direction of the first directional axis DR1 and the direction of the second directional axis DR2.

The arrangement structure of the light emitting regions PXA-B, PXA-G, and PXA-R shown in FIG. 2 may be referred to as a PenTile™ structure. However, the arrangement structure of the light emitting regions PXA-B, PXA-G, and PXA-R in the display device DD according to an embodiment is not limited to the arrangement structure shown in FIG. 2. For example, the light emitting regions PXA-B, PXA-G, and PXA-R in an embodiment may have a stripe structure in which the blue light emitting region PXA-B, the green light emitting region PXA-G, and the red light emitting region PXA-R are arranged by turns along the first directional axis DR1.

FIG. 4 is schematic cross-sectional view showing a light emitting diode of an embodiment. FIG. 5 is a schematic cross-sectional view showing a portion of the light emitting diode according to an embodiment. FIG. 5 is a schematic cross-sectional view showing a portion corresponding to region AA in FIG. 4. As described above, each of multiple light emitting diodes OEL-1, OEL-2, and OEL-3 included in the display device DD shown in FIG. 3, etc., may have the structure of the light emitting diode OEL shown in FIG. 4 and FIG. 5.

The light emitting diode OEL of an embodiment includes a first electrode EL1, a hole transport region HTR disposed on the first electrode EL1, an emission layer EML disposed on the hole transport region HTR, an electron transport region ETR disposed on the emission layer EML, and a second electrode EL2 disposed on the electron transport region ETR. In the light emitting diode OEL of an embodiment, the hole transport region may include a first hole transport layer HTL1 disposed adjacent to the first electrode EL1, a second hole transport layer HTL 2 disposed adjacent to the emission layer EML, and a third hole transport layer HTL3 disposed between the first hole transport layer HTL1 and the second hole transport layer HTL2.

In an embodiment, the first hole transport layer HTL1 and the second hole transport layer HTL2 may be layers having lower refractive indexes than the third hole transport layer HTL3. The first refractive index of the first hole transport layer HTL1 may be less than the third refractive index of the third hole transport layer HTL3, and the second refractive index of the second hole transport layer HTL2 may be less than the third refractive index of the third hole transport layer HTL3.

In the light emitting diode OEL of an embodiment, the first electrode EL1 has conductivity. The first electrode EL1 may be formed using a metal alloy or a conductive compound. The first electrode EL1 may be an anode. The first electrode EL1 may be a pixel electrode. The first electrode EL1 may be a reflective electrode. If the first electrode EL1 is the reflective electrode, the first electrode EL1 may include Ag, Mg, Cu, Al, Pt, Pd, Au, Ni, Nd, Ir, Cr, Li, Ca, LiF/Ca, LiF/Al, Mo, Ti, compounds thereof, or mixtures thereof (for example, a mixture of Ag and Mg). In an embodiment, the first electrode EL1 may have a stacked structure of multiple layers. If the first electrode EL1 has the stacked structure of multiple layers, at least one layer may be a reflective layer formed using the reflective electrode material. If the first electrode EL1 has the stacked structure of multiple layers, at least one layer may include a transparent conductive layer formed by using indium tin oxide (ITO), indium zinc oxide (IZO), zinc oxide (ZnO), indium tin zinc oxide (ITZO), etc. For example, the first electrode EL1 may have a three-layer structure of ITO/Ag/ITO. However, embodiments are not limited thereto. A thickness of the first electrode EL1 may be in a range of about 1,000 Å to about 10,000 Å. For example, the thickness of the first electrode EL1 may be in a range of about 1,000 Å to about 3,000 Å.

The hole transport region HTR is provided on the first electrode EL1. The hole transport region HTR may include the first to third hole transport layers HTL1, HTL2, and HTL3. Based on the third hole transport layer HTL3 which has a relatively higher refractive index than the other hole transport layers HTL1 and HTL2, the first hole transport layer HTL1 may be disposed below the third hole transport layer HTL3, and the second hole transport layer HTL2 may be disposed above the third hole transport layer HTL3. In the light emitting diode OEL of an embodiment, the hole transport region HTR may include multiple hole transport layers HTL1, HTL2, and HTL3 disposed in the order of hole transport layer of a low refractive index/hole transport layer of a high refractive index/hole transport layer of a low refractive index in a thickness direction.

At a wavelength of about 460 nm, a difference between the third refractive index of the third hole transport layer HTL3 and the first refractive index of the first hole transport layer HTL1 may be greater than about 0.1. For example, at about 460 nm, the difference between the third refractive index and the first refractive index may be equal to or greater than about 0.2.

At a wavelength of about 460 nm, a difference between the third refractive index of the third hole transport layer HTL3 and the second refractive index of the second hole transport layer HTL2 may be greater than about 0.1. For example, at about 460 nm, the difference between the third refractive index and the second refractive index may be equal to or greater than about 0.2.

At a wavelength of about 460 nm, the first refractive index of the first hole transport layer HTL1 and the second refractive index of the second hole transport layer HTL2 may each be in a range of about 1.30 to about 1.80. At a wavelength of about 460 nm, the third refractive index of the third hole transport layer HTL3 may be in a range of about 1.85 to about 2.40. For example, the first refractive index and the second refractive index of the second hole transport layer HTL2 may each be in a range of about 1.40 to about 1.60, and the third refractive index of the third hole transport layer HTL3 may be in a range of about 1.90 to about 2.00.

A thickness of the hole transport region HTR may be in a range of about 300 Å to about 15,000 Å. For example, the thickness of the hole transport region HTR may be in a range of about 300 Å to about 5,000 Å. Thicknesses D1, D2, and D3 of the first to third hole transport layers HTL1, HTL2, and HTL3, respectively, that are included in the hole transport region HTR may each be in a range of about 100 Å to about 1,000 Å.

The thickness ratio (D1:D3:D2) of the first to third hole transport layers HTL1, HTL3, and HTL2 included in the hole transport region HTR may be in a range of about 0.1:0.8:0.1 to about 0.45:0.1:0.45. For example, in an embodiment, the thickness D1 of the first hole transport layer and the thickness D2 of the second hole transport layer may be substantially the same, and the thickness D3 of the third hole transport layer may be different from the thickness D1 of the first hole transport layer and the thickness D2 of the second hole transport layer. However, embodiments are not limited thereto, and the thickness D1 of the first hole transport layer and the thickness D2 of the second hole transport layer may be different from each other. The thickness ratio (D1:D3:D2) of the first to third hole transport layers HTL1, HTL3, and HTL2 may be controlled to an optimum range according to the wavelength region of light emitted from the emission layer EML, display quality required for the display device DD (FIG. 2), and the type of the hole transport materials used in each of the hole transport layers HTL1, HTL2, and HTL3 of the hole transport region HTR.

For example, in a case where blue light having a central wavelength in a wavelength region in a range of about 430 nm to about 470 nm is emitted from the emission layer EML in the light emitting diode OEL of an embodiment, the thickness ratio (D1:D3:D2) of the first to third hole transport layers HTL1, HTL3, and HTL2 may be about 1:1:1.

The light emitting diode OEL of an embodiment may include multiple hole transport layers HTL1, HTL2, and HTL3 disposed in the order of hole transport layer of a low refractive index/hole transport layer of a high refractive index/hole transport layer of a low refractive index, to show improved emission efficiency properties. The light emitting diode OEL of an embodiment includes the hole transport layers HTL1, HTL2, and HTL3 of the hole transport region HTR, having refractive index differences, and may minimize the extinction of light emitted from inner functional layers through destructive interference and induce constructive interference by the hole transport layers HTL1, HTL2, and HTL3 having refractive index differences, thereby showing high light emission efficiency.

In an embodiment, the first hole transport layer HTL1 may be disposed just above the first electrode EL1. For example, the first hole transport layer HTL1 may be disposed directly above the first electrode EL1. The second hole transport layer HTL2 may be disposed just below the emission layer EML. For example, the second hole transport layer HTL2 may be disposed directly below the emission layer EML.

In the description, “disposed just” may mean that no additional layer, film, region, plate, or the like is present between a layer, a film, a region, a plate, or the like and another. For example, one element may be disposed directly on another element. For example, “disposed just” means two layers are disposed without using an additional member such as an adhesive member between the two layers.

In the light emitting diode OEL of an embodiment, at a wavelength of about 460 nm, the refractive index of the first electrode EL1 may be in a range of about 1.80 to about 2.40. For example, the refractive index of the first electrode EL1 may be in a range of about 1.90 to about 2.00. For example, the refractive index of the first electrode EL1 may be greater than the first refractive index of the first hole transport layer HTL1, and a refractive index difference between the adjacent first hole transport layer HTL1 and first electrode EL1 at about 460 nm may be greater than about 0.1.

In the light emitting diode OEL of an embodiment, at a wavelength of about 460 nm, the refractive index of the emission layer EML may be in a range of about 1.80 to about 2.40. For example, the refractive index of the emission layer EML may be in a range of about 1.90 to about 2.00. For example, the refractive index of the emission layer EML may be greater than the second refractive index of the second hole transport layer, and a refractive index difference between the adjacent second hole transport layer HTL2 and emission layer EML at about 460 nm may be greater than about 0.1.

For example, the light emitting diode OEL of an embodiment includes the hole transport region HTR in which hole transport layers HTL1 and HTL2 having refractive index differences from adjacent first electrode EL1 and emission layer EML, and may show high light extraction efficiency properties and improved emission efficiency properties.

The first hole transport layer HTL1 and the second hole transport layer HTL2 may each independently include an amine compound represented by Formula 1 below. The amine compound represented by Formula 1 may have a refractive index value in a range of about 1.30 to about 1.80 at a wavelength of about 460 nm. The first hole transport layer HTL1 and the second hole transport layer HTL2 may be each independently formed using any one among the amine compounds represented by Formula 1 below or mixtures thereof.

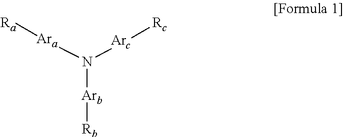

In Formula 1, Arₐ to Ar_{c} may be each independently a substituted or unsubstituted aryl group of 6 to 30 ring-forming carbon atoms, or a substituted or unsubstituted heteroaryl group of 3 to 30 ring-forming carbon atoms. At least two of Rₐ to R_{c} may each independently be an adamantyl group or a cyclohexyl group, and the remainder of Rₐ to R_{c} may be a hydrogen atom, a deuterium atom, a halogen atom, a cyano group, a substituted or unsubstituted oxy group, a substituted or unsubstituted thio group, a substituted or unsubstituted amine group, or a substituted or unsubstituted alkyl group of 1 to 20 carbon atoms.

In the amine compound represented by Formula 1, Arₐ to Ar_{c} may each independently be a substituted or unsubstituted phenyl group, a substituted or unsubstituted naphthyl group, a substituted or unsubstituted biphenyl group, a substituted or unsubstituted terphenyl group, a substituted or unsubstituted fluorenyl group, a substituted or unsubstituted carbazole group, a substituted or unsubstituted dibenzofuranyl group, or a substituted or unsubstituted dibenzothiophenyl group. However, embodiments are not limited thereto.

Two selected among Rₐ to R_{c}, or Rₐ to R_{c} may be each independently an unsubstituted adamantyl group, or an unsubstituted cyclohexyl group. For example, two selected among Rₐ to R_{c} may be adamantyl groups, or two of Rₐ to R_{c} may be cyclohexyl groups. In another embodiment, one of the two selected among Rₐ to R_{c} may be an adamantyl group, and the remainder may be a cyclohexyl group.

In an embodiment, all of Rₐ to R_{c} may be adamantyl groups or cyclohexyl groups. Two selected among Rₐ to R_{c} may be adamantyl groups, and the remainder of Rₐ to R_{c} may be a cyclohexyl group, or two selected among Rₐ to R_{c} may be cyclohexyl groups, and the remainder may be an adamantyl group.

In an embodiment, the first hole transport layer and the second hole transport layer may each independently include at least one among the amine compounds represented in Compound Group 1 below.

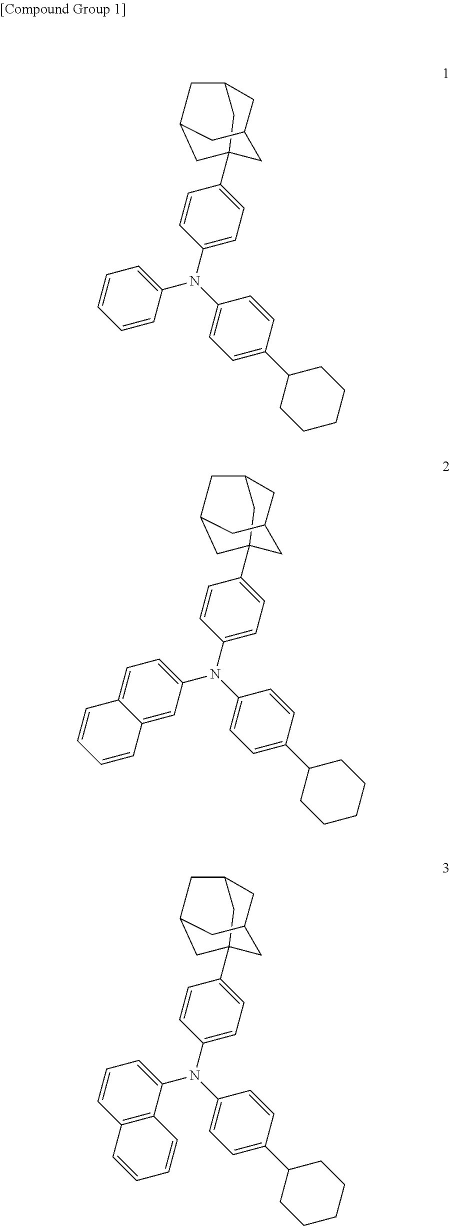 [SMILES: c1ccc(N(c2ccc(C3CCCCC3)cc2)c2ccc(C34CC5CC(CC(C5)C3)C4)cc2)cc1 ; c1ccc2cc(N(c3ccc(C4CCCCC4)cc3)c3ccc(C45CC6CC(CC(C6)C4)C5)cc3)ccc2c1 ; c1ccc2c(N(c3ccc(C4CCCCC4)cc3)c3ccc(C45CC6CC(CC(C6)C4)C5)cc3)cccc2c1]

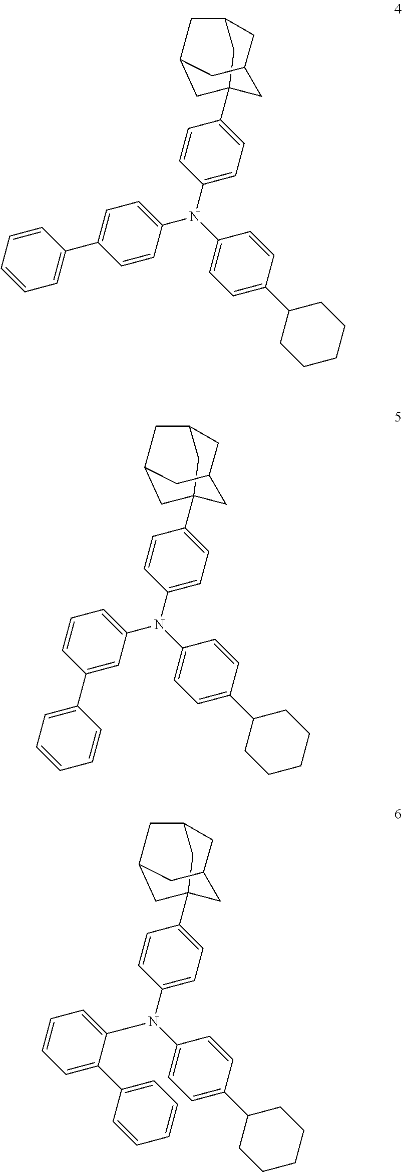 [SMILES: c1ccc(-c2ccc(N(c3ccc(C4CCCCC4)cc3)c3ccc(C45CC6CC(CC(C6)C4)C5)cc3)cc2)cc1 ; c1ccc(-c2cccc(N(c3ccc(C4CCCCC4)cc3)c3ccc(C45CC6CC(CC(C6)C4)C5)cc3)c2)cc1 ; c1ccc(-c2ccccc2N(c2ccc(C3CCCCC3)cc2)c2ccc(C34CC5CC(CC(C5)C3)C4)cc2)cc1]

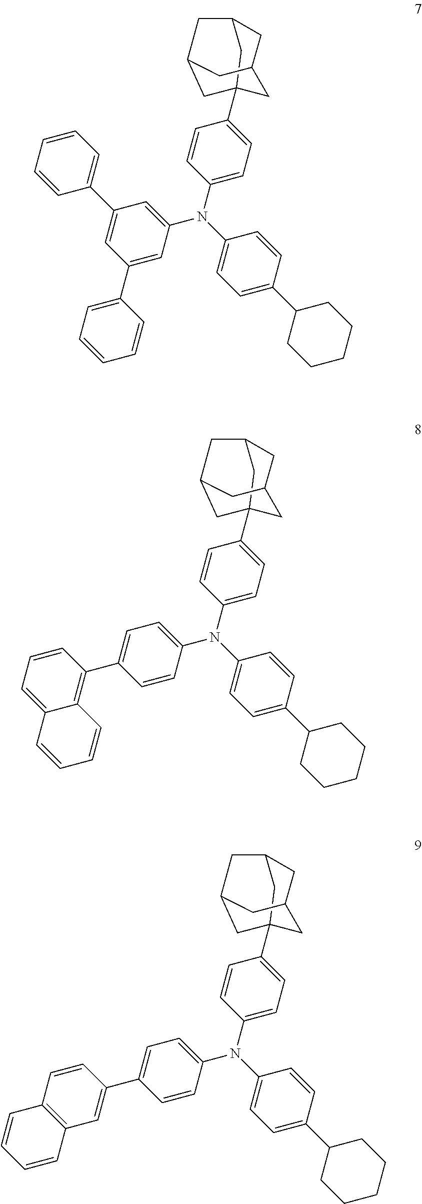 [SMILES: c1ccc(-c2cc(-c3ccccc3)cc(N(c3ccc(C4CCCCC4)cc3)c3ccc(C45CC6CC(CC(C6)C4)C5)cc3)c2)cc1 ; c1ccc2c(-c3ccc(N(c4ccc(C5CCCCC5)cc4)c4ccc(C56CC7CC(CC(C7)C5)C6)cc4)cc3)cccc2c1 ; c1ccc2cc(-c3ccc(N(c4ccc(C5CCCCC5)cc4)c4ccc(C56CC7CC(CC(C7)C5)C6)cc4)cc3)ccc2c1]

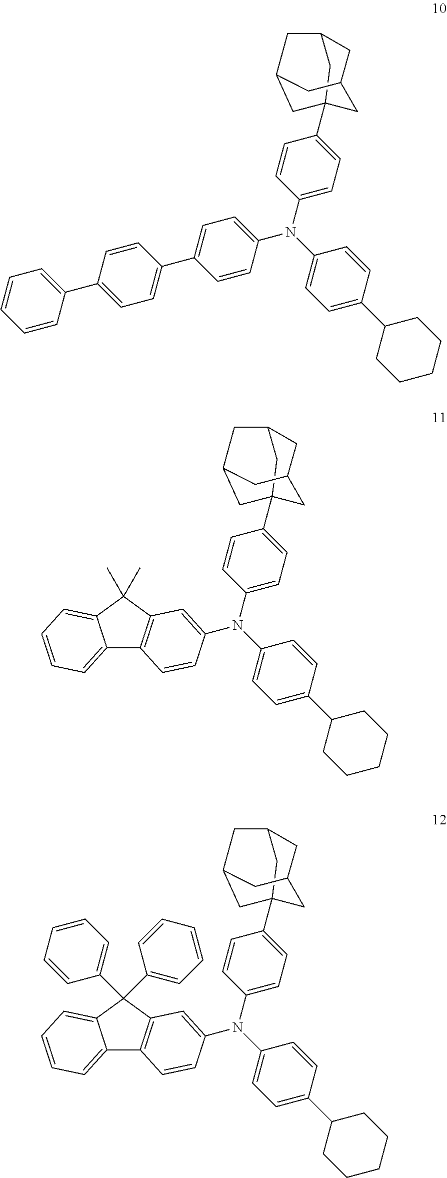 [SMILES: c1ccc(-c2ccc(-c3ccc(N(c4ccc(C5CCCCC5)cc4)c4ccc(C56CC7CC(CC(C7)C5)C6)cc4)cc3)cc2)cc1 ; CC1(C)c2ccccc2-c2ccc(N(c3ccc(C4CCCCC4)cc3)c3ccc(C45CC6CC(CC(C6)C4)C5)cc3)cc21 ; c1ccc(C2(c3ccccc3)c3ccccc3-c3ccc(N(c4ccc(C5CCCCC5)cc4)c4ccc(C56CC7CC(CC(C7)C5)C6)cc4)cc32)cc1]

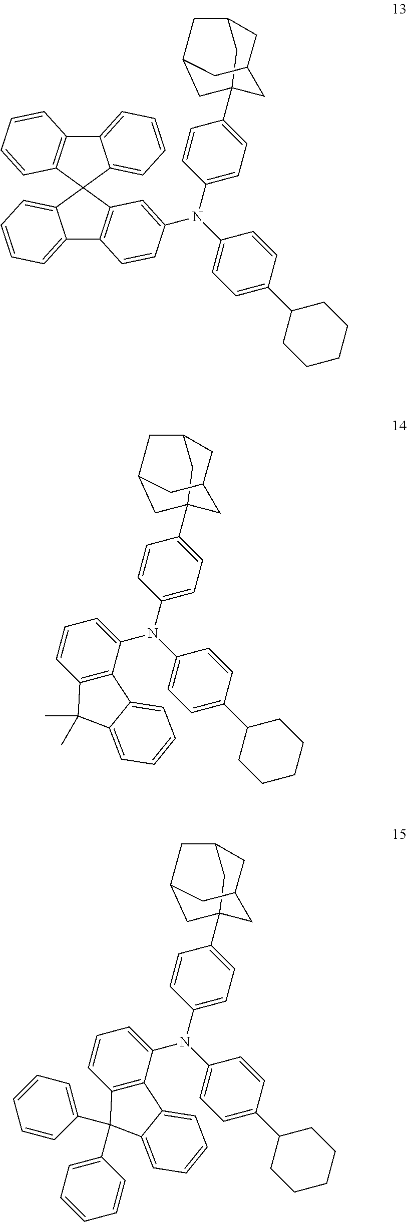 [SMILES: c1ccc2c(c1)-c1ccccc1C21c2ccccc2-c2ccc(N(c3ccc(C4CCCCC4)cc3)c3ccc(C45CC6CC(CC(C6)C4)C5)cc3)cc21 ; CC1(C)c2ccccc2-c2c(N(c3ccc(C4CCCCC4)cc3)c3ccc(C45CC6CC(CC(C6)C4)C5)cc3)cccc21 ; c1ccc(C2(c3ccccc3)c3ccccc3-c3c(N(c4ccc(C5CCCCC5)cc4)c4ccc(C56CC7CC(CC(C7)C5)C6)cc4)cccc32)cc1]

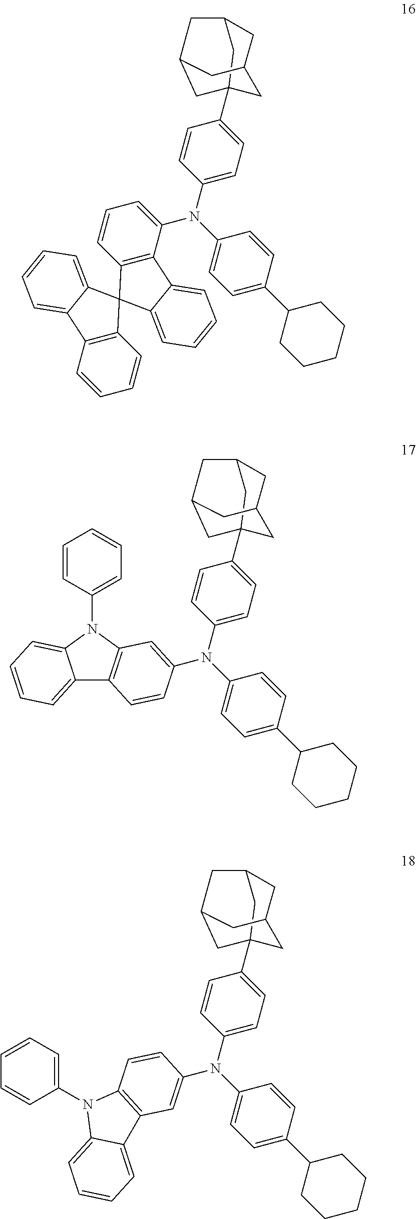 [SMILES: c1ccc2c(c1)-c1ccccc1C21c2ccccc2-c2c(N(c3ccc(C4CCCCC4)cc3)c3ccc(C45CC6CC(CC(C6)C4)C5)cc3)cccc21 ; c1ccc(-n2c3ccccc3c3ccc(N(c4ccc(C5CCCCC5)cc4)c4ccc(C56CC7CC(CC(C7)C5)C6)cc4)cc32)cc1 ; c1ccc(-n2c3ccccc3c3cc(N(c4ccc(C5CCCCC5)cc4)c4ccc(C56CC7CC(CC(C7)C5)C6)cc4)ccc32)cc1]

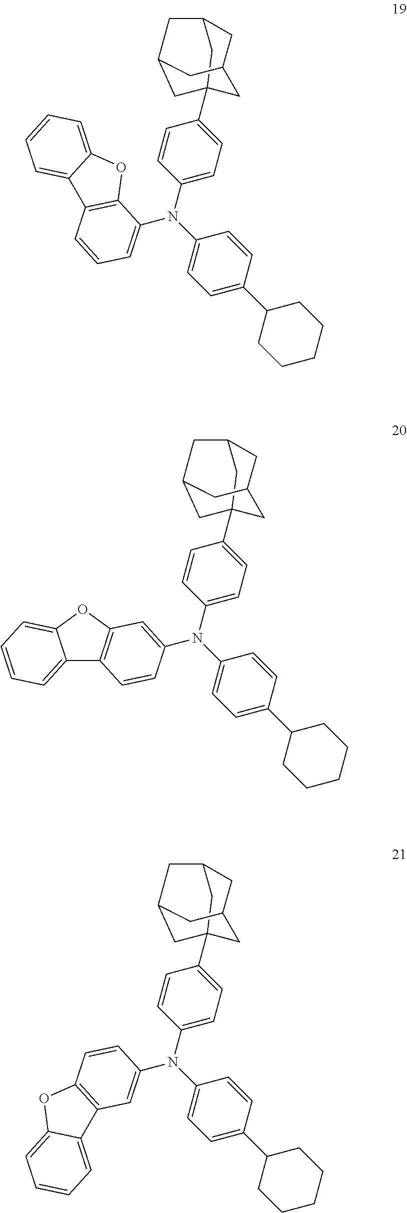 [SMILES: c1ccc2c(c1)oc1c(N(c3ccc(C4CCCCC4)cc3)c3ccc(C45CC6CC(CC(C6)C4)C5)cc3)cccc12 ; c1ccc2c(c1)oc1cc(N(c3ccc(C4CCCCC4)cc3)c3ccc(C45CC6CC(CC(C6)C4)C5)cc3)ccc12 ; c1ccc2c(c1)oc1ccc(N(c3ccc(C4CCCCC4)cc3)c3ccc(C45CC6CC(CC(C6)C4)C5)cc3)cc12]

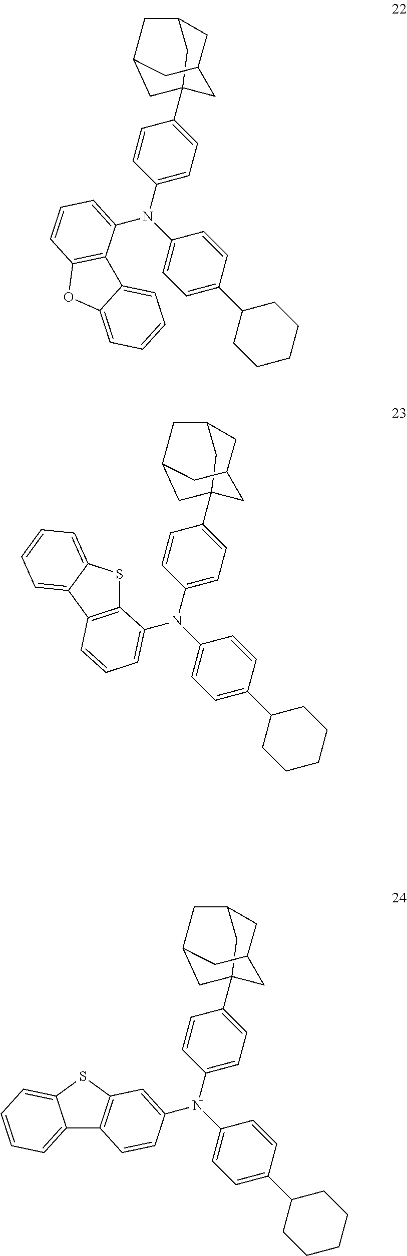 [SMILES: c1ccc2c(c1)oc1cccc(N(c3ccc(C4CCCCC4)cc3)c3ccc(C45CC6CC(CC(C6)C4)C5)cc3)c12 ; c1ccc2c(c1)sc1c(N(c3ccc(C4CCCCC4)cc3)c3ccc(C45CC6CC(CC(C6)C4)C5)cc3)cccc12 ; c1ccc2c(c1)sc1cc(N(c3ccc(C4CCCCC4)cc3)c3ccc(C45CC6CC(CC(C6)C4)C5)cc3)ccc12]

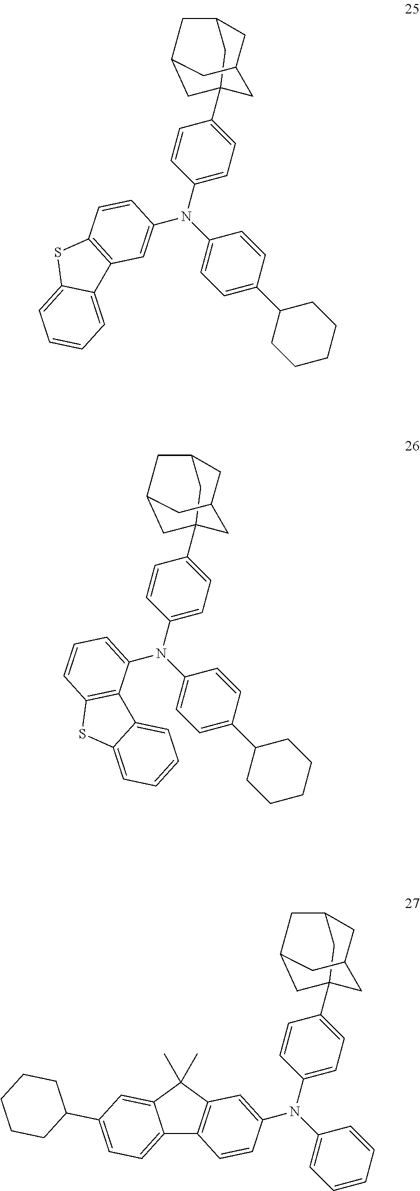 [SMILES: c1ccc2c(c1)sc1ccc(N(c3ccc(C4CCCCC4)cc3)c3ccc(C45CC6CC(CC(C6)C4)C5)cc3)cc12 ; c1ccc2c(c1)sc1cccc(N(c3ccc(C4CCCCC4)cc3)c3ccc(C45CC6CC(CC(C6)C4)C5)cc3)c12 ; CC1(C)c2cc(C3CCCCC3)ccc2-c2ccc(N(c3ccccc3)c3ccc(C45CC6CC(CC(C6)C4)C5)cc3)cc21]

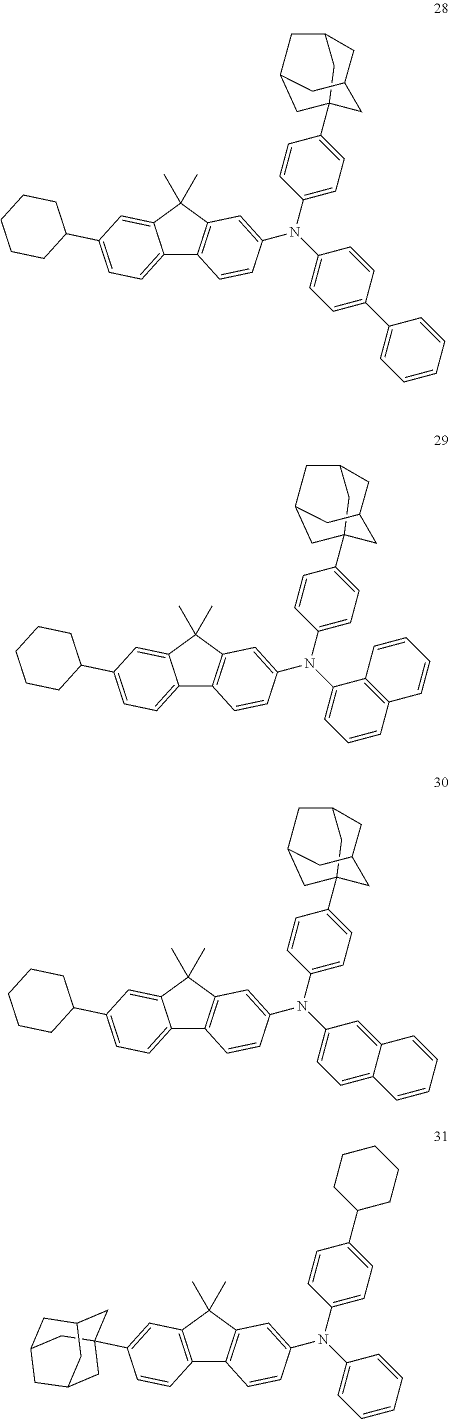 [SMILES: CC1(C)c2cc(C3CCCCC3)ccc2-c2ccc(N(c3ccc(-c4ccccc4)cc3)c3ccc(C45CC6CC(CC(C6)C4)C5)cc3)cc21 ; CC1(C)c2cc(C3CCCCC3)ccc2-c2ccc(N(c3ccc(C45CC6CC(CC(C6)C4)C5)cc3)c3cccc4ccccc34)cc21 ; CC1(C)c2cc(C3CCCCC3)ccc2-c2ccc(N(c3ccc(C45CC6CC(CC(C6)C4)C5)cc3)c3ccc4ccccc4c3)cc21 ; CC1(C)c2cc(N(c3ccccc3)c3ccc(C4CCCCC4)cc3)ccc2-c2ccc(C34CC5CC(CC(C5)C3)C4)cc21]

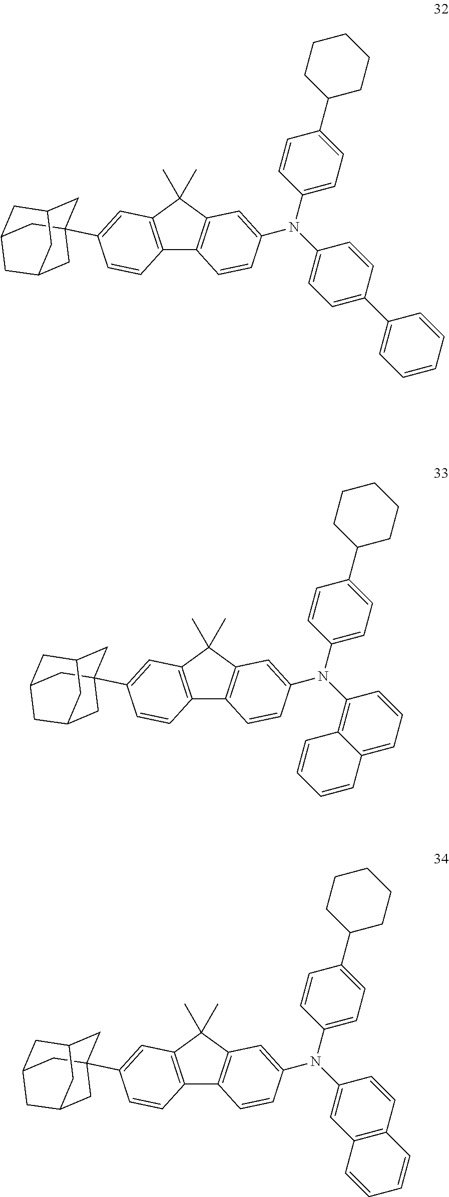 [SMILES: CC1(C)c2cc(N(c3ccc(-c4ccccc4)cc3)c3ccc(C4CCCCC4)cc3)ccc2-c2ccc(C34CC5CC(CC(C5)C3)C4)cc21 ; CC1(C)c2cc(N(c3ccc(C4CCCCC4)cc3)c3cccc4ccccc34)ccc2-c2ccc(C34CC5CC(CC(C5)C3)C4)cc21 ; CC1(C)c2cc(N(c3ccc(C4CCCCC4)cc3)c3ccc4ccccc4c3)ccc2-c2ccc(C34CC5CC(CC(C5)C3)C4)cc21]

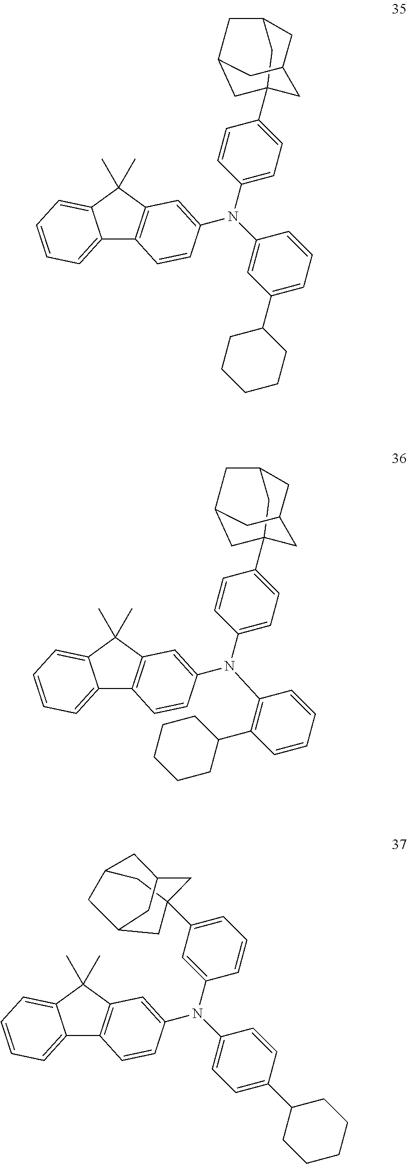 [SMILES: CC1(C)c2ccccc2-c2ccc(N(c3ccc(C45CC6CC(CC(C6)C4)C5)cc3)c3cccc(C4CCCCC4)c3)cc21 ; CC1(C)c2ccccc2-c2ccc(N(c3ccc(C45CC6CC(CC(C6)C4)C5)cc3)c3ccccc3C3CCCCC3)cc21 ; CC1(C)c2ccccc2-c2ccc(N(c3ccc(C4CCCCC4)cc3)c3cccc(C45CC6CC(CC(C6)C4)C5)c3)cc21]

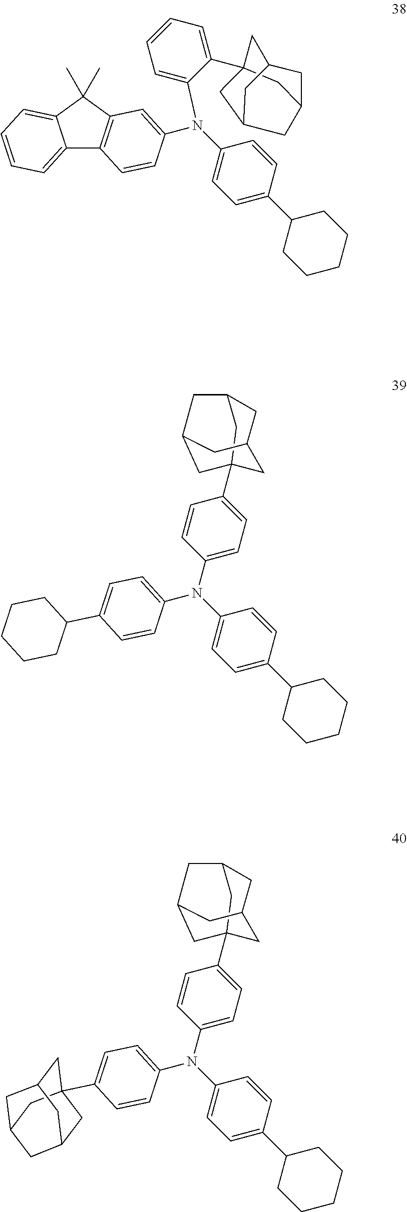 [SMILES: CC1(C)c2ccccc2-c2ccc(N(c3ccc(C4CCCCC4)cc3)c3ccccc3C34CC5CC(CC(C5)C3)C4)cc21 ; c1cc(N(c2ccc(C3CCCCC3)cc2)c2ccc(C34CC5CC(CC(C5)C3)C4)cc2)ccc1C1CCCCC1 ; c1cc(N(c2ccc(C34CC5CC(CC(C5)C3)C4)cc2)c2ccc(C34CC5CC(CC(C5)C3)C4)cc2)ccc1C1CCCCC1]

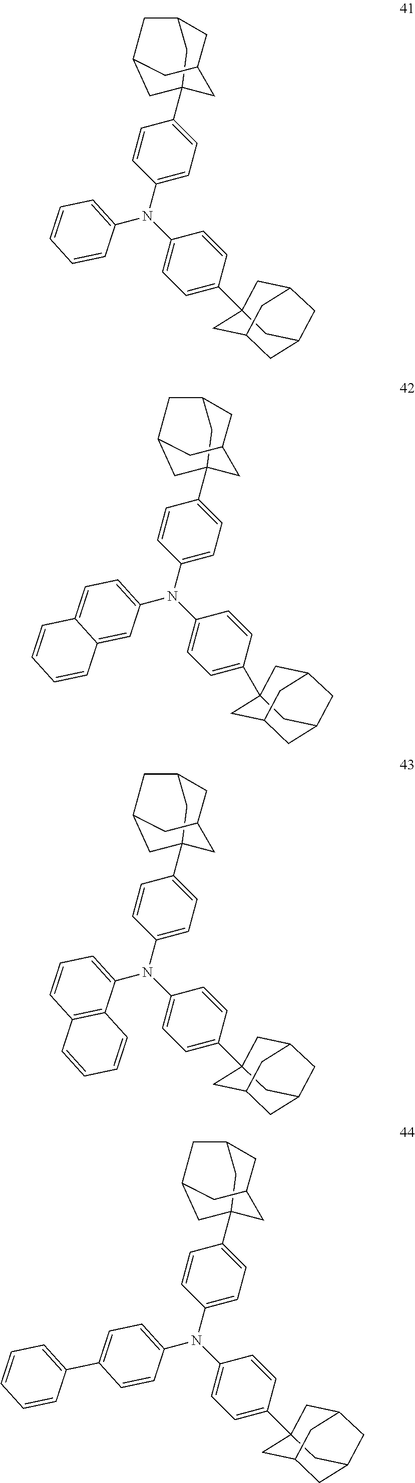 [SMILES: c1ccc(N(c2ccc(C34CC5CC(CC(C5)C3)C4)cc2)c2ccc(C34CC5CC(CC(C5)C3)C4)cc2)cc1 ; c1ccc2cc(N(c3ccc(C45CC6CC(CC(C6)C4)C5)cc3)c3ccc(C45CC6CC(CC(C6)C4)C5)cc3)ccc2c1 ; c1ccc2c(N(c3ccc(C45CC6CC(CC(C6)C4)C5)cc3)c3ccc(C45CC6CC(CC(C6)C4)C5)cc3)cccc2c1 ; c1ccc(-c2ccc(N(c3ccc(C45CC6CC(CC(C6)C4)C5)cc3)c3ccc(C45CC6CC(CC(C6)C4)C5)cc3)cc2)cc1]

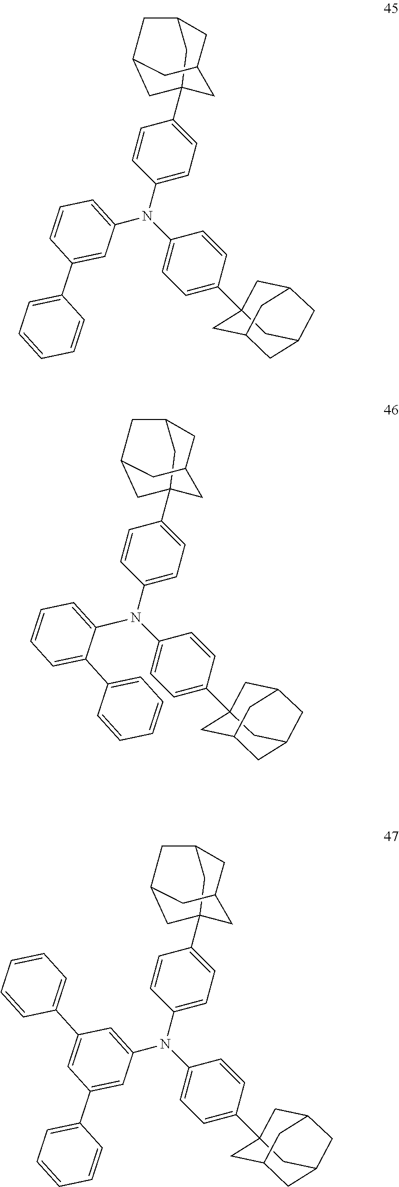 [SMILES: c1ccc(-c2cccc(N(c3ccc(C45CC6CC(CC(C6)C4)C5)cc3)c3ccc(C45CC6CC(CC(C6)C4)C5)cc3)c2)cc1 ; c1ccc(-c2ccccc2N(c2ccc(C34CC5CC(CC(C5)C3)C4)cc2)c2ccc(C34CC5CC(CC(C5)C3)C4)cc2)cc1 ; c1ccc(-c2cc(-c3ccccc3)cc(N(c3ccc(C45CC6CC(CC(C6)C4)C5)cc3)c3ccc(C45CC6CC(CC(C6)C4)C5)cc3)c2)cc1]

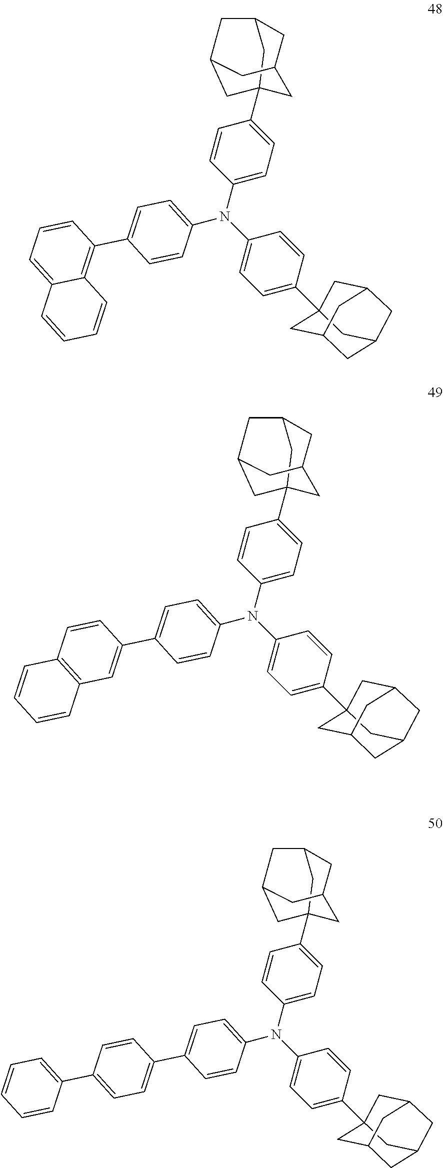 [SMILES: c1ccc2c(-c3ccc(N(c4ccc(C56CC7CC(CC(C7)C5)C6)cc4)c4ccc(C56CC7CC(CC(C7)C5)C6)cc4)cc3)cccc2c1 ; c1ccc2cc(-c3ccc(N(c4ccc(C56CC7CC(CC(C7)C5)C6)cc4)c4ccc(C56CC7CC(CC(C7)C5)C6)cc4)cc3)ccc2c1 ; c1ccc(-c2ccc(-c3ccc(N(c4ccc(C56CC7CC(CC(C7)C5)C6)cc4)c4ccc(C56CC7CC(CC(C7)C5)C6)cc4)cc3)cc2)cc1]

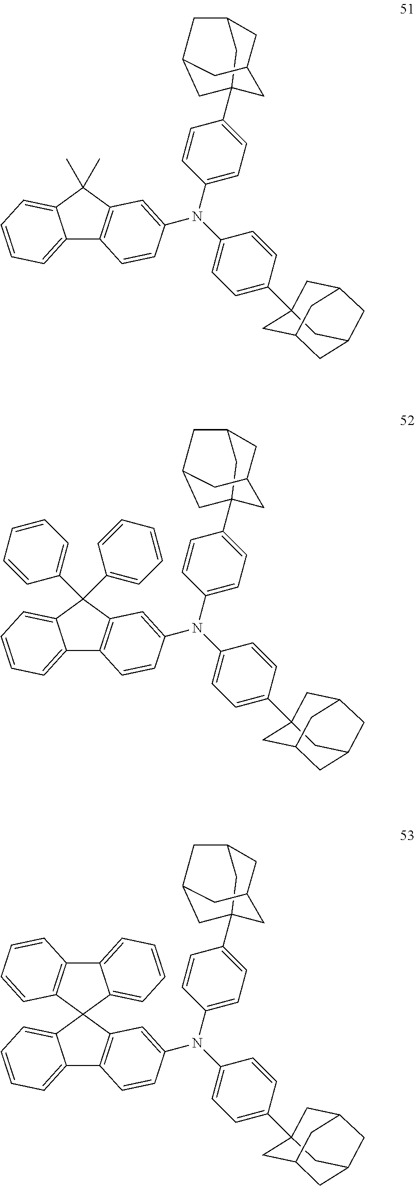 [SMILES: CC1(C)c2ccccc2-c2ccc(N(c3ccc(C45CC6CC(CC(C6)C4)C5)cc3)c3ccc(C45CC6CC(CC(C6)C4)C5)cc3)cc21 ; c1ccc(C2(c3ccccc3)c3ccccc3-c3ccc(N(c4ccc(C56CC7CC(CC(C7)C5)C6)cc4)c4ccc(C56CC7CC(CC(C7)C5)C6)cc4)cc32)cc1 ; c1ccc2c(c1)-c1ccccc1C21c2ccccc2-c2ccc(N(c3ccc(C45CC6CC(CC(C6)C4)C5)cc3)c3ccc(C45CC6CC(CC(C6)C4)C5)cc3)cc21]

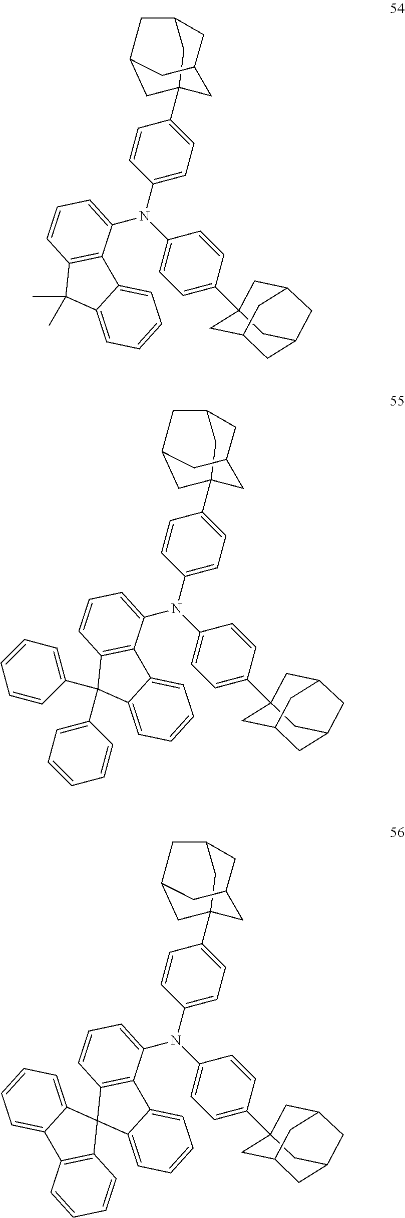 [SMILES: CC1(C)c2ccccc2-c2c(N(c3ccc(C45CC6CC(CC(C6)C4)C5)cc3)c3ccc(C45CC6CC(CC(C6)C4)C5)cc3)cccc21 ; c1ccc(C2(c3ccccc3)c3ccccc3-c3c(N(c4ccc(C56CC7CC(CC(C7)C5)C6)cc4)c4ccc(C56CC7CC(CC(C7)C5)C6)cc4)cccc32)cc1 ; c1ccc2c(c1)-c1ccccc1C21c2ccccc2-c2c(N(c3ccc(C45CC6CC(CC(C6)C4)C5)cc3)c3ccc(C45CC6CC(CC(C6)C4)C5)cc3)cccc21]

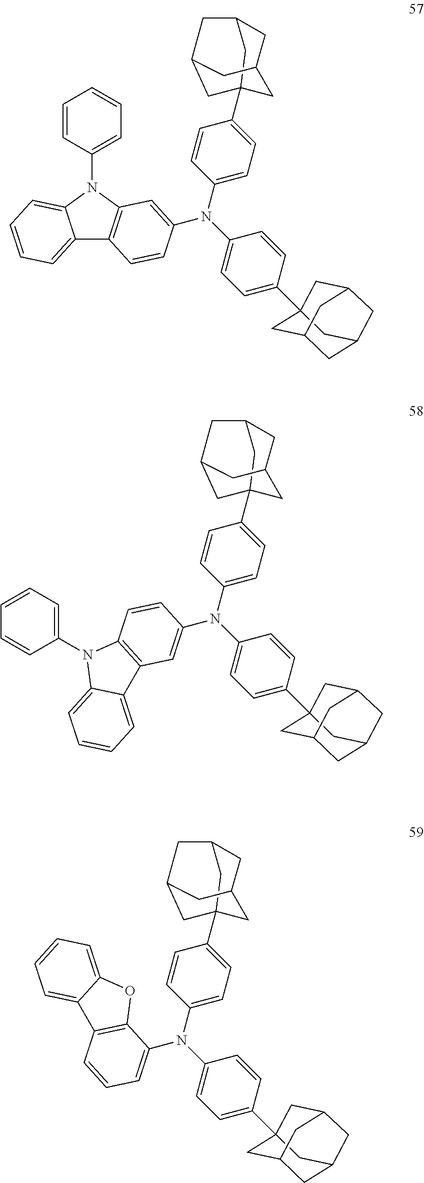 [SMILES: c1ccc(-n2c3ccccc3c3ccc(N(c4ccc(C56CC7CC(CC(C7)C5)C6)cc4)c4ccc(C56CC7CC(CC(C7)C5)C6)cc4)cc32)cc1 ; c1ccc(-n2c3ccccc3c3cc(N(c4ccc(C56CC7CC(CC(C7)C5)C6)cc4)c4ccc(C56CC7CC(CC(C7)C5)C6)cc4)ccc32)cc1 ; c1ccc2c(c1)oc1c(N(c3ccc(C45CC6CC(CC(C6)C4)C5)cc3)c3ccc(C45CC6CC(CC(C6)C4)C5)cc3)cccc12]

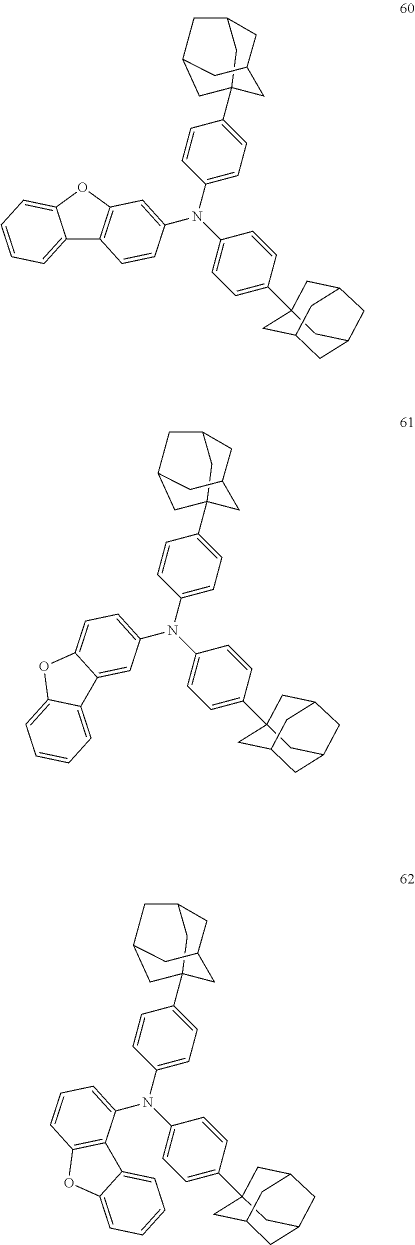 [SMILES: c1ccc2c(c1)oc1cc(N(c3ccc(C45CC6CC(CC(C6)C4)C5)cc3)c3ccc(C45CC6CC(CC(C6)C4)C5)cc3)ccc12 ; c1ccc2c(c1)oc1ccc(N(c3ccc(C45CC6CC(CC(C6)C4)C5)cc3)c3ccc(C45CC6CC(CC(C6)C4)C5)cc3)cc12 ; c1ccc2c(c1)oc1cccc(N(c3ccc(C45CC6CC(CC(C6)C4)C5)cc3)c3ccc(C45CC6CC(CC(C6)C4)C5)cc3)c12]

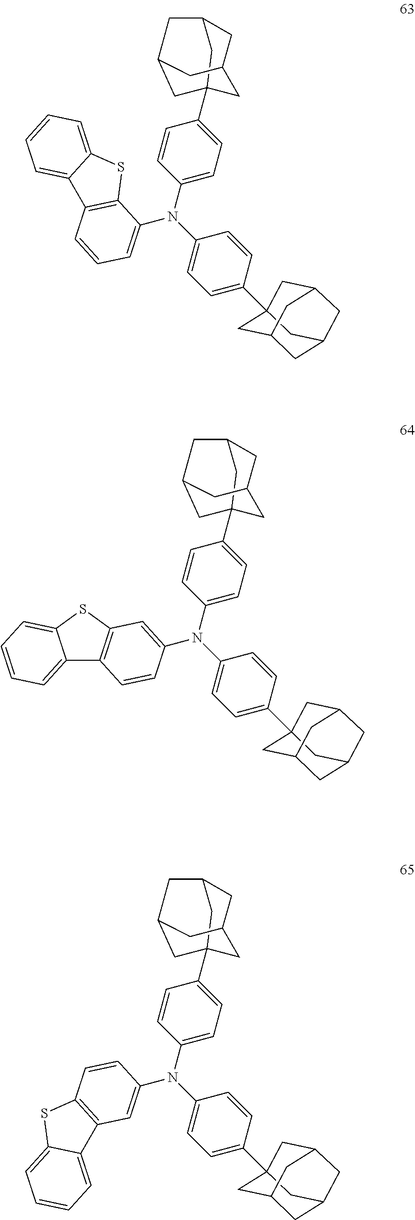 [SMILES: c1ccc2c(c1)sc1c(N(c3ccc(C45CC6CC(CC(C6)C4)C5)cc3)c3ccc(C45CC6CC(CC(C6)C4)C5)cc3)cccc12 ; c1ccc2c(c1)sc1cc(N(c3ccc(C45CC6CC(CC(C6)C4)C5)cc3)c3ccc(C45CC6CC(CC(C6)C4)C5)cc3)ccc12 ; c1ccc2c(c1)sc1ccc(N(c3ccc(C45CC6CC(CC(C6)C4)C5)cc3)c3ccc(C45CC6CC(CC(C6)C4)C5)cc3)cc12]

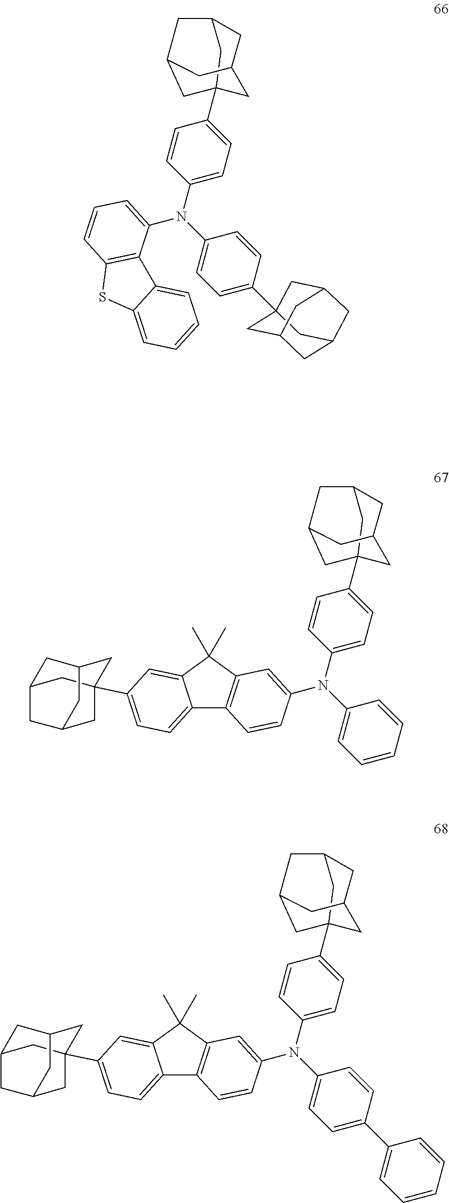 [SMILES: c1ccc2c(c1)sc1cccc(N(c3ccc(C45CC6CC(CC(C6)C4)C5)cc3)c3ccc(C45CC6CC(CC(C6)C4)C5)cc3)c12 ; CC1(C)c2cc(N(c3ccccc3)c3ccc(C45CC6CC(CC(C6)C4)C5)cc3)ccc2-c2ccc(C34CC5CC(CC(C5)C3)C4)cc21 ; CC1(C)c2cc(N(c3ccc(-c4ccccc4)cc3)c3ccc(C45CC6CC(CC(C6)C4)C5)cc3)ccc2-c2ccc(C34CC5CC(CC(C5)C3)C4)cc21]

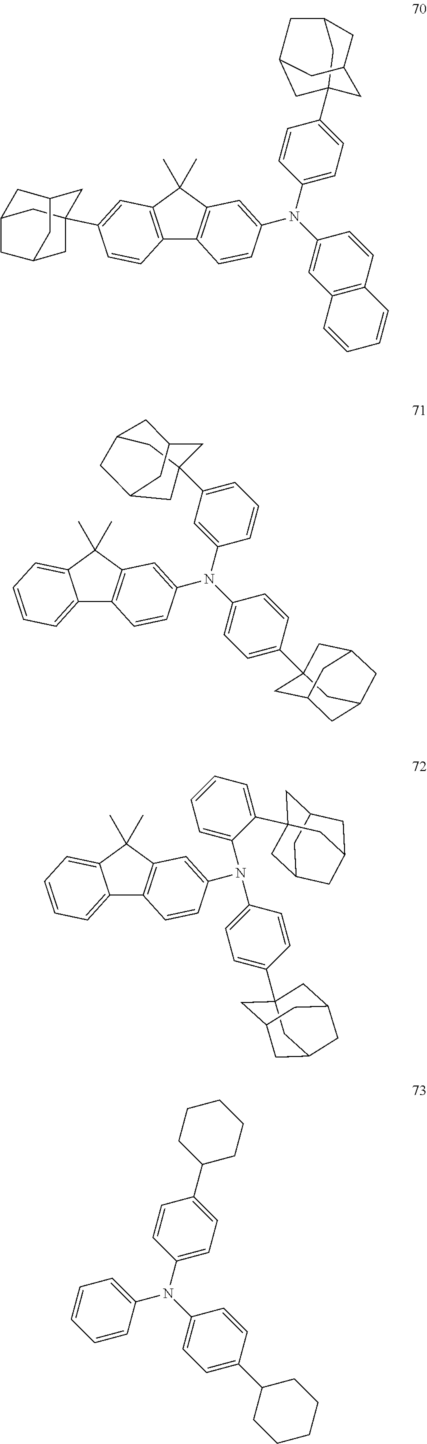 [SMILES: CC1(C)c2cc(N(c3ccc(C45CC6CC(CC(C6)C4)C5)cc3)c3ccc4ccccc4c3)ccc2-c2ccc(C34CC5CC(CC(C5)C3)C4)cc21 ; CC1(C)c2ccccc2-c2ccc(N(c3ccc(C45CC6CC(CC(C6)C4)C5)cc3)c3cccc(C45CC6CC(CC(C6)C4)C5)c3)cc21 ; CC1(C)c2ccccc2-c2ccc(N(c3ccc(C45CC6CC(CC(C6)C4)C5)cc3)c3ccccc3C34CC5CC(CC(C5)C3)C4)cc21 ; c1ccc(N(c2ccc(C3CCCCC3)cc2)c2ccc(C3CCCCC3)cc2)cc1]

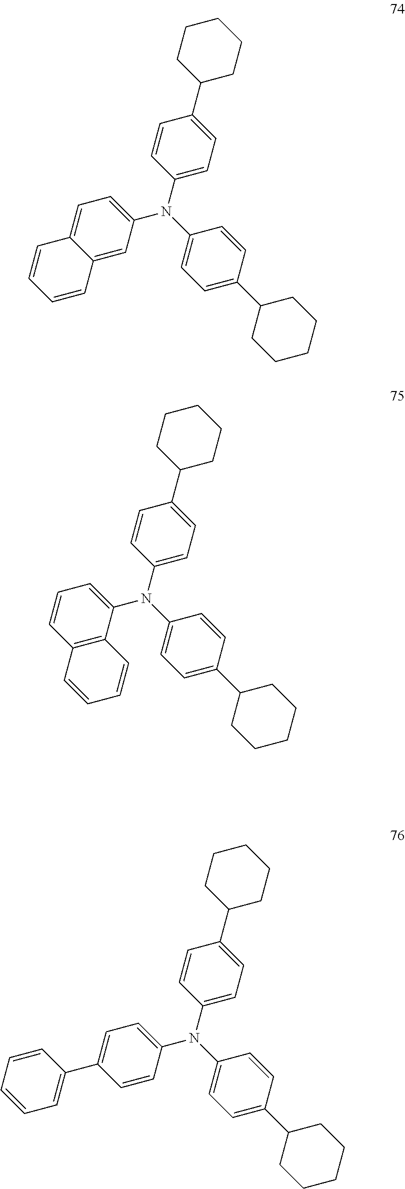 [SMILES: c1ccc2cc(N(c3ccc(C4CCCCC4)cc3)c3ccc(C4CCCCC4)cc3)ccc2c1 ; c1ccc2c(N(c3ccc(C4CCCCC4)cc3)c3ccc(C4CCCCC4)cc3)cccc2c1 ; c1ccc(-c2ccc(N(c3ccc(C4CCCCC4)cc3)c3ccc(C4CCCCC4)cc3)cc2)cc1]

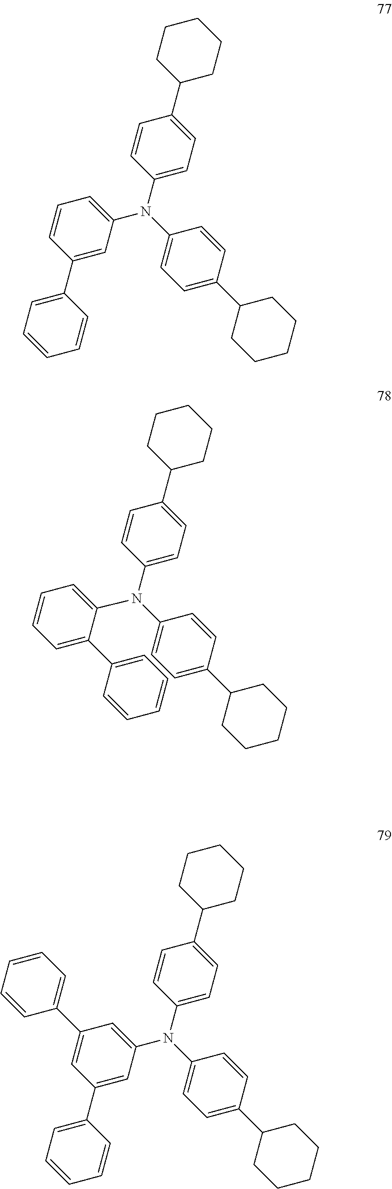 [SMILES: c1ccc(-c2cccc(N(c3ccc(C4CCCCC4)cc3)c3ccc(C4CCCCC4)cc3)c2)cc1 ; c1ccc(-c2ccccc2N(c2ccc(C3CCCCC3)cc2)c2ccc(C3CCCCC3)cc2)cc1 ; c1ccc(-c2cc(-c3ccccc3)cc(N(c3ccc(C4CCCCC4)cc3)c3ccc(C4CCCCC4)cc3)c2)cc1]

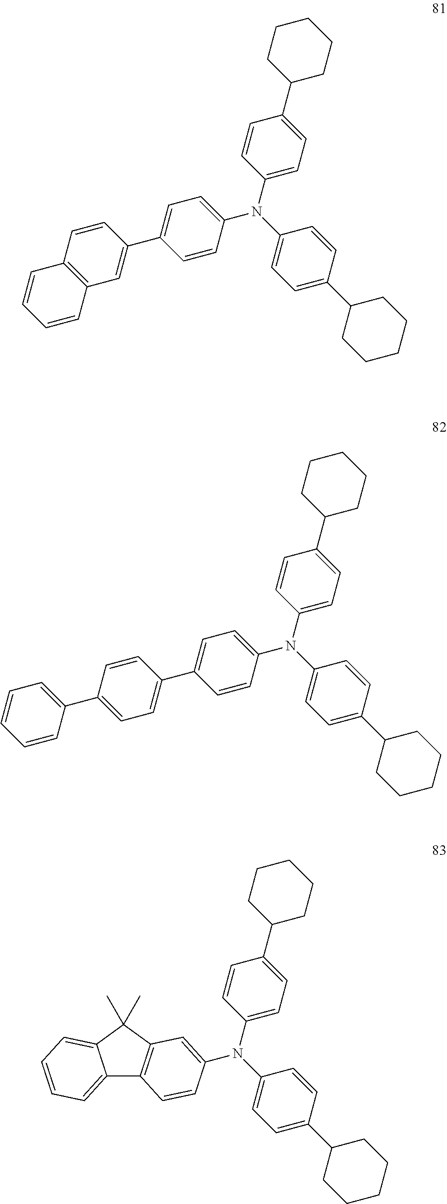 [SMILES: c1ccc2cc(-c3ccc(N(c4ccc(C5CCCCC5)cc4)c4ccc(C5CCCCC5)cc4)cc3)ccc2c1 ; c1ccc(-c2ccc(-c3ccc(N(c4ccc(C5CCCCC5)cc4)c4ccc(C5CCCCC5)cc4)cc3)cc2)cc1 ; CC1(C)c2ccccc2-c2ccc(N(c3ccc(C4CCCCC4)cc3)c3ccc(C4CCCCC4)cc3)cc21]

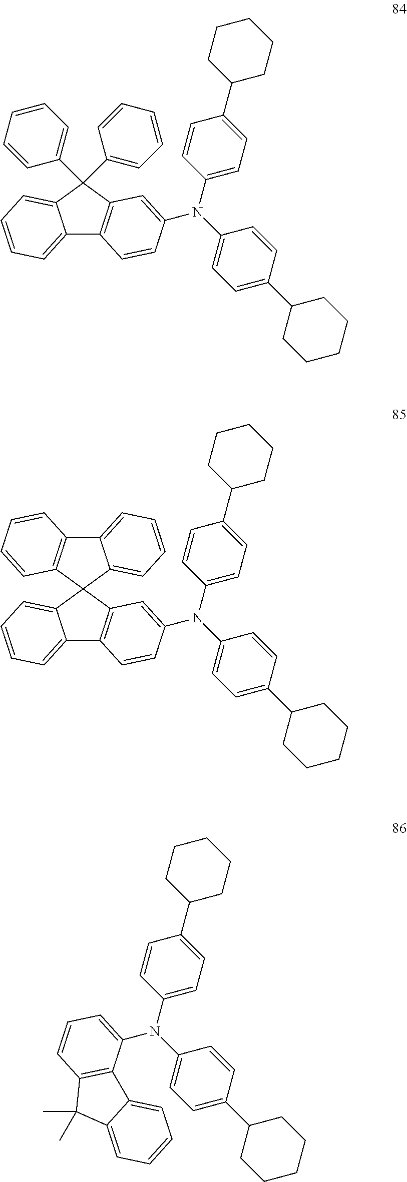 [SMILES: c1ccc(C2(c3ccccc3)c3ccccc3-c3ccc(N(c4ccc(C5CCCCC5)cc4)c4ccc(C5CCCCC5)cc4)cc32)cc1 ; c1ccc2c(c1)-c1ccccc1C21c2ccccc2-c2ccc(N(c3ccc(C4CCCCC4)cc3)c3ccc(C4CCCCC4)cc3)cc21 ; CC1(C)c2ccccc2-c2c(N(c3ccc(C4CCCCC4)cc3)c3ccc(C4CCCCC4)cc3)cccc21]

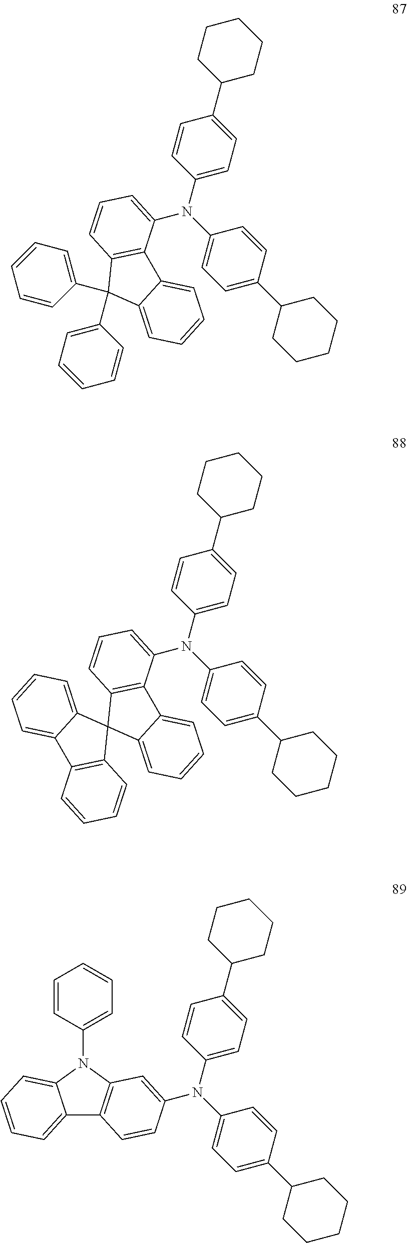 [SMILES: c1ccc(C2(c3ccccc3)c3ccccc3-c3c(N(c4ccc(C5CCCCC5)cc4)c4ccc(C5CCCCC5)cc4)cccc32)cc1 ; c1ccc2c(c1)-c1ccccc1C21c2ccccc2-c2c(N(c3ccc(C4CCCCC4)cc3)c3ccc(C4CCCCC4)cc3)cccc21 ; c1ccc(-n2c3ccccc3c3ccc(N(c4ccc(C5CCCCC5)cc4)c4ccc(C5CCCCC5)cc4)cc32)cc1]

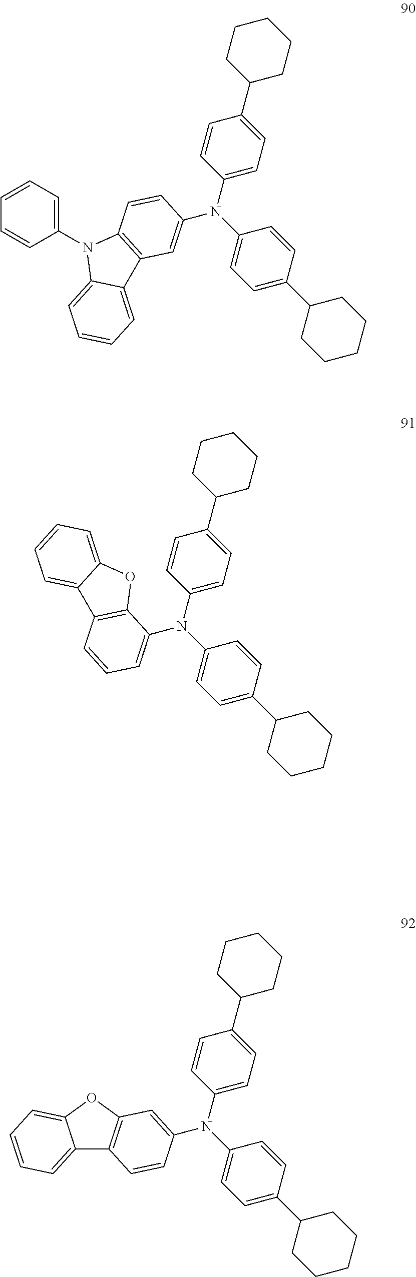 [SMILES: c1ccc(-n2c3ccccc3c3cc(N(c4ccc(C5CCCCC5)cc4)c4ccc(C5CCCCC5)cc4)ccc32)cc1 ; c1ccc2c(c1)oc1c(N(c3ccc(C4CCCCC4)cc3)c3ccc(C4CCCCC4)cc3)cccc12 ; c1ccc2c(c1)oc1cc(N(c3ccc(C4CCCCC4)cc3)c3ccc(C4CCCCC4)cc3)ccc12]

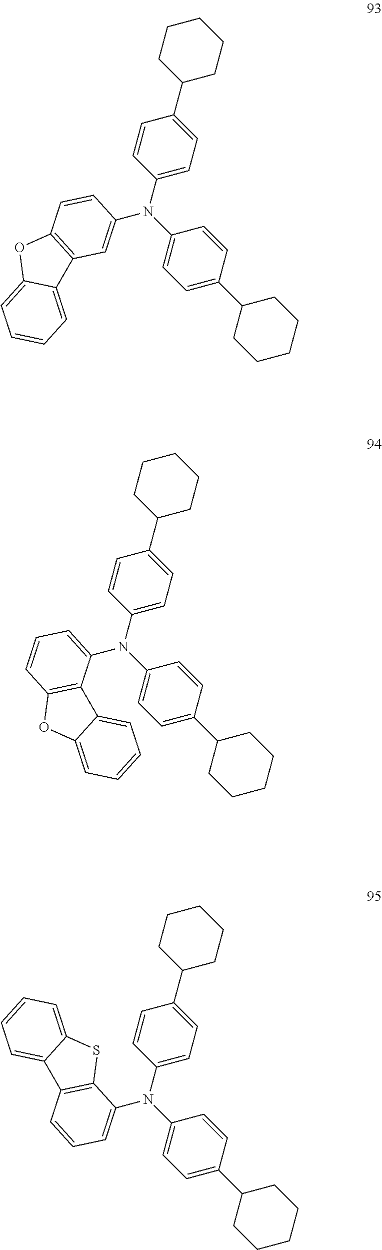 [SMILES: c1ccc2c(c1)oc1ccc(N(c3ccc(C4CCCCC4)cc3)c3ccc(C4CCCCC4)cc3)cc12 ; c1ccc2c(c1)oc1cccc(N(c3ccc(C4CCCCC4)cc3)c3ccc(C4CCCCC4)cc3)c12 ; c1ccc2c(c1)sc1c(N(c3ccc(C4CCCCC4)cc3)c3ccc(C4CCCCC4)cc3)cccc12]

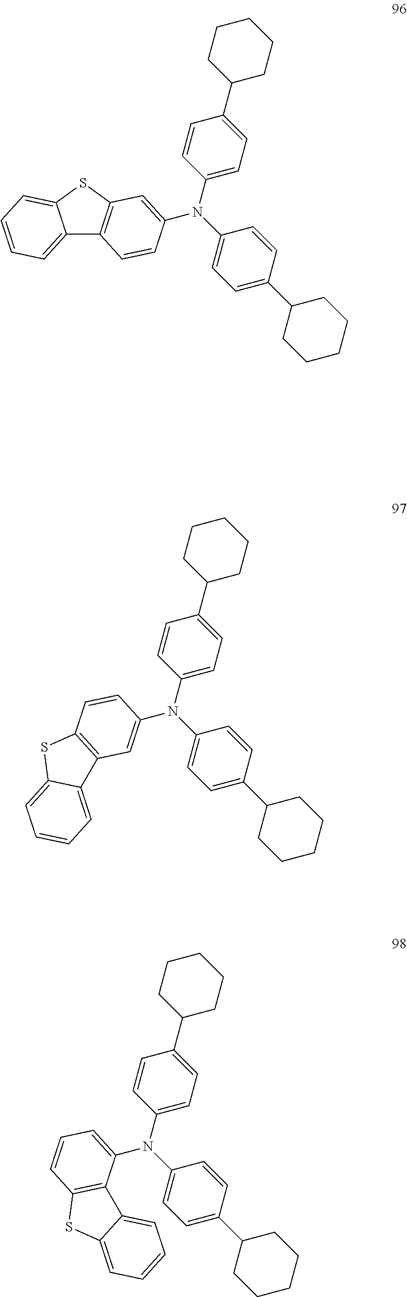 [SMILES: c1ccc2c(c1)sc1cc(N(c3ccc(C4CCCCC4)cc3)c3ccc(C4CCCCC4)cc3)ccc12 ; c1ccc2c(c1)sc1ccc(N(c3ccc(C4CCCCC4)cc3)c3ccc(C4CCCCC4)cc3)cc12 ; c1ccc2c(c1)sc1cccc(N(c3ccc(C4CCCCC4)cc3)c3ccc(C4CCCCC4)cc3)c12]

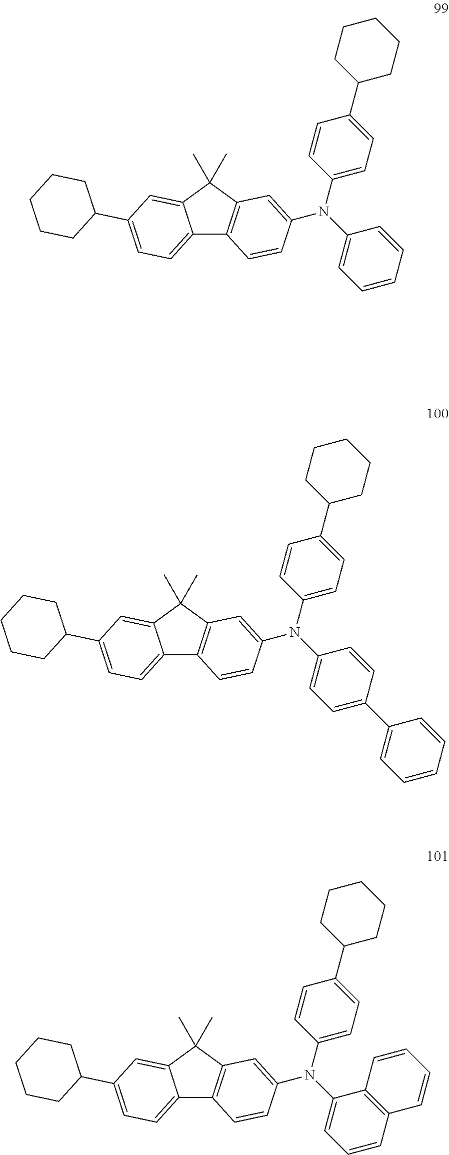 [SMILES: CC1(C)c2cc(C3CCCCC3)ccc2-c2ccc(N(c3ccccc3)c3ccc(C4CCCCC4)cc3)cc21 ; CC1(C)c2cc(C3CCCCC3)ccc2-c2ccc(N(c3ccc(-c4ccccc4)cc3)c3ccc(C4CCCCC4)cc3)cc21 ; CC1(C)c2cc(C3CCCCC3)ccc2-c2ccc(N(c3ccc(C4CCCCC4)cc3)c3cccc4ccccc34)cc21]

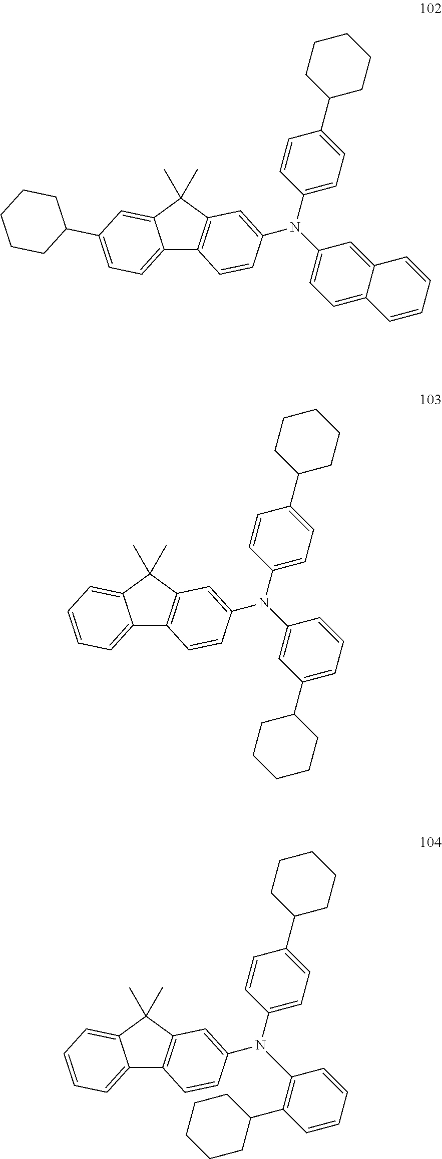 [SMILES: CC1(C)c2cc(C3CCCCC3)ccc2-c2ccc(N(c3ccc(C4CCCCC4)cc3)c3ccc4ccccc4c3)cc21 ; CC1(C)c2ccccc2-c2ccc(N(c3ccc(C4CCCCC4)cc3)c3cccc(C4CCCCC4)c3)cc21 ; CC1(C)c2ccccc2-c2ccc(N(c3ccc(C4CCCCC4)cc3)c3ccccc3C3CCCCC3)cc21]

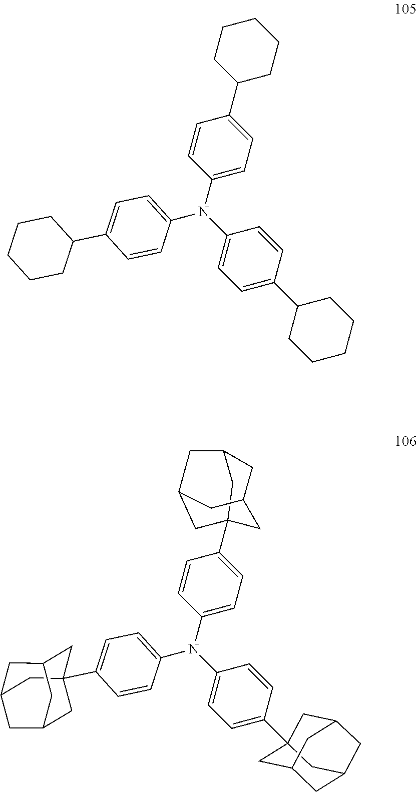 [SMILES: c1cc(N(c2ccc(C3CCCCC3)cc2)c2ccc(C3CCCCC3)cc2)ccc1C1CCCCC1 ; c1cc(C23CC4CC(CC(C4)C2)C3)ccc1N(c1ccc(C23CC4CC(CC(C4)C2)C3)cc1)c1ccc(C23CC4CC(CC(C4)C2)C3)cc1]

In the description, the term “substituted or unsubstituted” corresponds to substituted or unsubstituted with one or more substituents selected from the group consisting of a deuterium atom, a halogen atom, a cyano group, a nitro group, an amino group, a silyl group, an oxy group, a thio group, a sulfinyl group, a sulfonyl group, a carbonyl group, a boron group, a phosphine oxide group, a phosphine sulfide group, an alkyl group, an alkenyl group, an alkynyl group, an alkoxy group, a hydrocarbon ring group, an aryl group, and a heterocyclic group. Each of the listed substituents may be substituted or unsubstituted. For example, a biphenyl group may be interpreted as an aryl group, or a phenyl group substituted with a phenyl group.

In the description, the term “combined with an adjacent group to form a ring” may mean forming a substituted or unsubstituted hydrocarbon ring, or a substituted or unsubstituted heterocycle via the combination with an adjacent group. The hydrocarbon ring includes an aliphatic hydrocarbon ring and an aromatic hydrocarbon ring. The heterocycle includes an aliphatic heterocycle and an aromatic heterocycle. The hydrocarbon ring and the heterocycle may be monocyclic rings or polycyclic rings. The ring formed via the combination with an adjacent group may be combined with another ring to form a spiro structure.

In the description, the term “adjacent group” may mean a substituent substituted for an atom which is directly combined with an atom substituted with a corresponding substituent, another substituent substituted for an atom which is substituted with a corresponding substituent, or a substituent sterically positioned at the nearest position to a corresponding substituent. For example, in 1,2-dimethylbenzene, two methyl groups may be interpreted as “adjacent groups” to each other, and in 1,1-diethylcyclopentane, two ethyl groups may be interpreted as “adjacent groups” to each other.

In the description, the halogen atom may be a fluorine atom, a chlorine atom, a bromine atom, or an iodine atom.

In the description, the alkyl may be a linear, branched, or cyclic type. The carbon number of the alkyl may be 1 to 50, 1 to 30, 1 to 20, 1 to 10, or 1 to 6. Examples of the alkyl may include methyl, ethyl, n-propyl, isopropyl, n-butyl, s-butyl, t-butyl, i-butyl, 2-ethylbutyl, 3,3-dimethylbutyl, n-pentyl, i-pentyl, neopentyl, t-pentyl, cyclopentyl, 1-methylpentyl, 3-methylpentyl, 2-ethylpentyl, 4-methyl-2-pentyl, n-hexyl, 1-methylhexyl, 2-ethylhexyl, 2-butylhexyl, cyclohexyl, 4-methylcyclohexyl, 4-t-butylcyclohexyl, n-heptyl, 1-methylheptyl, 2,2-dimethylheptyl, 2-ethylheptyl, 2-butylheptyl, n-octyl, t-octyl, 2-ethyloctyl, 2-butyloctyl, 2-hexyloctyl, 3,7-dimethyloctyl, cyclooctyl, n-nonyl, n-decyl, adamantyl, 2-ethyldecyl, 2-butyldecyl, 2-hexyldecyl, 2-octyldecyl, n-undecyl, n-dodecyl, 2-ethyldodecyl, 2-butyldodecyl, 2-hexyldodecyl, 2-octyldodecyl, n-tridecyl, n-tetradecyl, n-pentadecyl, n-hexadecyl, 2-ethylhexadecyl, 2-butylhexadecyl, 2-hexylhexadecyl, 2-octylhexadecyl, n-heptadecyl, n-octadecyl, n-nonadecyl, n-eicosyl, 2-ethyleicosyl, 2-butyleicosyl, 2-hexyleicosyl, 2-octyleicosyl, n-heneicosyl, n-docosyl, n-tricosyl, n-tetracosyl, n-pentacosyl, n-hexacosyl, n-heptacosyl, n-octacosyl, n-nonacosyl, n-triacontyl, etc., without limitation.

In the description, the aryl group means an optional functional group or substituent derived from an aromatic hydrocarbon ring. The aryl group may be a monocyclic aryl group or a polycyclic aryl group. The carbon number for forming rings in the aryl group may be 6 to 30, 6 to 20, or 6 to 15. Examples of the aryl group may include phenyl, naphthyl, fluorenyl, anthracenyl, phenanthryl, biphenyl, terphenyl, quaterphenyl, quinquephenyl, sexiphenyl, triphenylenyl, pyrenyl, benzofluoranthenyl, chrysenyl, etc., without limitation.

In the description, the fluorenyl group may be substituted, and two substituents may be combined with each other to form a spiro structure. Examples of a substituted fluorenyl group are as follows. However, embodiments are not limited thereto.

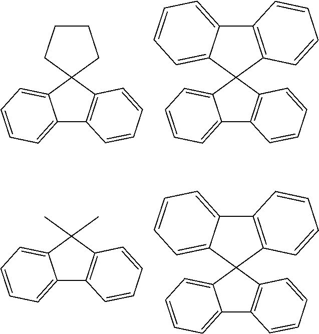

In the description, the heteroaryl group may include one or more among B, O, N, P, Si, and S as heteroatoms. If the heteroaryl group includes two or more heteroatoms, two or more heteroatoms may be the same or different. The heteroaryl group may be a monocyclic heterocyclic group or polycyclic heterocyclic group. The carbon number for forming rings of the heteroaryl group may be 2 to 30, 2 to 20, or 2 to 10. Examples of the heteroaryl group may include thiophene, furan, pyrrole, imidazole, triazole, pyridine, bipyridine, pyrimidine, triazine, acridyl, pyridazine, pyrazinyl, quinoline, quinazoline, quinoxaline, phenoxazine, phthalazine, pyrido pyrimidine, pyrido pyrazine, pyrazino pyrazine, isoquinoline, indole, carbazole, N-arylcarbazole, N-heteroarylcarbazole, N-alkylcarbazole, benzoxazole, benzoimidazole, benzothiazole, benzocarbazole, benzothiophene, dibenzothiophene, thienothiophene, benzofuran, phenanthroline, thiazole, isooxazole, oxazole, oxadiazole, thiadiazole, phenothiazine, dibenzosilole, dibenzofuran, etc., without limitation.

In the description, the explanation on the aryl group may be applied to the arylene group except that the arylene group is a divalent group. The explanation on the heteroaryl group may be applied to the heteroarylene group except that the heteroarylene group is a divalent group.

In the description, the silyl group includes an alkyl silyl group and an aryl silyl group. Examples of the silyl group may include a trimethylsilyl group, a triethylsilyl group, a t-butyldimethylsilyl group, a vinyldimethylsilyl group, a propyldimethylsilyl group, a triphenylsilyl group, a diphenylsilyl group, a phenylsilyl group, etc. However, embodiments are not limited thereto.

In the description, the carbon number of the amino group is not specifically limited, but may be 1 to 30. The amino group may include an alkyl amino group, an aryl amino group, or a heteroaryl amino group. Examples of the amino group include a methylamino group, a dimethylamino group, a phenylamino group, a diphenylamino group, a 9-methyl-anthracenylamino group, etc., without limitation.

In the description, the thio group may include an alkyl thio group and an aryl thio group. The thio group may mean the above-defined alkyl group or aryl group combined with a sulfur atom. Examples of the thio group include a methylthio group, an ethylthio group, a propylthio group, a pentylthio group, a hexylthio group, an octylthio group, a dodecylthio group, a cyclopentylthio group, a cyclohexylthio group, a phenylthio group, a naphthylthio group, etc., without limitation.

In the description, the oxy group may mean the above-defined alkyl group or aryl group which is combined with an oxygen atom. The oxy group may include an alkoxy group and an aryl oxy group. The alkoxy group may be a linear, branched, or cyclic chain. The carbon number of the alkoxy group is not specifically limited but may be, for example, 1 to 20 or 1 to 10. Examples of the oxy group may include methoxy, ethoxy, n-propoxy, isopropoxy, butoxy, pentyloxy, hexyloxy, octyloxy, nonyloxy, decyloxy, benzyloxy, etc. However, embodiments are not limited thereto.

In the description, the carbon number of the amine group is not specifically limited, but may be 1 to 30. The amine group may include an alkyl amine group and an aryl amine group. Examples of the amine group include a methylamine group, a dimethylamine group, a phenylamine group, a diphenylamine group, a naphthylamine group, a 9-methyl-anthracenylamine group, etc., without limitation.

In the description, an alkyl group in the alkylthio group, alkylsulfoxy group, alkylaryl group, alkylamino group, alkylboron group, alkyl silyl group, and alkyl amine group may be the same as the examples of the above-described alkyl group.

In the description, an aryl group in the aryloxy group, arylthio group, arylsulfoxy group, arylamino group, arylboron group, aryl silyl group, and aryl amine group may be the same as the examples of the above-described aryl group.

The third hole transport layer HTL3 may include a compound represented by Formula 2 below. The compound represented by Formula 2 may have a refractive index value of about 1.85 to about 2.40 at a wavelength of about 460 nm.

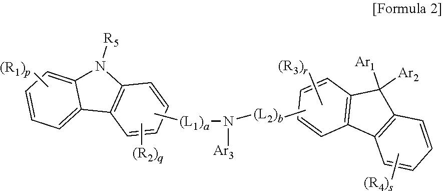

In Formula 2, An and Ar₂ may be each independently a hydrogen atom, a deuterium atom, a halogen atom, a substituted or unsubstituted alkyl group of 1 to 30 carbon atoms, a substituted or unsubstituted aryl group of 6 to 30 ring-forming carbon atoms, or a substituted or unsubstituted heteroaryl group of 2 to 30 ring-forming carbon atoms, or combined with an adjacent group to form a ring. Ar₃ may be a substituted or unsubstituted aryl group of 6 to 30 ring-forming carbon atoms, or a substituted or unsubstituted heteroaryl group of 2 to 30 ring-forming carbon atoms. In Formula 2, a and b may each independently be 0 or 1, and Li and L₂ may be each independently a substituted or unsubstituted cycloalkylene group of 3 to 10 ring-forming carbon atoms, a substituted or unsubstituted heterocycloalkylene group of 2 to 10 ring-forming carbon atoms, a substituted or unsubstituted cycloalkenylene group of 3 to 10 ring-forming carbon atoms, a substituted or unsubstituted arylene group of 6 to 30 ring-forming carbon atoms, or a substituted or unsubstituted heteroarylene group of 2 to 60 ring-forming carbon atoms. In Formula 2, p and s may each independently be an integer from 0 to 4, q and r may each independently be an integer from 0 to 3, and R₁ to R₅ may each independently be a hydrogen atom, a deuterium atom, a halogen atom, a hydroxyl group, a cyano group, a nitro group, an amino group, a substituted or unsubstituted silyl group, a substituted or unsubstituted oxy group, a substituted or unsubstituted alkyl group of 1 to 60 carbon atoms, a substituted or unsubstituted heterocycloalkyl group of 3 to 60 ring-forming carbon atoms, a substituted or unsubstituted aryl group of 6 to 60 ring-forming carbon atoms, or a substituted or unsubstituted heteroaryl group of 2 to 60 ring-forming carbon atoms.

The compound for the third hole transport layer HTL3, represented by Formula 2 may be selected from any one among the compounds in Compound Group 2 below. In the light emitting diode OEL of an embodiment, the third hole transport layer HTL3 may include at least one among the compounds in the Compound Group 2 below.

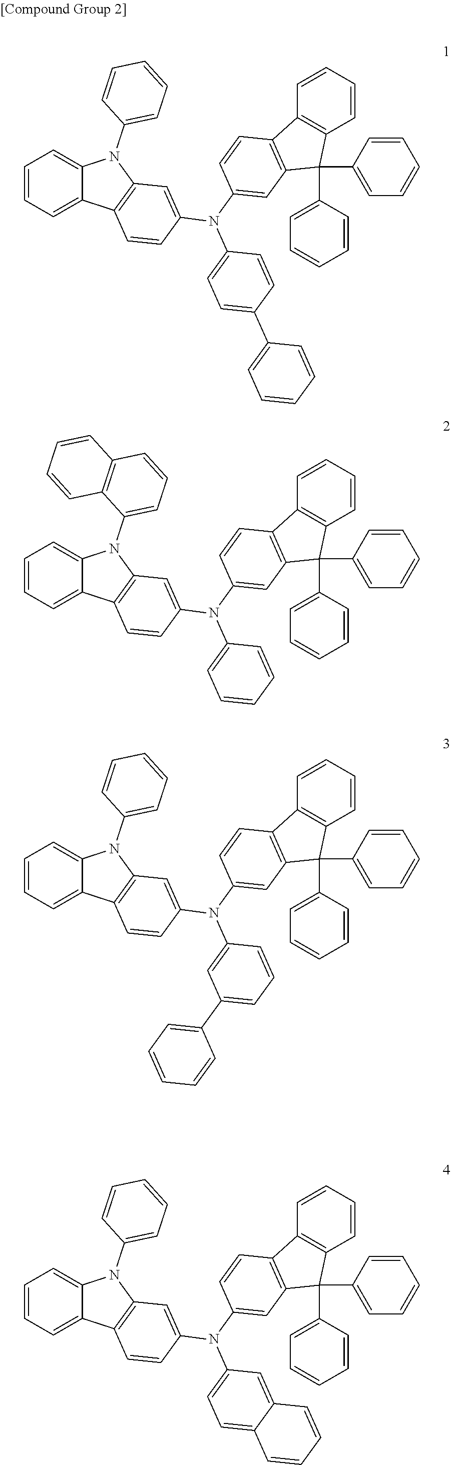 [SMILES: c1ccc(-c2ccc(N(c3ccc4c(c3)C(c3ccccc3)(c3ccccc3)c3ccccc3-4)c3ccc4c5ccccc5n(-c5ccccc5)c4c3)cc2)cc1 ; c1ccc(N(c2ccc3c(c2)C(c2ccccc2)(c2ccccc2)c2ccccc2-3)c2ccc3c4ccccc4n(-c4cccc5ccccc45)c3c2)cc1 ; c1ccc(-c2cccc(N(c3ccc4c(c3)C(c3ccccc3)(c3ccccc3)c3ccccc3-4)c3ccc4c5ccccc5n(-c5ccccc5)c4c3)c2)cc1 ; c1ccc(-n2c3ccccc3c3ccc(N(c4ccc5c(c4)C(c4ccccc4)(c4ccccc4)c4ccccc4-5)c4ccc5ccccc5c4)cc32)cc1]

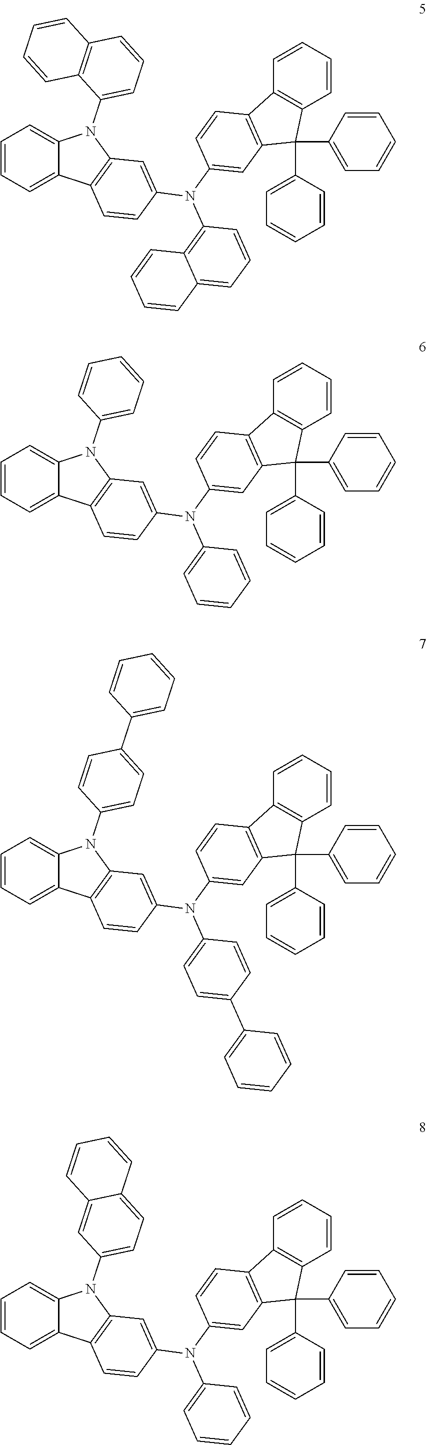 [SMILES: c1ccc(C2(c3ccccc3)c3ccccc3-c3ccc(N(c4ccc5c6ccccc6n(-c6cccc7ccccc67)c5c4)c4cccc5ccccc45)cc32)cc1 ; c1ccc(N(c2ccc3c(c2)C(c2ccccc2)(c2ccccc2)c2ccccc2-3)c2ccc3c4ccccc4n(-c4ccccc4)c3c2)cc1 ; c1ccc(-c2ccc(N(c3ccc4c(c3)C(c3ccccc3)(c3ccccc3)c3ccccc3-4)c3ccc4c5ccccc5n(-c5ccc(-c6ccccc6)cc5)c4c3)cc2)cc1 ; c1ccc(N(c2ccc3c(c2)C(c2ccccc2)(c2ccccc2)c2ccccc2-3)c2ccc3c4ccccc4n(-c4ccc5ccccc5c4)c3c2)cc1]

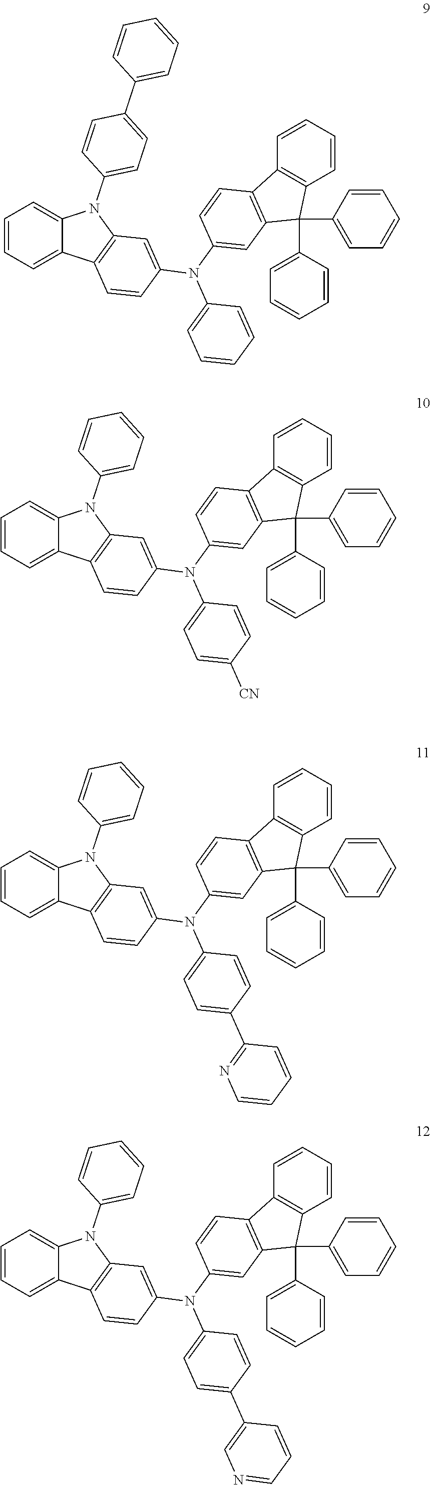 [SMILES: c1ccc(-c2ccc(-n3c4ccccc4c4ccc(N(c5ccccc5)c5ccc6c(c5)C(c5ccccc5)(c5ccccc5)c5ccccc5-6)cc43)cc2)cc1 ; N#Cc1ccc(N(c2ccc3c(c2)C(c2ccccc2)(c2ccccc2)c2ccccc2-3)c2ccc3c4ccccc4n(-c4ccccc4)c3c2)cc1 ; c1ccc(-n2c3ccccc3c3ccc(N(c4ccc(-c5ccccn5)cc4)c4ccc5c(c4)C(c4ccccc4)(c4ccccc4)c4ccccc4-5)cc32)cc1 ; c1ccc(-n2c3ccccc3c3ccc(N(c4ccc(-c5cccnc5)cc4)c4ccc5c(c4)C(c4ccccc4)(c4ccccc4)c4ccccc4-5)cc32)cc1]

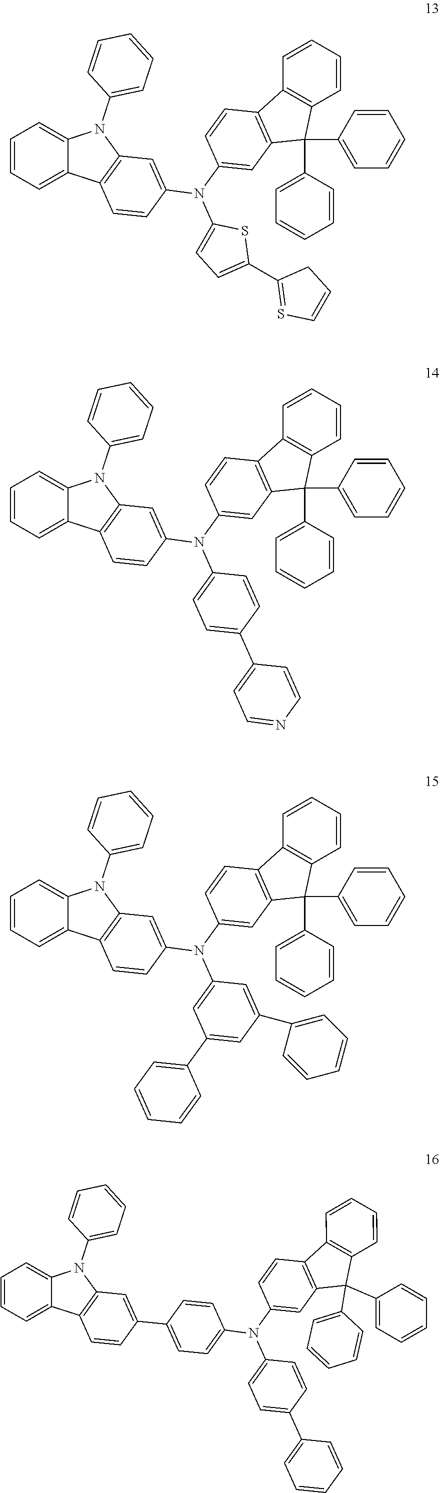

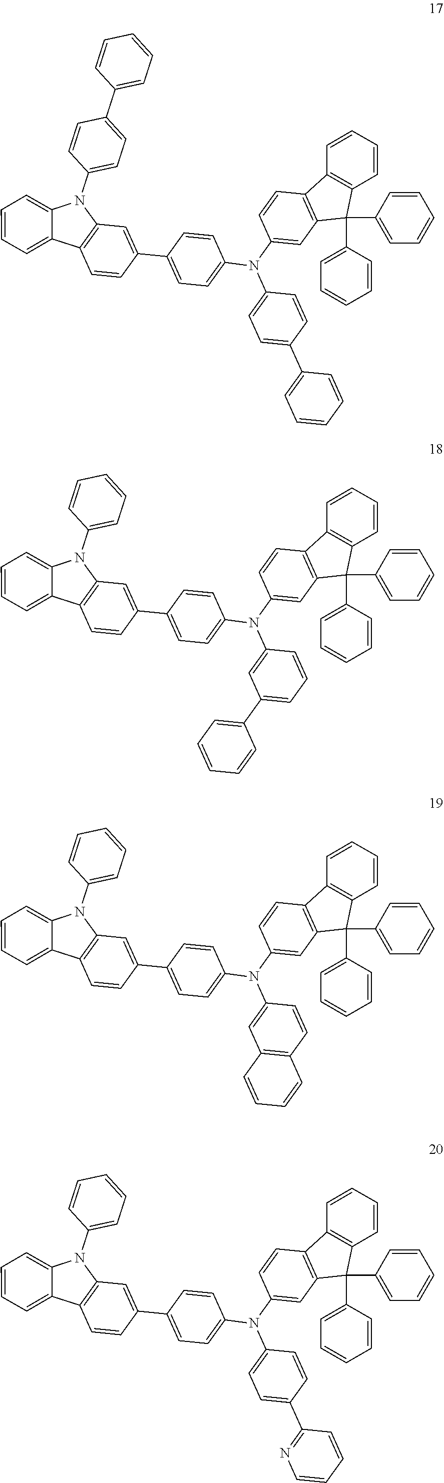 [SMILES: c1ccc(-c2ccc(N(c3ccc(-c4ccc5c6ccccc6n(-c6ccc(-c7ccccc7)cc6)c5c4)cc3)c3ccc4c(c3)C(c3ccccc3)(c3ccccc3)c3ccccc3-4)cc2)cc1 ; c1ccc(-c2cccc(N(c3ccc(-c4ccc5c6ccccc6n(-c6ccccc6)c5c4)cc3)c3ccc4c(c3)C(c3ccccc3)(c3ccccc3)c3ccccc3-4)c2)cc1 ; c1ccc(-n2c3ccccc3c3ccc(-c4ccc(N(c5ccc6c(c5)C(c5ccccc5)(c5ccccc5)c5ccccc5-6)c5ccc6ccccc6c5)cc4)cc32)cc1 ; c1ccc(-n2c3ccccc3c3ccc(-c4ccc(N(c5ccc(-c6ccccn6)cc5)c5ccc6c(c5)C(c5ccccc5)(c5ccccc5)c5ccccc5-6)cc4)cc32)cc1]

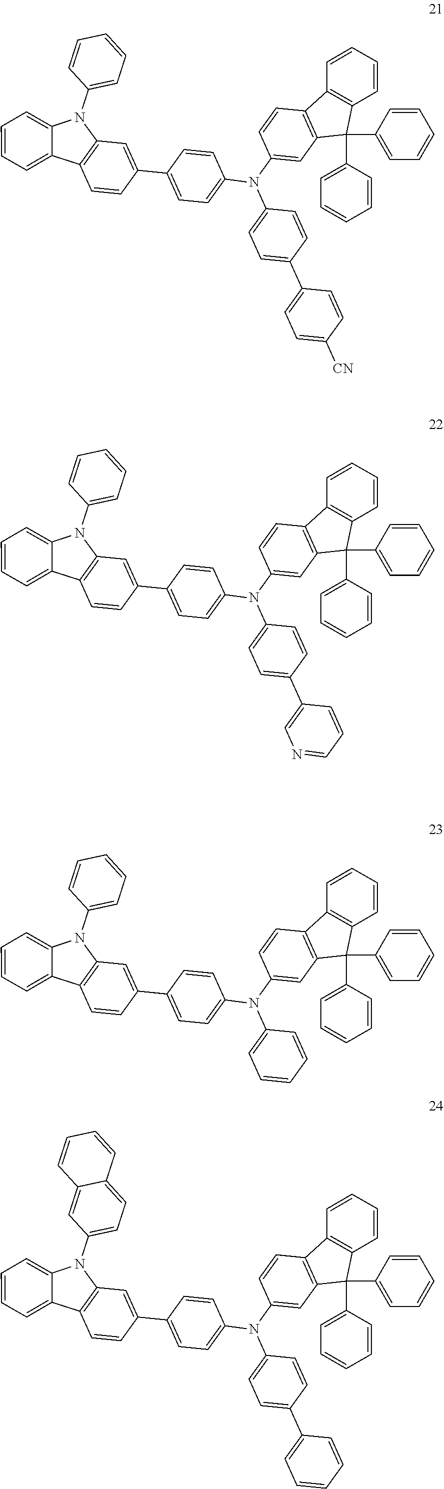 [SMILES: N#Cc1ccc(-c2ccc(N(c3ccc(-c4ccc5c6ccccc6n(-c6ccccc6)c5c4)cc3)c3ccc4c(c3)C(c3ccccc3)(c3ccccc3)c3ccccc3-4)cc2)cc1 ; c1ccc(-n2c3ccccc3c3ccc(-c4ccc(N(c5ccc(-c6cccnc6)cc5)c5ccc6c(c5)C(c5ccccc5)(c5ccccc5)c5ccccc5-6)cc4)cc32)cc1 ; c1ccc(N(c2ccc(-c3ccc4c5ccccc5n(-c5ccccc5)c4c3)cc2)c2ccc3c(c2)C(c2ccccc2)(c2ccccc2)c2ccccc2-3)cc1 ; c1ccc(-c2ccc(N(c3ccc(-c4ccc5c6ccccc6n(-c6ccc7ccccc7c6)c5c4)cc3)c3ccc4c(c3)C(c3ccccc3)(c3ccccc3)c3ccccc3-4)cc2)cc1]

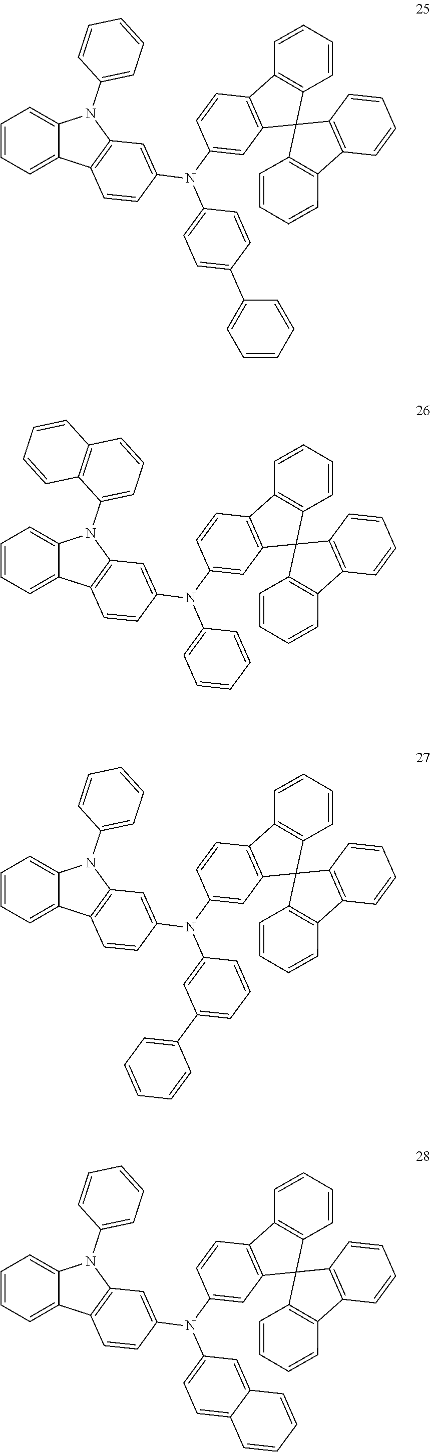 [SMILES: c1ccc(-c2ccc(N(c3ccc4c(c3)C3(c5ccccc5-c5ccccc53)c3ccccc3-4)c3ccc4c5ccccc5n(-c5ccccc5)c4c3)cc2)cc1 ; c1ccc(N(c2ccc3c(c2)C2(c4ccccc4-c4ccccc42)c2ccccc2-3)c2ccc3c4ccccc4n(-c4cccc5ccccc45)c3c2)cc1 ; c1ccc(-c2cccc(N(c3ccc4c(c3)C3(c5ccccc5-c5ccccc53)c3ccccc3-4)c3ccc4c5ccccc5n(-c5ccccc5)c4c3)c2)cc1 ; c1ccc(-n2c3ccccc3c3ccc(N(c4ccc5c(c4)C4(c6ccccc6-c6ccccc64)c4ccccc4-5)c4ccc5ccccc5c4)cc32)cc1]

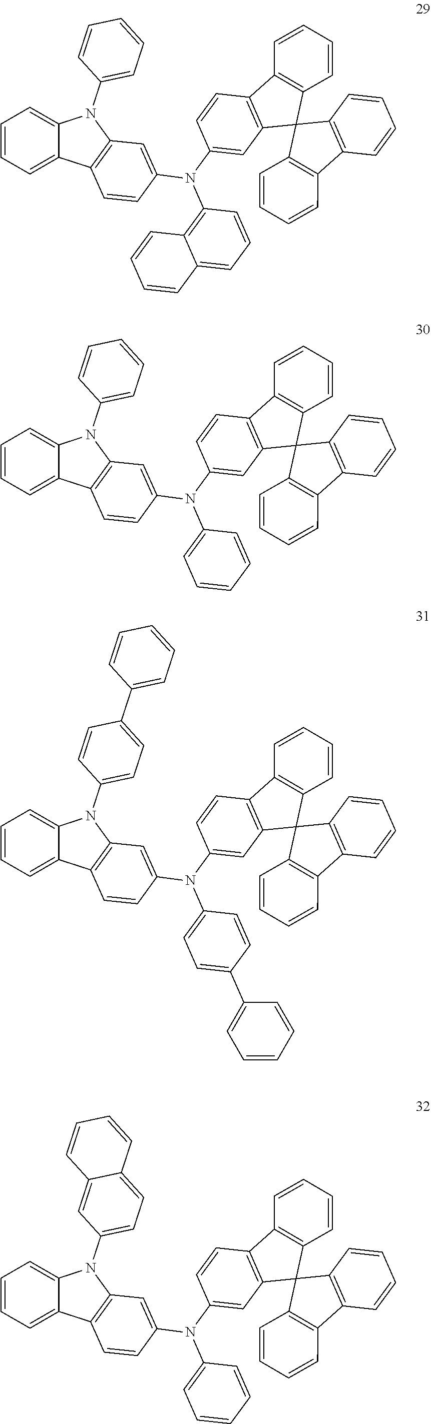 [SMILES: c1ccc(-n2c3ccccc3c3ccc(N(c4ccc5c(c4)C4(c6ccccc6-c6ccccc64)c4ccccc4-5)c4cccc5ccccc45)cc32)cc1 ; c1ccc(N(c2ccc3c(c2)C2(c4ccccc4-c4ccccc42)c2ccccc2-3)c2ccc3c4ccccc4n(-c4ccccc4)c3c2)cc1 ; c1ccc(-c2ccc(N(c3ccc4c(c3)C3(c5ccccc5-c5ccccc53)c3ccccc3-4)c3ccc4c5ccccc5n(-c5ccc(-c6ccccc6)cc5)c4c3)cc2)cc1 ; c1ccc(N(c2ccc3c(c2)C2(c4ccccc4-c4ccccc42)c2ccccc2-3)c2ccc3c4ccccc4n(-c4ccc5ccccc5c4)c3c2)cc1]

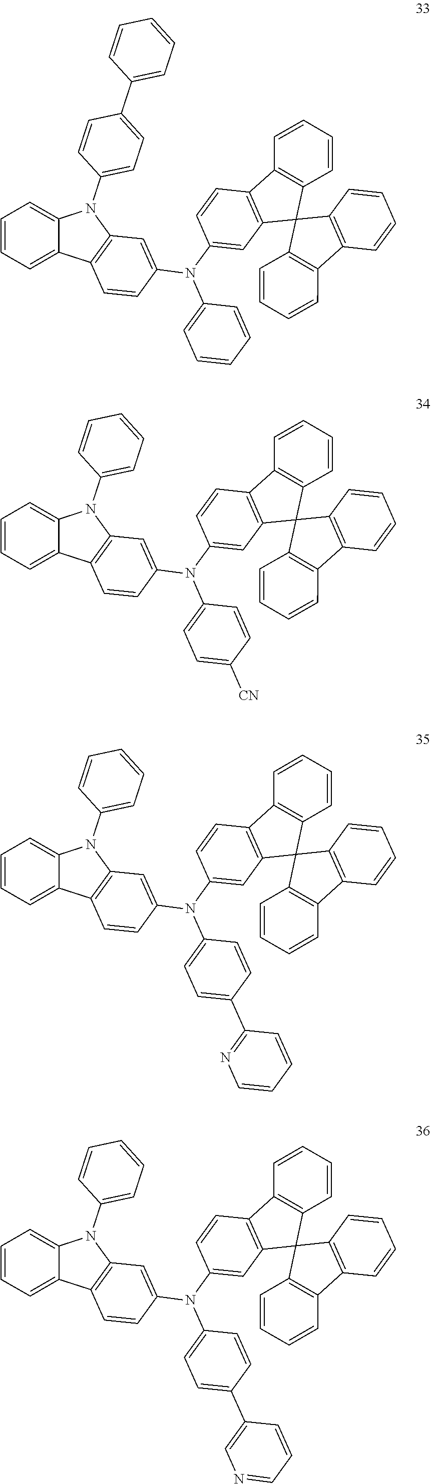 [SMILES: c1ccc(-c2ccc(-n3c4ccccc4c4ccc(N(c5ccccc5)c5ccc6c(c5)C5(c7ccccc7-c7ccccc75)c5ccccc5-6)cc43)cc2)cc1 ; N#Cc1ccc(N(c2ccc3c(c2)C2(c4ccccc4-c4ccccc42)c2ccccc2-3)c2ccc3c4ccccc4n(-c4ccccc4)c3c2)cc1 ; c1ccc(-n2c3ccccc3c3ccc(N(c4ccc(-c5ccccn5)cc4)c4ccc5c(c4)C4(c6ccccc6-c6ccccc64)c4ccccc4-5)cc32)cc1 ; c1ccc(-n2c3ccccc3c3ccc(N(c4ccc(-c5cccnc5)cc4)c4ccc5c(c4)C4(c6ccccc6-c6ccccc64)c4ccccc4-5)cc32)cc1]

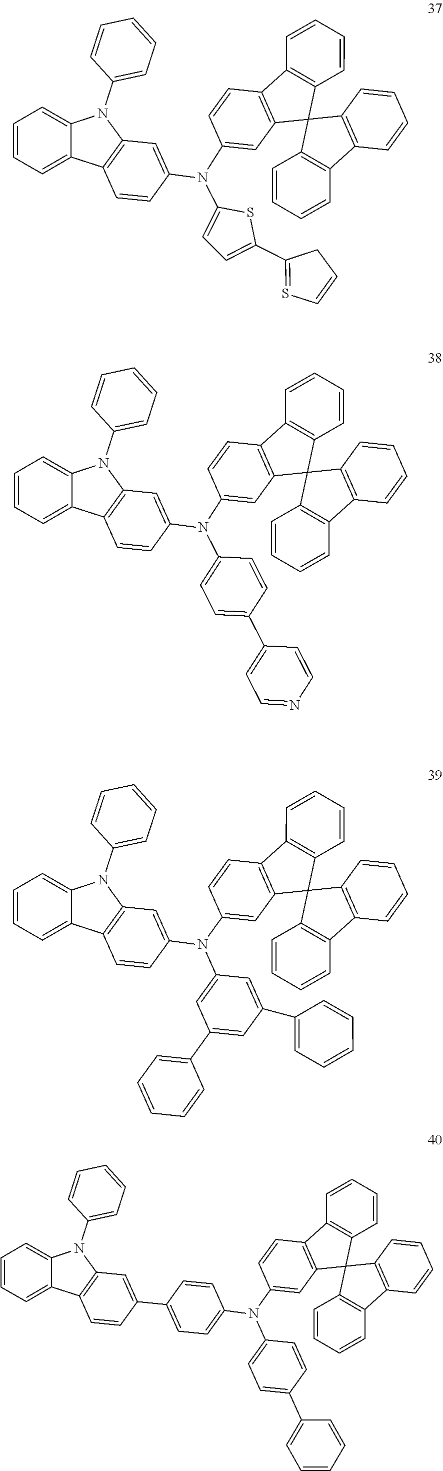

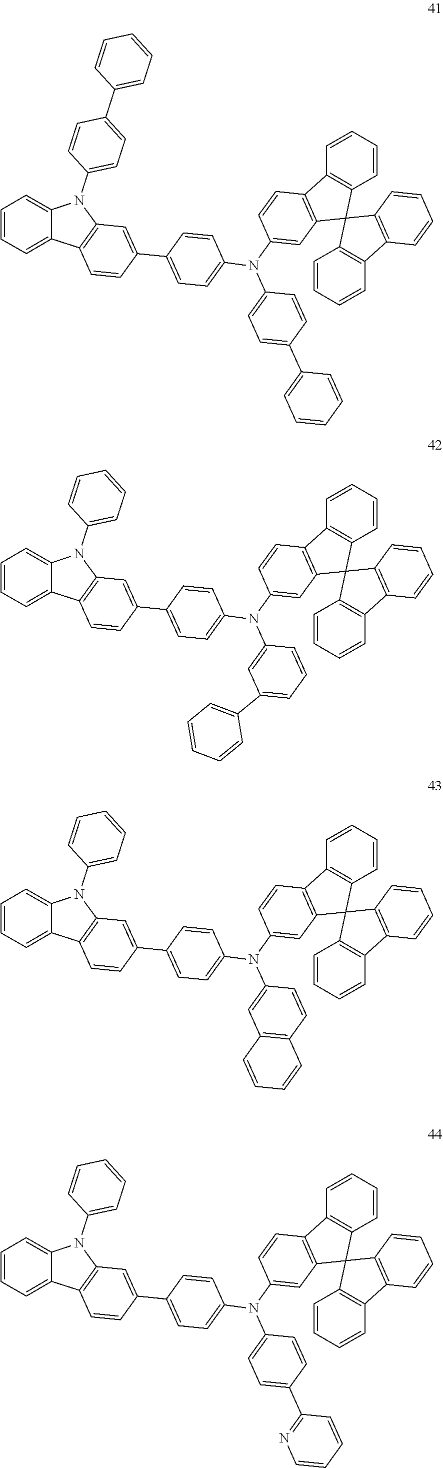 [SMILES: c1ccc(-c2ccc(N(c3ccc(-c4ccc5c6ccccc6n(-c6ccc(-c7ccccc7)cc6)c5c4)cc3)c3ccc4c(c3)C3(c5ccccc5-c5ccccc53)c3ccccc3-4)cc2)cc1 ; c1ccc(-c2cccc(N(c3ccc(-c4ccc5c6ccccc6n(-c6ccccc6)c5c4)cc3)c3ccc4c(c3)C3(c5ccccc5-c5ccccc53)c3ccccc3-4)c2)cc1 ; c1ccc(-n2c3ccccc3c3ccc(-c4ccc(N(c5ccc6c(c5)C5(c7ccccc7-c7ccccc75)c5ccccc5-6)c5ccc6ccccc6c5)cc4)cc32)cc1 ; c1ccc(-n2c3ccccc3c3ccc(-c4ccc(N(c5ccc(-c6ccccn6)cc5)c5ccc6c(c5)C5(c7ccccc7-c7ccccc75)c5ccccc5-6)cc4)cc32)cc1]

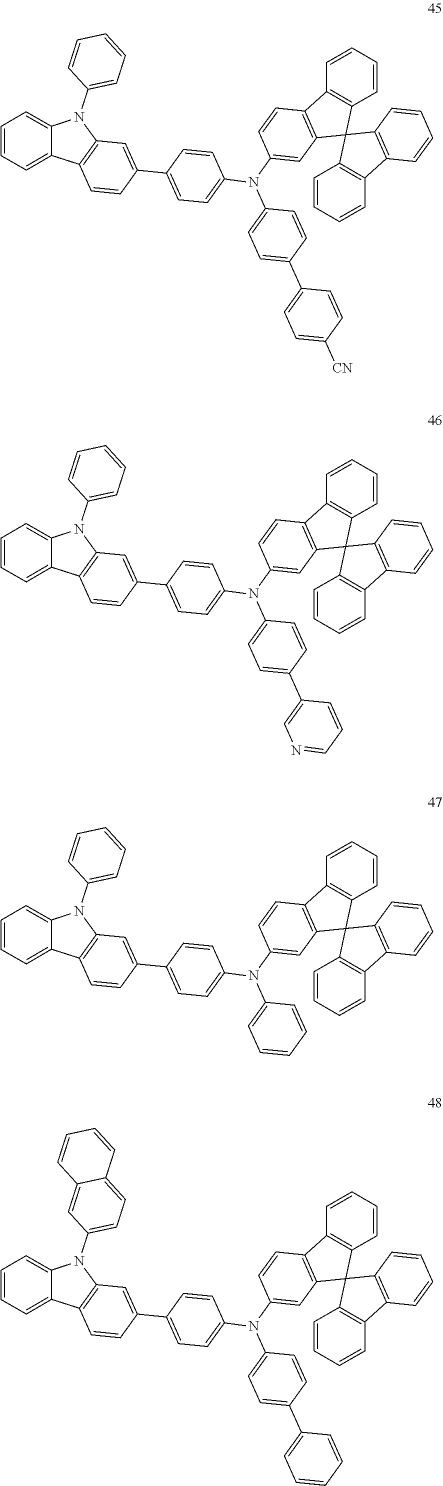 [SMILES: N#Cc1ccc(-c2ccc(N(c3ccc(-c4ccc5c6ccccc6n(-c6ccccc6)c5c4)cc3)c3ccc4c(c3)C3(c5ccccc5-c5ccccc53)c3ccccc3-4)cc2)cc1 ; c1ccc(-n2c3ccccc3c3ccc(-c4ccc(N(c5ccc(-c6cccnc6)cc5)c5ccc6c(c5)C5(c7ccccc7-c7ccccc75)c5ccccc5-6)cc4)cc32)cc1 ; c1ccc(N(c2ccc(-c3ccc4c5ccccc5n(-c5ccccc5)c4c3)cc2)c2ccc3c(c2)C2(c4ccccc4-c4ccccc42)c2ccccc2-3)cc1 ; c1ccc(-c2ccc(N(c3ccc(-c4ccc5c6ccccc6n(-c6ccc7ccccc7c6)c5c4)cc3)c3ccc4c(c3)C3(c5ccccc5-c5ccccc53)c3ccccc3-4)cc2)cc1]

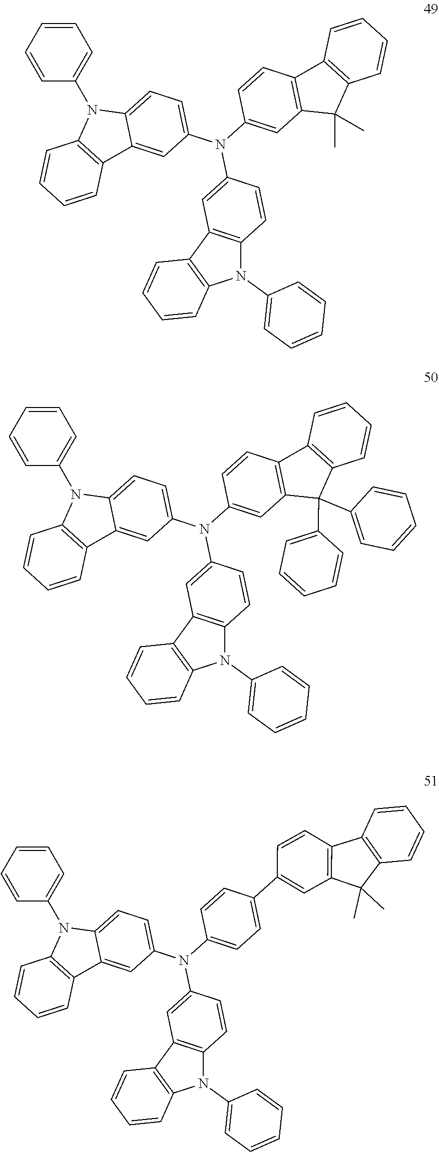 [SMILES: CC1(C)c2ccccc2-c2ccc(N(c3ccc4c(c3)c3ccccc3n4-c3ccccc3)c3ccc4c(c3)c3ccccc3n4-c3ccccc3)cc21 ; c1ccc(-n2c3ccccc3c3cc(N(c4ccc5c(c4)C(c4ccccc4)(c4ccccc4)c4ccccc4-5)c4ccc5c(c4)c4ccccc4n5-c4ccccc4)ccc32)cc1 ; CC1(C)c2ccccc2-c2ccc(-c3ccc(N(c4ccc5c(c4)c4ccccc4n5-c4ccccc4)c4ccc5c(c4)c4ccccc4n5-c4ccccc4)cc3)cc21]

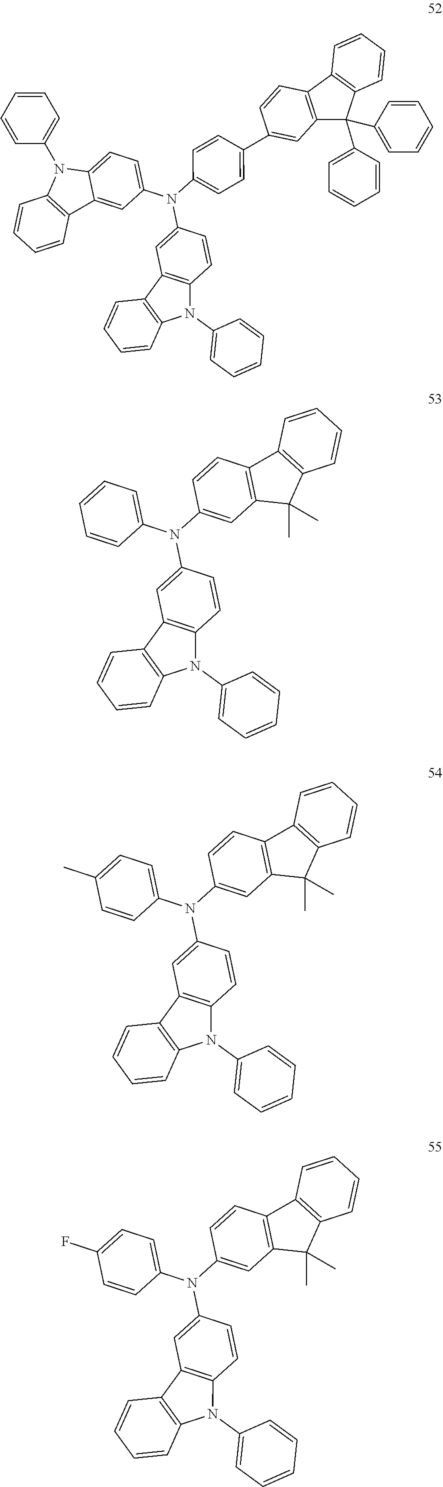 [SMILES: c1ccc(-n2c3ccccc3c3cc(N(c4ccc(-c5ccc6c(c5)C(c5ccccc5)(c5ccccc5)c5ccccc5-6)cc4)c4ccc5c(c4)c4ccccc4n5-c4ccccc4)ccc32)cc1 ; CC1(C)c2ccccc2-c2ccc(N(c3ccccc3)c3ccc4c(c3)c3ccccc3n4-c3ccccc3)cc21 ; Cc1ccc(N(c2ccc3c(c2)C(C)(C)c2ccccc2-3)c2ccc3c(c2)c2ccccc2n3-c2ccccc2)cc1 ; CC1(C)c2ccccc2-c2ccc(N(c3ccc(F)cc3)c3ccc4c(c3)c3ccccc3n4-c3ccccc3)cc21]

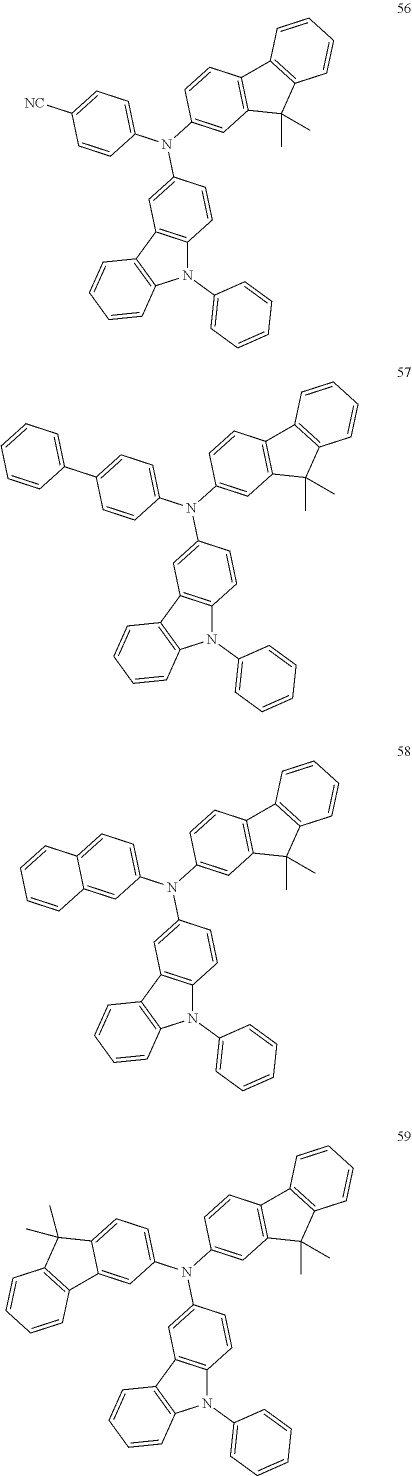 [SMILES: CC1(C)c2ccccc2-c2ccc(N(c3ccc(C#N)cc3)c3ccc4c(c3)c3ccccc3n4-c3ccccc3)cc21 ; CC1(C)c2ccccc2-c2ccc(N(c3ccc(-c4ccccc4)cc3)c3ccc4c(c3)c3ccccc3n4-c3ccccc3)cc21 ; CC1(C)c2ccccc2-c2ccc(N(c3ccc4ccccc4c3)c3ccc4c(c3)c3ccccc3n4-c3ccccc3)cc21 ; CC1(C)c2ccccc2-c2cc(N(c3ccc4c(c3)C(C)(C)c3ccccc3-4)c3ccc4c(c3)c3ccccc3n4-c3ccccc3)ccc21]

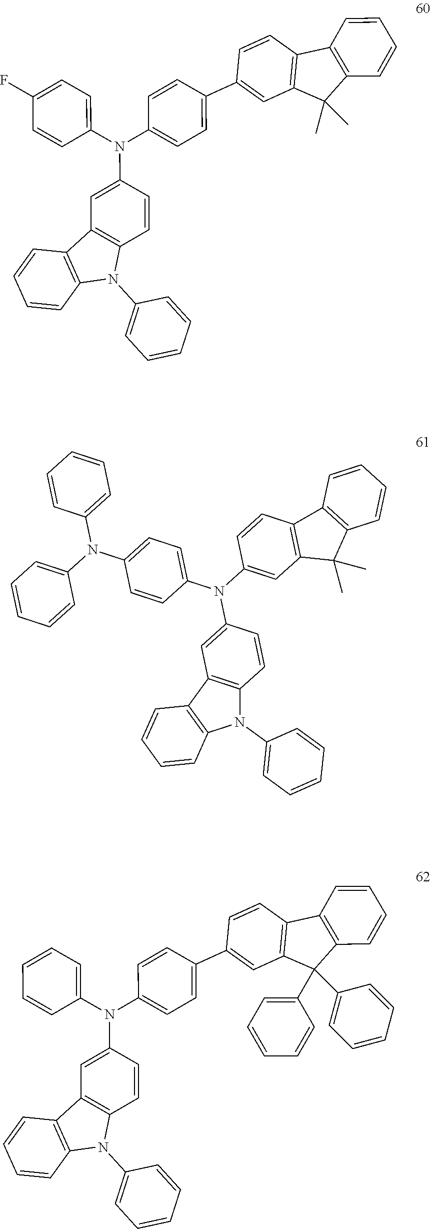 [SMILES: CC1(C)c2ccccc2-c2ccc(-c3ccc(N(c4ccc(F)cc4)c4ccc5c(c4)c4ccccc4n5-c4ccccc4)cc3)cc21 ; CC1(C)c2ccccc2-c2ccc(N(c3ccc(N(c4ccccc4)c4ccccc4)cc3)c3ccc4c(c3)c3ccccc3n4-c3ccccc3)cc21 ; c1ccc(N(c2ccc(-c3ccc4c(c3)C(c3ccccc3)(c3ccccc3)c3ccccc3-4)cc2)c2ccc3c(c2)c2ccccc2n3-c2ccccc2)cc1]

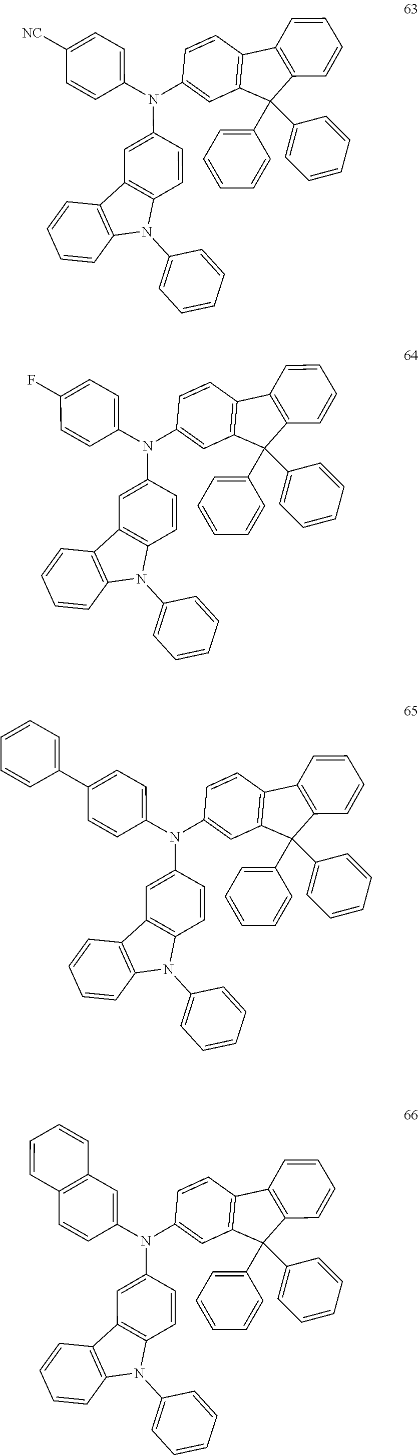 [SMILES: N#Cc1ccc(N(c2ccc3c(c2)C(c2ccccc2)(c2ccccc2)c2ccccc2-3)c2ccc3c(c2)c2ccccc2n3-c2ccccc2)cc1 ; Fc1ccc(N(c2ccc3c(c2)C(c2ccccc2)(c2ccccc2)c2ccccc2-3)c2ccc3c(c2)c2ccccc2n3-c2ccccc2)cc1 ; c1ccc(-c2ccc(N(c3ccc4c(c3)C(c3ccccc3)(c3ccccc3)c3ccccc3-4)c3ccc4c(c3)c3ccccc3n4-c3ccccc3)cc2)cc1 ; c1ccc(-n2c3ccccc3c3cc(N(c4ccc5c(c4)C(c4ccccc4)(c4ccccc4)c4ccccc4-5)c4ccc5ccccc5c4)ccc32)cc1]

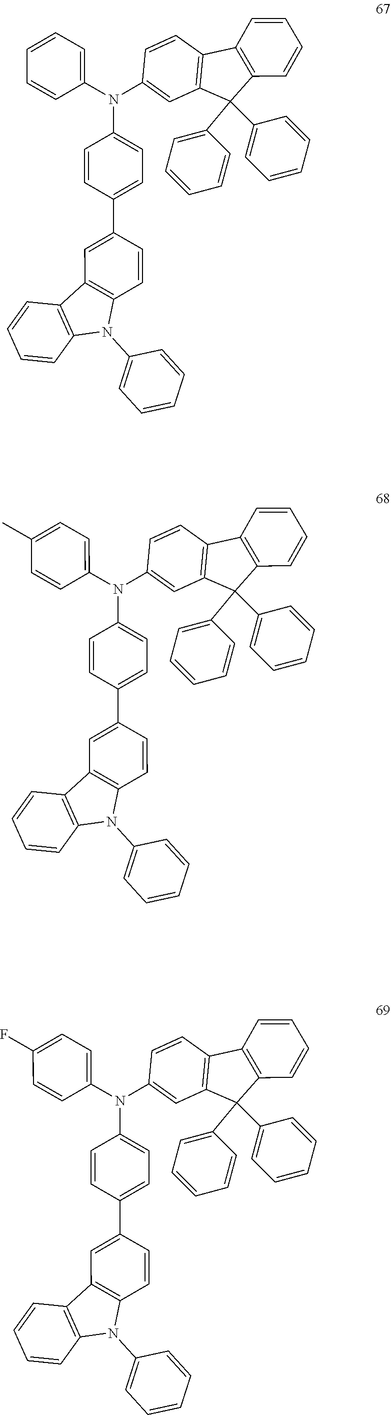 [SMILES: c1ccc(N(c2ccc(-c3ccc4c(c3)c3ccccc3n4-c3ccccc3)cc2)c2ccc3c(c2)C(c2ccccc2)(c2ccccc2)c2ccccc2-3)cc1 ; Cc1ccc(N(c2ccc(-c3ccc4c(c3)c3ccccc3n4-c3ccccc3)cc2)c2ccc3c(c2)C(c2ccccc2)(c2ccccc2)c2ccccc2-3)cc1 ; Fc1ccc(N(c2ccc(-c3ccc4c(c3)c3ccccc3n4-c3ccccc3)cc2)c2ccc3c(c2)C(c2ccccc2)(c2ccccc2)c2ccccc2-3)cc1]

The hole transport region HTR of the light emitting diode OEL of an embodiment may include three hole transport layers HTL1, HTL2, and HTL3. The light emitting diode OEL of an embodiment may include hole transport layers obtained by stacking first hole transport layer HTL1/third hole transport layer HTL3/second hole transport layer HTL2 in order between the first electrode EL1 and the emission layer EML, and may show excellent emission efficiency properties. In an embodiment, the refractive indexes of the first hole transport layer HTL1 and the second hole transport layer HTL2 may each be less than the refractive index of the third hole transport layer HTL3, and a refractive index difference may be greater than about 0.1.

The emission layer EML is provided on the hole transport region HTR. The emission layer EML may have a thickness in a range of, for example, about 100 Å to about 1,000 Å. For example, the thickness of the emission layer EML may be in a range of about 100 Å to about 300 Å. The emission layer EML may be a single layer formed using a single material, a single layer formed using multiple different materials, or have a multilayer structure having multiple layers formed using multiple different materials.

The emission layer EML may emit any one among red light, green light, blue light, white light, yellow light, and cyan light. The emission layer EML may include a fluorescence emitting material or a phosphorescence emitting material. In an embodiment, the emission layer EML may include a quantum dot.

In the light emitting diode OEL of an embodiment, the emission layer EML may include anthracene derivatives, pyrene derivatives, fluoranthene derivatives, chrysene derivatives, dihydrobenzanthracene derivatives, or triphenylene derivatives. For example, the emission layer EML may include anthracene derivatives or pyrene derivatives. However, embodiments are not limited thereto, and the emission layer EML may include other light emitting materials used in the art.

In the light emitting diode OEL of an embodiment, the electron transport region ETR is provided on the emission layer EML. The electron transport region ETR may include at least one of a hole blocking layer, an electron transport layer, and an electron injection layer, but embodiments are not limited thereto.

The electron transport region ETR may have a single layer formed using a single material, a single layer formed using multiple different materials, or a multilayer structure having multiple layers formed using multiple different materials.

For example, the electron transport region ETR may have a single layer structure of an electron injection layer or an electron transport layer, or a single layer structure formed using an electron injection material and an electron transport material. Also, the electron transport region ETR may have a single layer structure formed using multiple different materials, or a structure stacked from the emission layer EML of electron transport layer/electron injection layer, or hole blocking layer/electron transport layer/electron injection layer, without limitation. A thickness of the electron transport region ETR may be, for example, in a range of about 1,000 Å to about 1,500 Å.

If the electron transport region ETR includes the electron injection layer, the electron transport region ETR may include a metal halide such as LiF, NaCl, CsF, RbCl, RbI, and CuI, a lanthanide metal such as Yb, a metal oxide such as Li₂O and BaO, or lithium quinolate (LiQ). However, embodiments are not limited thereto. The electron injection layer may also be formed using a mixture material of an electron transport material and an insulating organo-metal salt. The organo-metal salt may be a material having an energy band gap of about 4 eV or more. For example, the organo-metal salt may include, for example, metal acetates, metal benzoates, metal acetoacetates, metal acetylacetonates, or metal stearates. If the electron transport region ETR includes the electron transport layer, the electron transport region ETR may include an anthracene-based compound. However, embodiments are not limited thereto. The electron transport region ETR may include other electron transport materials used in the art.

The second electrode EL2 is provided on the electron transport region ETR. The second electrode EL2 may be a common electrode or a cathode. The second electrode EL2 may be a transmissive electrode or a transflective electrode. If the second electrode EL2 is the transmissive electrode, the second electrode EL2 may be formed using a transparent metal oxide, for example, ITO, IZO, ZnO, ITZO, etc. If the second electrode EL2 is the transflective electrode, the second electrode EL2 may include Ag, Mg, Cu, Al, Pt, Pd, Au, Ni, Nd, Ir, Cr, Li, Ca, LiF/Ca, LiF/Al, Mo, Ti, compounds including thereof, or mixtures thereof (for example, a mixture of Ag and Mg). Otherwise, the second electrode EL2 may have a multilayered structure including a reflective layer or a transflective layer formed using the above-described materials and a transparent conductive layer formed using ITO, IZO, ZnO, ITZO, etc.

On the second electrode EL2 of the light emitting diode OEL of an embodiment, a capping layer (not shown) may be further disposed. The capping layer (not shown) may include, for example, α-NPD, NPB, TPD, m-MTDATA, Alq₃, CuPc, N4,N4,N4′,N4′-tetra(biphenyl-4-yl) biphenyl-4,4′-diamine (TPD15), 4,4′,4″-tris(carbazol-9-yl) triphenylamine (TCTA), etc.

The display device of an embodiment includes multiple light emitting diodes, and at least one light emitting diode among the multiple light emitting diodes may have the above-described configuration of the light emitting diode according to an embodiment.

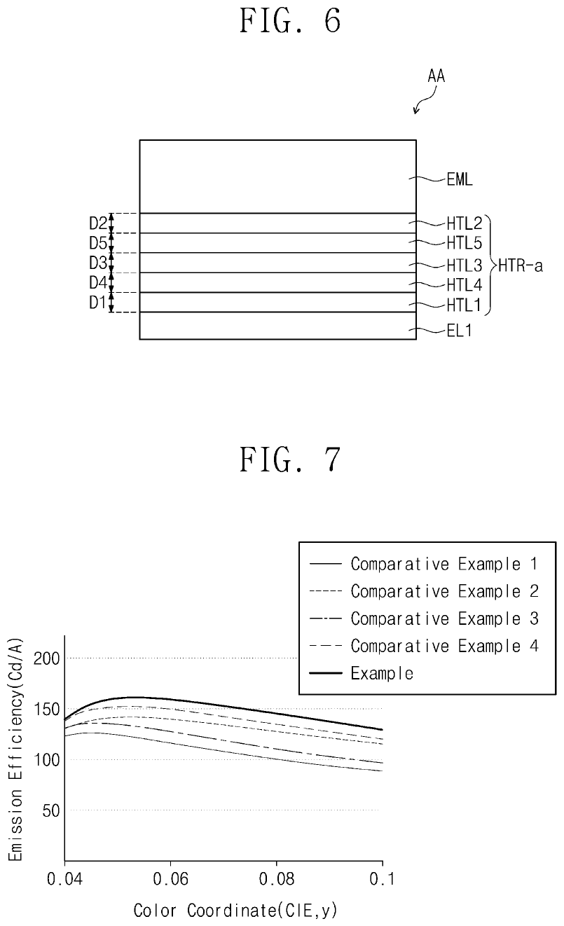

FIG. 6 is a schematic cross-sectional view of a portion of a light emitting diode according to an embodiment. When compared with a portion of the light emitting diode shown in FIG. 5, the light emitting diode according to an embodiment, shown in FIG. 6 is different only in the configuration of a hole transport region.

Referring to FIG. 6, in an embodiment, a hole transport region HTR-a may include first to fifth hole transport layers HTL1 to HTL5. For example, when compared with an embodiment shown in FIG. 5, the hole transport region HTR-a in the light emitting diode according to an embodiment may further include a fourth hole transport layer HTL4 and a fifth hole transport layer HTL5.

The fourth hole transport layer HTL4 may be disposed between the first hole transport layer HTL1 and the third hole transport layer HTL3, and the fifth hole transport layer HTL5 may be disposed between the second hole transport layer HTL2 and the third hole transport layer HTL3.

The fourth hole transport layer HTL4 may include both the amine compound represented by Formula 1 and included in the first hole transport layer HTL1, and the compound represented by Formula 2 and included in the third hole transport layer HTL3. In the fourth hole transport layer HTL4, the amount of the amine compound represented by Formula 1 at a portion adjacent to the first hole transport layer HTL1 may be greater than the amount of the amine compound represented by Formula 1 at a portion adjacent to the third hole transport layer HTL3. In the fourth hole transport layer HTL4, the amount of the compound represented by Formula 2 at a portion adjacent to the third hole transport layer HTL3 may be greater than the amount of the compound represented by Formula 2 at a portion adjacent to the first hole transport layer HTL1. For example, the fourth hole transport layer HTL4 is a layer including both the compound forming the first hole transport layer HTL1 and the compound forming the third hole transport layer HTL3, and in the fourth hole transport layer HTL4, the amount of the amine compound represented by Formula 1 among the amount of the total fourth hole transport layer HTL4 may be gradually reduced in a direction from the first hole transport layer HTL1 toward the third hole transport layer HTL3. In the fourth hole transport layer HTL4, the amount of the compound represented by Formula 2 among the amount of the total fourth hole transport layer HTL4 may be gradually reduced in a direction from the third hole transport layer HTL3 toward the first hole transport layer HTL1.

The fourth hole transport layer HTL4 may have a value between the first refractive index of the first hole transport layer HTL1 and the third refractive index of the third hole transport layer HTL3 at a wavelength of about 460 nm. The refractive index of the fourth hole transport layer HTL4 may be gradually increased in a direction from the first hole transport layer HTL1 to the third hole transport layer HTL3.

In an embodiment, the fifth hole transport layer HTL5 may include both the amine compound represented by Formula 1 and included in the second hole transport layer HTL2, and the compound represented by Formula 2 and included in the third hole transport layer HTL3. In the fifth hole transport layer HTL5, the amount of the amine compound represented by Formula 1 at a portion adjacent to the second hole transport layer HTL2 may be greater than the amount of the amine compound represented by Formula 1 at a portion adjacent to the third hole transport layer HTL3. In the fifth hole transport layer HTL5, the amount of the compound represented by Formula 2 at a portion adjacent to the third hole transport layer HTL3 may be greater than the amount of the compound represented by Formula 2 at a portion adjacent to the second hole transport layer HTL2. For example, the fifth hole transport layer HTL5 is a layer including both the compound forming the second hole transport layer HTL2 and the compound forming the third hole transport layer HTL3, and in the fifth hole transport layer HTL5, the amount of the amine compound represented by Formula 1 among the amount of the total fifth hole transport layer HTL5 may be gradually reduced in a direction from the second hole transport layer HTL2 toward the third hole transport layer HTL3. In the fifth hole transport layer HTL5, the amount of the compound represented by Formula 2 among the amount of the total fifth hole transport layer HTL5 may be gradually reduced in a direction from the third hole transport layer HTL3 toward the second hole transport layer HTL2.

The fifth hole transport layer HTL5 may have a value between the second refractive index of the second hole transport layer HTL2 and the third refractive index of the third hole transport layer HTL3 at a wavelength of about 460 nm. The refractive index of the fifth hole transport layer HTL5 may be gradually increased in a direction from the second hole transport layer HTL2 to the third hole transport layer HTL3.

In an embodiment including the first to fifth hole transport layers HTL1 to HTL5, a thickness of each of the first to fifth hole transport layers HTL1 to HTL 5 may each be in a range of about 100 Å to about 1,000 Å. The thicknesses of the first to fifth hole transport layers HTL1 to HTL5 may be the same, or at least one thereof may be different from the thicknesses of the remainder. The thicknesses of the first to fifth hole transport layers HTL1 to HTL5 may be the combination of various types according to the properties of the light emitting diode required.

Referring to FIG. 3 again, the display device DD of an embodiment may include the first to third light emitting diodes OEL-1, OEL-2, and OEL-3 divided by the pixel defining layer PDL, and the first to third light emitting diodes OEL-1, OEL-2, and OEL-3 may have different configurations of the emission layers EML-B, EML-G, and EML-R and may emit light in different wavelength regions. One light emitting diode among the first to third light emitting diodes OEL-1, OEL-2, and OEL-3 may have the configurations of the light emitting diodes of FIG. 4 to FIG. 6. In another embodiment, two light emitting diodes or three light emitting diodes selected among the first to third light emitting diodes OEL-1, OEL-2, and OEL-3 may have the configurations of the light emitting diodes of FIG. 4 to FIG. 6.

In the display device DD of an embodiment, in a case where all three light emitting diodes OEL-1, OEL-2, and OEL-3 have the configuration of the light emitting diode of FIG. 4 and FIG. 5, the hole transport region HTR may be provided as a common layer for all the first to third light emitting diodes OEL-1, OEL-2, and OEL-3. For example, the hole transport region HTR provided as the common layer may have a structure including the first to third hole transport layers HTL1, HTL2, and HTL3.

In the display device of an embodiment, in contrast to FIG. 3, the hole transport region HTR may be disposed in the opening portion OH defined in the pixel defining layer PDL and may be separately provided corresponding to emission layers EML-B, EML-G, and EML-R.

The hole transport region HTR included in each of the light emitting diodes OEL-1, OEL-2, and OEL-3 may also have a structure including the first to third hole transport layers HTL1, HTL2, and HTL3. If the hole transport region HTR is provided not as a common layer but separately corresponding to the light emitting diodes OEL-1, OEL-2, and OEL-3, a thickness ratio of the first to third hole transport layers HTL1, HTL2, and HTL3 included in the light emitting diodes OEL-1, OEL-2, and OEL-3 may be controlled differently according to the wavelength region of light emitted from each of the light emitting diodes OEL-1, OEL-2, and OEL-3.

In another embodiment, in the display device DD of an embodiment, the first light emitting diode OEL-1 emitting blue light may have a light emitting diode structure including the first to third hole transport layers HTL1, HTL2 and HTL3. However, embodiments are not limited thereto.

In the display device DD of an embodiment shown in FIG. 3, the hole transport region HTR may have the structure of the hole transport region HTR-a shown in FIG. 6. The hole transport region HTR-a including the first to fifth hole transport layers HTL1 to HTL5 may be provided as a common layer for the whole first to third light emitting diodes OEL-1, OEL-2, and OEL-3. In another embodiment, the display device of an embodiment may include the hole transport region HTR-a disposed in the opening portion OH defined by the pixel defining layer PDL and separately provided corresponding to the emission layers EMLL-B, EML-G, and EML-R. For example, the hole transport region HTR-a included in each of the light emitting diodes OEL-1, OEL-2, and OEL-3 may have a structure including the first to fifth hole transport layers HTL1 to HTL5.

FIG. 7 is a graph comparing and showing emission efficiency of the Comparative Examples and the Example. The Example corresponds to evaluation results on a light emitting diode having the above-described hole transport region structure of the light emitting diode of an embodiment, and Comparative Example 1 to Comparative Example 4 correspond to evaluation results on light emitting diodes having different configurations of the hole transport regions from the Example. Except for the different configurations of the hole transport regions, the configurations of other functional layers were the same in the Comparative Examples and the Example. The Comparative Examples and the Example correspond to light emitting diodes emitting blue light having a central wavelength around 464 nm.

Comparative Example 1 and Comparative Example 2 correspond to cases where the hole transport region is formed of one hole transport layer. Comparative Example 1 corresponds to a case of including only one hole transport layer having a refractive index of about 1.9, and Comparative Example 2 corresponds to a case of including only one hole transport layer having a refractive index of about 1.4.

Comparative Example 3 and Comparative Example 4 correspond to cases where the hole transport region is formed of two hole transport layers. Comparative Example 3 corresponds to a case where the refractive index of a hole transport layer adjacent to a first electrode is about 1.4, and the refractive index of a hole transport layer adjacent to an emission layer is about 1.9. Comparative Example 4 corresponds to a case where the refractive index of a hole transport layer adjacent to a first electrode is about 1.9, and the refractive index of a hole transport layer adjacent to an emission layer is about 1.4. Comparative Example 3 and Comparative Example 4 correspond to cases having different stacking order of the hole transport layer of a low refractive index and the hole transport layer of a high refractive index.

The Example corresponds to a case of including the aforementioned hole transport region structure of the light emitting diode and includes three hole transport layers, wherein the refractive indexes of a first hole transport layer adjacent to a first electrode and a second hole transport layer adjacent to an emission layer are about 1.4, and the refractive index of a third hole transport layer disposed between the first hole transport layer and the second hole transport layer is about 1.9.

In FIG. 7, the horizontal axis represents color coordinate values and corresponds to “y” values of the color coordinate of light emitted from a light emitting diode. In FIG. 7, the value shown in the horizontal axis corresponds to the y value in a CIE color coordinate. The graph of FIG. 7 represents emission efficiency according to the color coordinates of light emitted. Referring to the results of FIG. 7, it could be found that the light emitting diode of the Example shows higher emission efficiency when compared with the Comparative Examples in a range of a color coordinate value of about 0.04 to about 0.1. The Example showed improving effects of emission efficiency by about 34% when compared with Comparative Example 1.

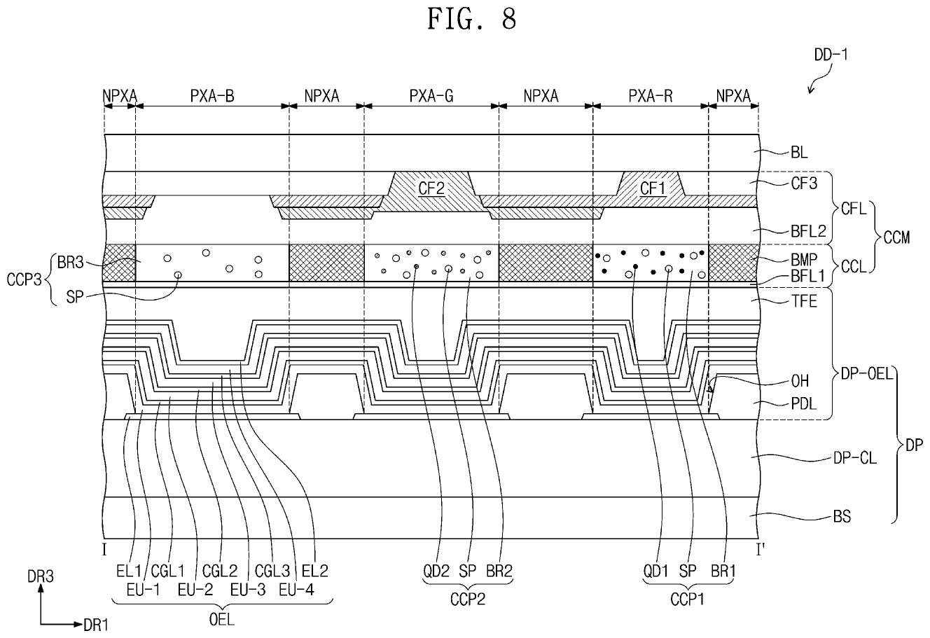

Hereinafter, in FIG. 8 to FIG. 12, a light emitting diode according to an embodiment, and a display device including the light emitting diode will be explained. In an embodiment explained with to FIG. 8 to FIG. 12, the features which overlap with the explanation of FIG. 1 to FIG. 7 will not be explained again, and the differing features will be explained.

FIG. 8 is a schematic cross-sectional view showing a portion of the display device corresponding to line I-I′ in FIG. 2. As explained in reference to FIG. 2 and FIG. 3, a display device DD-1 according to an embodiment may include a non-luminous area NPXA and luminous areas PXA-B, PXA-G and PXA-R.

Referring to FIG. 8, the display device DD-1 of an embodiment may include a display panel DP and a light controlling member CCM disposed on the display panel DP, and the display panel DP may provide first light. For example, the display panel DP may provide blue light as the first light, but embodiments are not limited thereto, and the display panel DP may emit white light.

The light controlling member CCM may convert the wavelength of the first light provided from the display panel DP, or may transmit the first light provided from the display panel DP. For example, the light controlling member CCM may include a light controlling layer CCL and a color filter layer CFL, but embodiments are not limited thereto. In an embodiment, the color filter layer CFL may be omitted, a polarization plate may be included instead of the color filter layer CFL, or an organic layer including a pigment or a dye, may be included instead of the color filter layer CFL which is divided into multiple filters CF1, CF2, and CF3.

The display device DD-1 may further include an upper base substrate BL. The upper base substrate BL may provide a base surface on which the light controlling member CCM is disposed. The upper base substrate BL may be a glass substrate, a metal substrate, a plastic substrate, etc. However, embodiments are not limited thereto, and the upper base substrate BL may include an inorganic layer, an organic layer, or a composite material layer. Although not shown in the drawings, in an embodiment, the upper base substrate BL may be omitted.

In an embodiment, the display panel DP may include a base substrate BS, a circuit layer DP-CL provided on the base substrate BS, and a display device layer DP-OEL. The display device layer DP-OEL may include a pixel definition layer PDL, a light emitting diode OEL, and an encapsulating layer TFE disposed on the light emitting diode OEL.

The light emitting diode OEL may include a first electrode EL1, a second electrode EL2 oppositely disposed to the first electrode EL1, and light emitting units EU-1, EU-2, EU-3, and EU-4 disposed between the first electrode EL1 and the second electrode EL2. The light emitting units EU-1, EU-2, EU-3, and EU-4 may be stacked in order between the first electrode EL1 and the second electrode EL2. The light emitting diode OEL according to an embodiment may further include charge generating layers CGL-1, CGL-2, and CGL-3 disposed between neighboring light emitting units EU-1, EU-2, EU-3, and EU-4. In an embodiment, the light emitting diode OEL may further include a capping layer CPL (not shown) disposed on the second electrode EL2. In the light emitting diode OEL of an embodiment, at least one of the light emitting units EU-1, EU-2, EU-3, and EU-4 may include the amine compound according to an embodiment. The light emitting diode OEL according to an embodiment will be explained in more detail with reference to the drawings.

The pixel definition layer PDL may define the luminous areas PXA-B, PXA-G, and PXA-R. By the pixel definition layer PDL, the luminous areas PXA-B, PXA-G, and PXA-R and the non-luminous area NPXA may be distinguished. Each of the luminous areas PXA-B, PXA-G, and PXA-R is an area separated by the pixel definition layer PDL and may overlap an opening portion OH defined in the pixel definition layer PDL. The non-luminous area NPXA may be an area between neighboring luminous areas PXA-B, PXA-G, and PXA-R, and may correspond to the pixel definition layer PDL. In the description, each of the luminous areas PXA-B, PXA-G, and PXA-R corresponds to a pixel.

Referring to FIG. 8, in the display device DD-1 of an embodiment, each of the light emitting units EU-1, EU-2, EU-3, and EU-4 included in the light emitting diode OEL may be provided as a common layer for all of the luminous areas PXA-B, PXA-G and PXA-R and the non-luminous area NPXA. For example, a portion of each of the light emitting units EU-1, EU-2, EU-3, and EU-4 included in the light emitting diode OEL may be disposed on the pixel definition layer PDL, and the parts of the light emitting units EU-1, EU-2, EU-3, and EU-4 disposed in the luminous areas PXA-B, PXA-G and PXA-R may be connected with each other on the pixel definition layer PDL to form common layers as one-body types. Accordingly, functional layers and emission layers disposed in the light emitting units EU-1, EU-2, EU-3, and EU-4 may also form common layers having one-body types in all of the luminous areas PXA-B, PXA-G, and PXA-R and the non-luminous area NPXA. However, embodiments are not limited thereto. In another embodiment and different from FIG. 3, at least a portion of the light emitting units EU-1, EU-2, EU-3, and EU-4 may be patterned in the opening portions OH defined in the pixel definition layer PDL and provided. At least a portion of the light emitting units EU-1, EU-2, EU-3, and EU-4, or at least a portion of the functional layers and emission layers, included in the light emitting units EU-1, EU-2, EU-3, and EU-4 may be patterned by an ink jet printing method and provided in the opening portion OH of the pixel definition layer PDL which overlaps the luminous areas PXA-B, PXA-G, and PXA-R.

The display panel DP according to an embodiment may include an encapsulating layer TFE covering the light emitting diode OEL. The encapsulating layer TFE may encapsulate the light emitting diode OEL. The encapsulating layer TFE may be disposed on the light emitting diode OEL and may be disposed to fill between the light emitting diode OEL and the light controlling layer CCL.

The encapsulating layer TFE may be a thin film encapsulating layer. The encapsulating layer TFE may be one layer or a stack of multiple layers. The encapsulating layer TFE includes at least one insulating layer. The encapsulating layer TFE according to an embodiment may include at least one inorganic layer (hereinafter, encapsulating inorganic layer). The encapsulating layer TFE according to an embodiment may include at least one organic layer (hereinafter, encapsulating organic layer) and at least one encapsulating inorganic layer.

The light controlling layer CCL may be disposed on the display panel DP. The light controlling layer CCL may include a light converter. The light converter may include a quantum dot or a phosphor. The light converter may transform the wavelength of a provided light and emit the transformed light. For example, the light controlling layer CCL may be a layer including a quantum dot or a layer including a phosphor.

The light controlling layer CCL may include light controlling parts CCP1, CCP2, and CCP3. The light controlling parts CCP1, CCP2 and CCP3 may be separated from one another.

Referring to FIG. 8, a partition pattern BMP may be disposed between the separated light controlling parts CCP1, CCP2, and CCP3, but embodiments are not limited thereto. In FIG. 8, the partition pattern BMP is shown not to overlap the light controlling parts CCP1, CCP2, and CCP3, but at least a portion of the edges of the light controlling parts CCP1, CCP2, and CCP3 may overlap the partition pattern BMP.

The light controlling layer CCL may include a first light controlling part CCP1 including a first quantum dot QD1 converting first color light provided from the light emitting diode OEL into third color light, a second light controlling part CCP2 including a second quantum dot QD2 converting first color light into second color light, and a third light controlling part CCP3 transmitting first color light.

In an embodiment, the first light controlling part CCP1 may provide red light which is the third color light, and the second light controlling part CCP2 may provide green light which is the second color light. The third color controlling part CCP3 may transmit and provide blue light which is the first color light provided from the light emitting diode OEL. For example, the first quantum dot QD1 may be a red quantum dot, and the second quantum dot QD2 may be a green quantum dot.

The quantum dots QD1 and QD2 may be selected from a II-VI group compound, a III-V group compound, a I-III-VI group compound, a III-V group compound, a III-II-V group compound, a IV-VI group compound, a IV group element, a IV group compound, and combinations thereof.

The II-VI group compound may be selected from the group consisting of: a binary compound selected from the group consisting of CdSe, CdTe, CdS, ZnS, ZnSe, ZnTe, ZnO, HgS, HgSe, HgTe, MgSe, MgS, and mixtures thereof, a ternary compound selected from the group consisting of CdSeS, CdSeTe, CdSTe, ZnSeS, ZnSeTe, ZnSTe, HgSeS, HgSeTe, HgSTe, CdZnS, CdZnSe, CdZnTe, CdHgS, CdHgSe, CdHgTe, HgZnS, HgZnSe, HgZnTe, MgZnSe, MgZnS, and mixtures thereof, and a quaternary compound selected from the group consisting of HgZnTeS, CdZnSeS, CdZnSeTe, CdZnSTe, CdHgSeS, CdHgSeTe, CdHgSTe, HgZnSeS, HgZnSeTe, HgZnSTe, and mixtures thereof.

The III-V group compound may include a binary compound such as In₂S₃, and In₂Se₃, a ternary compound such as InGaS₃, and InGaSe₃, or any combinations thereof.

The I-III-VI group compound may be selected from a ternary compound selected from the group consisting of AgInS, AgInS₂, CuInS, CuInS₂, AgGaS₂, CuGaS₂, CuGaO₂, AgGaO₂, AgAlO₂ and mixtures thereof, or a quaternary compound such as AgInGaS₂, and CuInGaS₂.

The III-V group compound may be selected from the group consisting of a binary compound selected from the group consisting of GaN, GaP, GaAs, GaSb, AlN, AlP, AlAs, AlSb, InN, InP, InAs, InSb, and mixtures thereof, a ternary compound selected from the group consisting of GaNP, GaNAs, GaNSb, GaPAs, GaPSb, AlNP, AlNAs, AlNSb, AlPAs, AlPSb, InGaP, InAlP, InNP, InNAs, InNSb, InPAs, InPSb, and mixtures thereof, and a quaternary compound selected from the group consisting of GaAlNP, GaAlNAs, GaAlNSb, GaAlPAs, GaAlPSb, GaInNP, GaInNAs, GaInNSb, GaInPAs, GaInPSb, InAlNP, InAlNAs, InAlNSb, InAlPAs, InAlPSb, and mixtures thereof. In an embodiment, the III-V group compound may further include a II group metal. For example, InZnP, etc. may be selected as a III-II-V group compound.

The IV-VI group compound may be selected from the group consisting of a binary compound selected from the group consisting of SnS, SnSe, SnTe, PbS, PbSe, PbTe, and mixtures thereof, a ternary compound selected from the group consisting of SnSeS, SnSeTe, SnSTe, PbSeS, PbSeTe, PbSTe, SnPbS, SnPbSe, SnPbTe, and mixtures thereof, and a quaternary compound selected from the group consisting of SnPbSSe, SnPbSeTe, SnPbSTe, and mixtures thereof. The IV group element may be selected from the group consisting of Si, Ge, and a mixture thereof. The IV group compound may be a binary compound selected from the group consisting of SiC, SiGe, and a mixture thereof.

A binary compound, a ternary compound, or a quaternary compound may be present in a quantum dot at a uniform concentration or may be present at a partially different concentration distribution state. In an embodiment, the quantum dot may have a core/shell structure in which one quantum dot surrounds another quantum dot. An interface of the core and the shell may have a concentration gradient in which the concentration of a material that is present in the shell decreases toward the core.

In embodiments, the quantum dots QD1 and QD2 may have the above-described core-shell structure including a core including a nanocrystal and a shell surrounding the core. The shell of the quantum dot may serve as a protection layer for preventing the chemical deformation of the core to maintain semiconductor properties and/or may serve as a charging layer for imparting the quantum dot with electrophoretic properties. The shell may be a single layer or a multilayer. Examples of the shell of the quantum dot may include a metal oxide, a non-metal oxide, a semiconductor compound, or combinations thereof.

For example, the metal or non-metal oxide may include a binary compound such as SiO₂, Al₂O₃, TiO₂, ZnO, MnO, Mn₂O₃, Mn₃O₄, CuO, FeO, Fe₂O₃, Fe₃O₄, CoO, CO₃O₄ and NiO, or a ternary compound such as MgAl₂O₄, CoFe₂O₄, NiFe₂O₄ and CoMn₂O₄, but embodiments are not limited thereto.

The semiconductor compound may include CdS, CdSe, CdTe, ZnS, ZnSe, ZnTe, ZnSeS, ZnTeS, GaAs, GaP, GaSb, HgS, HgSe, HgTe, InAs, InP, InGaP, InSb, AlAs, AlP, AlSb, etc., but embodiments are not limited thereto.

The quantum dots QD1 and QD2 may have a full width at half maximum (FWHM) of an emission wavelength spectrum equal to or less than about 45 nm. For example, the quantum dots QD1 and QD2 may have a FWHM of an emission wavelength spectrum equal to or less than about 40 nm. For example, the quantum dots QD1 and QD2 may have a FWHM of an emission wavelength spectrum equal to or less than about 30 nm. Within these ranges, color purity or color reproducibility may be improved. Light emitted through a quantum dot may be emitted in all directions, so that light viewing angle properties may be improved.

The shape of the quantum dots QD1 and QD2 may be generally used shapes in the related art, without limitation. For example, the quantum dot may have a spherical shape, a pyramidal shape, a multi-arm shape, or a cubic shape, or the quantum dot may be in the form of a nanoparticle, a nanotube, a nanowire, a nanofiber, a nanoplate, etc.

The quantum dots QD1 and QD2 may control the color of light emitted according to a particle size thereof, and accordingly, the quantum dot may have various emission colors such as blue, red, and green.

The light controlling layer CCL may further include a scatterer SP. The first light controlling part CCP1 may include the first quantum dot QD1 and the scatterer SP, the second light controlling part CCP2 may include the second quantum dot QD2 and the scatterer SP, and the third light controlling part CCP3 may not include a quantum dot but may include the scatterer SP.

The scatterer SP may be an inorganic particle. For example, the scatterer SP may include at least one of TiO₂, ZnO, Al₂O₃, SiO₂, and hollow silica. The scatterer SP may include at least one of TiO₂, ZnO, Al₂O₃, SiO₂, and hollow silica, or may be a mixture of two or more materials selected among TiO₂, ZnO, Al₂O₃, SiO₂, and hollow silica.

Each of the first light controlling part CCP1, the second light controlling part CCP2, and the third light controlling part CCP3 may include base resins BR1, BR2, and BR3 dispersing the quantum dots QD1 and QD2 and the scatterer SP. In an embodiment, the first light controlling part CCP1 may include the first quantum dot QD1 and the scatterer SP dispersed in the first base resin BR1, the second light controlling part CCP2 may include the second quantum dot QD2 and the scatterer SP dispersed in the second base resin BR2, and the third light controlling part CCP3 may include the scatterer particle SP dispersed in the third base resin BR3. The base resins BR1, BR2, and BR3 may each be a medium in which the quantum dots QD1 and QD2 and the scatterer SP are dispersed, and may be composed of various resin compositions which may be generally referred to as a binder. For example, the base resins BR1, BR2, and BR3 may be acrylic resins, urethane-based resins, silicone-based resins, epoxy-based resins, etc. The base resins BR1, BR2, and BR3 may be transparent resins. In an embodiment, the first base resin BR1, the second base resin BR2 and the third base resin BR3 may be the same as or different from each other.

The light controlling layer CCL may include a barrier layer BFL1. The barrier layer BFL1 may block the penetration of moisture and/or oxygen (hereinafter, will be referred to as “humidity/oxygen”). The barrier layer BFL1 may be disposed on the light controlling parts CCP1, CCP2, and CCP3 to block the exposure of the light controlling parts CCP1, CCP2, and CCP3 to humidity/oxygen. The barrier layer BFL1 may cover the light controlling parts CCP1, CCP2, and CCP3. The barrier layer BFL2 may be provided on the light controlling parts CCP1, CCP2, and CCP3.

The barrier layers BFL1 and BFL2 may each include at least one inorganic layer. In an embodiment, the barrier layers BFL1 and BFL2 may each be formed by including an inorganic material. For example, the barrier layers BFL1 and BFL2 may be formed by including silicon nitride, aluminum nitride, zirconium nitride, titanium nitride, hafnium nitride, tantalum nitride, silicon oxide, aluminum oxide, titanium oxide, tin oxide, cerium oxide, silicon oxynitride, or a metal thin film securing light transmittance. The barrier layers BFL1 and BFL2 may further include an organic layer. The barrier layers BFL1 and BFL2 may be composed of a single layer or of multiple layers.

In the display device DD-1 of an embodiment, the color filter layer CFL may be disposed on the light controlling layer CCL. In an embodiment, the color filter layer CFL may be disposed directly on the light controlling layer CCL. For example, the barrier layer BFL2 may be omitted.

The color filter layer CFL may include filters CF1, CF2, and CF3. The color filter layer CFL may include a first filter CF1 transmitting third color light, a second filter CF2 transmitting second color light, and a third filter CF3 transmitting first color light. For example, the first filter CF1 may be a red filter, the second filter CF2 may be a green filter, and the third filter CF3 may be a blue filter. Each of the filters CF1, CF2, and CF3 may include a polymer photosensitive resin and a pigment or dye. The first filter CF1 may include a red pigment or dye, the second filter CF2 may include a green pigment or dye, and the third filter CF3 may include a blue pigment or dye. However, embodiments are not limited thereto, and the third filter CF3 may not include a pigment or dye. The third filter CF3 may include a polymer photosensitive resin and may not include a pigment or dye. The third filter CF3 may be transparent. The third filter CF3 may be formed of a transparent photosensitive resin.

In an embodiment, the first filter CF1 and the second filter CF2 may each be a yellow filter. The first filter CF1 and the second filter CF2 may be provided in one body without distinction. The first to third filters CF1, CF2, and CF3 may be provided corresponding to a red luminous area PXA-R, a green luminous area PXA-G and a blue luminous area PXA-B, respectively.

Although not shown, the color filter layer CFL may include a light blocking part (not shown). The color filter layer CFL may include the light blocking part (not shown) disposed to overlap the boundaries of neighboring filters CF1, CF2, and CF3. The light blocking part (not shown) may be a black matrix. The light blocking part (not shown) may be formed of an organic light blocking material or of an inorganic light blocking material, including a black pigment or black dye. The light blocking part (not shown) may separate the boundaries among adjacent filters CF1, CF2, and CF3. In an embodiment, the light blocking part (not shown) may be formed as a blue filter.

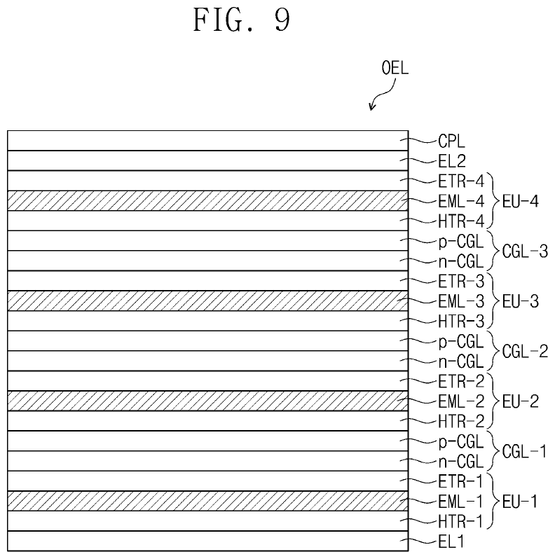

FIG. 9 is a schematic cross-sectional view showing a light emitting diode according to an embodiment. In FIG. 9, shown is an embodiment of a light emitting diode OEL included in the display device DD-1 of an embodiment, shown in FIG. 8. The light emitting diode OEL of an embodiment may include oppositely disposed first electrode EL1 and second electrode EL2, and first to fourth light emitting units EU-1, EU-2, EU-3, and EU-4 stacked in order between the first electrode EL1 and the second electrode EL2.

Referring to FIG. 9, each of the light emitting units EU-1, EU-2, EU-3, and EU-4 in the light emitting diode OEL may respectively include emission layers EML-1, EML-2, EML-3 and EML-4, hole transport regions HTR-1, HTR-2, HTR-3, and HTR-4 respectively disposed under the emission layers EML-1, EML-2, EML-3, and EML-4, and electron transport regions ETR-1, ETR-2, ETR-3, and ETR-4 respectively disposed on the emission layers EML-1, EML-2, EML-3, and EML-4. For example, the light emitting diode OEL included in the display device DD-1 of an embodiment may be a light emitting diode having a tandem structure including multiple emission layers.

The light emitting diode OEL of an embodiment may emit light in a direction from the first electrode EL1 to the second electrode EL2. In the structure of the light emitting diode OEL of an embodiment, the hole transport regions HTR-1, HTR-2, HTR-3, and HTR-4 may be respectively disposed under the emission layers EMLL-1, EML-2, EML-3, and EML-4, and the electron transport regions ETR-1, ETR-2, ETR-3, and ETR-4 may be respectively disposed on the emission layers EMLL-1, EML-2, EML-3, and EML-4, based on a direction of emitted light. However, embodiments are not limited thereto, and the light emitting diode OEL may have an inverted diode structure wherein the electron transport regions ETR-1, ETR-2, ETR-3, and ETR-4 may be respectively disposed under the emission layers EMLL-1, EMLL-2, EMLL-3, and EMLL-4, and the hole transport regions HTR-1, HTR-2, HTR-3, and HTR-4 may be respectively disposed on the emission layers EMLL-1, EMLL-2, EML-3, and EMLL-4.

In the light emitting diode OEL of an embodiment, at least one of the light emitting units EU-1, EU-2, EU-3, and EU-4 may be a blue light emitting unit which emits blue light. In an embodiment, at least one of the light emitting units EU-1, EU-2, EU-3, and EU-4 may be a blue light emitting unit emitting blue light, and the remaining light emitting units may be blue light emitting units or light emitting units emitting light of other than blue.

In an embodiment, each of the first light emitting unit EU-1, the second light emitting unit EU-2, and the third light emitting unit EU-3 may emit light having a first wavelength in a range of about 430 nm to about 470 nm, and the fourth light emitting unit EU-4 may emit light having a second wavelength in a range of about 520 nm to about 600 nm. For example, each of the first light emitting unit EU-1, the second light emitting unit EU-2, and the third light emitting unit EU-3 may be a blue light emitting unit emitting blue light, and the fourth light emitting unit EU-4 may be a green light emitting unit emitting green light.

In an embodiment, light emitted from the light emitting units EU-1, EU-2, EU-3, and EU-4 may be all blue light. However, embodiments are not limited thereto, and the wavelength regions of light emitted from the light emitting units EU-1, EU-2, EU-3, and EU-4 may be different each other. For example, the light emitting diode OEL including the light emitting units EU-1, EU-2, EU-3, and EU-4 emitting light in different wavelength regions from each other, may emit white light.

In FIG. 9, the light emitting diode OEL according to an embodiment is shown to include four light emitting units, but embodiments are not limited thereto, and the light emitting diode OEL may include two or more light emitting units, and the number of light emitting units stacked may be two, three, five, or more.

Charge generating layers CGL1, CGL2, and CGL3 may be disposed between adjacent light emitting units EU-1, EU-2, EU-3, and EU-4.

The light emitting diode OEL according to an embodiment may include a first charge generating layer CGL-1 disposed between the first light emitting unit EU-1 and the second light emitting unit EU-2, a second charge generating layer CGL-2 disposed between the second light emitting unit EU-2 and the third light emitting unit EU-3, and a third charge generating layer CGL-3 disposed between the third light emitting unit EU-3 and the fourth light emitting unit EU-4.

If a voltage is applied to the charge generating layers CGL-1, CGL-2, and CGL-3, a complex may be formed through an oxidation-reduction reaction to produce charges (electrons and holes). The charge generating layers CGL-1, CGL-2, and CGL-3 may provide the adjacent light emitting units EU-1, EU-2, EU-3, and EU-4 with the charges produced. The charge generating layers CGL-1, CGL-2, and CGL-3 may improve the efficiency of current produced in each of the adjacent light emitting units EU-1, EU-2, EU-3, and EU-4, and may control charge balance between adjacent light emitting units EU-1, EU-2, EU-3, and EU-4.

The charge generating layers CGL-1, CGL-2, and CGL-3 may each independently include a p-type charge generating layer p-CGL and/or an n-type charge generating layer n-CGL. Each of the first to third charge generating layers CGL-1, CGL-2, and CGL-3 may have a stacked structure of the n-type charge generating layer n-CGL and/or the p-type charge generating layer p-CGL.

The n-type charge generating layer n-CGL may be a charge generating layer providing adjacent light emitting units EU-1, EU-2, EU-3, and EU-4 with electrons. The n-type charge generating layer n-CGL may include an n-dopant. The n-type charge generating layer n-CGL may be a layer of a base material doped with the n-dopant. The p-type charge generating layer p-CGL may be a charge generating layer providing adjacent light emitting units EU-1, EU-2, EU-3, and EU-4 with holes. The p-type charge generating layer p-CGL may include a p-dopant. The p-type charge generating layer p-CGL may be a layer of a base material doped with the p-dopant. Although not shown in the drawings, in an embodiment, a buffer layer may be further disposed between the n-type charge generating layer n-CGL and the p-type charge generating layer p-CGL.

Each of the charge generating layers CGL-1, CGL-2, and CGL-3 may include an n-type aryl amine-based material or a p-type metal oxide. For example, each of the charge generating layers CGL-1, CGL-2, and CGL-3 may include a charge generating compound consisting of an aryl amine-based organic compound, a carbazole-based compound, a metal, a metal oxide, a metal carbide, a metal fluoride, or mixtures thereof.

For example, the aryl amine-based organic compound may be N,N′-di(naphthalene-1-yl)-N,N′-diphenyl-benzidine (α-NPD), 4,4′,4″-tris[N(2-naphthyl)-N-phenylamino]-triphenylamine (2-TNATA), 4,4′4″-tris(N,N-diphenylamino)triphenylamine (TDATA), 4,4′,4″-[tris(3-methylphenyl)phenylamino] triphenylamine (m-TDATA), spiro-TAD, or spiro-NPB, the carbazole-based compound may be 4,4′-bis(carbazol-9-yl)biphenyl (CBP). For example, the metal may be cesium (Cs), molybdenum (Mo), vanadium (V), titanium (Ti), tungsten (W), barium (Ba), or lithium (Li). For example, the metal oxide, the metal carbide, and the metal fluoride, may be Re₂O₇, MoO₃, V₂O₅, WO₃, TiO₂, Cs₂CO₃, BaF, LiF, or CsF.

In the light emitting diode OEL according to an embodiment, the first electrode EL1 has conductivity. The first electrode EL1 may be formed of a metal alloy or a conductive compound. The first electrode EL1 may be an anode. For example, the first electrode EL1 may be a pixel electrode. The first electrode EL1, may be the same as the first electrode EL1, as explained with reference to FIG. 4.

The light emitting units EU-1, EU-2, EU-3, and EU-4 may be disposed on the first electrode EL1 in order. The hole transport regions HTR-1, HTR-2, HTR-3, and HTR-4 included in the light emitting units EU-1, EU-2, EU-3, and EU-4 may each independently be a single layer formed of a single material, or a single layer formed of different materials, or have a structure having multiple layers formed of different materials.

Each of the hole transport regions HTR-1, HTR-2, HTR-3, and HTR-4 may be formed using various methods such as a vacuum deposition method, a spin coating method, a cast method, a Langmuir-Blodgett (LB) method, an inkjet printing method, a laser printing method, and a laser induced thermal imaging (LITI) method.

The light emitting diode OEL of an embodiment may include the amine compound represented by Formula 1 in at least one of the hole transport regions HTR-1, HTR-2, HTR-3, and HTR-4 included in the light emitting units EU-1, EU-2, EU-3, and EU-4. At least one of the hole transport regions HTR-1, HTR-2, HTR-3, and HTR-4 may include any amine compound represented by Formula 1, or mixtures of amine compounds represented by Formula 1.

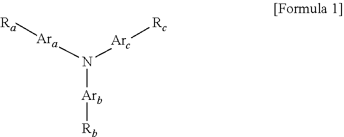

For the amine compound represented by Formula 1, the same explanation on the above-described amine compound according to an embodiment may be applied.

The amine compound represented by Formula 1 may be represented by any one of Formula 1A to Formula 1C.

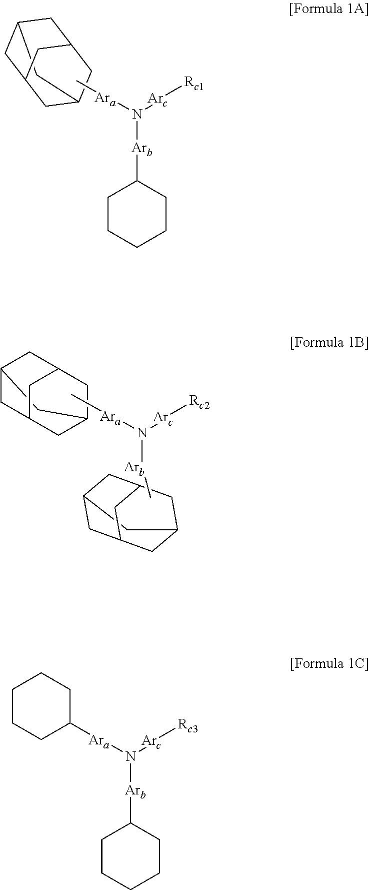

In Formula 1A, R_{c1} may be a hydrogen atom, a deuterium atom, a halogen atom, a cyano group, a substituted or unsubstituted oxy group, a substituted or unsubstituted thio group, a substituted or unsubstituted amine group, or a substituted or unsubstituted alkyl group of 1 to 20 carbon atoms. In Formula 1B and Formula 1C, R_{c2} and R_{c3} may be each independently a hydrogen atom, a deuterium atom, a halogen atom, a cyano group, a substituted or unsubstituted oxy group, a substituted or unsubstituted thio group, a substituted or unsubstituted amine group, a substituted or unsubstituted alkyl group of 1 to 20 carbon atoms, an adamantyl group, or a cyclohexyl group. In Formula 1A to Formula 1C, Arₐ to Ar_{c} are each the same as defined in Formula 1.

In an embodiment, at least one of the hole transport regions HTR-1, HTR-2, HTR-3, and HTR-4 may include at least one of the amine compounds selected from Compound Group 1.

The amine compound according to an embodiment, represented by Formula 1, may be a hole transport material having low refraction. The hole transport regions HTR-1, HTR-2, HTR-3, and HTR-4 including the amine compound according to an embodiment, represented by Formula 1, may each independently have a refractive index in a range of about 1.30 to about 1.80, with respect to light at a wavelength of about 460 nm. The refractive index of the hole transport regions HTR-1, HTR-2, HTR-3, and HTR-4 including the amine compound according to an embodiment may be smaller than the refractive index of adjacently disposed emission layers EMLL-1, EML-2, EML-3, and EML-4. The difference of the refractive indexes with respect to light at a wavelength of about 460 nm between the hole transport regions HTR-1, HTR-2, HTR-3, and HTR-4 and the adjacently disposed emission layers EML-1, EML-2, EML-3, and EML-4, may be greater than about 0.1.

The light emitting diode OEL of an embodiment includes a hole transport region including the amine compound according to an embodiment, and may show high light extraction efficiency properties and improved emission efficiency properties.

The remaining hole transport regions not including the amine compound represented by Formula 1, among the hole transport regions HTR-1, HTR-2, HTR-3, and HTR-4, may include a hole transport material of the related art. For example, the remaining hole transport regions not including the amine compound of an embodiment may further include a phthalocyanine compound such as copper phthalocyanine, N¹,N¹′-([1,1′-biphenyl]-4,4′-diyl)bis(N¹-phenyl-N⁴,N⁴-di-m-tolylbenzene-1,4-diamine) (DNTPD), 4,4′,4″-[tris(3-methylphenyl)phenylamino] triphenylamine (m-MTDATA), 4,4′,4″-tris(N,N-diphenylamino)triphenylamine (TDATA), 4,4′,4″-tris[N(1-naphthyl)-N-phenylamino]-triphenylamine (1-TNATA), 4,4′,4″-tris[N(2-naphthyl)-N-phenylamino]-triphenylamine (2-TNATA), poly(3,4-ethylenedioxythiophene)/poly(4-styrenesulfonate) (PEDOT/PSS), polyaniline/dodecylbenzenesulfonic acid (PANI/DBSA), polyaniline/camphor sulfonic acid (PANI/CSA), polyaniline/poly(4-styrenesulfonate) (PANI/PSS), N,N′-di(1-naphthalene-1-yl)-N,N′-diphenyl-benzidine (NPB or NPD, α-NPD), triphenylamine-containing polyetherketone (TPAPEK), 4-isopropyl-4′-methyldiphenyliodonium [tetrakis(pentafluorophenyl)borate], and dipyrazino[2,3-f: 2′,3′-h] quinoxaline-2,3,6,7,10,11-hexacarbonitrile (HAT-CN).

The hole transport regions HTR-1, HTR-2, HTR-3, and HTR-4 may include carbazole derivatives such as N-phenyl carbazole and polyvinyl carbazole, fluorene-based derivatives, triphenylamine-based derivatives such as 4,4′,4″-tris(N-carbazolyl)triphenylamine (TCTA), N,N′-bis(3-methylphenyl)-N,N′-diphenyl-[1,1′-biphenyl]-4,4′-diamine (TPD), N,N′-di(naphthalene-1-yl)-N,N′-diphenyl-benzidine (NPB), 4,4′-cyclohexylidene bis[N,N-bis(4-methylphenyl)benzeneamine (TAPC), 4,4′-bis[N,N′-(3-tolyl)amino]-3,3′-dimethylbiphenyl (HMTPD), 1,3-bis(N-carbazolyl)benzene (mCP), etc.

The hole transport regions HTR-1, HTR-2, HTR-3, and HTR-4 may include 9-(4-tert-butylphenyl)-3,6-bis(triphenylsilyl)-9H-carbazole (CzSi), 9-phenyl-9H-3,9′-bicarbazole (CCP), or 1,3-bis(1,8-dimethyl-9H-carbazol-9-yl)benzene (mDCP), etc.

The hole transport regions HTR-1, HTR-2, HTR-3, and HTR-4 may further include a charge generating material to increase conductivity in addition to the above-described materials.

The charge generating material may be dispersed uniformly or non-uniformly in the hole transport regions HTR-1, HTR-2, HTR-3, and HTR-4. The charge generating material may be, for example, a p-dopant. The p-dopant may include at least one of metal halide compounds, quinone derivatives, metal oxides, or cyano group-containing compounds, without limitation.

For example, the p-dopant may include metal halide compounds such as CuI and RbI, quinone derivatives such as tetracyanoquinodimethane (TCNQ) and 2,3,5,6-tetrafluoro-7,7′,8,8-tetracyanoquinodimethane (F4-TCNQ), metal oxides such as tungsten oxide and molybdenum oxide, cyano group-containing compounds such as dipyrazino[2,3-f. 2′,3′-h] quinoxaline-2,3,6,7,10,11-hexacarbonitrile (HATCN) and 4-[[2,3-bis[cyano-(4-cyano-2,3,5,6-tetrafluorophenyl)methylidene]cyclopropylidene]-cyanomethyl]-2,3,5,6-tetrafluorobenzonitrile (NDP9), etc., but embodiments are not limited thereto.

In the light emitting diode OEL according to an embodiment, the hole transport region of the light emitting unit adjacent to the second electrode EL2 among the multiple light emitting units may include the amine compound represented by Formula 1. In an embodiment, the amine compound represented by Formula 1 may be included in the light emitting unit adjacent to the second electrode EL2, and the amine compound represented by Formula 1 may be additionally included in at least one light emitting unit among the remaining light emitting units. For example, in an embodiment shown in FIG. 9, the amine compound represented by Formula 1 may be included in the hole transport region HTR-4 of the fourth light emitting unit EU-4, and hole transport materials of the related art may be included in the remaining hole transport regions HTR-1, HTR-2, and HTR-3, or the amine compound represented by Formula 1 may be included in the hole transport region HTR-4 of the fourth light emitting unit EU-4 and in the hole transport region HTR-1, HTR-2, or HTR-3 of at least one of the first to third light emitting units EU-1, EU-2, and EU-3.

The light emitting diode OEL of an embodiment may include the amine compound of an embodiment, having low refraction properties in the hole transport region HTR-4 disposed adjacent to the second electrode EL2, and may show high light extraction efficiency and excellent emission efficiency properties. The light emitting diode OEL of an embodiment may additionally include the amine compound of an embodiment, having low refraction properties in the remaining hole transport regions HTR-1, HTR-2, and HTR-3, and may show high light extraction efficiency and excellent emission efficiency properties.

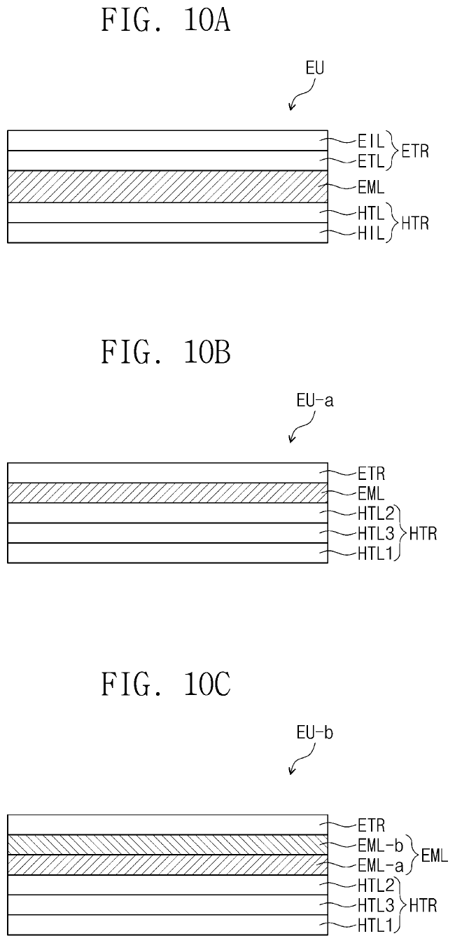

In the light emitting units EU-1, EU-2, EU-3, and EU-4 included in the light emitting diode OEL according to an embodiment, the hole transport regions HTR-1, HTR-2, HTR-3, and HTR-4 may each have a multilayer structure having multiple functional layers. In FIG. 10A, an embodiment of a light emitting unit included in the light emitting diode according to an embodiment is shown. The structure of the light emitting unit EU shown in FIG. 10A may be provided to at least one of the first to fourth light emitting units EU-1, EU-2, EU-3, and EU-4.

Referring to FIG. 10A, the hole transport region HTR may include a hole injection layer HIL and a hole transport layer HTL. The hole transport region HTR may further include at least one of a hole injection layer HIL, a hole transport layer HTL, a buffer layer (not shown), an emission auxiliary layer (not shown), and an electron blocking layer (not shown). In an embodiment, the hole transport region HTR may have a single layer structure formed of different materials, or may have a structure of hole injection layer HIL/hole transport layer HTL, hole injection layer HIL/hole transport layer HTL/buffer layer (not shown), hole injection layer HIL/buffer layer (not shown), or hole transport layer HTL/buffer layer (not shown), stacked in order, but embodiments are not limited thereto.

In an embodiment, the hole transport layer HTL of the hole transport region HTR may include the amine compound of an embodiment, but embodiments are not limited thereto.

In the light emitting diode OEL of an embodiment, at least one of the hole transport regions HTR-1, HTR-2, HTR-3, and HTR-4 may include multiple hole transport layers. In FIG. 10B and FIG. 10C, embodiments of a light emitting unit included in the light emitting diode according to an embodiment are shown. To at least one of the first to fourth light emitting units EU-1, EU-2, EU-3, and EU-4, shown in FIG. 9, the structure of the light emitting unit EU-a shown in FIG. 10B or the structure of the light emitting unit EU-b shown in FIG. 10C may be provided.

Referring to FIG. 10B and FIG. 10C, in the light emitting units EU-a and EU-b according to embodiments, the hole transport region HTR may include hole transport layers HTL1, HTL3, and HTL2. Referring to FIG. 10B and FIG. 10C, the hole transport region HTR may include a first hole transport layer HTL1, a second hole transport layer HTL2 disposed adjacent to an emission layer EML, and a third hole transport layer HTL3 disposed between the first hole transport layer HTL1 and the second hole transport layer HTL2. The third hole transport layer HTL3 has a third refractive index which may be greater than each of a first refractive index of the first hole transport layer HTL1 and a second refractive index of the second hole transport layer HTL2. In an embodiment, the hole transport region HTR may include hole transport layers HTL1, HTL3, and HTL2 disposed in the order of a low refractive hole transport layer/a high refractive hole transport layer/a low refractive hole transport layer, in a thickness direction.

The first hole transport layer HTL1 and the second hole transport layer HTL2 may each independently include the amine compound of an embodiment, represented by Formula 1. Each of the first hole transport layer HTL1 and the second hole transport layer HTL2, including the amine compound of an embodiment, may each have a refractive index in a range of about 1.30 to about 1.80, with respect to light at a wavelength of about 460 nm.

In an embodiment, the third hole transport layer HTL3 may include the compound represented by Formula 2 below. The third hole transport layer HTL3 including the compound represented by Formula 2 may have a refractive index in a range of about 1.85 to about 2.40, with respect to light at a wavelength of about 460 nm.

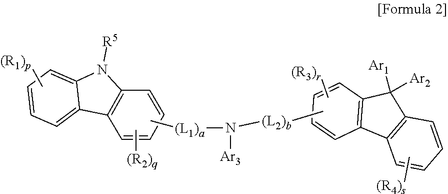

With respect to Formula 2, the same explanation on the above-described Formula 2 may be applied.

The compound for the third hole transport layer HTL3, represented by Formula 2 may be selected from Compound Group 2 above. The third hole transport layer HTL3 of the light emitting units EU-a and EU-b according to embodiments may include at least one compound selected from Compound Group 2.

Referring to FIG. 9, FIG. 10B, and FIG. 10C, at least one of the hole transport regions HTR-1, HTR-2, HTR-3, and HTR-4 in the light emitting units EU-1, EU-2, EU-3, and EU-4 included in the light emitting diode OEL of an embodiment may include three hole transport layers HTL1, HTL2, and HTL3. The light emitting diode OEL of an embodiment may include hole transport layers of first hole transport layer HTL1/third hole transport layer HTL3/second hole transport layer HTL2 stacked in the order and may show excellent emission efficiency properties. In an embodiment, the refractive indexes of the first hole transport layer HTL1 and the second hole transport layer HTL2 may be smaller than the refractive index of the third hole transport layer HTL3, and a difference of the refractive index of the third hole transport layer HTL3 from the refractive index of the first hole transport layer HTL1 and a difference of the refractive of the third hole transport layer HTL3 from the refractive index of the second hole transport layer HTL2 may each independently be greater than about 0.1.

In the light emitting diode OEL of an embodiment shown in FIG. 9, the remaining hole transport regions not including the amine compound represented by Formula 1 may include the compound represented by Formula 2.

In the light emitting diode OEL of an embodiment, shown in FIG. 9, emission layers EMLL-1, EMLL-2, EMLL-3, and EMLL-4 may be disposed on hole transport regions HTR-1, HTR-2, HTR-3, and HTR-4, respectively. The emission layers EMLL-1, EMLL-2, EMLL-3, and EML-4 may each independently have a thickness, for example, in a range of about 100 Å to about 1,000 Å. For example, the emission layers EML-1, EMLL-2, EMLL-3, and EMLL-4 may each independently have a thickness in a range of about 100 Å to about 300 Å. The emission layers EML-1, EML-2, EML-3, and EML-4 may each independently be a layer formed of a single material, or a layer formed of different materials, or a structure having multiple layers including different materials.

The emission layers EML-1, EML-2, EML-3, and EML-4 may include a fluorescence emitting material or a phosphorescence emitting material. In an embodiment, the emission layers EML-1, EML-2, EML-3, and EML-4 may include quantum dots. With respect to the quantum dot, the above-described explanation on the quantum dot may be applied.

In the light emitting diode OEL of an embodiment, the emission layers EML-1, EML-2, EML-3, and EML-4 may include anthracene derivatives, pyrene derivatives, fluoranthene derivatives, chrysene derivatives, dihydrobenzanthracene derivatives, or triphenylene derivatives. For example, the emission layer EML may include anthracene derivatives or pyrene derivatives. However, embodiments are not limited thereto, and the emission layer EML may include light emitting materials of the related art.

In the light emitting diode OEL of an embodiment, at least one of the emission layers EML-1, EML-2, EML-3, and EML-4 of the first to fourth light emitting units EU-1, EU-2, EU-3, and EU-4 may be a blue emission layer which emits blue light. For example, the emission layer EML-1 of the first light emitting unit EU-1, the emission layer EML-2 of the second light emitting unit EU-2, and the emission layer EML-3 of the third light emitting unit EU-3 may be blue emission layers. In the light emitting diode OEL of an embodiment, the emission layer EML-4 of the fourth light emitting unit EU-4 may be a green emission layer which emits green light.

For example, the light emitting diode OEL of an embodiment, may have a tandem structure including multiple emission layers EML-1, EML-2, and EML-3 emitting light having a first wavelength in a range of about 430 nm to about 470 nm, and an emission layer EML-4 emitting light having a second wavelength in a range of about 520 nm to about 600 nm.

In the light emitting diode OEL of an embodiment, at least one of the emission layers EML-1, EML-2, EML-3, and EML-4 in the light emitting units EU-1, EU-2, EU-3, and EU-4, may include multiple sub-emission layers. For example, at least one of the emission layers EML-1, EML-2, EML-3, and EML-4 may have a double layer structure.

Referring to FIG. 10C showing the light emitting unit EU-b according to an embodiment, the emission layer EML may include a first sub-emission layer EML-a and a second sub-emission layer EML-b. In an embodiment, at least one of the emission layers EML-1, EML-2, EML-3, and EML-4 shown in FIG. 9, may have a double layer structure of the first sub-emission layer EML-a and the second sub-emission layer EML-b. In an embodiment, at least one of the first to third light emitting units EU-1, EU-2, and EU-3 may include an emission layer EML including the first sub-emission layer EML-a and the second sub-emission layer EML-b. For example, each of the first to third light emitting units EU-1, EU-2 and EU-3 may include an emission layer EML including the first sub-emission layer EML-a and the second sub-emission layer EML-b.

The first sub-emission layer EML-a may include a first host and a first dopant emitting light having a first wavelength, and the second sub-emission layer EML-b may include a second host and a second dopant emitting light having a first wavelength.

In an embodiment, the first host of the first sub-emission layer EML-a and the second host of the second sub-emission layer EML-b may be different host materials from each other. At least one of the first host and the second host may be a hole transport host, and the remaining one may be an electron transport host. The hole transport host may be a material including a hole transport moiety in a molecular structure. The electron transport host may be a material including an electron transport moiety in a molecular structure. The first sub-emission layer EML-a and the second sub-emission layer EML-b may contact each other without any additional layer disposed therebetween.

The first dopant of the first sub-emission layer EML-a and the second dopant of the second sub-emission layer EML-b may be the same. Each of the first dopant and the second dopant may be a blue fluorescence dopant. However, embodiments are not limited thereto, and the first dopant and the second dopant may be different from each other.

In an embodiment, the emission layer EML may include any hole transport host selected from H1-1 to H1-7 below. For example, in an embodiment, the first sub-emission layer EML-a may include any hole transport host selected from H1-1 to H1-7 below. However, the hole transport host material is not limited to H1-1 to H1-7.

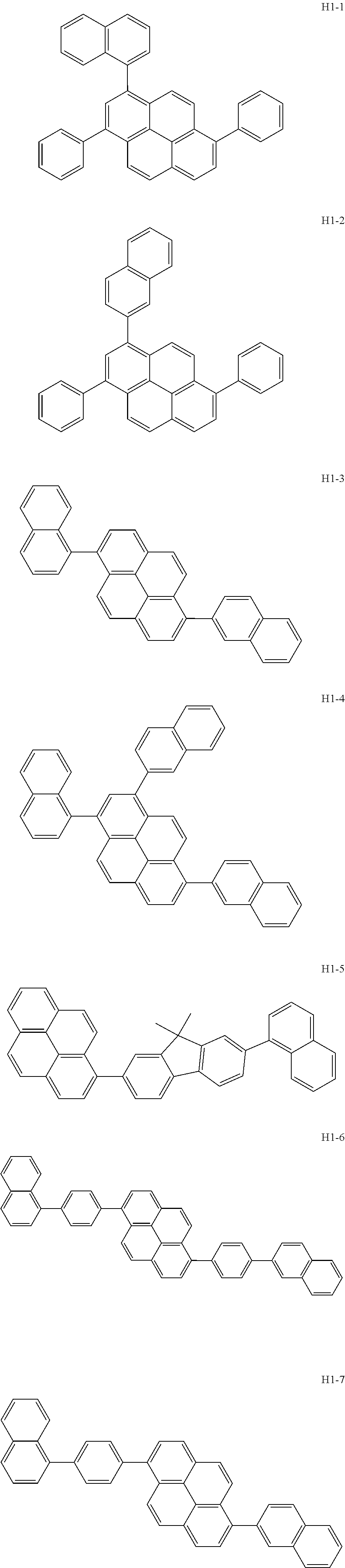

In an embodiment, the emission layer EML may include any one electron transport host selected from H2-1 to H2-7 below. For example, the second sub-emission layer EML-b may include any electron transport host selected from H2-1 to H2-7 below. However, the electron transport host material is not limited to H2-1 to H2-7.

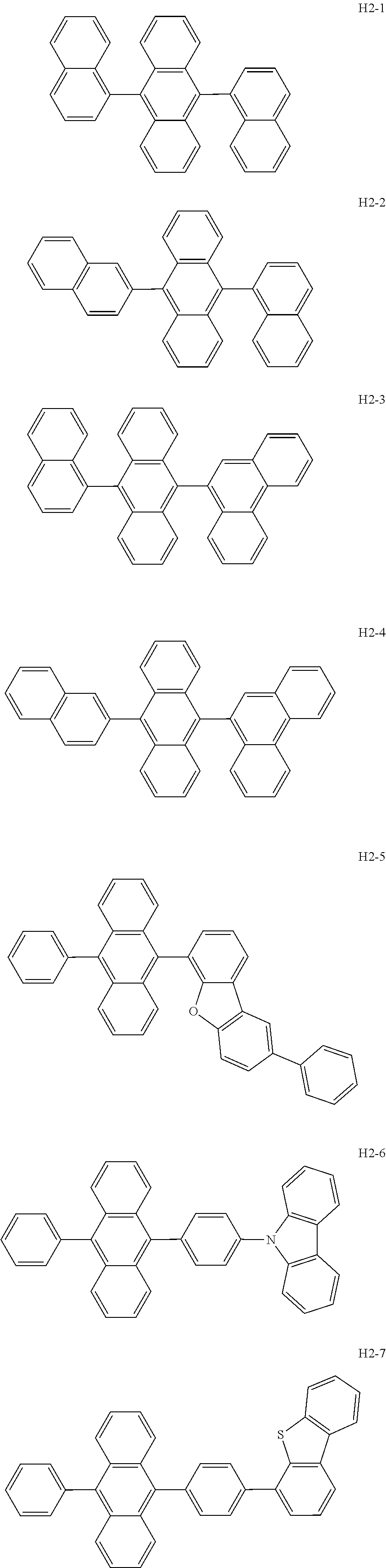

In the light emitting diode OEL according to an embodiment, at least one of the emission layers E-L-1, E-L-2, E-L-3, and EML-4 may include any dopant selected from FD1 to FD32 below. For example, in an embodiment, each of the emission layers E-L-1, E-L-2, and E-L-3 of the first to third light emitting units EU-1, EU-2, and EU-3 may include any dopant selected from FD1 to FD32 below. Each of the emission layers E-L-1, E-L-2, and E-L-3 of the first to third light emitting units EU-1, EU-2, and EU-3 may include any dopant selected from FD1 to FD32 below and emit blue light. However, the dopant material included in the emission layers EML-1, EML-2, and EML-3 of the first to third light emitting units EU-1, EU-2, and EU-3 is not limited to FD1 to FD32.

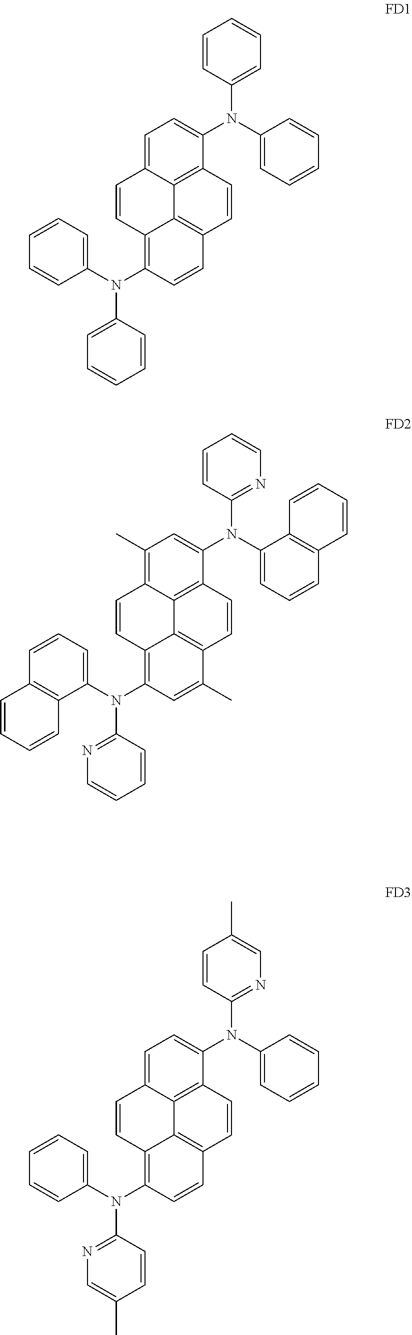 [SMILES: c1ccc(N(c2ccccc2)c2ccc3ccc4c(N(c5ccccc5)c5ccccc5)ccc5ccc2c3c54)cc1 ; Cc1cc(N(c2ccccn2)c2cccc3ccccc23)c2ccc3c(C)cc(N(c4ccccn4)c4cccc5ccccc45)c4ccc1c2c34 ; Cc1ccc(N(c2ccccc2)c2ccc3ccc4c(N(c5ccccc5)c5ccc(C)cn5)ccc5ccc2c3c54)nc1]

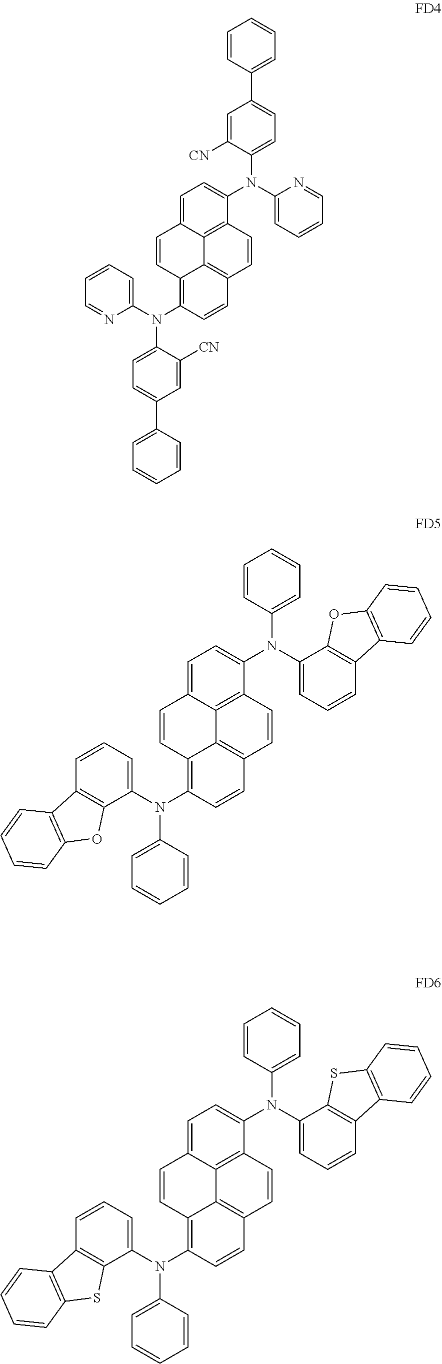

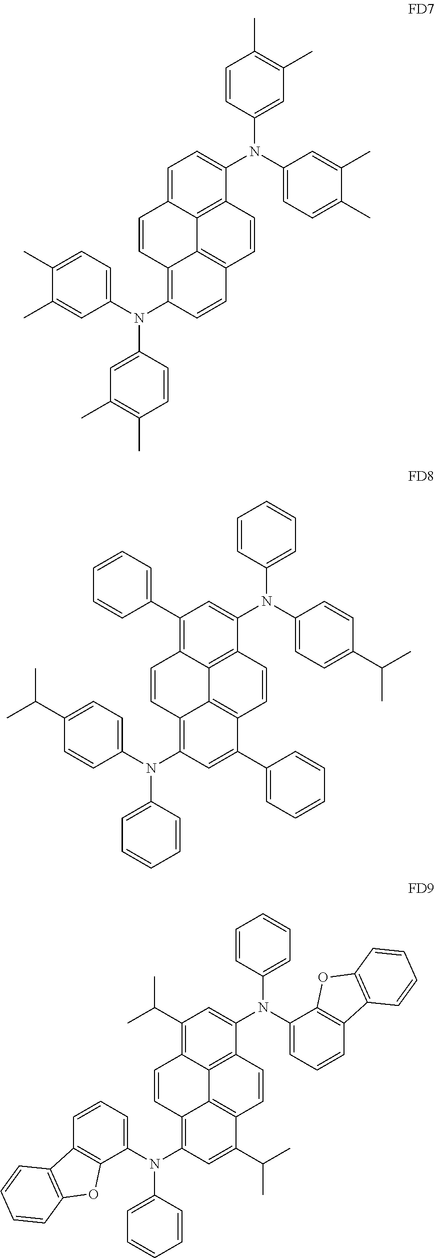

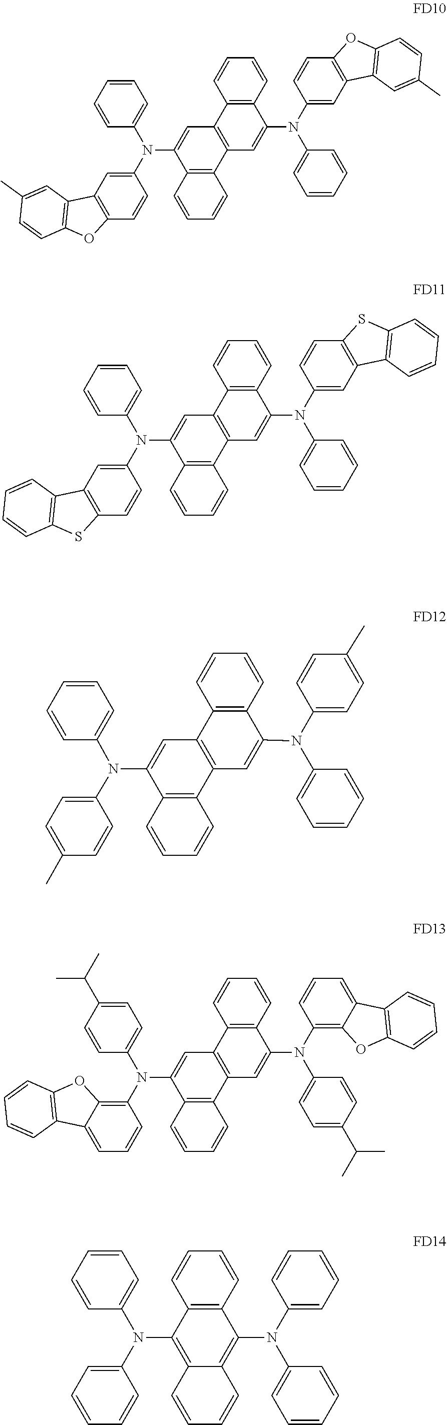

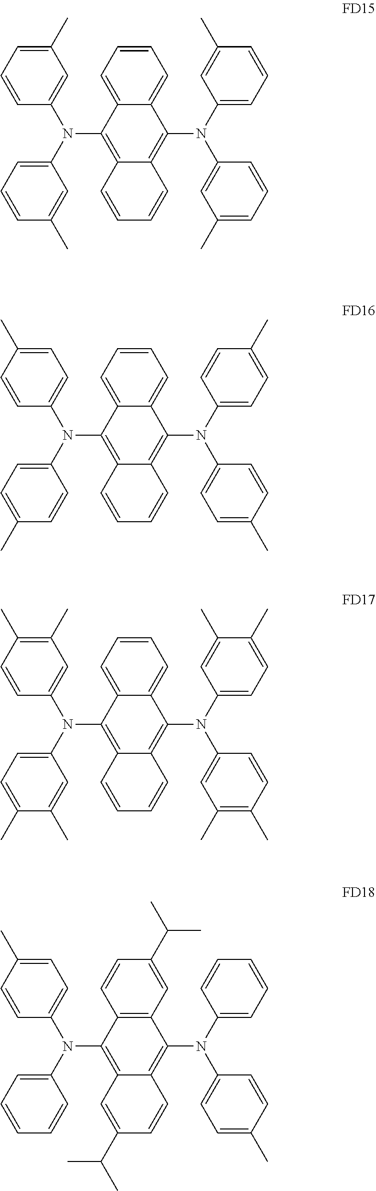 [SMILES: Cc1cccc(N(c2cccc(C)c2)c2c3ccccc3c(N(c3cccc(C)c3)c3cccc(C)c3)c3ccccc23)c1 ; Cc1ccc(N(c2ccc(C)cc2)c2c3ccccc3c(N(c3ccc(C)cc3)c3ccc(C)cc3)c3ccccc23)cc1 ; Cc1ccc(N(c2ccc(C)c(C)c2)c2c3ccccc3c(N(c3ccc(C)c(C)c3)c3ccc(C)c(C)c3)c3ccccc23)cc1C ; Cc1ccc(N(c2ccccc2)c2c3ccc(C(C)C)cc3c(N(c3ccccc3)c3ccc(C)cc3)c3ccc(C(C)C)cc23)cc1]

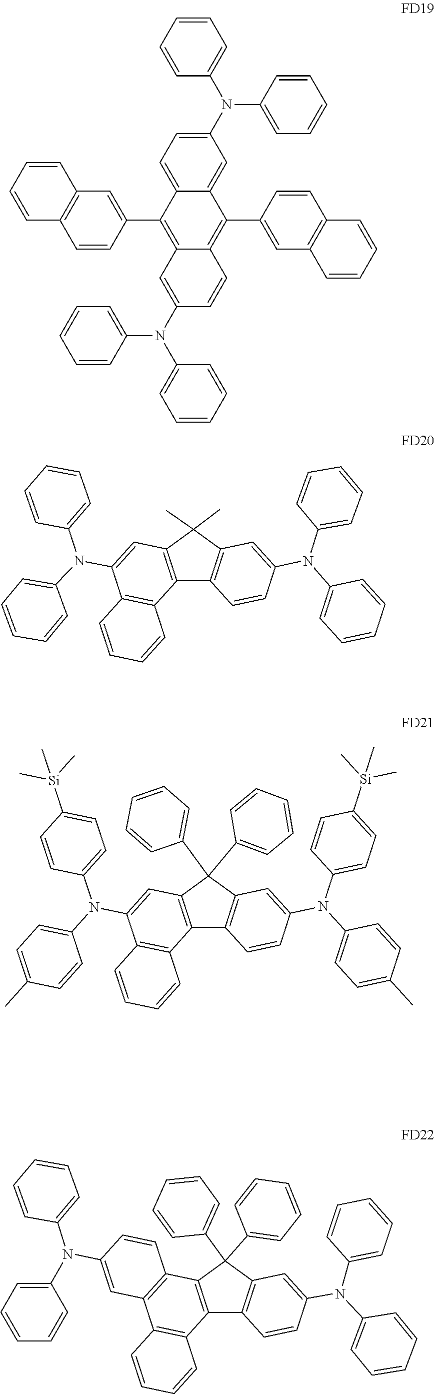

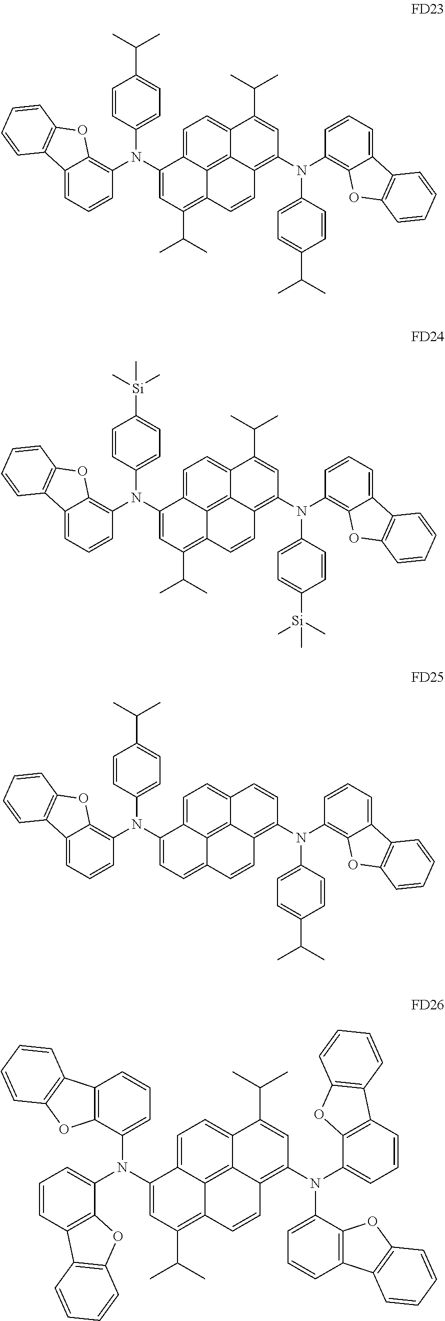 [SMILES: CC(C)c1ccc(N(c2cc(C(C)C)c3ccc4c(N(c5ccc(C(C)C)cc5)c5cccc6c5oc5ccccc56)cc(C(C)C)c5ccc2c3c54)c2cccc3c2oc2ccccc23)cc1 ; CC(C)c1cc(N(c2ccc([Si](C)(C)C)cc2)c2cccc3c2oc2ccccc23)c2ccc3c(C(C)C)cc(N(c4ccc([Si](C)(C)C)cc4)c4cccc5c4oc4ccccc45)c4ccc1c2c34 ; CC(C)c1ccc(N(c2ccc3ccc4c(N(c5ccc(C(C)C)cc5)c5cccc6c5oc5ccccc56)ccc5ccc2c3c54)c2cccc3c2oc2ccccc23)cc1 ; CC(C)c1cc(N(c2cccc3c2oc2ccccc23)c2cccc3c2oc2ccccc23)c2ccc3c(C(C)C)cc(N(c4cccc5c4oc4ccccc45)c4cccc5c4oc4ccccc45)c4ccc1c2c34]

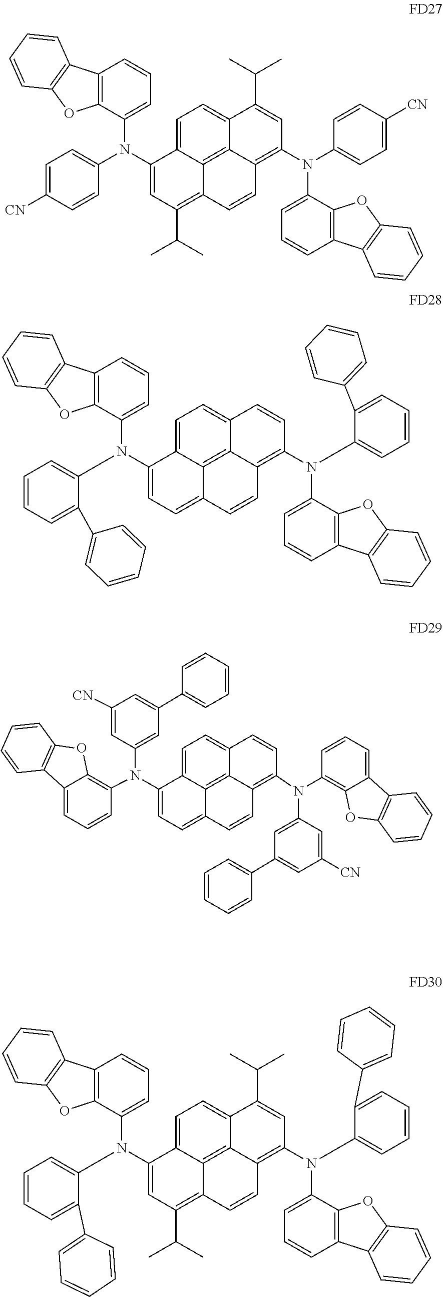

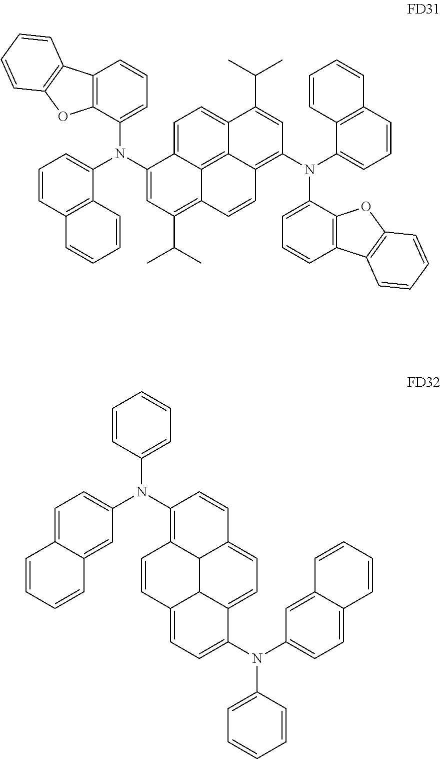 [SMILES: CC(C)c1cc(N(c2cccc3ccccc23)c2cccc3c2oc2ccccc23)c2ccc3c(C(C)C)cc(N(c4cccc5ccccc45)c4cccc5c4oc4ccccc45)c4ccc1c2c34 ; C1=CC2=C(N(c3ccccc3)c3ccc4ccccc4c3)C=CC3=CC=C4C(N(c5ccccc5)c5ccc6ccccc6c5)=CC=C1C4C32]

In the light emitting diode OEL according to an embodiment, shown in FIG. 9, the fourth light emitting unit EU-4 may include an emission layer EML-4 emitting light having a second wavelength. The light having the second wavelength may be light in a green wavelength region. In an embodiment, the second wavelength may be in a range about 520 nm to about 600 nm.

The emission layer EML-4 of the fourth light emitting unit EU-4 may have a single layer structure. For example, the emission layers EML-1, EML-2, and EML-3 of the first to third light emitting units EU-1, EU-2, and EU-3 emitting light having the first wavelength in a range of about 430 nm to about 470 nm may have a double layer structure as explained referring to FIG. 10C, and the emission layer EML-4 of the fourth light emitting unit EU-4 may have a single layer structure.

The emission layer EML-4 of the fourth light emitting unit EU-4 may have a structure of a single layer in which two or more different host materials are mixed. In an embodiment, the emission layer EML-4 of the fourth light emitting unit EU-4 has a mixed structure of a hole transport host material and an electron transport host material in one layer.

In an embodiment, the emission layer EML-4 of the fourth light emitting unit EU-4 may include a hole transport host, an electron transport host, and a third dopant. The emission layer EML-4 of the fourth light emitting unit EU-4 may be a mixed layer of a hole transport host and an electron transport host, doped with a third dopant emitting light having a second wavelength. In an embodiment, the hole transport host included in the emission layer EML-4 of the fourth light emitting unit EU-4 may be a material different from the first host. The electron transport host included in the emission layer EML-4 of the fourth light emitting unit EU-4 may be a material different from the second host. In an embodiment, the third dopant may be a phosphorescence dopant. For example, the third dopant may be a green phosphorescence dopant. However, embodiments are not limited thereto, and the emission layer EML-4 of the fourth light emitting unit EU-4 may include a fluorescence dopant.

In an embodiment, the hole transport host included in the emission layer of the light emitting unit emitting light having the second wavelength may include any host selected from H4-1 to H4-11 below. However, the second hole transport material included in the emission layer of the light emitting unit emitting light having the second wavelength is not limited to H4-1 to H4-11.

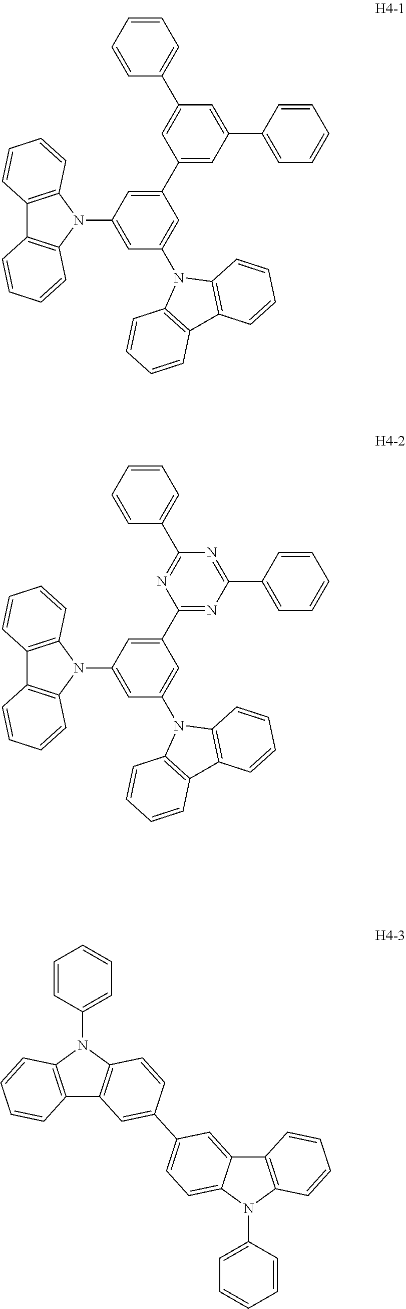 [SMILES: c1ccc(-c2cc(-c3ccccc3)cc(-c3cc(-n4c5ccccc5c5ccccc54)cc(-n4c5ccccc5c5ccccc54)c3)c2)cc1 ; c1ccc(-c2nc(-c3ccccc3)nc(-c3cc(-n4c5ccccc5c5ccccc54)cc(-n4c5ccccc5c5ccccc54)c3)n2)cc1 ; c1ccc(-n2c3ccccc3c3cc(-c4ccc5c(c4)c4ccccc4n5-c4ccccc4)ccc32)cc1]

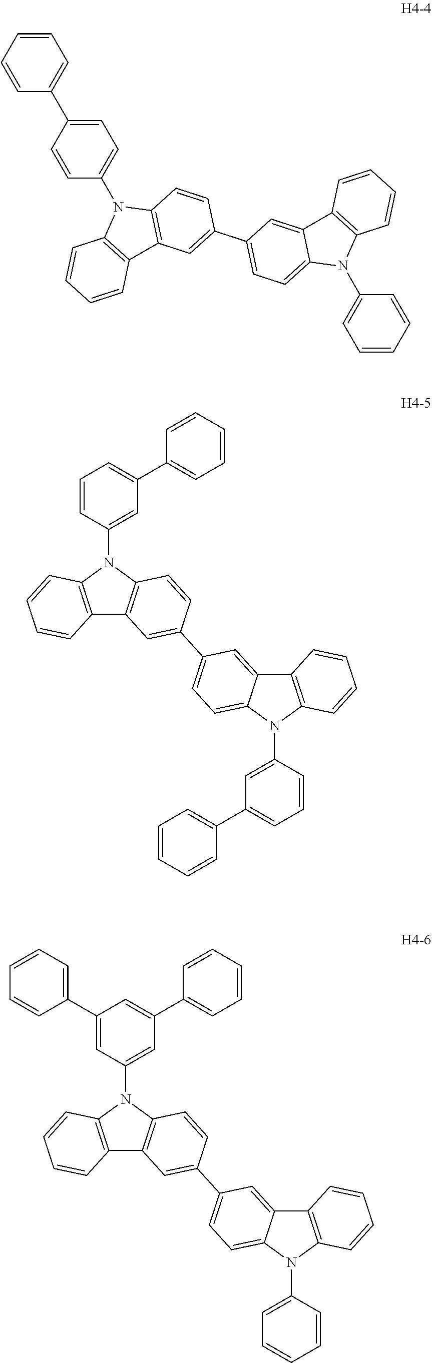 [SMILES: c1ccc(-c2ccc(-n3c4ccccc4c4cc(-c5ccc6c(c5)c5ccccc5n6-c5ccccc5)ccc43)cc2)cc1 ; c1ccc(-c2cccc(-n3c4ccccc4c4cc(-c5ccc6c(c5)c5ccccc5n6-c5cccc(-c6ccccc6)c5)ccc43)c2)cc1 ; c1ccc(-c2cc(-c3ccccc3)cc(-n3c4ccccc4c4cc(-c5ccc6c(c5)c5ccccc5n6-c5ccccc5)ccc43)c2)cc1]

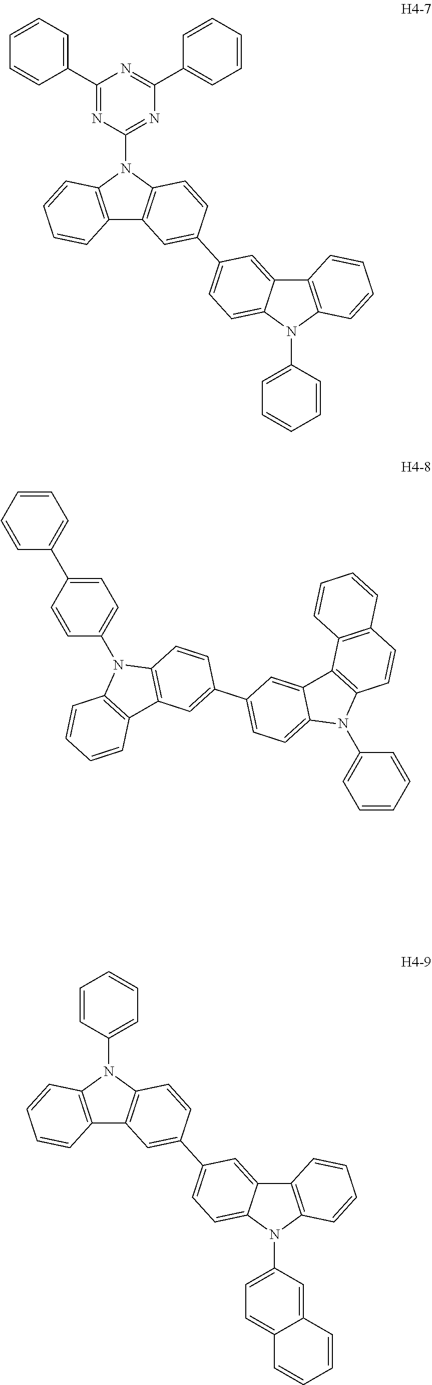 [SMILES: c1ccc(-c2nc(-c3ccccc3)nc(-n3c4ccccc4c4cc(-c5ccc6c(c5)c5ccccc5n6-c5ccccc5)ccc43)n2)cc1 ; c1ccc(-c2ccc(-n3c4ccccc4c4cc(-c5ccc6c(c5)c5c7ccccc7ccc5n6-c5ccccc5)ccc43)cc2)cc1 ; c1ccc(-n2c3ccccc3c3cc(-c4ccc5c(c4)c4ccccc4n5-c4ccc5ccccc5c4)ccc32)cc1]

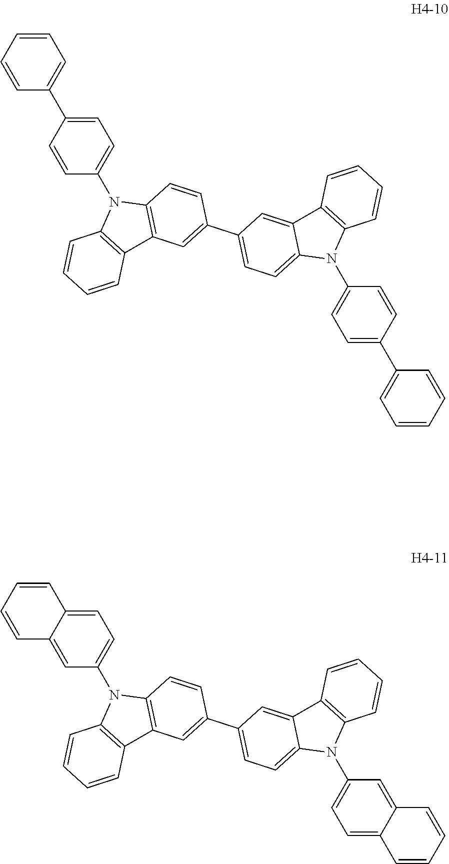 [SMILES: c1ccc(-c2ccc(-n3c4ccccc4c4cc(-c5ccc6c(c5)c5ccccc5n6-c5ccc(-c6ccccc6)cc5)ccc43)cc2)cc1 ; c1ccc2cc(-n3c4ccccc4c4cc(-c5ccc6c(c5)c5ccccc5n6-c5ccc6ccccc6c5)ccc43)ccc2c1]

In an embodiment, the electron transport host included in the emission layer of the light emitting unit emitting light with the second wavelength may include any host selected from H-3-1 to H-3-22 below. However, the electron transport host material is not limited to H3-1 to H-3-22.

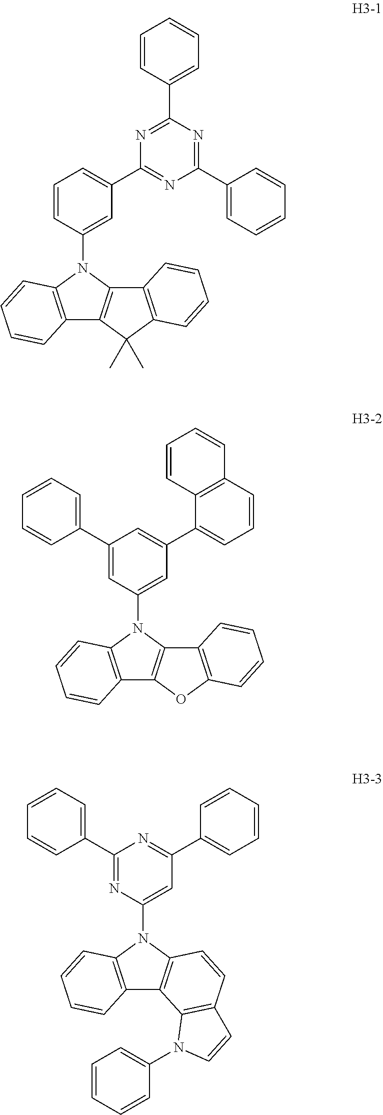 [SMILES: CC1(C)c2ccccc2-c2c1c1ccccc1n2-c1cccc(-c2nc(-c3ccccc3)nc(-c3ccccc3)n2)c1 ; c1ccc(-c2cc(-c3cccc4ccccc34)cc(-n3c4ccccc4c4oc5ccccc5c43)c2)cc1 ; c1ccc(-c2cc(-n3c4ccccc4c4c5c(ccc43)ccn5-c3ccccc3)nc(-c3ccccc3)n2)cc1]

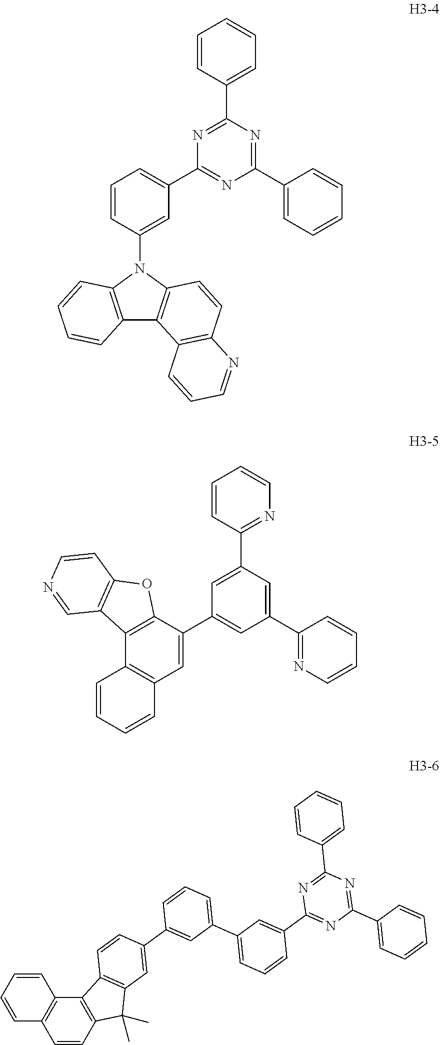 [SMILES: c1ccc(-c2nc(-c3ccccc3)nc(-c3cccc(-n4c5ccccc5c5c6cccnc6ccc54)c3)n2)cc1 ; c1ccc(-c2cc(-c3ccccn3)cc(-c3cc4ccccc4c4c3oc3ccncc34)c2)nc1 ; CC1(C)c2cc(-c3cccc(-c4cccc(-c5nc(-c6ccccc6)nc(-c6ccccc6)n5)c4)c3)ccc2-c2c1ccc1ccccc21]

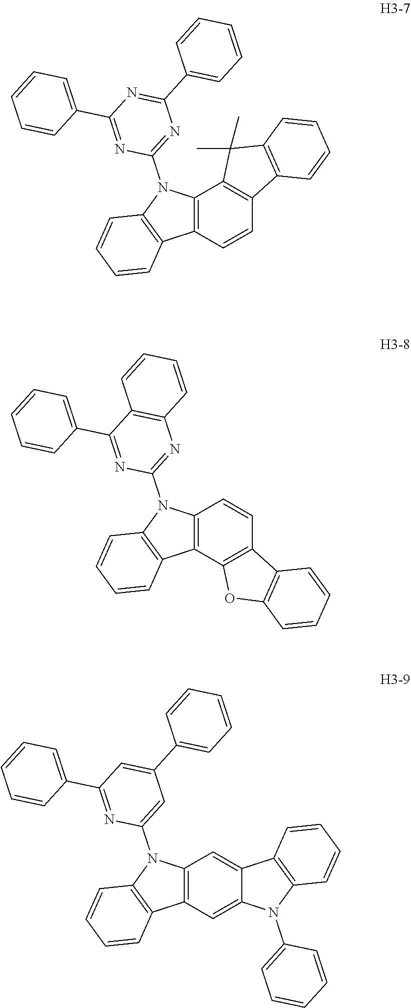 [SMILES: CC1(C)c2ccccc2-c2ccc3c4ccccc4n(-c4nc(-c5ccccc5)nc(-c5ccccc5)n4)c3c21 ; c1ccc(-c2nc(-n3c4ccccc4c4c5oc6ccccc6c5ccc43)nc3ccccc23)cc1 ; c1ccc(-c2cc(-c3ccccc3)nc(-n3c4ccccc4c4cc5c(cc43)c3ccccc3n5-c3ccccc3)c2)cc1]

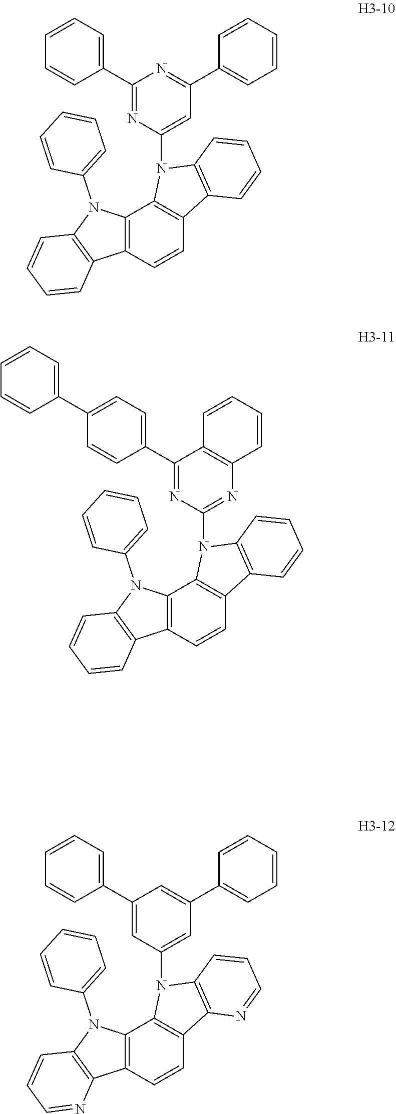 [SMILES: c1ccc(-c2cc(-n3c4ccccc4c4ccc5c6ccccc6n(-c6ccccc6)c5c43)nc(-c3ccccc3)n2)cc1 ; c1ccc(-c2ccc(-c3nc(-n4c5ccccc5c5ccc6c7ccccc7n(-c7ccccc7)c6c54)nc4ccccc34)cc2)cc1 ; c1ccc(-c2cc(-c3ccccc3)cc(-n3c4cccnc4c4ccc5c6ncccc6n(-c6ccccc6)c5c43)c2)cc1]

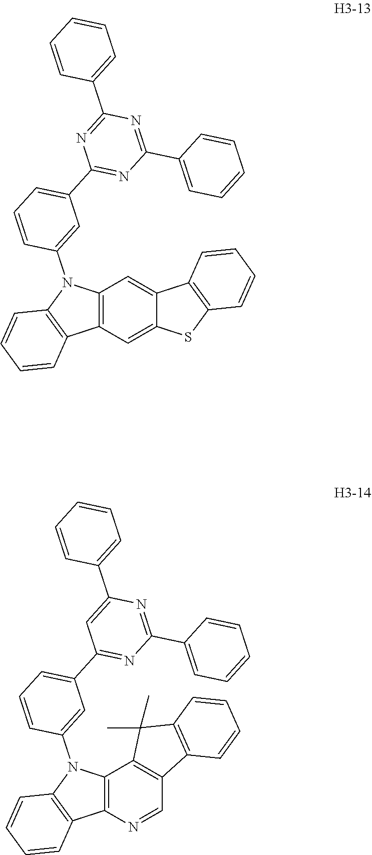 [SMILES: c1ccc(-c2nc(-c3ccccc3)nc(-c3cccc(-n4c5ccccc5c5cc6sc7ccccc7c6cc54)c3)n2)cc1 ; CC1(C)c2ccccc2-c2cnc3c4ccccc4n(-c4cccc(-c5cc(-c6ccccc6)nc(-c6ccccc6)n5)c4)c3c21]

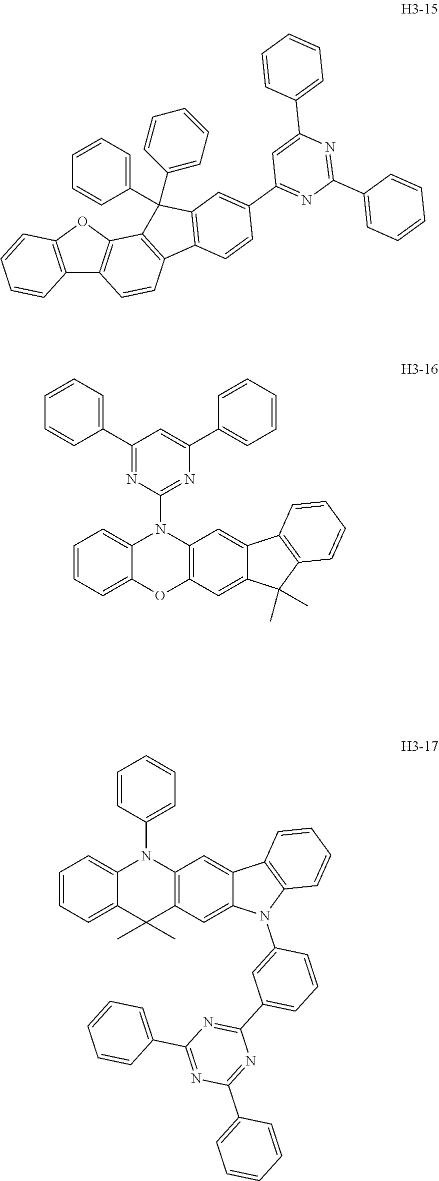 [SMILES: c1ccc(-c2cc(-c3ccc4c(c3)C(c3ccccc3)(c3ccccc3)c3c-4ccc4c3oc3ccccc34)nc(-c3ccccc3)n2)cc1 ; CC1(C)c2ccccc2-c2cc3c(cc21)Oc1ccccc1N3c1nc(-c2ccccc2)cc(-c2ccccc2)n1 ; CC1(C)c2ccccc2N(c2ccccc2)c2cc3c4ccccc4n(-c4cccc(-c5nc(-c6ccccc6)nc(-c6ccccc6)n5)c4)c3cc21]

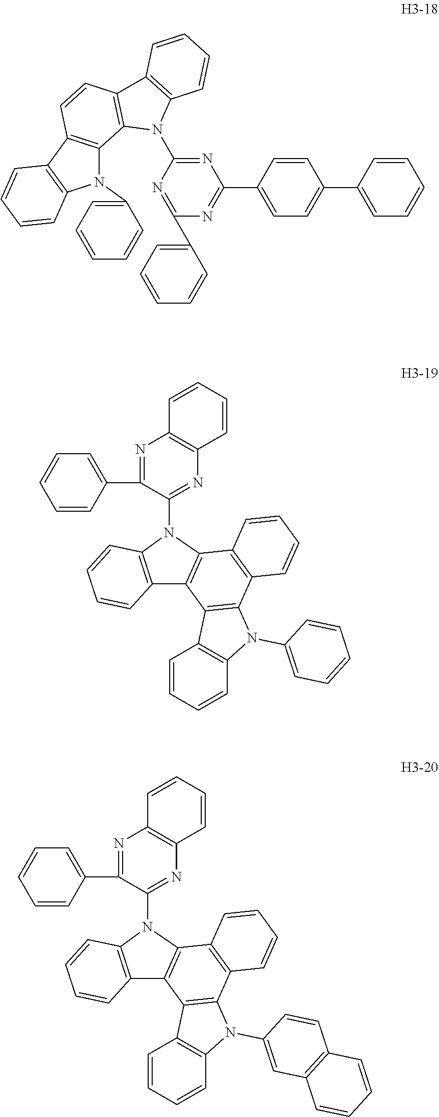 [SMILES: c1ccc(-c2ccc(-c3nc(-c4ccccc4)nc(-n4c5ccccc5c5ccc6c7ccccc7n(-c7ccccc7)c6c54)n3)cc2)cc1 ; c1ccc(-c2nc3ccccc3nc2-n2c3ccccc3c3c4c5ccccc5n(-c5ccccc5)c4c4ccccc4c32)cc1 ; c1ccc(-c2nc3ccccc3nc2-n2c3ccccc3c3c4c5ccccc5n(-c5ccc6ccccc6c5)c4c4ccccc4c32)cc1]

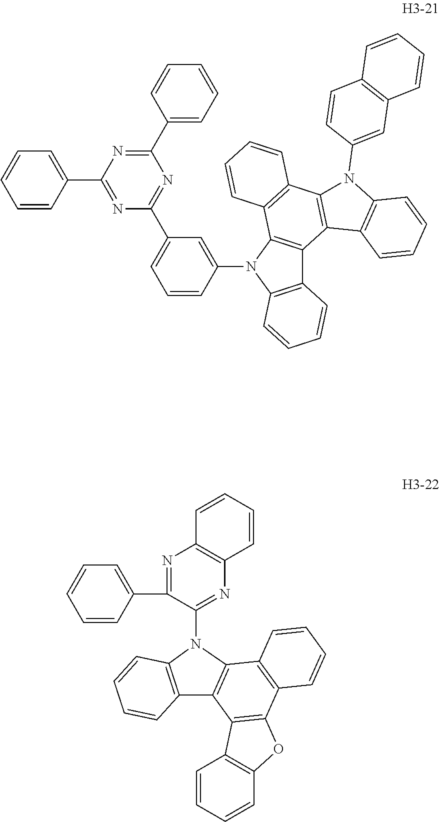 [SMILES: c1ccc(-c2nc(-c3ccccc3)nc(-c3cccc(-n4c5ccccc5c5c6c7ccccc7n(-c7ccc8ccccc8c7)c6c6ccccc6c54)c3)n2)cc1 ; c1ccc(-c2nc3ccccc3nc2-n2c3ccccc3c3c4c5ccccc5oc4c4ccccc4c32)cc1]

In an embodiment, the third dopant included in the emission layer of the light emitting unit emitting light having the second wavelength may include any dopant selected from PD1 to PD25 below. However, the third dopant material is not limited to PD1 to PD25.

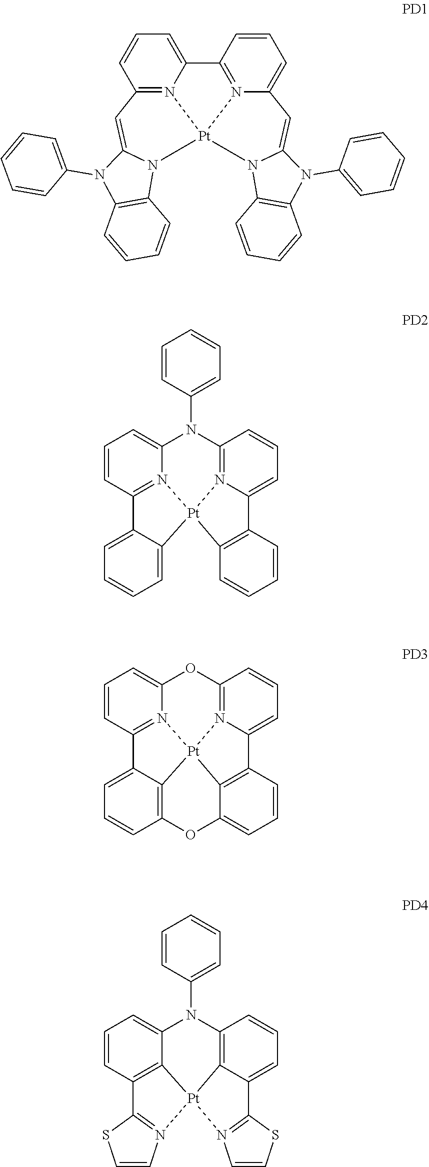

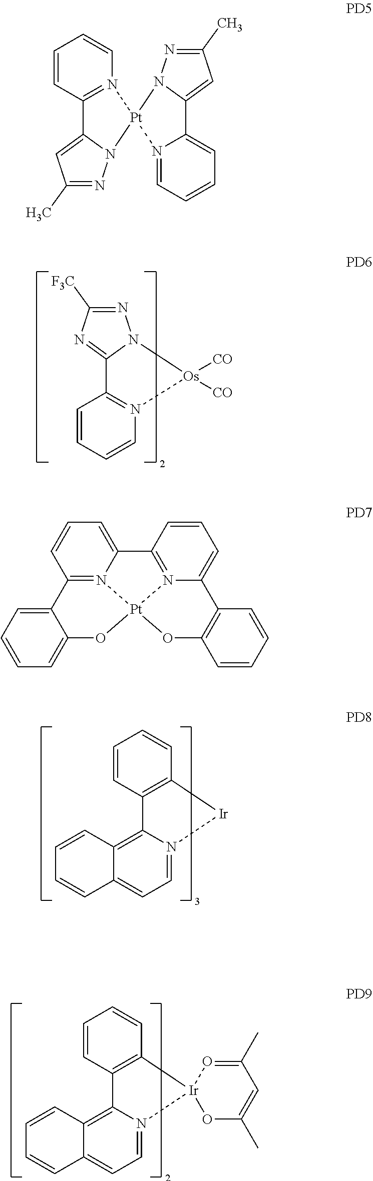

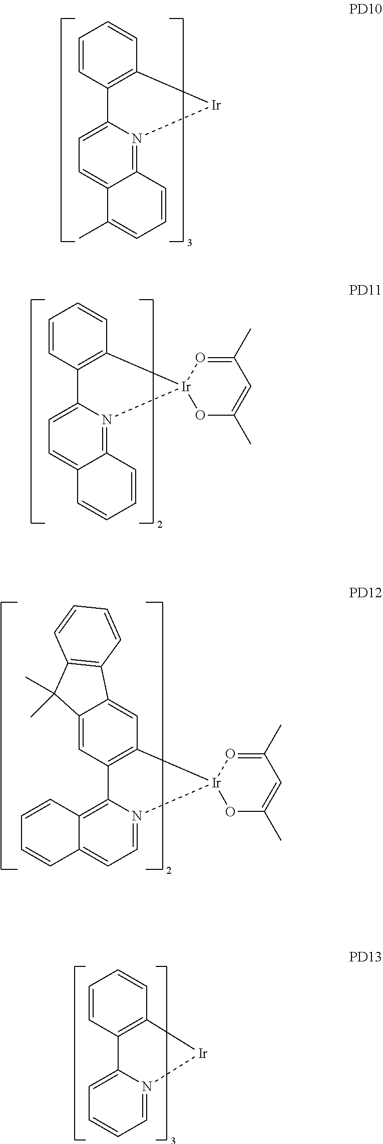

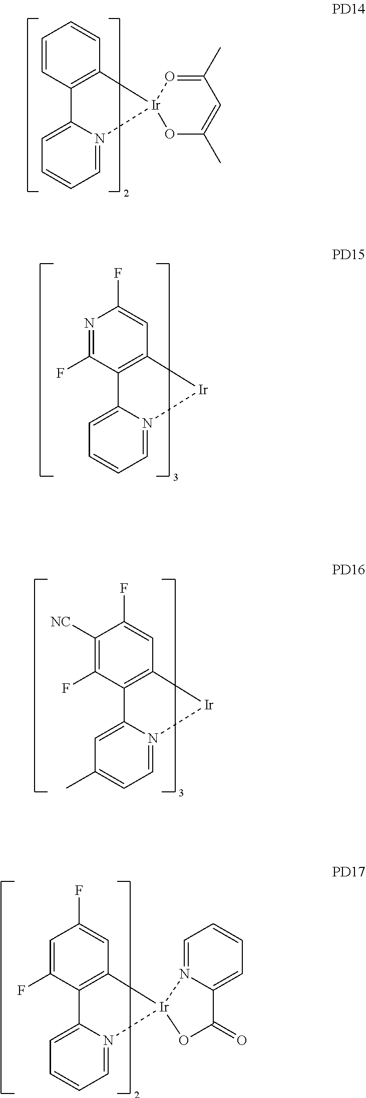

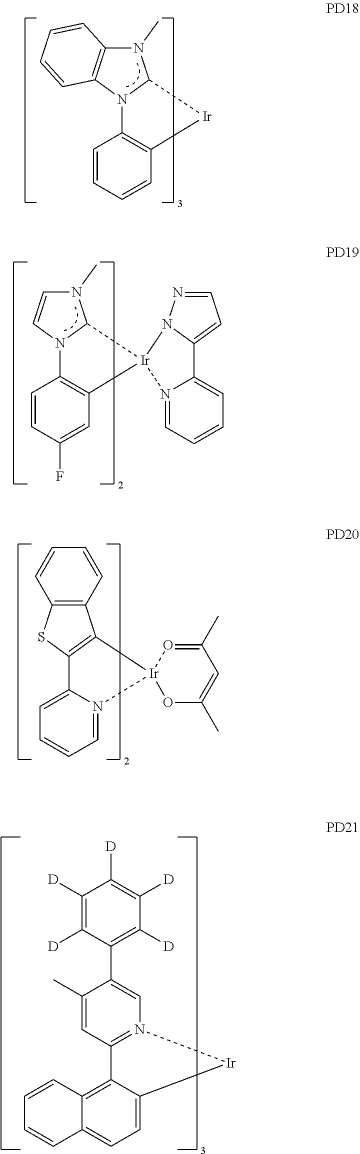

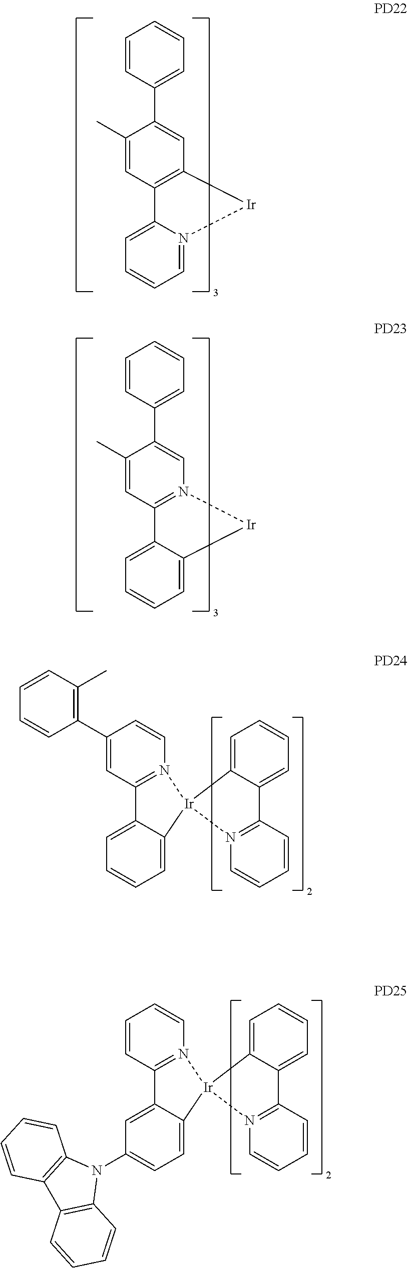

Referring to FIG. 9 again, the light emitting units EU-1, EU-2, EU-3, and EU-4 of the light emitting diode OEL of an embodiment may include electron transport regions ETR-1, ETR-2, ETR-3, and ETR-4. Each of the electron transport regions ETR-1, ETR-2, ETR-3, and ETR-4 included in the light emitting units EU-1, EU-2, EU-3, and EU-4 may be a single layer formed of a single material, a single layer formed of different materials, or a structure having multiple layers formed of different materials.

Each of the electron transport regions ETR-1, ETR-2, ETR-3, and ETR-4 may be formed using various methods such as a vacuum deposition method, a spin coating method, a cast method, a Langmuir-Blodgett (LB) method, an inkjet printing method, a laser printing method, and a laser induced thermal imaging (LITI) method.

The electron transport regions ETR-1, ETR-2, ETR-3, and ETR-4 may include an anthracene-based compound. However, embodiments are not limited thereto, and the electron transport regions ETR-1, ETR-2, ETR-3, and ETR-4 may include, for example, tris(8-hydroxyquinolinato)aluminum (Alq₃), 1,3,5-tri[(3-pyridyl)-phen-3-yl]benzene, 2,4,6-tris(3′-(pyridin-3-yl)biphenyl-3-yl)-1,3,5-triazine, 2-(4-(N-phenylbenzoimidazolyl-1-ylphenyl)-9,10-dinaphthylanthracene, 1,3,5-tri(1-phenyl-1H-benzo[d]imidazol-2-yl)benzene (TPBi), 2,9-dimethyl-4,7-diphenyl-1,10-phenanthroline (BCP), 4,7-diphenyl-1,10-phenanthroline (Bphen), 3-(4-biphenylyl)-4-phenyl-5-tert-butylphenyl-1,2,4-triazole (TAZ), 4-(naphthalen-1-yl)-3,5-diphenyl-4H-1,2,4-triazole (NTAZ), 2-(4-biphenylyl)-5-(4-tert-butylphenyl)-1,3,4-oxadiazole (tBu-PBD), bis(2-methyl-8-quinolinolato-N1,O8)-(1,1′-biphenyl-4-olato)aluminum (BAlq), berylliumbis(benzoquinolin-10-olate (Bebq₂), 9,10-di(naphthalene-2-yl)anthracene (ADN), 1,3-bis[3,5-di(pyridin-3-yl)phenyl]benzene (BmPyPhB), diphenyl(4-(triphenylsilyl)phenyl)phosphine oxide (TSPTO1), and mixtures thereof, without limitation.

The electron transport regions ETR-1, ETR-2, ETR-3, and ETR-4 may include a metal halide such as LiF, NaCl, CsF, RbCl, RbI, CuI and KI, a lanthanide metal such as Yb, or a co-depositing material of the metal halide and the lanthanide metal. For example, the electron transport regions ETR-1, ETR-2, ETR-3, and ETR-4 may include KI:Yb, RbI:Yb, LiF:Yb, etc., as the co-depositing material. The electron transport regions ETR-1, ETR-2, ETR-3, and ETR-4 may use a metal oxide such as Li₂O and BaO, or 8-hydroxy-lithium quinolate (Liq). However, embodiments are not limited thereto. The electron transport regions ETR-1, ETR-2, ETR-3, and ETR-4 may also be formed using a mixture material of an electron transport material and an insulating organo metal salt. The organo metal salt may be a material having an energy band gap equal to or greater than about 4 eV. For example, the organo metal salt may include metal acetates, metal benzoates, metal acetoacetates, metal acetylacetonates, or metal stearates.

The electron transport regions ETR-1, ETR-2, ETR-3, and ETR-4 may further include at least one of 2,9-dimethyl-4,7-diphenyl-1,10-phenanthroline (BCP), diphenyl(4-(triphenylsilyl)phenyl)phosphine oxide (TSPO1) or 4,7-diphenyl-1,10-phenanthroline (Bphen) in addition to the aforementioned materials. However, embodiments are not limited thereto.

Referring to FIG. 10A, the electron transport region ETR may include an electron transport layer ETL and an electron injection layer EIL. The electron transport region ETR may include at least one of an electron injection layer EIL, an electron transport layer ETL, and a hole blocking layer (not shown). In an embodiment, the electron transport region ETR may have a stacked structure of electron transport layer ETL/electron injection layer EIL, hole blocking layer (not shown)/electron transport layer ETL/electron injection layer EIL, electron transport layer ETL/buffer layer (not shown)/electron injection layer EIL, etc., but embodiments are not limited thereto. In at least one of the electron injection layer EIL, electron transport layer ETL and hole blocking layer (not shown), the above-described materials of the hole transport region may be included.

Referring to FIG. 9 again, the second electrode EL2 is provided on the fourth light emitting unit EU-4. The second electrode EL2 may be a common electrode. The second electrode EL2 may be a cathode or an anode, but embodiments are not limited thereto. For example, if the first electrode EL1 is an anode, the second cathode EL2 may be a cathode, and if the first electrode EL1 is a cathode, the second electrode EL2 may be an anode. The second electrode may include at least one of Ag, Mg, Cu, Al, Pt, Pd, Au, Ni, Nd, Ir, Cr, Li, Ca, LiF, Mo, Ti, W, In, Sn, Zn, oxides thereof, compounds thereof, or mixtures thereof.

The second electrode EL2 may be a transmissive electrode, a transflective electrode, or a reflective electrode. If the second electrode EL2 is a transmissive electrode, the second electrode EL2 may include a transparent metal oxide, for example, indium tin oxide (ITO), indium zinc oxide (IZO), zinc oxide (ZnO), indium tin zinc oxide (ITZO), etc.

If the second electrode EL2 is a transflective electrode or a reflective electrode, the second electrode EL2 may include Ag, Mg, Cu, Al, Pt, Pd, Au, Ni, Nd, Ir, Cr, Li, Ca, LiF/Ca (a stacked structure of LiF and Ca), LiF/Al (a stacked structure of LiF and Al), Mo, Ti, Yb, W, compounds thereof, or mixtures thereof (for example, AgMg, AgYb, or MgYb). In another embodiment, the second electrode EL2 may have a multilayered structure including a reflective layer or a transflective layer formed using the above-described materials and a transparent conductive layer formed using ITO, IZO, ZnO, ITZO, etc. For example, the second electrode EL2 may include the aforementioned metal materials, combinations of two or more metal materials selected from the aforementioned metal materials, or oxides of the aforementioned metal materials.

In an embodiment, the light emitting diode OEL may further include a capping layer CPL disposed on the second electrode EL2. The capping layer CPL may be a multilayer or a single layer.

In an embodiment, the capping layer CPL may be an organic layer or an inorganic layer. For example, if the capping layer CPL includes an inorganic material, the inorganic material may include an alkali metal compound such as LiF, an alkaline earth metal compound such as MgF₂, SiON, SiNₓ, SiO_{y}, etc.

For example, if the capping layer CPL includes an organic material, the organic material may include α-NPD, NPB, TPD, m-MTDATA, Alq₃, CuPc, N4,N4,N4′,N4′-tetra(biphenyl-4-yl) biphenyl-4,4′-diamine (TPD15), 4,4′,4″-tris(carbazol-9-yl) triphenylamine (TCTA), etc., or may include an epoxy resin, or acrylate such as methacrylate. The capping layer CPL may include at least one of Compounds P1 to P5 below, but embodiments are not limited thereto.

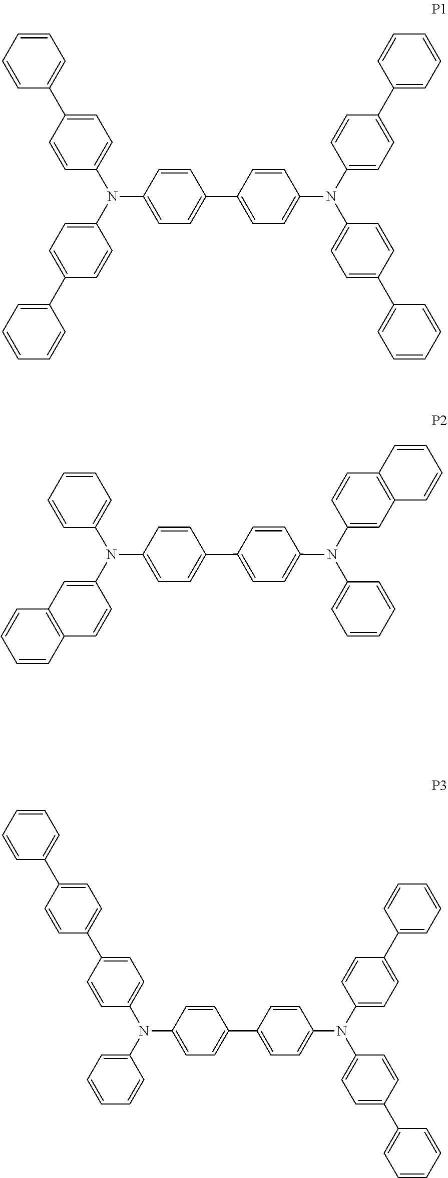 [SMILES: c1ccc(-c2ccc(N(c3ccc(-c4ccccc4)cc3)c3ccc(-c4ccc(N(c5ccc(-c6ccccc6)cc5)c5ccc(-c6ccccc6)cc5)cc4)cc3)cc2)cc1 ; c1ccc(N(c2ccc(-c3ccc(N(c4ccccc4)c4ccc5ccccc5c4)cc3)cc2)c2ccc3ccccc3c2)cc1 ; c1ccc(-c2ccc(-c3ccc(N(c4ccccc4)c4ccc(-c5ccc(N(c6ccc(-c7ccccc7)cc6)c6ccc(-c7ccccc7)cc6)cc5)cc4)cc3)cc2)cc1]

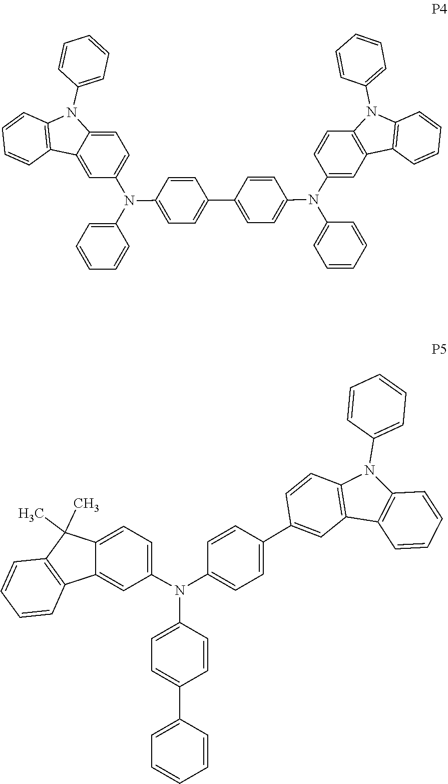 [SMILES: c1ccc(N(c2ccc(-c3ccc(N(c4ccccc4)c4ccc5c(c4)c4ccccc4n5-c4ccccc4)cc3)cc2)c2ccc3c(c2)c2ccccc2n3-c2ccccc2)cc1 ; CC1(C)c2ccccc2-c2cc(N(c3ccc(-c4ccccc4)cc3)c3ccc(-c4ccc5c(c4)c4ccccc4n5-c4ccccc4)cc3)ccc21]

A refractive index of the capping layer CPL may be equal to or greater than about 1.6. For example, a refractive index of the capping layer CPL may be equal to or greater than about 1.6, with respect to light in a wavelength range of about 550 nm to about 660 nm.

The light emitting diode OEL of an embodiment includes multiple light emitting units and includes a hole transport region including the amine compound according to an embodiment in at least one of the light emitting units, thereby showing high light extraction efficiency properties and improved emission efficiency properties. The display device DD-1 of an embodiment includes the light emitting diode OEL including the amine compound according to an embodiment in a hole transport region and may show excellent emission efficiency.

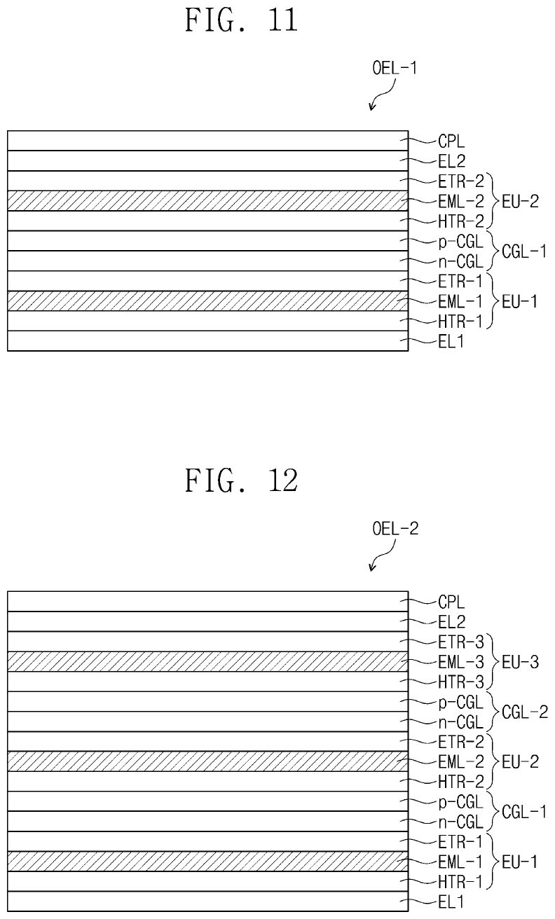

FIG. 11 and FIG. 12 are each a schematic cross-sectional view of a light emitting diode according to embodiments. Hereinafter, in the explanation on the light emitting diodes of embodiments, referring to FIG. 11 and FIG. 12, the features which overlap with the explanation of FIG. 1 to FIG. 10C will not be explained again, and the differing features will be explained.

The light emitting diodes OEL-1 and OEL-2 of embodiments shown in FIG. 11 and FIG. 12 may be included in the display device DD-1 shown in FIG. 8. The light emitting diodes OEL-1 and OEL-2 of embodiments shown in FIG. 11 and FIG. 12 have different numbers of light emitting units stacked when compared to the light emitting diode OEL shown in FIG. 9.

Referring to FIG. 11, the light emitting diode OEL-1 of an embodiment may include a first electrode EL1, a second electrode EL2 oppositely disposed to the first electrode EL1, and a first light emitting unit EU-1 and a second light emitting unit EU-2 disposed between the first electrode EL1 and the second electrode EL2. The light emitting diode OEL-1 may include a charge generating layer CGL-1 disposed between the first light emitting unit EU-1 and the second light emitting unit EU-2. The light emitting diode OEL-1 may further include a capping layer CPL disposed on the second electrode EL2.

The first light emitting unit EU-1 and the second light emitting unit EU-2 may respectively include hole transport regions HTR-1 and HTR-2, emission layers EMLL-1 and EMLL-2, and electron transport regions ETR-1 and ETR-2. In an embodiment, the first light emitting unit EU-1 and the second light emitting unit EU-2 may emit light having a first wavelength in a range of about 430 nm to about 470 nm. For example, each of the first light emitting unit EU-1 and the second light emitting unit EU-2 may be blue light emitting unit emitting blue light. However, embodiments are not limited thereto, and the first light emitting unit EU-1 and the second light emitting unit EU-2 may emit light in different wavelength regions from each other.

In the light emitting diode OEL-1 of an embodiment, the hole transport regions HTR-1 and HTR-2 included in at least one light emitting unit among the first light emitting unit EU-1 and the second light emitting unit EU-2 may include the amine compound of an embodiment, represented by Formula 1. For example, the light emitting diode OEL-1 of an embodiment may include the amine compound of an embodiment only in the hole transport region HTR-2 of the second light emitting unit EU-2 among the light emitting units, or may include the amine compound of an embodiment both in the hole transport region HTR-2 of the second light emitting unit EU-2 and in the hole transport region HTR-1 of the first light emitting diode EU-1.

Referring to FIG. 12, the light emitting diode OEL-2 of an embodiment may include a first electrode EL1, a second electrode EL2 oppositely disposed to the first electrode EL1, and first to third light emitting units EU-1, EU-2, and EU-3, disposed between the first electrode EL1 and the second electrode EL2. The light emitting diode OEL-2 may include a first charge generating layer CGL-1 disposed between the first light emitting unit EU-1 and the second light emitting unit EU-2, and a second charge generating layer CGL-2 disposed between the second light emitting unit EU-2 and the third light emitting unit EU-3. The light emitting diode OEL-2 may further include a capping layer CPL disposed on the second electrode EL2.

The first to third light emitting units EU-1, EU-2, and EU-3 may respectively include hole transport regions HTR-1, HTR-2, and HTR-3, emission layers EMLL-1, EMLL-2, and EML-3, and electron transport regions ETR-1, ETR-2, and ETR-3, stacked in order. In an embodiment, the first to third light emitting units EU-1, EU-2, and EU-3 may emit light having a first wavelength in a range of about 430 nm to about 470 nm. In another embodiment, the first light emitting unit EU-1 and the second light emitting unit EU-2 may emit light having a first wavelength in a range of about 430 nm to about 470 nm, and the third light emitting unit EU-3 may emit light having a second wavelength in a range of about 520 nm to about 600 nm. For example, each of the first light emitting unit EU-1 and the second light emitting unit EU-2 may be a blue light emitting unit, and the third light emitting unit EU-3 may be a green light emitting unit emitting green light. However, embodiments are not limited thereto, and the first to third light emitting units EU-1, EU-2, and EU-3 may emit light in different wavelength regions.

In the light emitting diode OEL-2 of an embodiment, the hole transport regions HTR-1, HTR-2, and HTR-3 included in at least one light emitting unit among the first to third light emitting units EU-1, EU-2, and EU-3 may include the amine compound of an embodiment, represented by Formula 1. For example, the light emitting diode OEL-2 of an embodiment may include the amine compound of an embodiment only in the hole transport region HTR-3 of the third light emitting unit EU-3 among the light emitting units, or may include the amine compound of an embodiment in all the hole transport regions HTR-1, HTR-2, and HTR-3 of the first to third light emitting units EU-1, EU-2, and EU-3.

In the light emitting diodes OEL-1 and OEL-2 according to embodiments, shown in FIG. 11 and FIG. 12, at least one of the light emitting units may have the structures of the light emitting units explained referring to FIG. 10A to FIG. 10C.

The light emitting diodes OEL-1 and OEL-2 according to embodiments, shown in FIG. 11 and FIG. 12 include the amine compound of an embodiment, represented by Formula 1 in at least one light emitting unit among the light emitting units, and may show excellent light extraction efficiency and improved emission efficiency properties.

The light emitting diode of an embodiment includes multiple light emitting units, and includes the amine compound of an embodiment having a low refractive index in at least one of the light emitting units, thereby showing high light extraction efficiency and improved emission efficiency. The display device of an embodiment includes a hole transport region including the amine compound of an embodiment having a low refractive index and may show excellent emission efficiency.

Hereinafter, referring to embodiments and comparative embodiments, the light emitting diode according to an embodiment will be explained. The following embodiments are only illustrations to assist the understanding of the disclosure, and the scope thereof is not limited thereto.

### EXAMPLES

1. Synthesis of Amine Compound

The synthesis method of an amine compound according to an embodiment will be explained to illustrate the synthesis methods of the Example Compounds. The synthesis methods of the amine compounds explained hereinafter are embodiments, and the synthesis method of the amine compound according to an embodiment is not limited thereto.

<Synthesis of Compound 7>

Amine Compound 7 according to an embodiment may be synthesized, for example, by the steps of Reaction 1 below.

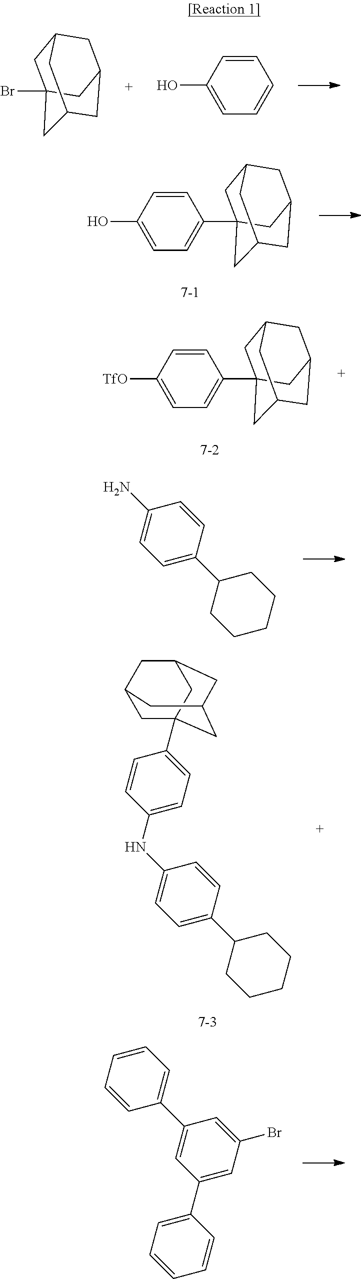

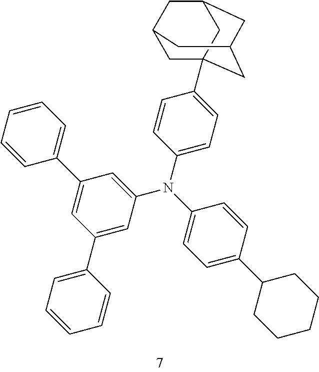 [SMILES: c1ccc(-c2cc(-c3ccccc3)cc(N(c3ccc(C4CCCCC4)cc3)c3ccc(C45CC6CC(CC(C6)C4)C5)cc3)c2)cc1]

(Synthesis of Intermediate Compound 7-1)

2.15 g (10 mmol) of 1-bromoadamantane and 7.5 g (80 mmol) of phenol were added to a flask and stirred at about 120° C. for about 12 hours. After cooling the reaction solution to room temperature, the reaction solution was added to 200 ml of hot water, precipitated, and filtered. After filtering, washing with 200 ml of hot water was performed three times to obtain 1.82 g (yield 80%) of Intermediate Compound 7-1. The compound thus produced was identified through LC-MS. (C₁₆H₂₀O: M+228.1)

(Synthesis of Intermediate Compound 7-2)

To a flask in which 2.28 g (10 mmol) of Intermediate Compound 7-1 and 4.18 ml (30 mmol) of triethylamine were dissolved in 30 ml of dichloromethane (DCM), a reactant of 3.36 ml (20 mmol) of trifluoromethansulfonic anhydride dissolved in 20 ml of DCM was slowly added at about 0° C., and stirred at room temperature for about 5 hours. After that, 40 ml of water was added to the reaction solution, and extraction was performed with 50 ml of ethyl ether three times. The organic layer thus obtained was dried with MgSO₄, solvents were evaporated, and the residue thus obtained was separated by silica gel chromatography to obtain 2.88 g (yield 80%) of Intermediate Compound 7-2. The compound thus produced was identified through LC-MS. (C₁₇H₁₉F₃O₃S: M+360.1)

(Synthesis of Intermediate Compound 7-3)

3.60 g (10 mmol) of Intermediate Compound 7-2, 2.63 g (15 mmol) of 4-cyclohexylaniline, 0.46 g (0.5 mmol) of tris(dibenzylideneacetone)dipalladium(0) (Pd₂dba₃), and 2.88 g (30 mmol) of sodium tert-butoxide were dissolved in 60 ml of toluene, and stirred at about 80° C. for about 3 hours. After cooling the reaction solution to room temperature, 40 ml of water was added, and extraction with 50 ml of ethyl ether was conducted three times. The organic layer thus collected was dried with MgSO₄, and solvents were evaporated. The residue thus obtained was separated by silica gel chromatography to obtain 2.70 g (yield 70%) of Intermediate Compound 7-3. The compound thus produced was identified through LC-MS. (C₂₈H₃₅N: M+385.2)

(Synthesis of Compound 7)

3.85 g (10 mmol) of Intermediate Compound 7-3, 3.09 g (10 mmol) of 5′-bromo-1,1′:3′,1″-terphenyl, 0.46 g (0.5 mmol) of tris(dibenzylideneacetone)dipalladium(0) (Pd₂dba₃), and 2.88 g (30 mmol) of sodium tert-butoxide were dissolved in 60 ml of toluene and stirred at about 80° C. for about 3 hours. After cooling the reaction solution to room temperature, 40 ml of water was added, and extraction with 50 ml of ethyl ether was performed three times. The organic layer thus obtained was dried with MgSO₄, and solvents were evaporated. The residue thus obtained was separated by silica gel chromatography to obtain 4.30 g (yield 70%) of Compound 7. The compound thus produced was identified through MS/FAB and 1H NMR. (C₄₆H₄₇N: M+cal.: 613.37, found: 613.27)

<Synthesis of Compound 22>

Amine Compound 22 according to an embodiment may be synthesized, for example, by the steps of Reaction 2 below.

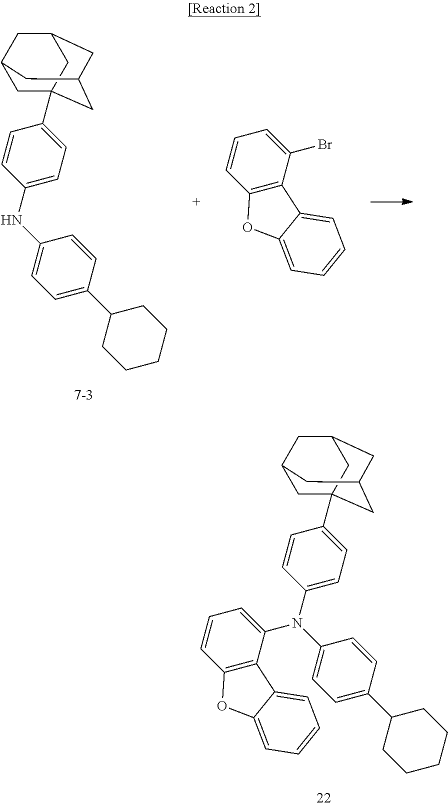 [SMILES: c1cc(C2CCCCC2)ccc1Nc1ccc(C23CC4CC(CC(C4)C2)C3)cc1 ; Brc1cccc2oc3ccccc3c12 ; c1ccc2c(c1)oc1cccc(N(c3ccc(C4CCCCC4)cc3)c3ccc(C45CC6CC(CC(C6)C4)C5)cc3)c12]

Amine Compound 22 according to an embodiment was synthesized by the same synthesis method of Compound 7 except for using 1-bromodibenzo[b,d]furan instead of 5′-bromo-1,1′:3′,1″-terphenyl in the synthesis method of Compound 7. The compound thus produced was identified through MS/FAB and 1H NMR. (C₄₀H₄₁NO: M+cal.: 551.32, found: 551.22)

<Synthesis of Compound 38>

Amine Compound 38 according to an embodiment may be synthesized, for example, by the steps of Reaction 3 below.

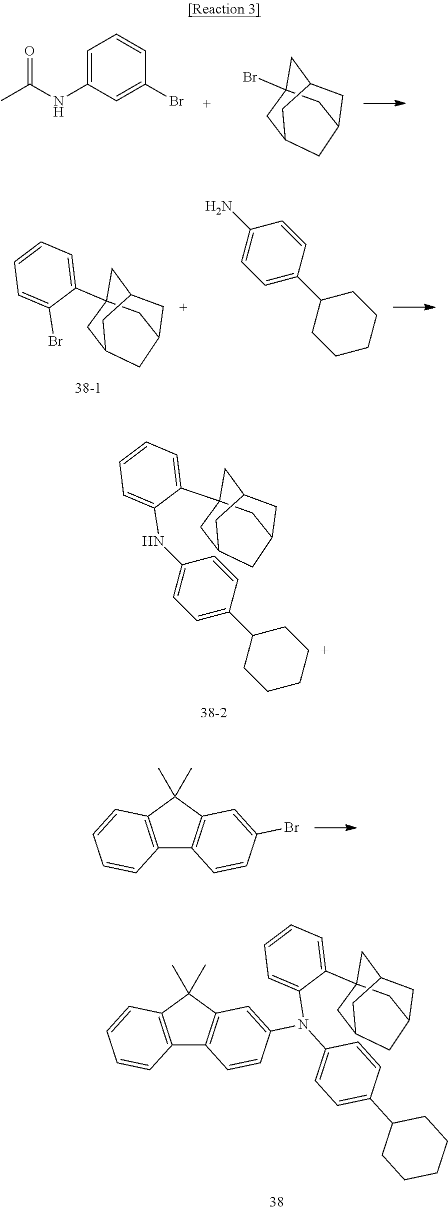

(Synthesis of Intermediate Compound 38-1)

2.15 g (10 mmol) of 1-bromoadamantane and 10.70 g (50 mmol) of N-(3-bromophenyl)acetamide were added to a flask and stirred at about 170° C. for about 18 hours. After cooling the reaction solution to room temperature, HCl (10 ml, 6 N) was added thereto and stirred at about 100° C. After about 4 hours, the reaction solution was cooled to room temperature and neutralized with NaHCO₃. The reaction solution was extracted with 50 ml of ethyl ether three times. The organic layer thus obtained was dried with MgSO₄, and solvents were evaporated. The residue thus obtained was dissolved in THE (20 ml), and isoamylnitride (1.34 ml, 10 mmol) was slowly added thereto. Stirring was conducted at about 60° C. for about 3 hours, the reaction solution was cooled to room temperature, solvents were evaporated, and the residue thus obtained was separated by silica gel chromatography to obtain 0.87 g (yield 30%) of Intermediate Compound 38-1. The compound thus produced was identified through LC-MS. (C₁₆H₁₉Br: M+290.0)

(Synthesis of Intermediate Compound 38-2)

2.90 g (10 mmol) of Intermediate Compound 38-1, 2.63 g (15 mmol) of 4-cyclohexylaniline, 0.46 g (0.5 mmol) of tris(dibenzylideneacetone)dipalladium(0) (Pd₂dba₃), and 2.88 g (30 mmol) of sodium tert-butoxide were dissolved in 60 ml of toluene and stirred at about 80° C. for about 3 hours. After cooling the reaction solution to room temperature, 40 ml of water was added, and extraction with 50 ml of ethyl ether was performed three times. The organic layer thus obtained was dried with MgSO₄, and solvents were evaporated. The residue thus obtained was separated by silica gel chromatography to obtain 2.70 g (yield 70%) of Intermediate Compound 38-2. The compound thus produced was identified through LC-MS. (C₂₈H₃₅N: M+385.2)

(Synthesis of Compound 38)

3.85 g (10 mmol) of Intermediate Compound 38-2, 2.37 g (10 mmol) of 2-bromo-9,9-dimethyl-9H-fluorene, 0.46 g (0.5 mmol) of tris(dibenzylideneacetone)dipalladium(0) (Pd₂dba₃), and 2.88 g (30 mmol) of sodium tert-butoxide were dissolved in 60 ml of toluene and stirred at about 80° C. for about 3 hours. After cooling the reaction solution to room temperature, 40 ml of water was added, and extraction with 50 ml of ethyl ether was performed three times. The organic layer thus obtained was dried with MgSO₄, and solvents were evaporated. The residue thus obtained was separated by silica gel chromatography to obtain 4.05 g (yield 70%) of Compound 38. The compound thus produced was identified through MS/FAB and 1H NMR. (C₄₃H₄₇N: M+cal.: 577.37, found: 577.27)

<Synthesis of Compound 51>

Amine Compound 51 according to an embodiment may be synthesized, for example, by the steps of Reaction 4 below.

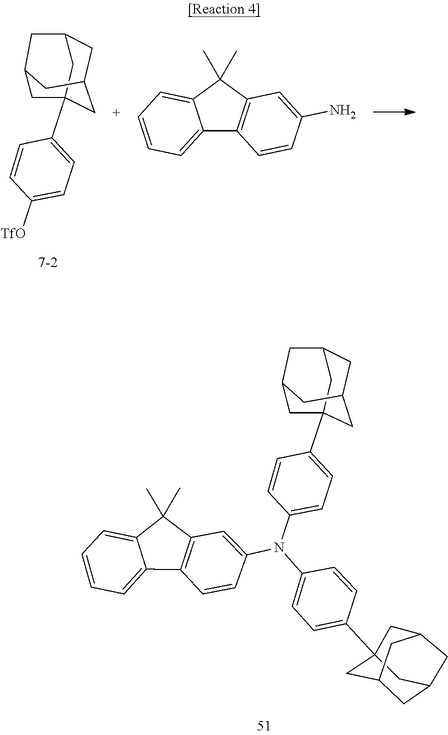

7.20 g (20 mmol) of Intermediate Compound 7-2, 2.09 g (10 mmol) of 2-amino-9,9-dimethyl-9H-fluorene, 0.46 g (0.5 mmol) of tris(dibenzylideneacetone)dipalladium(0) (Pd₂dba₃), and 2.88 g (30 mmol) of sodium tert-butoxide were dissolved in 60 ml of toluene, and stirred at about 80° C. for about 3 hours. After cooling the reaction solution to room temperature, 40 ml of water was added, and extraction with 50 ml of ethyl ether was performed three times. The organic layer thus obtained was dried with MgSO₄, and solvents were evaporated. The residue thus obtained was separated by silica gel chromatography to obtain 4.36 g (yield 70%) of Compound 51. The compound thus produced was identified through MS/FAB and 1H NMR. (C₄₇H₅₁N: M+cal.: 629.40, found: 629.30)

<Synthesis of Compound 57>

Amine Compound 57 according to an embodiment may be synthesized, for example, by the steps of Reaction 5 below.

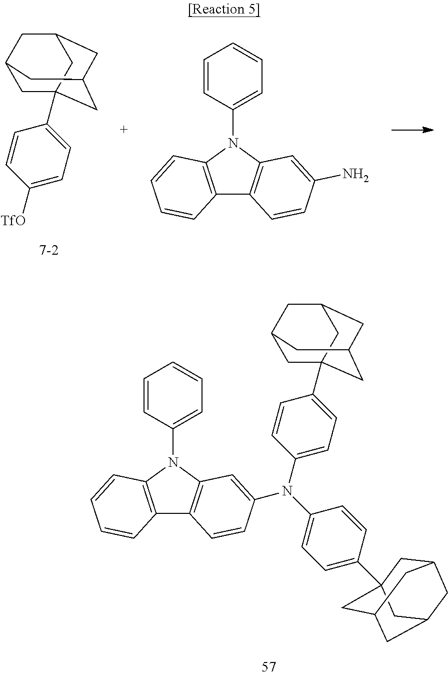 [SMILES: O=S(=O)(Oc1ccc(C23CC4CC(CC(C4)C2)C3)cc1)C(F)(F)F ; Nc1ccc2c3ccccc3n(-c3ccccc3)c2c1 ; c1ccc(-n2c3ccccc3c3ccc(N(c4ccc(C56CC7CC(CC(C7)C5)C6)cc4)c4ccc(C56CC7CC(CC(C7)C5)C6)cc4)cc32)cc1]

Amine Compound 57 was synthesized by the same synthesis method of Compound 51 except for using 9-phenyl-9H-carbazol-2-amine instead of 2-amino-9,9-dimethyl-9H-fluorene in the synthesis method of Compound 51. The compound thus produced was identified through MS/FAB and 1H NMR. (C₅₀H₅₀N₂: M+cal.: 678.40, found: 678.40)

<Synthesis of Compound 72>

Amine Compound 72 according to an embodiment may be synthesized, for example, by the steps of Reaction 6 below.

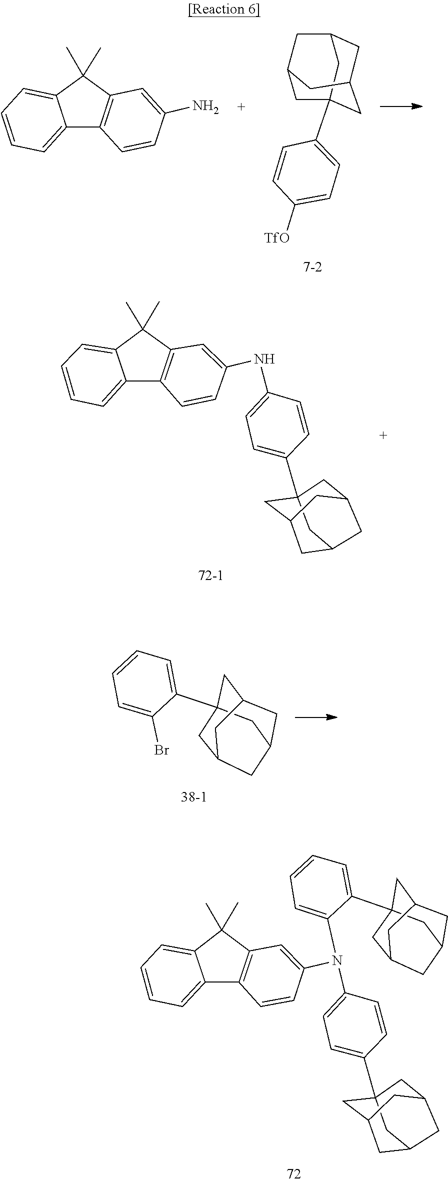 [SMILES: CC1(C)c2ccccc2-c2ccc(N)cc21 ; O=S(=O)(Oc1ccc(C23CC4CC(CC(C4)C2)C3)cc1)C(F)(F)F ; CC1(C)c2ccccc2-c2ccc(Nc3ccc(C45CC6CC(CC(C6)C4)C5)cc3)cc21 ; Brc1ccccc1C12CC3CC(CC(C3)C1)C2 ; CC1(C)c2ccccc2-c2ccc(N(c3ccc(C45CC6CC(CC(C6)C4)C5)cc3)c3ccccc3C34CC5CC(CC(C5)C3)C4)cc21]

(Synthesis of Intermediate Compound 72-1)

3.60 g (10 mmol) of Intermediate Compound 7-2, 3.14 g (15 mmol) of 2-amino-9,9-dimethyl-9H-fluorene, 0.46 g (0.5 mmol) of tris(dibenzylideneacetone)dipalladium(0) (Pd₂dba₃), and 2.88 g (30 mmol) of sodium tert-butoxide were dissolved in 60 ml of toluene and stirred at about 80° C. for about 3 hours. After cooling the reaction solution to room temperature, 40 ml of water was added, and extraction with 50 ml of ethyl ether was performed three times. The organic layer thus obtained was dried with MgSO₄, and solvents were evaporated. The residue thus obtained was separated by silica gel chromatography to obtain 2.94 g (yield 70%) of Intermediate Compound 72-1. The compound thus produced was identified through LC-MS. (C₃₁H₃₃N: M+419.2)

(Synthesis of Compound 72)

4.20 g (10 mmol) of Intermediate Compound 72-1, 2.91 g (10 mmol) of Intermediate Compound 38-1, 0.46 g (0.5 mmol) of tris(dibenzylideneacetone)dipalladium(0) (Pd₂dba₃), and 2.88 g (30 mmol) of sodium tert-butoxide were dissolved in 60 ml of toluene and stirred at about 80° C. for about 3 hours. After cooling the reaction solution to room temperature, 40 ml of water was added, and extraction with 50 ml of ethyl ether was performed three times. The organic layer thus obtained was dried with MgSO₄, and solvents were evaporated. The residue thus obtained was separated by silica gel chromatography to obtain 4.41 g (yield 70%) of Compound 72. The compound thus produced was identified through MS/FAB and 1H NMR. (C₄₇H₅₁N: M+cal.: 629.40, found: 629.30)

<Synthesis of Compound 83>

Amine Compound 83 according to an embodiment may be synthesized, for example, by the steps of Reaction 7 below.

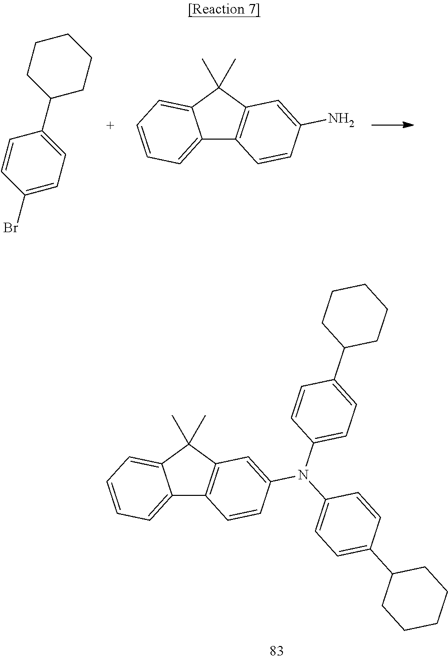 [SMILES: Brc1ccc(C2CCCCC2)cc1 ; CC1(C)c2ccccc2-c2ccc(N)cc21 ; CC1(C)c2ccccc2-c2ccc(N(c3ccc(C4CCCCC4)cc3)c3ccc(C4CCCCC4)cc3)cc21]

4.78 g (20 mmol) of 1-bromo-4-cyclohexylbenzene, 2.09 g (10 mmol) of 2-amino-9,9-dimethyl-9H-fluorene, 0.46 g (0.5 mmol) of tris(dibenzylideneacetone)dipalladium(0) (Pd₂dba₃), and 2.88 g (30 mmol) of sodium tert-butoxide were dissolved in 60 ml of toluene and stirred at about 80° C. for about 3 hours. After cooling the reaction solution to room temperature, 40 ml of water was added, and extraction with 50 ml of ethyl ether was performed three times. The organic layer thus obtained was dried with MgSO₄, and solvents were evaporated. The residue thus obtained was separated by silica gel chromatography to obtain 3.68 g (yield 70%) of Compound 83. The compound thus produced was identified through MS/FAB and 1H NMR. (C₃₉H₄₃N: M+cal.: 525.34, found: 525.24)

<Synthesis of Compound 89>

Amine Compound 89 according to an embodiment may be synthesized, for example, by the steps of Reaction 8 below.

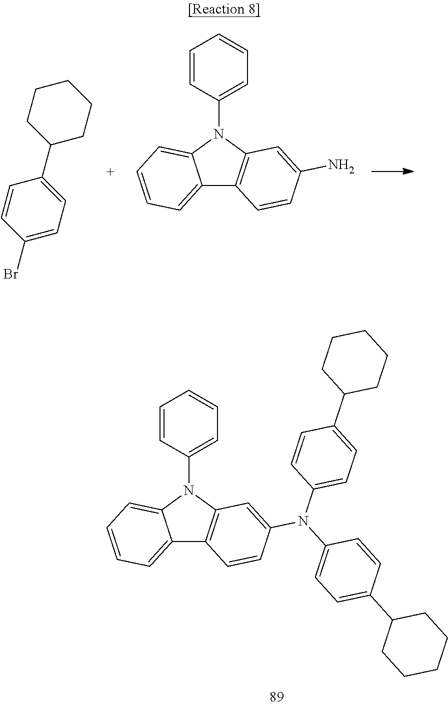 [SMILES: Brc1ccc(C2CCCCC2)cc1 ; Nc1ccc2c3ccccc3n(-c3ccccc3)c2c1 ; c1ccc(-n2c3ccccc3c3ccc(N(c4ccc(C5CCCCC5)cc4)c4ccc(C5CCCCC5)cc4)cc32)cc1]

Amine Compound 89 was synthesized by the same synthesis method of Compound 83 except for using 9-phenyl-9H-carbazol-2-amine instead of 2-amino-9,9-dimethyl-9H-fluorene. The compound thus produced was identified through MS/FAB and 1H NMR. (C₄₂H₄₂N₂: M+cal.: 574.33, found: 574.23)

<Synthesis of Compound 95>

Amine Compound 95 according to an embodiment may be synthesized, for example, by the steps of Reaction 9 below.

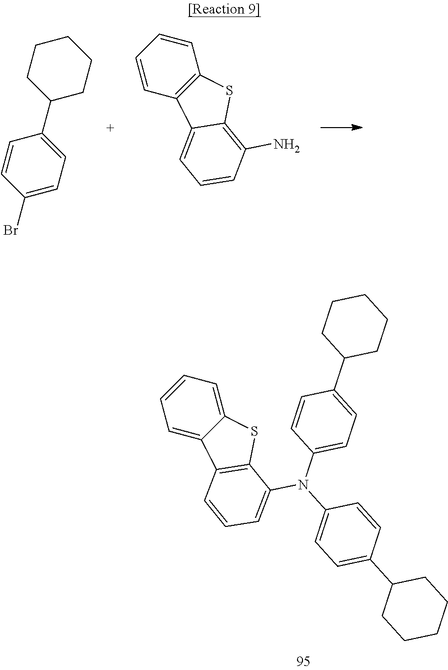

Amine Compound 95 was synthesized by the same synthesis method of Compound 83 except for using dibenzo[b,d]thiophen-4-amine instead of 2-amino-9,9-dimethyl-9H-fluorene. The compound thus produced was identified through MS/FAB and 1H NMR. (C₃₆H₃₇NS: M+cal.: 515.26, found: 515.16)

<Synthesis of Compound 11>

Amine Compound 11 according to an embodiment may be synthesized, for example, by the steps of Reaction 10 below.

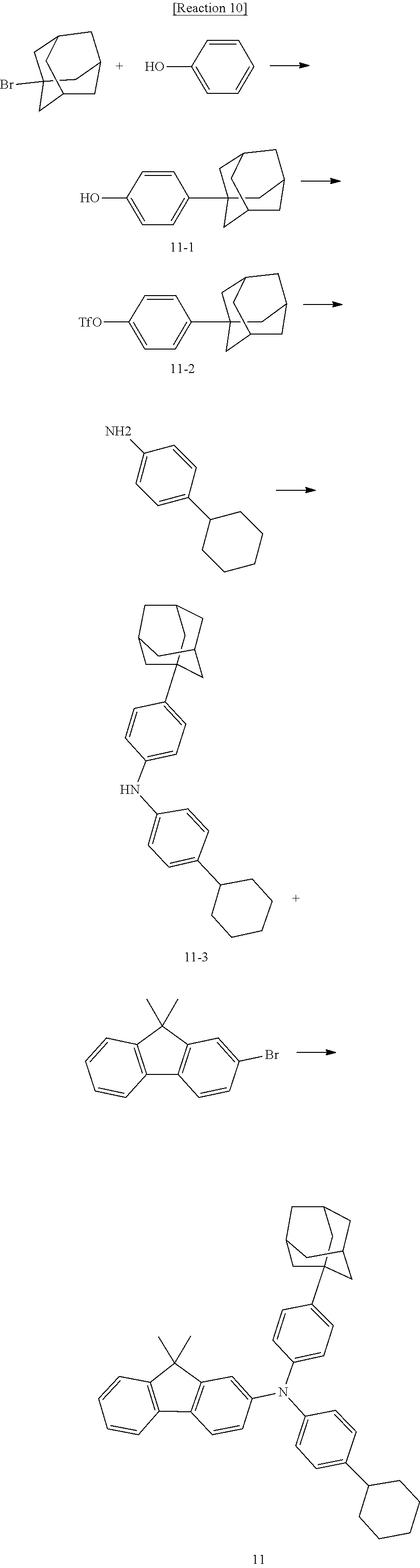

(Synthesis of Intermediate 11-1)

2.15 g (10 mmol) of 1-bromoadamantane and 7.5 g (80 mmol) of phenol were added to a flask and stirred at about 120° C. for about 12 hours. After cooling the reaction solution to room temperature, the reaction solution was added to 200 ml of hot water, precipitated, and filtered. After filtering, washing with 200 ml of hot water was performed three times to obtain 1.82 g (yield 80%) of Intermediate 11-1. The compound thus produced was identified through LC-MS. (C₁₆H₂₀O: M+228.1)

(Synthesis of Intermediate 11-2)

To a flask in which 2.28 g (10 mmol) of Intermediate 11-1 and 4.18 ml (30 mmol) of triethylamine were dissolved in 30 ml of dichloromethane (DCM), 3.36 ml (20 mmol) of trifluoromethanesulfonic anhydride dissolved in 20 ml of DCM was slowly added at about 0° C., and stirred at room temperature for about 5 hours. After that, 40 ml of water was added to the reaction solution, and extraction was performed with 50 ml of ethyl ether three times. The organic layer thus collected was dried over MgSO₄, solvents were evaporated, and the residue thus obtained was separated and purified by silica gel chromatography to obtain 2.88 g (yield 80%) of Intermediate 11-2. The compound thus produced was identified through LC-MS. (C₁₇H₁₉F₃O₃S: M+360.1)

(Synthesis of Intermediate 11-3)

3.60 g (10 mmol) of Intermediate 11-2, 2.63 g (15 mmol) of 4-cyclohexylaniline, 0.46 g (0.5 mmol) of tris(dibenzylideneacetone)dipalladium(0) (Pd₂dba₃), and 2.88 g (30 mmol) of sodium tert-butoxide were dissolved in 60 ml of toluene, and stirred at about 80° C. for about 3 hours. After cooling the reaction solution to room temperature, 40 ml of water was added, and extraction with 50 ml of ethyl ether was conducted three times. The organic layer thus collected was dried over MgSO₄, and solvents were evaporated. The residue thus obtained was separated and purified by silica gel chromatography to obtain 2.70 g (yield 70%) of Intermediate 11-3. The compound thus produced was identified through LC-MS. (C₂₈H₃₅N: M+385.2)

(Synthesis of Compound 11)

3.85 g (10 mmol) of Intermediate 11-3, 2.73 g (10 mmol) of 2-bromo-9,9-dimethyl-9H-fluorene, 0.46 g (0.5 mmol) of tris(dibenzylideneacetone)dipalladium(0) (Pd₂dba₃), and 2.88 g (30 mmol) of sodium tert-butoxide were dissolved in 60 ml of toluene and stirred at about 80° C. for about 3 hours. After cooling the reaction solution to room temperature, 40 ml of water was added, and extraction with 50 ml of ethyl ether was performed three times. The organic layer thus collected was dried over MgSO₄, and solvents were evaporated. The residue thus obtained was separated and purified by silica gel chromatography to obtain 4.04 g (yield 70%) of Compound 11. The compound thus produced was identified through MS/FAB. (C₄₃H₄₇N: M+cal.: 577.37, found: 577.27)

<Synthesis of Compound 12>

Amine Compound 12 according to an embodiment may be synthesized, for example, by the steps of Reaction 11 below.

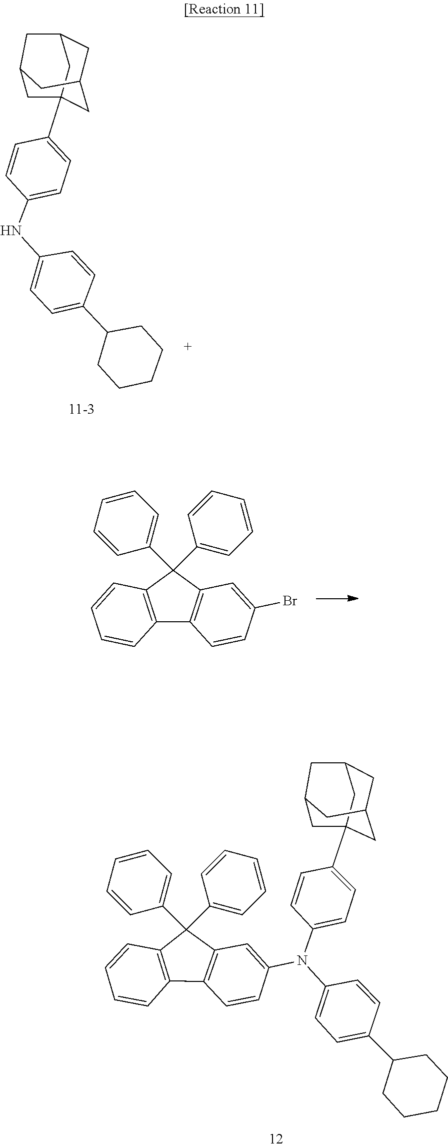 [SMILES: c1cc(C2CCCCC2)ccc1Nc1ccc(C23CC4CC(CC(C4)C2)C3)cc1 ; Brc1ccc2c(c1)C(c1ccccc1)(c1ccccc1)c1ccccc1-2 ; c1ccc(C2(c3ccccc3)c3ccccc3-c3ccc(N(c4ccc(C5CCCCC5)cc4)c4ccc(C56CC7CC(CC(C7)C5)C6)cc4)cc32)cc1]

Amine Compound 12 was synthesized by using 2-bromo-9,9-diphenyl-9H-fluorene instead of 2-bromo-9,9-dimethyl-9H-fluorene in the synthetic method of Compound 11. The compound thus produced was identified through MS/FAB. (C₅₃H₅₁N: M+cal.: 701.40, found: 701.35)

<Synthesis of Compound 53>

Amine Compound 53 according to an embodiment may be synthesized, for example, by the steps of Reaction 12 below.

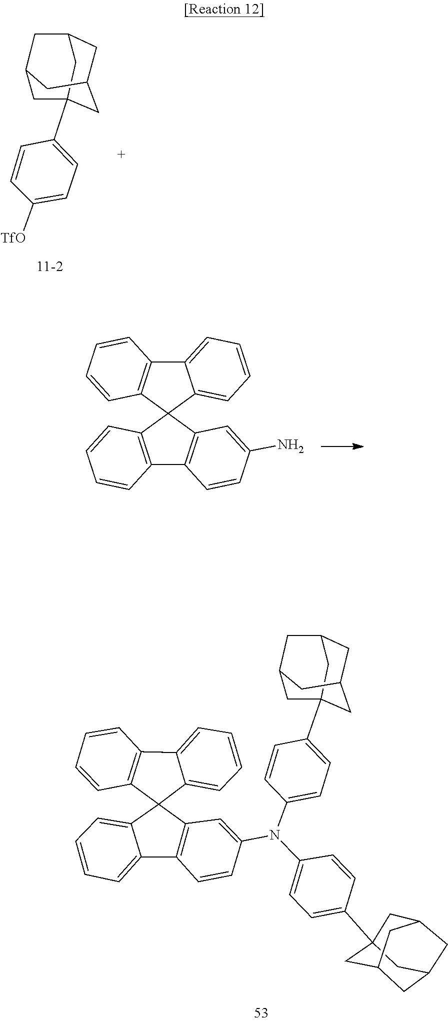 [SMILES: O=S(=O)(Oc1ccc(C23CC4CC(CC(C4)C2)C3)cc1)C(F)(F)F ; Nc1ccc2c(c1)C1(c3ccccc3-c3ccccc31)c1ccccc1-2 ; c1ccc2c(c1)-c1ccccc1C21c2ccccc2-c2ccc(N(c3ccc(C45CC6CC(CC(C6)C4)C5)cc3)c3ccc(C45CC6CC(CC(C6)C4)C5)cc3)cc21]

Amine Compound 53 was synthesized by using 9,9′-spirobi[fluoren]-2-amine instead of 9,9-dimethyl-9H-fluoren-2-amine in the synthetic method of Compound 11. The compound thus produced was identified through MS/FAB. (C₅₇H₅₃N: M+cal.: 751.42, found: 751.44)

<Synthesis of Compound 84>

Amine Compound 84 according to an embodiment may be synthesized, for example, by the steps of Reaction 13 below.

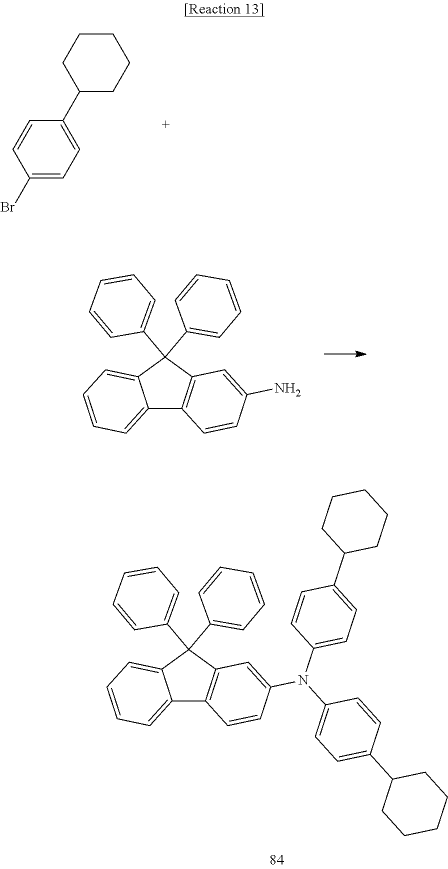 [SMILES: Brc1ccc(C2CCCCC2)cc1 ; Nc1ccc2c(c1)C(c1ccccc1)(c1ccccc1)c1ccccc1-2 ; c1ccc(C2(c3ccccc3)c3ccccc3-c3ccc(N(c4ccc(C5CCCCC5)cc4)c4ccc(C5CCCCC5)cc4)cc32)cc1]

4.78 g (20 mmol) of 1-bromo-4-cyclohexylbenzene, 3.33 g (10 mmol) of 9,9-diphenyl-9H-fluoren-2-amine, 0.46 g (0.5 mmol) of tris(dibenzylideneacetone)dipalladium(0) (Pd₂dba₃), and 2.88 g (30 mmol) of sodium tert-butoxide were dissolved in 60 ml of toluene, and stirred at about 80° C. for about 3 hours. After cooling the reaction solution to room temperature, 40 ml of water was added, and extraction with 50 ml of ethyl ether was performed three times. The organic layer thus collected was dried over MgSO₄, and the solvents were vaporized. The residue thus obtained was separated and purified by silica gel column chromatography to obtain 4.54 g (yield 70%) of Compound 84. The compound thus produced was identified through MS/FAB. (C₄₉H₄₇N: M+cal.: 649.37, found: 649.38)

<NMR Results of Synthesized Compounds>

In Table 1 below, 1H NMR results on the compounds synthesized by the above-described synthesis methods of the compounds are shown.

TABLE 1

| Compound | ¹H NMR (CDCl₃, 400 MHz) |
|---|---|
| Compound 7 | 7.75(d, 4H), 7.60(s, 1H), 7.49-7.37(m, 8H), 7.18(d, 2H), |
| | 7.10-7.06(m, 6H), 2.72(m, 1H), 2.05(d, 3H), 1.87-1.43(m, |
| | 22H) |
| Compound 11 | 7.90(d, 1H), 7.86(d, 1H), 7.55(d, 1H), 7.38-7.28 (m, 3H), |
| | 7.18-7.06(m, 9H), 2.72(m, 1H), 2.05(d, 3H), 1.87-1.43(m, |
| | 28H) |
| Compound 22 | 7.98(d, 1H), 7.54(d, 1H), 7.9-7.18(m, 6H), 7.10-7.06(m, |
| | 6H), 6.91(d 1H), 2.72(m, 1H), 2.05(d, 3H), 1.87-1.43(m, |
| | 22H) |
| Compound 38 | 7.90(d, 1H), 7.86(d, 1H), 7.55(d, 1H), 7.40-7.06(m, 12H), |
| | 2.72(m, 1H), 2.05(d, 3H), 1.87-1.43(m, 28H) |
| Compound 51 | 7.90(d, 1H), 7.86(d, 1H), 7.55(d, 1H), 7.38-7.28 (m, 3H), |
| | 7.16 (d, 1H), 7.10(s, 8H), 2.05(d, 6H), 1.87-1.69(m, 30H) |
| Compound 57 | 8.55(d, 1H), 8.24(d, 1H), 7.94(d, 1H), 7.62-7.50(m, 5H), |
| | 7.35-7.25(m, 3H), 7.16-7.10(m, 9H), 2.05(d, 6H), 1.87- |
| | 1.72(m, 24H) |
| Compound 72 | 7.90(d, 1H), 7.86(d, 1H), 7.55(d, 1H), 7.38-28(m, 3H), |
| | 7.40-7.09 (m, 9H), 2.05(d, 6H), 1.87-1.69(m, 30H) |
| Compound 83 | 7.90(d, 1H), 7.86(d, 1H), 7.55(d, 1H), 7.38-7.28 (m, 3H), |
| | 7.18-7.16(m, 5H), 7.06(d, 4H), 2.72(m, 2H), 1.86-1.43(m, |
| | 26H) |
| Compound 89 | 8.55(d, 1H), 8.24(d, 1H), 7.94(d, 1H), 7.62-7.50(m, 5H), |
| | 7.35-7.25(m, 3H), 7.18-7.16(m, 5H), 7.06(d, 4H), 2.72(m, |
| | 2H), 1.86-1.43(m, 20H) |
| Compound 95 | 8.45(d, 1H), 8.11(d, 1H), 7.93(d, 1H), 7.56-7.41(m, 4H), |
| | 7.18(d, 4H), 7.06(d, 4H), 2.72(m, 2H), 1.86-1.43(m, 20H) |

2. Manufacture and Evaluation of Light Emitting Diode-I

(Manufacture of Light Emitting Diode-I)

On a glass substrate, a first electrode having a stacked structure of ITO/Ag/ITO was formed. A first hole transport layer was formed using the amine compound of an embodiment, represented by Formula 1, a third hole transport layer was formed using the compound represented by Formula 2, and a second hole transport layer was formed using the amine compound of an embodiment, represented by Formula 1 to form a hole transport region. The first hole transport layer was formed into a thickness of about 300 Å, the second hole transport layer was formed into a thickness of about 300 Å, and the third hole transport layer was formed into a thickness of about 800 Å.

An emission layer was formed using 9,10-di(naphthalene-2-yl)anthracene (ADN) doped with 3% of 2,5,8,11-tetra-t-butylperylene (TBP) into a thickness of about 250 Å. Alq3 was deposited to a thickness of about 250 Å to form an electron transport layer, and LiF was deposited to a thickness of about 10 Å to form an electron injection layer.

AgMg was provided to a thickness of about 1,000 Å to form a second electrode. On the second electrode, a capping layer including a compound of P4 below was formed to a thickness of about 600 Å.

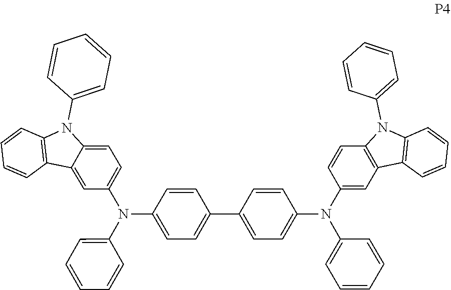

In the Example, the first electrode, the hole transport region, the emission layer, the electron transport layer, the electron injection layer and the second electrode were formed using a vacuum deposition apparatus.

(Evaluation of Properties of Light Emitting Diode-I)

In Table 2, with respect to the Examples including first to third hole transport layers, evaluation results of light emitting diodes according to the change of the refractive index values of the first to third hole transport layers are compared with those of the Comparative Example and shown. In Table 2, the emission efficiency, driving voltage, and device life of the light emitting diodes manufactured are compared with the Comparative Example and shown. In the evaluation results on properties of the Examples shown in Table 2, the emission efficiency represents relative values in case where the emission efficiency of the Comparative Example is set to 100%. The driving voltage represents relative values with respect to the driving voltage value of the Comparative Example (Ref.). The device life represents relative time until the luminance decreases to a level of about 97% of the initial luminance based on the Comparative Example.

Example 1 to Example 5 included a hole transport region having a stacked structure of first hole transport layer/third hole transport layer/second hole transport layer, and the Comparative Example corresponded to a case of including only the third hole transport layer in the hole transport region. In the configurations of the Comparative Example and the Examples, other elements except for the hole transport region were the same.

The refractive index of the third hole transport layer used in the Comparative Example and the Examples was about 1.95. The refractive index values shown in Table 2 below correspond to the refractive index values of the first and second hole transport layers in the Examples. In Example 1 to Example 5, the refractive indexes of the first and second hole transport layers were the same.

TABLE 2

| | | Emission | Driving | |
|---|---|---|---|---|
| | Refractive | efficiency ratio | voltage | Life |
| | index | (%) | difference (V) | ratio (%) |
| Comparative | — | 100 | Ref. | 100 |
| Example | | | | |
| Example 1 | 1.70 | 116 | −0.1 | 267 |
| Example 2 | 1.75 | 117 | +0.3 | 58 |
| Example 3 | 1.76 | 119 | +0.3 | 28 |
| Example 4 | 1.77 | 117 | +0.2 | 159 |
| Example 5 | 1.73 | 119 | 0 | 100 |

Referring to the results of Table 2, it could be confirmed that the Examples including hole transport layers having different refractive indexes showed improved device properties of emission efficiency or device life when compared with the Comparative Example. With respect to the emission efficiency properties of the light emitting diodes, the Examples including multiple hole transport layers showed improved effects by about 16-19% when compared with the Comparative Example including one hole transport layer of a high refractive index.

3. Manufacture and Evaluation of Light Emitting Diode-II

Evaluation on light emitting diodes including the Example Compounds and Comparative Compound in a hole transport region were performed by a method below. The manufacturing method of a light emitting diode for diode evaluation is described below.

(Manufacture of Light Emitting Diode-II)

On a glass substrate, a first electrode having a stacked structure of ITO/Ag/ITO was formed. On the first electrode, first to fourth light emitting units were formed in order.

On the first electrode, HATCN was deposited to a thickness of about 50 Å to form a hole injection layer, a hole transport material was deposited to a thickness of about 600 Å to form a hole transport layer, and a first sub-emission layer including a first host (H1-5) and a first dopant (FD32), and a second sub-emission layer including a second host (H2-2) and a first dopant (FD32) were formed in order. T2T was deposited to a thickness of about 50 Å to form an electron transport region, to provide a first light emitting unit.

On the first light emitting unit, TPM-TAZ and Liq were deposited in a weight ratio of about 5:5 to a thickness of about 200 Å to form an n-type charge generating layer, and a p-type charge generating layer was formed by including CBP and Li to form a first charge generating layer.

After that, on the first charge generating layer, a second light emitting unit including a hole injection layer, a hole transport layer, a first sub-emission layer, a second sub-emission layer, and an electron transport region, with the same structure as the first light emitting unit was provided.

On the second light emitting unit, a second charge generating layer was formed with the same structure as the first charge generating layer, and on the second charge generating layer, a third light emitting unit including a hole injection layer, a hole transport layer, a first sub-emission layer, a second sub-emission layer, and an electron transport region, with the same structure as the first light emitting unit was provided. After that, a third charge generating layer with the same structure as the second charge generating layer was formed on the third light emitting unit.

On the third charge generating layer, HATCN was deposited to a thickness of about 50 Å, a hole transport material was deposited to a thickness of about 600 Å, and TCTA was deposited to a thickness of about 50 Å to form a hole transport region. On the hole transport region, an emission layer including a first host, a second host and a dopant was formed. TPM-TAZ and Liq were deposited in a weight ratio of about 5:5 to a thickness of about 300 Å, and Yb was deposited to a thickness of about 10 Å to form an electron transport region, thereby providing a fourth light emitting unit.

On the fourth light emitting unit, AgMg was provided to a thickness of about 100 Å to form a second electrode. On the second electrode, a capping layer including a P4 compound below was formed to a thickness of about 700 Å.

The light emitting diodes of the Examples were manufactured so as to include the amine compound of an embodiment the hole transport layer of the fourth light emitting unit or the first to fourth light emitting units, and the light emitting diode of the Comparative Example was manufactured so as to include Comparative Compound (HT211) in the hole transport layer of the first to fourth light emitting units.

The materials of the layers used for the manufacture of the light emitting diodes are as follows.

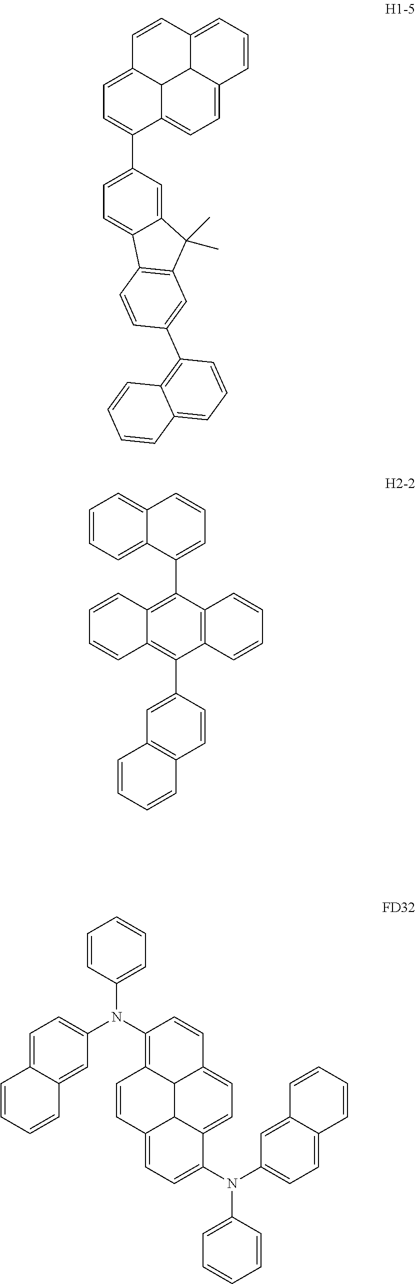

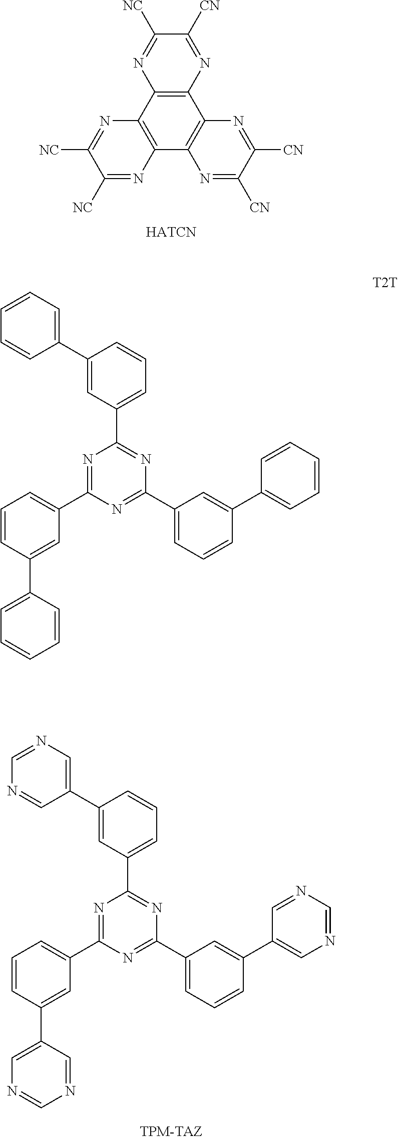

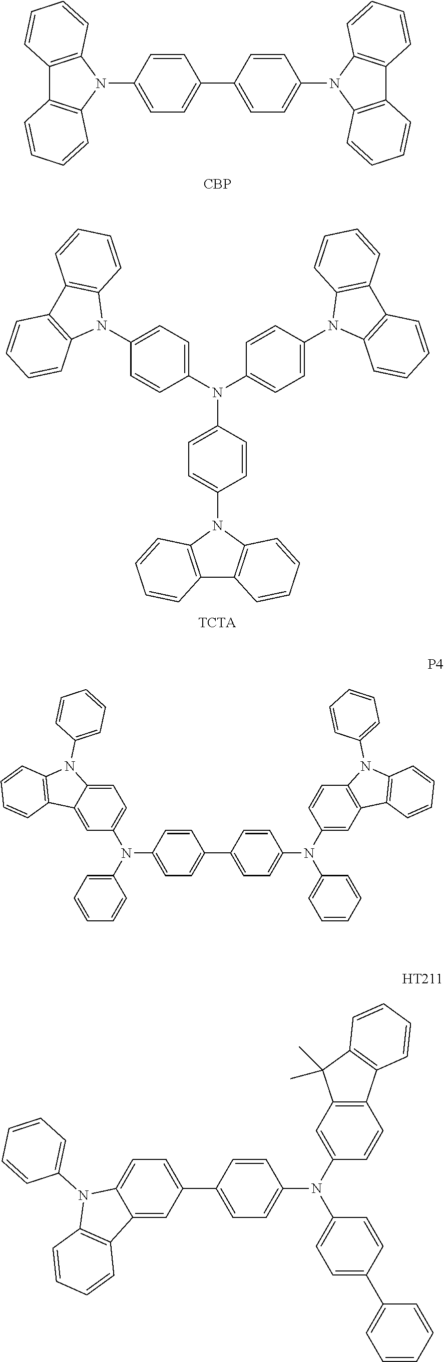 [SMILES: c1ccc2c(c1)c1ccccc1n2-c1ccc(-c2ccc(-n3c4ccccc4c4ccccc43)cc2)cc1 ; "CBP" ; c1ccc2c(c1)c1ccccc1n2-c1ccc(N(c2ccc(-n3c4ccccc4c4ccccc43)cc2)c2ccc(-n3c4ccccc4c4ccccc43)cc2)cc1 ; c1ccc(N(c2ccc(-c3ccc(N(c4ccccc4)c4ccc5c(c4)c4ccccc4n5-c4ccccc4)cc3)cc2)c2ccc3c(c2)c2ccccc2n3-c2ccccc2)cc1 ; CC1(C)c2ccccc2-c2ccc(N(c3ccc(-c4ccccc4)cc3)c3ccc(-c4ccc5c(c4)c4ccccc4n5-c4ccccc4)cc3)cc21]

(Evaluation of Properties of Light Emitting Diode-II)

In Table 3 below, evaluation results of the light emitting diodes of the Comparative Example and the Examples are shown. In Table 1, the driving voltages and emission efficiency of the light emitting diodes of the Comparative Example and the Examples are relatively compared and shown. The driving voltages and emission efficiency in the light emitting diodes of the Example are shown as relative values when the driving voltage and emission efficiency in the light emitting diode of the Comparative Example were set to 100%.

Although not shown in Table 3, the diode life and luminance for all light emitting diodes of the Comparative Example and the Examples were evaluated as similar levels.

In Table 3, the hole transport material corresponds to a hole transport material included in each of the hole transport regions HTR-1, HTR-2, HTR-3, and HTR-4 of the first to fourth light emitting units EU-1, EU-2, EU-3, and EU-4 in the Example of the light emitting diode shown in FIG. 9.

The Comparative Example was manufactured by including a compound of HT211 in each of the hole transport regions HTR-1, HTR-2, HTR-3, and HTR-4 of the first to fourth light emitting units EU-1, EU-2, EU-3, and EU-4.

Examples 1-1, 2-1, 3-1, 4-1, and 5-1 were manufactured by including the compounds of the Examples in each of the hole transport regions HTR-1, HTR-2, HTR-3, and HTR-4 of the first to fourth light emitting units EU-1, EU-2, EU-3, and EU-4. Examples 1-2, 2-2, 3-2, 4-2, and 5-2 were manufactured by including the Example Compounds in the hole transport region HTR-4 of the fourth light emitting unit EU-4, and including the compound of HT211 in each of the hole transport regions HTR-1, HTR-2, and HTR-3 of the first to third light emitting units EU-1, EU-2, and EU-3.

TABLE 3

| | Hole transport material | Driving | |
|---|---|---|---|
| Division | (HTR-1/HTR-2/HTR-3/HTR-4) | voltage | Efficiency |
| Comparative | HT211/HT211/HT211/HT211 | 100% | 100% |
| Example | | | |
| Example 1-1 | Compound 11/Compound | 103% | 107% |
| | 11/Compound 11/Compound 11 | | |
| Example 1-2 | HT211/HT211/HT211/Compound | 100% | 108% |
| | 11 | | |
| Example 2-1 | Compound 12/Compound | 104% | 105% |
| | 12/Compound 12/Compound 12 | | |
| Example 2-2 | HT211/HT211/HT211/Compound | 101% | 105% |
| | 12 | | |
| Example 3-1 | Compound 51/Compound | 104% | 107% |
| | 51/Compound 51/Compound 51 | | |
| Example 3-2 | HT211/HT211/HT211/Compound | 101% | 107% |
| | 51 | | |
| Example 4-1 | Compound 53/Compound | 105% | 107% |
| | 53/Compound 53/Compound 53 | | |
| Example 4-2 | HT211/HT211/HT211/Compound | 102% | 108% |
| | 53 | | |
| Example 5-1 | Compound 84/Compound | 104% | 107% |
| | 84/Compound 84/Compound 84 | | |
| Example 5-2 | HT211/HT211/HT211/Compound | 100% | 108% |
| | 84 | | |

Referring to the results of Table 3, the light emitting diodes of the Examples included the amine compound of an embodiment in a hole transport region of at least one light emitting units among the multiple light emitting units, and showed similar driving voltage characteristics as the Comparative Example and improved emission efficiency properties.

The light emitting diodes of Examples 1-1, 2-1, 3-1, 4-1, and 5-1 included the amine compound of an embodiment in the hole transport region of all light emitting units stacked, and showed similar driving voltage characteristics as the Comparative Example and improved emission efficiency properties.

The light emitting diodes of Examples 1-2, 2-2, 3-2, 4-2, and 5-2 included the amine compound of an embodiment in the hole transport region of a light emitting unit adjacent to a second electrode among multiple light emitting units, and showed similar driving voltage characteristics as the Comparative Example and improved emission efficiency properties.

The light emitting diodes of Examples 1-2, 2-2, 3-2, 4-2 and 5-2 showed reduced driving voltages when compared to corresponding light emitting diodes of Examples 1-1, 2-1, 3-1, 4-1, and 5-1 including the same amine compounds of the Examples in the hole transport region.

It could be found that the light emitting diodes of the Examples include the amine compound of an embodiment, having low refraction properties in at least one light emitting unit and may show improved emission efficiency properties. It could be found that the amine compound of an embodiment is included in the hole transport region of a light emitting unit adjacent to the second electrode, and excellent emission efficiency and reduced driving voltage characteristics were shown.

The light emitting diode of an embodiment includes a hole transport region having a stacked structure of hole transport layer with low refraction/hole transport layer with high refraction/hole transport layer with low refraction, and may show high light extraction effects, and accordingly, excellent emission efficiency properties. The display device of an embodiment includes a light emitting diode having a hole transport region in which hole transport layers having different refractive indexes are stacked, and may show high luminance properties.

The light emitting diode of an embodiment includes multiple light emitting units, and a hole transport material with low refraction properties in at least one light emitting unit, thereby showing high light extraction efficiency and excellent emission efficiency properties. The display device of an embodiment includes the light emitting diode including a hole transport material with low refraction properties, and may show high luminance properties.

The light emitting diode of an embodiment includes multiple hole transport layers having different refractive indexes and may show improved light extraction properties.

The display device of an embodiment includes a light emitting diode including multiple hole transport layers having different refractive indexes and may show excellent emission efficiency.

Embodiments have been disclosed herein, and although terms are employed, they are used and are to be interpreted in a generic and descriptive sense only and not for purpose of limitation. In some instances, as would be apparent by one of ordinary skill in the art, features, characteristics, and/or elements described in connection with an embodiment may be used singly or in combination with features, characteristics, and/or elements described in connection with other embodiments unless otherwise specifically indicated. Accordingly, it will be understood by those of ordinary skill in the art that various changes in form and details may be made without departing from the spirit and scope of the disclosure as set forth in the claims.

What is claimed is:

1. A light emitting diode, comprising:
a first electrode;
a hole transport region disposed on the first electrode;
an emission layer disposed on the hole transport region;
an electron transport region disposed on the emission layer; and
a second electrode disposed on the electron transport region,
wherein the hole transport region comprises:
a first hole transport layer disposed adjacent to the first electrode, the first hole transport layer having a first refractive index;
a second hole transport layer disposed adjacent to the emission layer, the second hole transport layer having a second refractive index; and
a third hole transport layer disposed between the first hole transport layer and the second hole transport layer, the third hole transport layer having a third refractive index which is greater than each of the first refractive index and the second refractive index.

2. The light emitting diode of claim 1, wherein
a difference between the third refractive index and the first refractive index is greater than about 0.1, and
a difference between the third refractive index and the second refractive index is greater than about 0.1.

3. The light emitting diode of claim 2, wherein
the first refractive index and the second refractive index are each in a range of about 1.30 to about 1.80 at a wavelength of about 460 nm, and
the third refractive index is in a range of about 1.85 to about 2.40 at a wavelength of about 460 nm.

4. The light emitting diode of claim 2, wherein the first refractive index and the second refractive index are the same.

5. The light emitting diode of claim 1, wherein the second hole transport layer is disposed directly below the emission layer.

6. The light emitting diode of claim 5, wherein
a refractive index of the emission layer is greater than the second refractive index of the second hole transport layer, and
a difference between the refractive index of the emission layer and the second refractive index is greater than about 0.1 at a wavelength of about 460 nm.

7. The light emitting diode of claim 6, wherein the refractive index of the emission layer is in a range of about 1.80 to about 2.40 at a wavelength of about 460 nm.

8. The light emitting diode of claim 1, wherein the first hole transport layer is disposed directly above the first electrode.

9. The light emitting diode of claim 8, wherein
a refractive index of the first electrode is greater than the first refractive index of the first hole transport layer, and
a difference between the refractive index of the first electrode and the first refractive index is greater than about 0.1 at a wavelength of about 460 nm.

10. The light emitting diode of claim 9, wherein the refractive index of the first electrode is in a range of about 1.80 to about 2.40 at a wavelength of about 460 nm.

11. The light emitting diode of claim 1, wherein a thickness ratio of the first hole transport layer, the third hole transport layer, and the second hole transport layer is in a range of about 0.1:0.8:0.1 to about 0.45:0.1:0.45.

12. The light emitting diode of claim 1, wherein
the first electrode is a reflective electrode, and
the second electrode is a transmissive electrode or a transflective electrode.

13. The light emitting diode of claim 1, wherein the emission layer emits light having a central wavelength in a range of about 430 nm to about 470 nm.

14. The light emitting diode of claim 13, wherein
a thickness of the first hole transport layer is in a range of about 100 Å to about 1,000 Å,
a thickness of the second hole transport layer is in a range of about 100 Å to about 1,000 Å, and
a thickness of the third hole transport layer is in a range of about 100 Å to about 1,000 Å about 100 Å to about 1,000 Å.

15. The light emitting diode of claim 1, wherein the first hole transport layer and the second hole transport layer each independently comprises an amine compound represented by Formula 1: 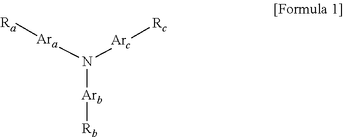
wherein in Formula 1,
Arₐ to Ar_{c} are each independently a substituted or unsubstituted aryl group of 6 to 30 ring-forming carbon atoms, or a substituted or unsubstituted heteroaryl group of 3 to 30 ring-forming carbon atoms,
at least two of Rₐ to R_{c} are each independently an adamantyl group or a cyclohexyl group, and
the remainder of Rₐ to R_{c} is a hydrogen atom, a deuterium atom, a halogen atom, a cyano group, a substituted or unsubstituted oxy group, a substituted or unsubstituted thio group, a substituted or unsubstituted amine group, or a substituted or unsubstituted alkyl group of 1 to 20 carbon atoms.

16. The light emitting diode of claim 15, wherein Arₐ to Ar_{c} are each independently a substituted or unsubstituted phenyl group, a substituted or unsubstituted naphthyl group, a substituted or unsubstituted biphenyl group, a substituted or unsubstituted terphenyl group, a substituted or unsubstituted fluorenyl group, a substituted or unsubstituted carbazole group, a substituted or unsubstituted dibenzofuranyl group, or a substituted or unsubstituted dibenzothiophenyl group.

17. The light emitting diode of claim 1, wherein the first hole transport layer and the second hole transport layer each independently comprises at least one amine compound selected from Compound Group 1:

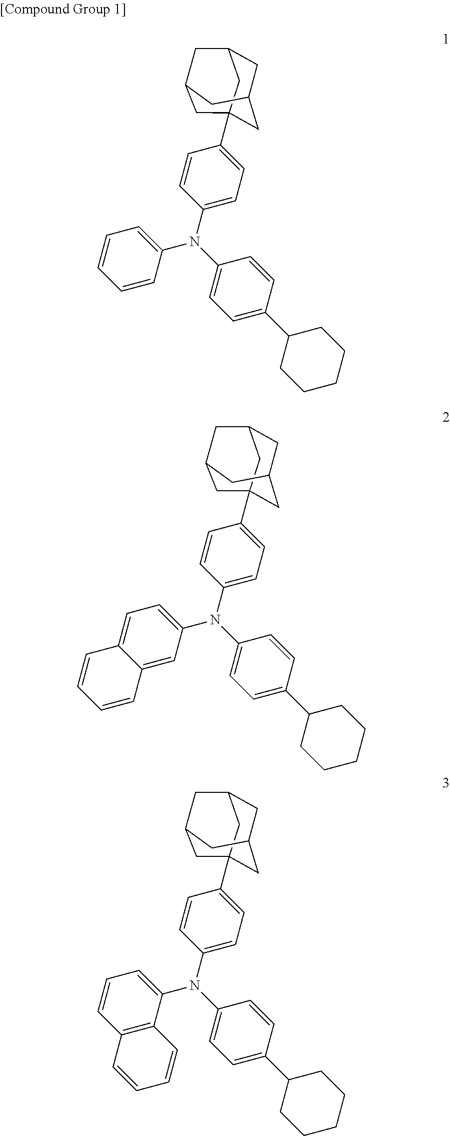 [SMILES: c1ccc(N(c2ccc(C3CCCCC3)cc2)c2ccc(C34CC5CC(CC(C5)C3)C4)cc2)cc1 ; c1ccc2cc(N(c3ccc(C4CCCCC4)cc3)c3ccc(C45CC6CC(CC(C6)C4)C5)cc3)ccc2c1 ; c1ccc2c(N(c3ccc(C4CCCCC4)cc3)c3ccc(C45CC6CC(CC(C6)C4)C5)cc3)cccc2c1]

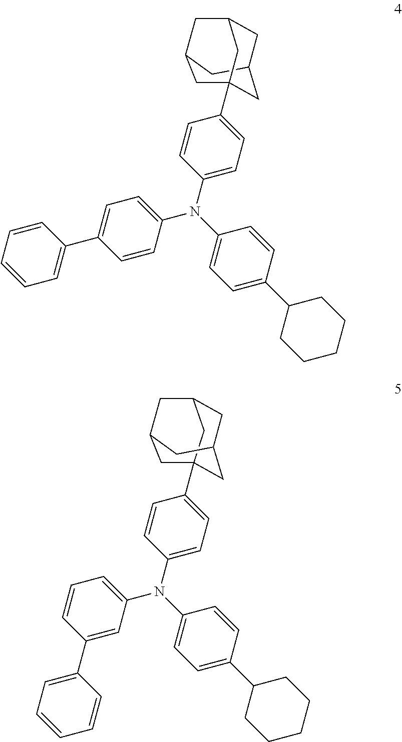 [SMILES: c1ccc(-c2ccc(N(c3ccc(C4CCCCC4)cc3)c3ccc(C45CC6CC(CC(C6)C4)C5)cc3)cc2)cc1 ; c1ccc(-c2cccc(N(c3ccc(C4CCCCC4)cc3)c3ccc(C45CC6CC(CC(C6)C4)C5)cc3)c2)cc1]

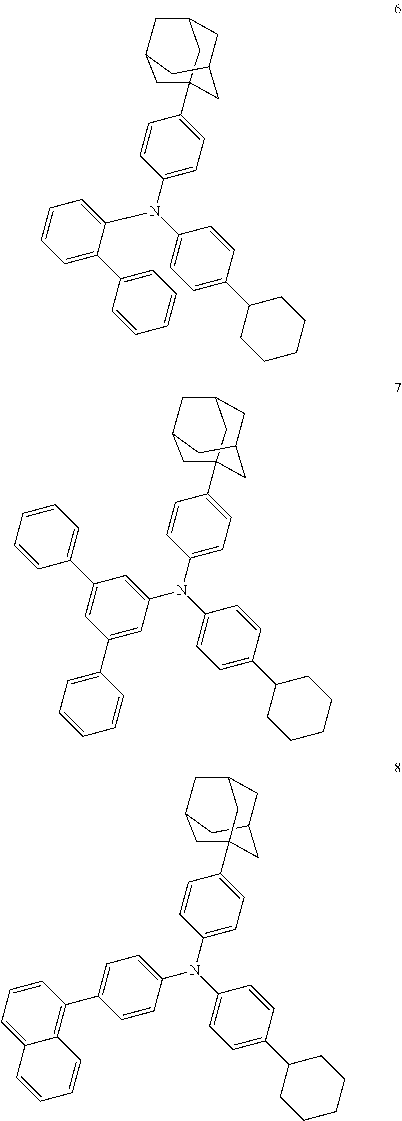 [SMILES: c1ccc(-c2ccccc2N(c2ccc(C3CCCCC3)cc2)c2ccc(C34CC5CC(CC(C5)C3)C4)cc2)cc1 ; c1ccc(-c2cc(-c3ccccc3)cc(N(c3ccc(C4CCCCC4)cc3)c3ccc(C45CC6CC(CC(C6)C4)C5)cc3)c2)cc1 ; c1ccc2c(-c3ccc(N(c4ccc(C5CCCCC5)cc4)c4ccc(C56CC7CC(CC(C7)C5)C6)cc4)cc3)cccc2c1]

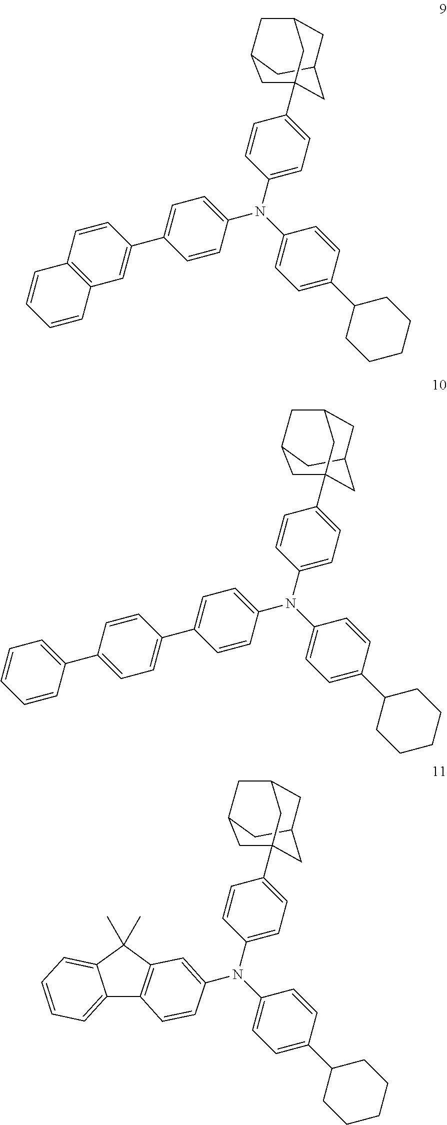 [SMILES: c1ccc2cc(-c3ccc(N(c4ccc(C5CCCCC5)cc4)c4ccc(C56CC7CC(CC(C7)C5)C6)cc4)cc3)ccc2c1 ; c1ccc(-c2ccc(-c3ccc(N(c4ccc(C5CCCCC5)cc4)c4ccc(C56CC7CC(CC(C7)C5)C6)cc4)cc3)cc2)cc1 ; CC1(C)c2ccccc2-c2ccc(N(c3ccc(C4CCCCC4)cc3)c3ccc(C45CC6CC(CC(C6)C4)C5)cc3)cc21]

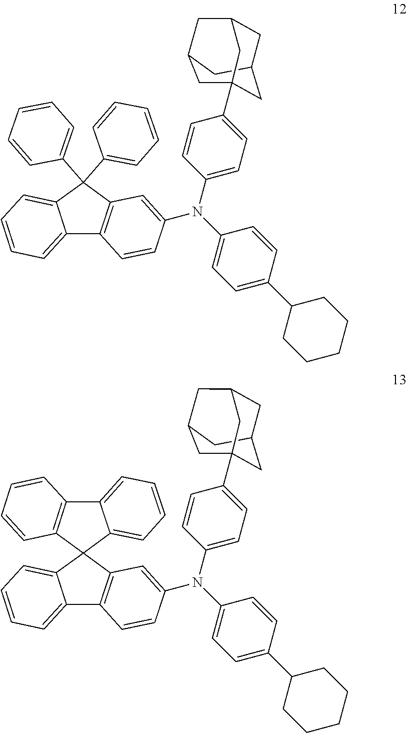 [SMILES: c1ccc(C2(c3ccccc3)c3ccccc3-c3ccc(N(c4ccc(C5CCCCC5)cc4)c4ccc(C56CC7CC(CC(C7)C5)C6)cc4)cc32)cc1 ; c1ccc2c(c1)-c1ccccc1C21c2ccccc2-c2ccc(N(c3ccc(C4CCCCC4)cc3)c3ccc(C45CC6CC(CC(C6)C4)C5)cc3)cc21]

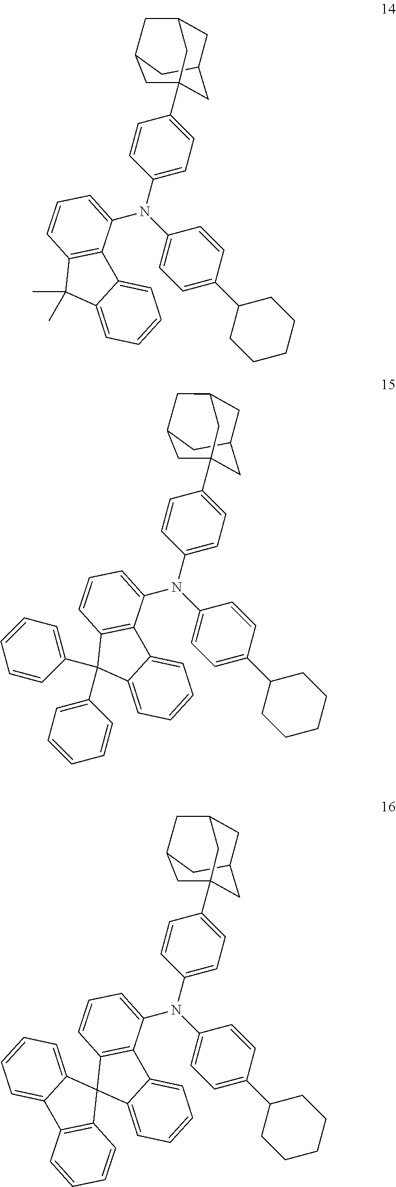 [SMILES: CC1(C)c2ccccc2-c2c(N(c3ccc(C4CCCCC4)cc3)c3ccc(C45CC6CC(CC(C6)C4)C5)cc3)cccc21 ; c1ccc(C2(c3ccccc3)c3ccccc3-c3c(N(c4ccc(C5CCCCC5)cc4)c4ccc(C56CC7CC(CC(C7)C5)C6)cc4)cccc32)cc1 ; c1ccc2c(c1)-c1ccccc1C21c2ccccc2-c2c(N(c3ccc(C4CCCCC4)cc3)c3ccc(C45CC6CC(CC(C6)C4)C5)cc3)cccc21]

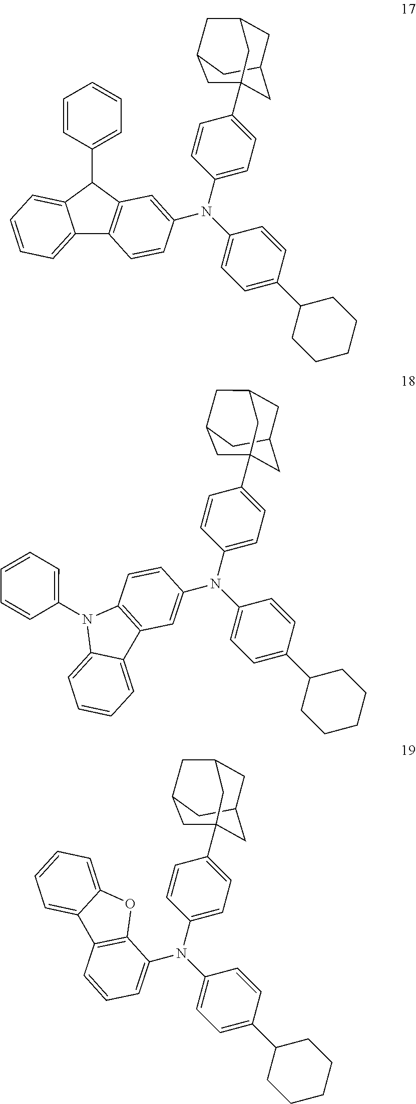 [SMILES: c1ccc(C2c3ccccc3-c3ccc(N(c4ccc(C5CCCCC5)cc4)c4ccc(C56CC7CC(CC(C7)C5)C6)cc4)cc32)cc1 ; c1ccc(-n2c3ccccc3c3cc(N(c4ccc(C5CCCCC5)cc4)c4ccc(C56CC7CC(CC(C7)C5)C6)cc4)ccc32)cc1 ; c1ccc2c(c1)oc1c(N(c3ccc(C4CCCCC4)cc3)c3ccc(C45CC6CC(CC(C6)C4)C5)cc3)cccc12]

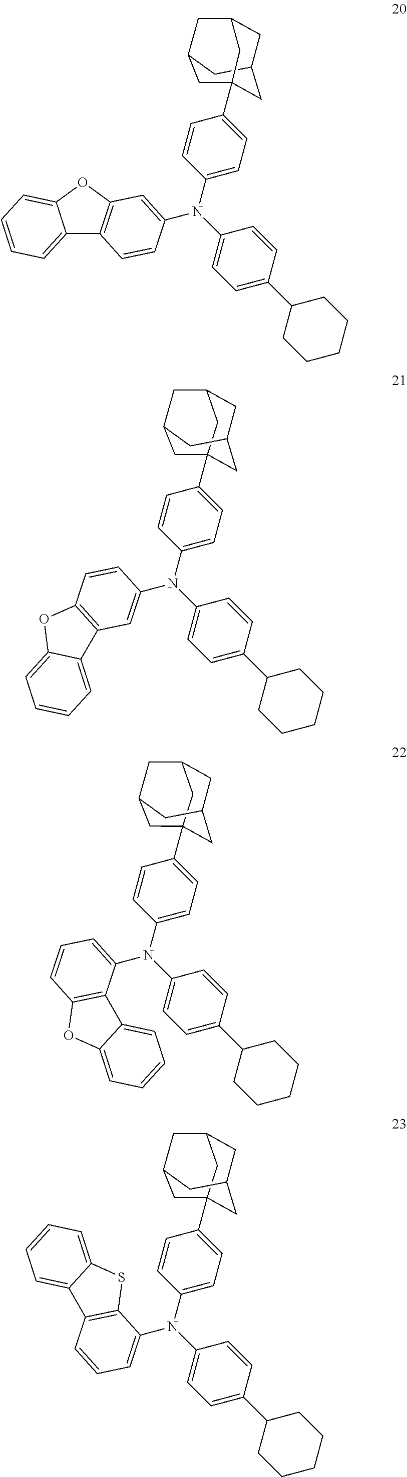 [SMILES: c1ccc2c(c1)oc1cc(N(c3ccc(C4CCCCC4)cc3)c3ccc(C45CC6CC(CC(C6)C4)C5)cc3)ccc12 ; c1ccc2c(c1)oc1ccc(N(c3ccc(C4CCCCC4)cc3)c3ccc(C45CC6CC(CC(C6)C4)C5)cc3)cc12 ; c1ccc2c(c1)oc1cccc(N(c3ccc(C4CCCCC4)cc3)c3ccc(C45CC6CC(CC(C6)C4)C5)cc3)c12 ; c1ccc2c(c1)sc1c(N(c3ccc(C4CCCCC4)cc3)c3ccc(C45CC6CC(CC(C6)C4)C5)cc3)cccc12]

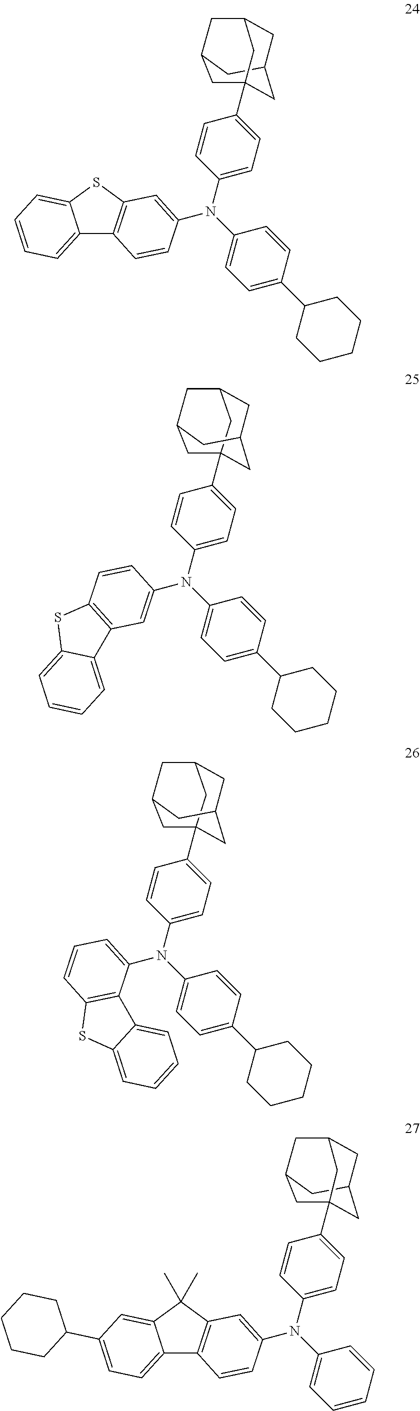

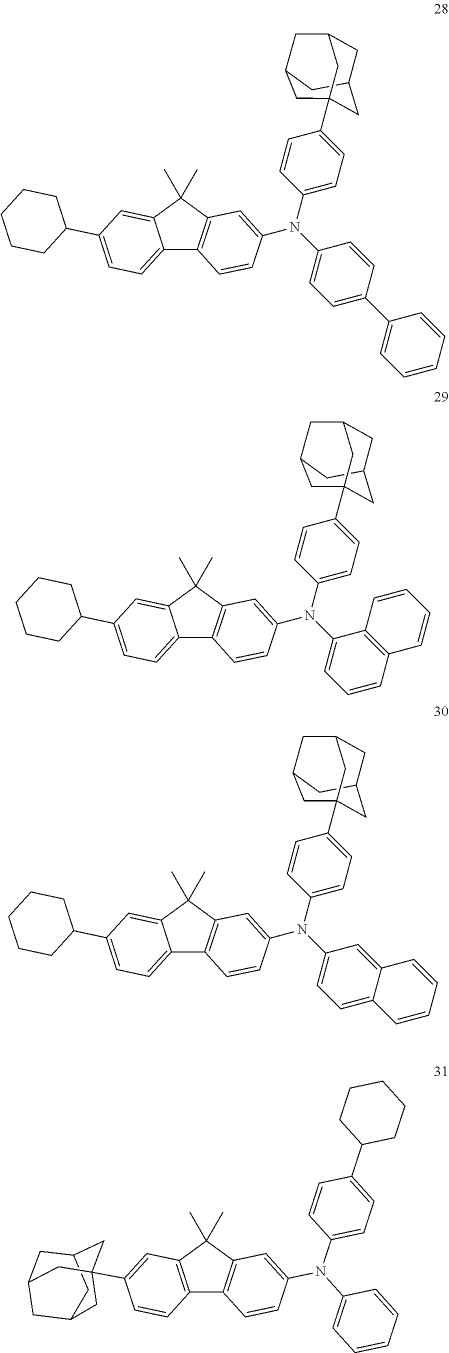

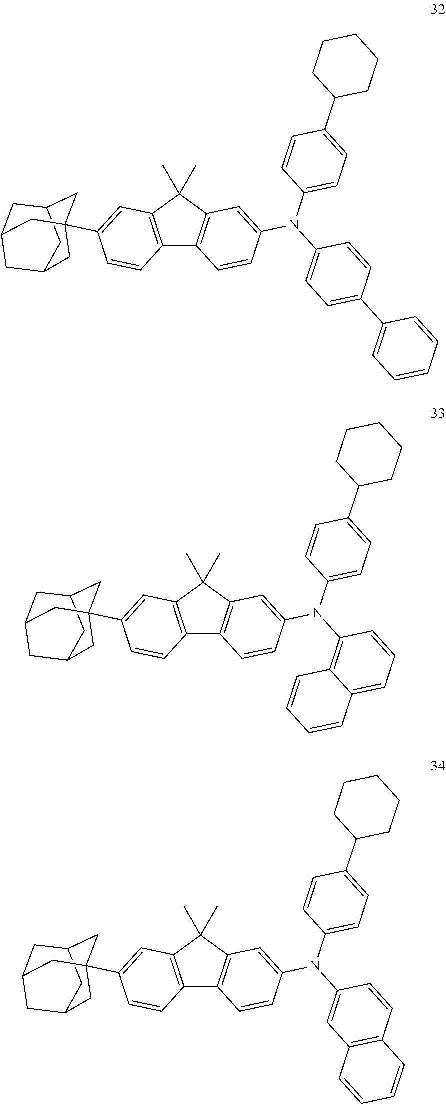

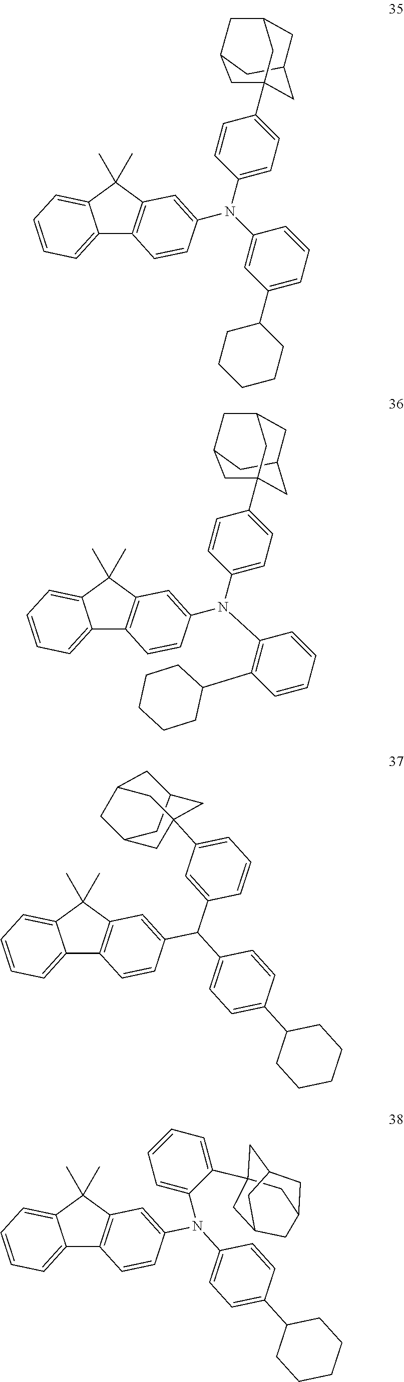

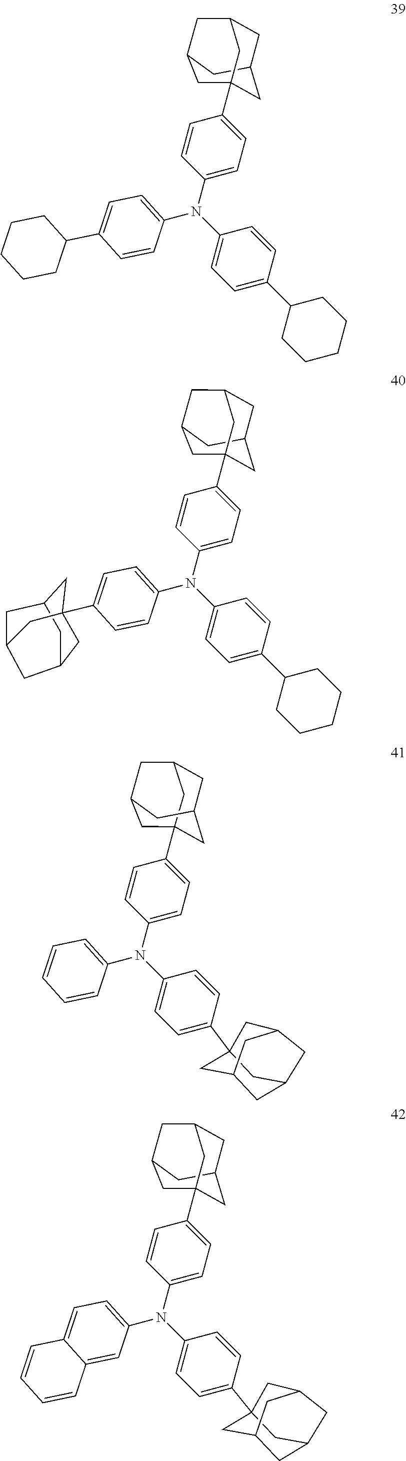 [SMILES: c1cc(N(c2ccc(C3CCCCC3)cc2)c2ccc(C34CC5CC(CC(C5)C3)C4)cc2)ccc1C1CCCCC1 ; c1cc(N(c2ccc(C34CC5CC(CC(C5)C3)C4)cc2)c2ccc(C34CC5CC(CC(C5)C3)C4)cc2)ccc1C1CCCCC1 ; c1ccc(N(c2ccc(C34CC5CC(CC(C5)C3)C4)cc2)c2ccc(C34CC5CC(CC(C5)C3)C4)cc2)cc1 ; c1ccc2cc(N(c3ccc(C45CC6CC(CC(C6)C4)C5)cc3)c3ccc(C45CC6CC(CC(C6)C4)C5)cc3)ccc2c1]

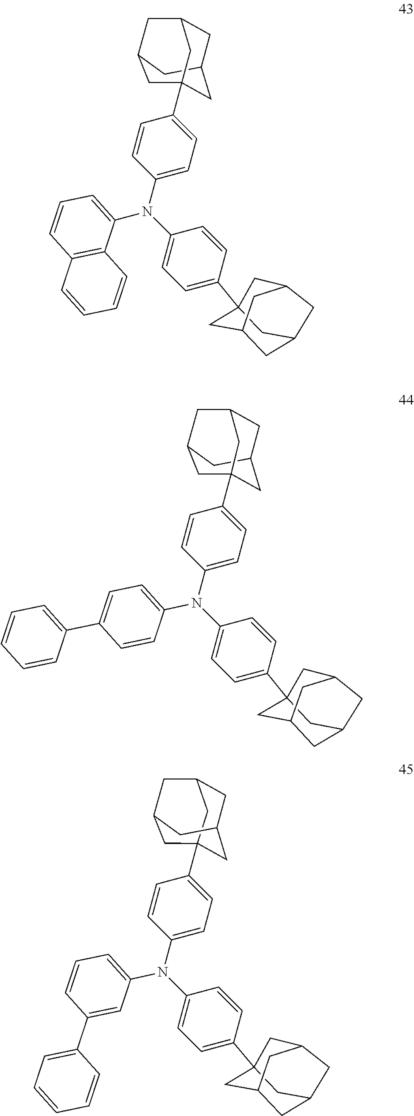 [SMILES: c1ccc2c(N(c3ccc(C45CC6CC(CC(C6)C4)C5)cc3)c3ccc(C45CC6CC(CC(C6)C4)C5)cc3)cccc2c1 ; c1ccc(-c2ccc(N(c3ccc(C45CC6CC(CC(C6)C4)C5)cc3)c3ccc(C45CC6CC(CC(C6)C4)C5)cc3)cc2)cc1 ; c1ccc(-c2cccc(N(c3ccc(C45CC6CC(CC(C6)C4)C5)cc3)c3ccc(C45CC6CC(CC(C6)C4)C5)cc3)c2)cc1]

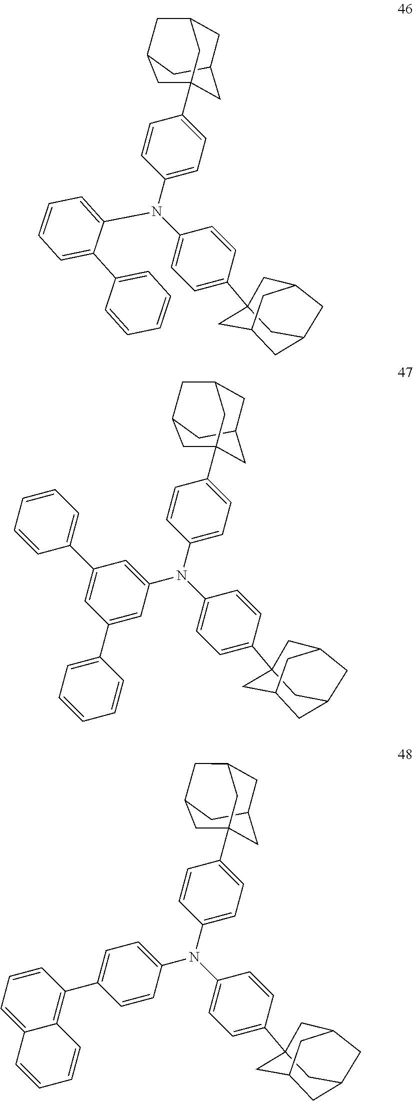 [SMILES: c1ccc(-c2ccccc2N(c2ccc(C34CC5CC(CC(C5)C3)C4)cc2)c2ccc(C34CC5CC(CC(C5)C3)C4)cc2)cc1 ; c1ccc(-c2cc(-c3ccccc3)cc(N(c3ccc(C45CC6CC(CC(C6)C4)C5)cc3)c3ccc(C45CC6CC(CC(C6)C4)C5)cc3)c2)cc1 ; c1ccc2c(-c3ccc(N(c4ccc(C56CC7CC(CC(C7)C5)C6)cc4)c4ccc(C56CC7CC(CC(C7)C5)C6)cc4)cc3)cccc2c1]

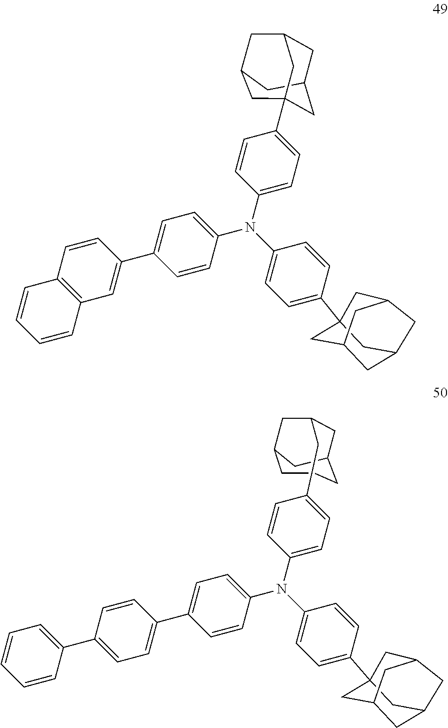 [SMILES: c1ccc2cc(-c3ccc(N(c4ccc(C56CC7CC(CC(C7)C5)C6)cc4)c4ccc(C56CC7CC(CC(C7)C5)C6)cc4)cc3)ccc2c1 ; c1ccc(-c2ccc(-c3ccc(N(c4ccc(CC5CC6CCCC(C6)C5)cc4)c4ccc(C56CC7CC(CC(C7)C5)C6)cc4)cc3)cc2)cc1]

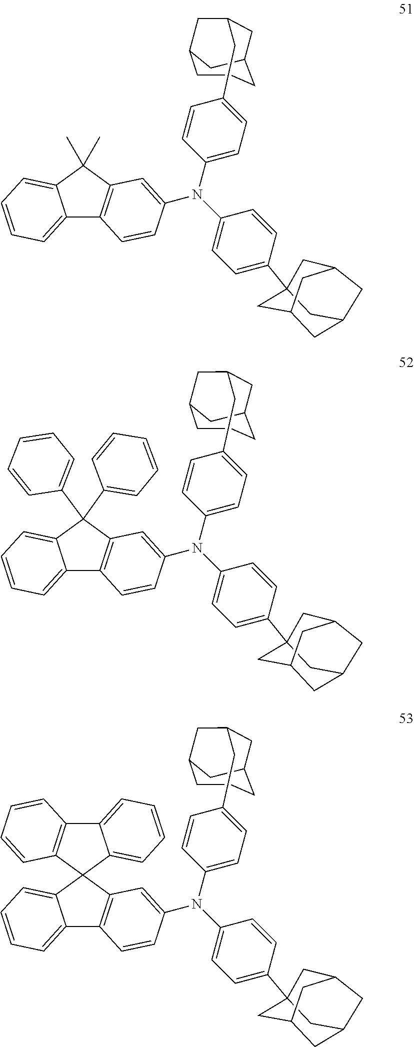 [SMILES: CC1(C)c2ccccc2-c2ccc(N(c3ccc(CC4CC5CCCC(C5)C4)cc3)c3ccc(C45CC6CC(CC(C6)C4)C5)cc3)cc21 ; c1ccc(C2(c3ccccc3)c3ccccc3-c3ccc(N(c4ccc(CC5CC6CCCC(C6)C5)cc4)c4ccc(C56CC7CC(CC(C7)C5)C6)cc4)cc32)cc1 ; c1ccc2c(c1)-c1ccccc1C21c2ccccc2-c2ccc(N(c3ccc(CC4CC5CCCC(C5)C4)cc3)c3ccc(C45CC6CC(CC(C6)C4)C5)cc3)cc21]

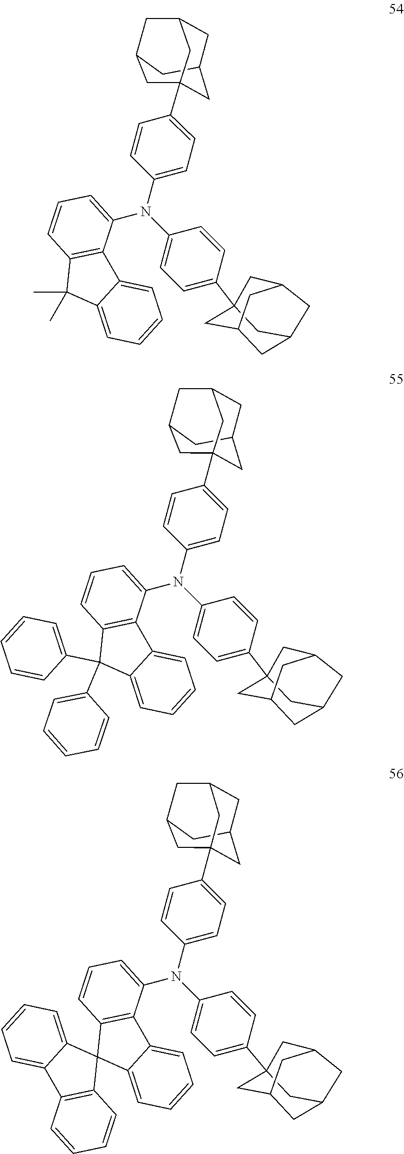 [SMILES: CC1(C)c2ccccc2-c2c(N(c3ccc(C45CC6CC(CC(C6)C4)C5)cc3)c3ccc(C45CC6CC(CC(C6)C4)C5)cc3)cccc21 ; c1ccc(C2(c3ccccc3)c3ccccc3-c3c(N(c4ccc(C56CC7CC(CC(C7)C5)C6)cc4)c4ccc(C56CC7CC(CC(C7)C5)C6)cc4)cccc32)cc1 ; c1ccc2c(c1)-c1ccccc1C21c2ccccc2-c2c(N(c3ccc(C45CC6CC(CC(C6)C4)C5)cc3)c3ccc(C45CC6CC(CC(C6)C4)C5)cc3)cccc21]

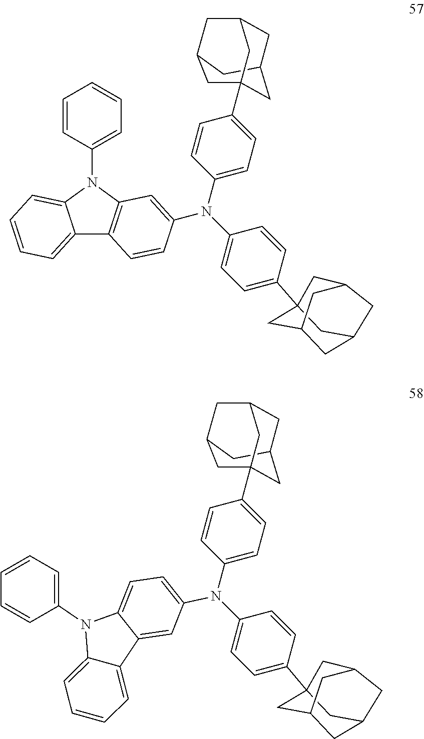 [SMILES: c1ccc(-n2c3ccccc3c3ccc(N(c4ccc(C56CC7CC(CC(C7)C5)C6)cc4)c4ccc(C56CC7CC(CC(C7)C5)C6)cc4)cc32)cc1 ; c1ccc(-n2c3ccccc3c3cc(N(c4ccc(C56CC7CC(CC(C7)C5)C6)cc4)c4ccc(C56CC7CC(CC(C7)C5)C6)cc4)ccc32)cc1]

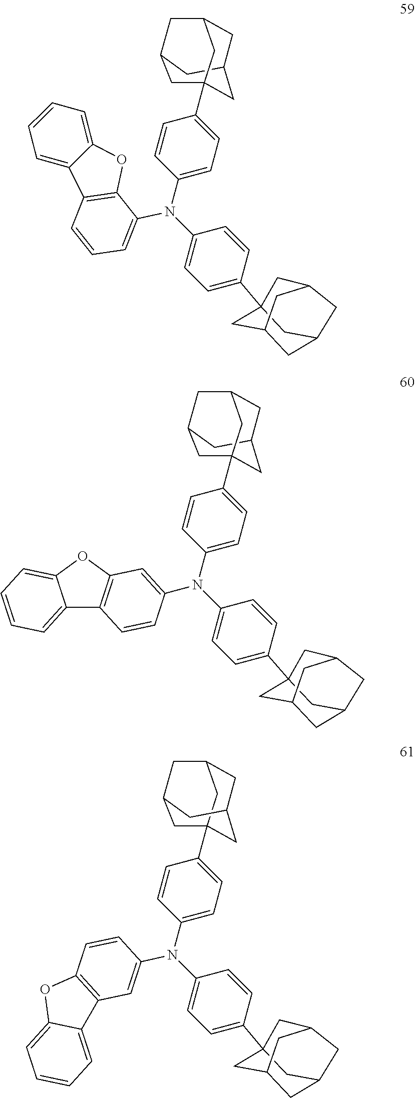 [SMILES: c1ccc2c(c1)oc1c(N(c3ccc(C45CC6CC(CC(C6)C4)C5)cc3)c3ccc(C45CC6CC(CC(C6)C4)C5)cc3)cccc12 ; c1ccc2c(c1)oc1cc(N(c3ccc(C45CC6CC(CC(C6)C4)C5)cc3)c3ccc(C45CC6CC(CC(C6)C4)C5)cc3)ccc12 ; c1ccc2c(c1)oc1ccc(N(c3ccc(C45CC6CC(CC(C6)C4)C5)cc3)c3ccc(C45CC6CC(CC(C6)C4)C5)cc3)cc12]

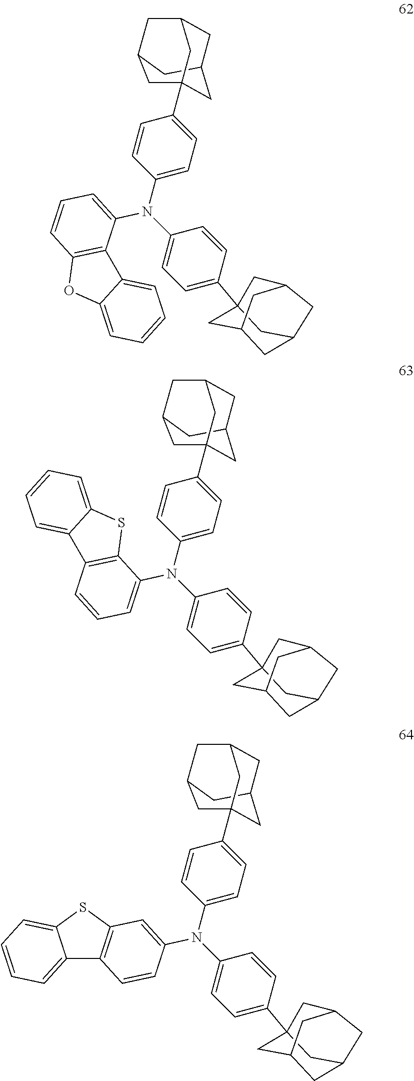 [SMILES: c1ccc2c(c1)oc1cccc(N(c3ccc(C45CC6CC(CC(C6)C4)C5)cc3)c3ccc(C45CC6CC(CC(C6)C4)C5)cc3)c12 ; c1ccc2c(c1)sc1c(N(c3ccc(C45CC6CC(CC(C6)C4)C5)cc3)c3ccc(C45CC6CC(CC(C6)C4)C5)cc3)cccc12 ; c1ccc2c(c1)sc1cc(N(c3ccc(C45CC6CC(CC(C6)C4)C5)cc3)c3ccc(C45CC6CC(CC(C6)C4)C5)cc3)ccc12]

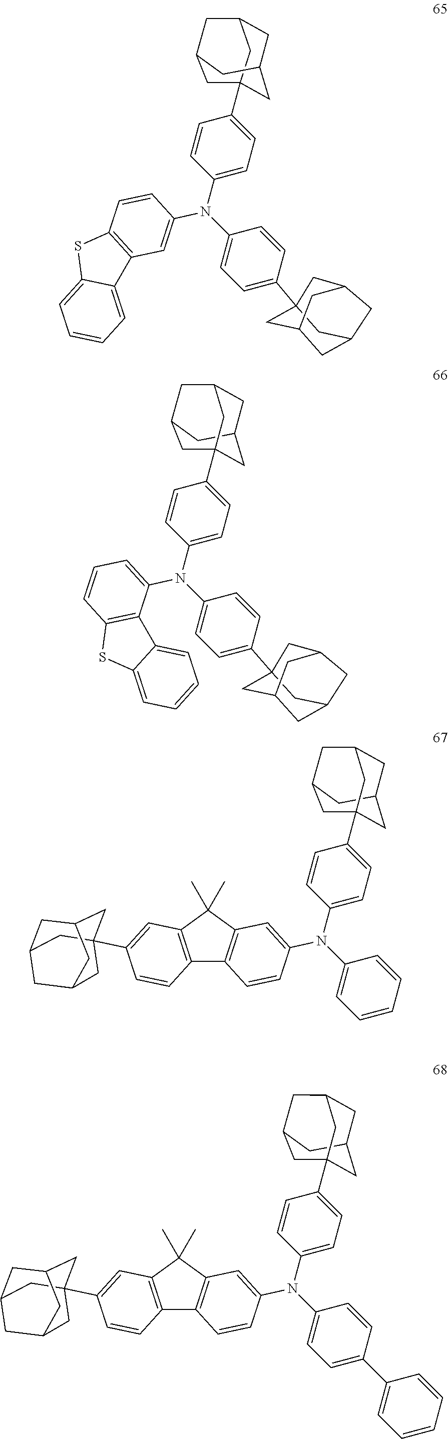

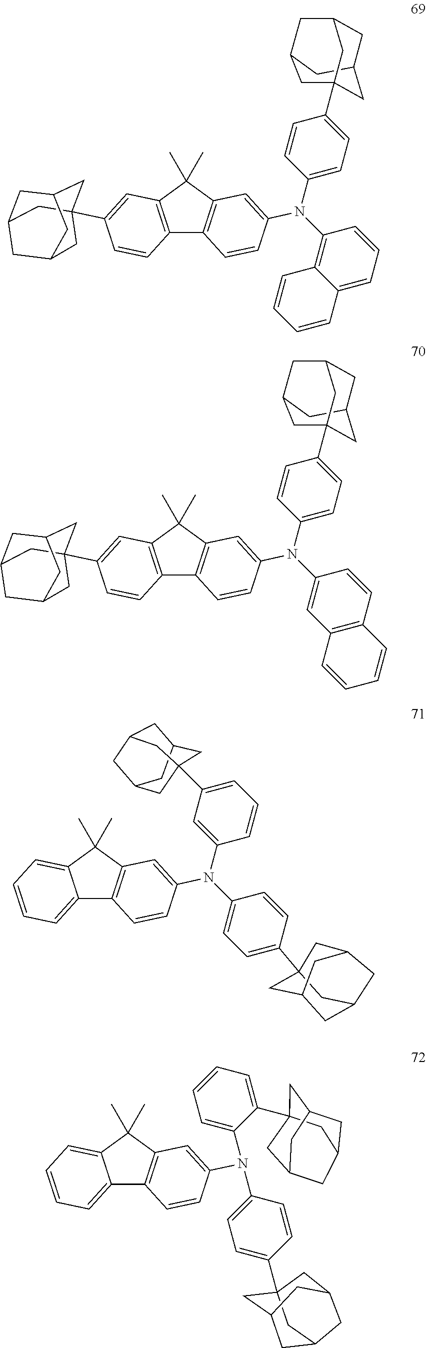

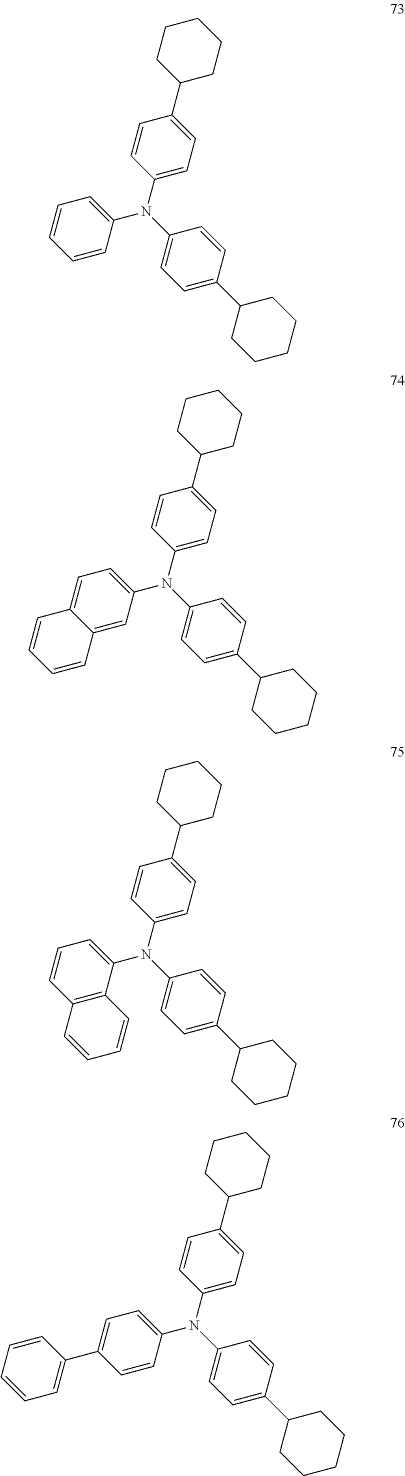

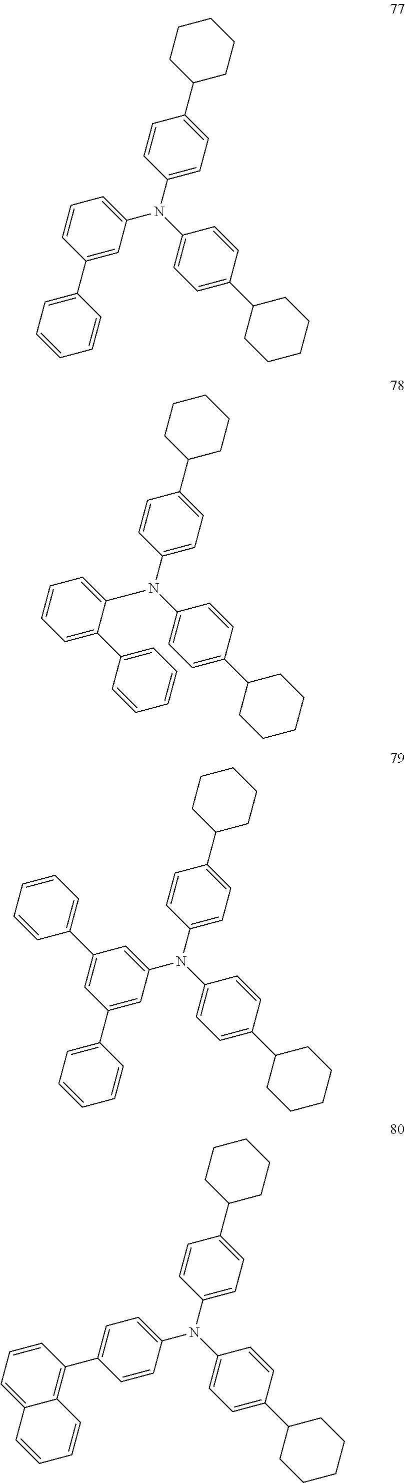

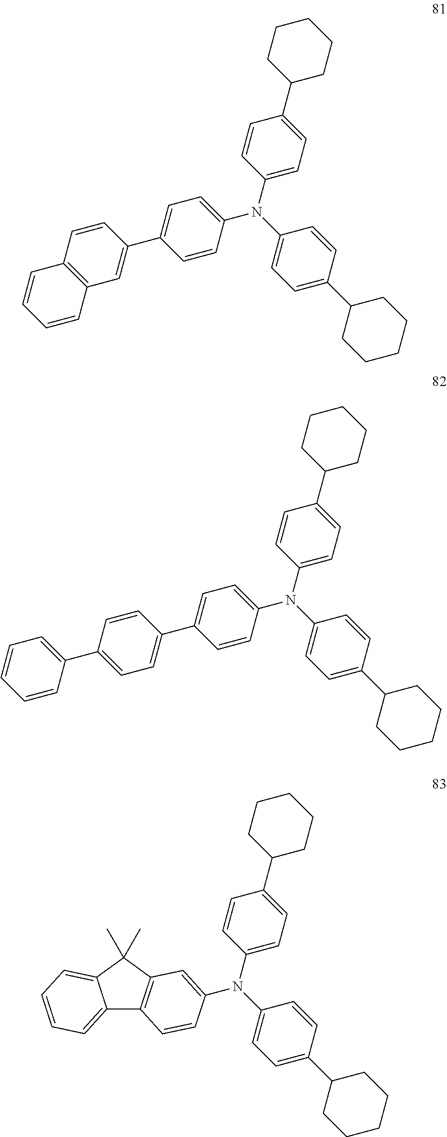 [SMILES: c1ccc2cc(-c3ccc(N(c4ccc(C5CCCCC5)cc4)c4ccc(C5CCCCC5)cc4)cc3)ccc2c1 ; c1ccc(-c2ccc(-c3ccc(N(c4ccc(C5CCCCC5)cc4)c4ccc(C5CCCCC5)cc4)cc3)cc2)cc1 ; CC1(C)c2ccccc2-c2ccc(N(c3ccc(C4CCCCC4)cc3)c3ccc(C4CCCCC4)cc3)cc21]

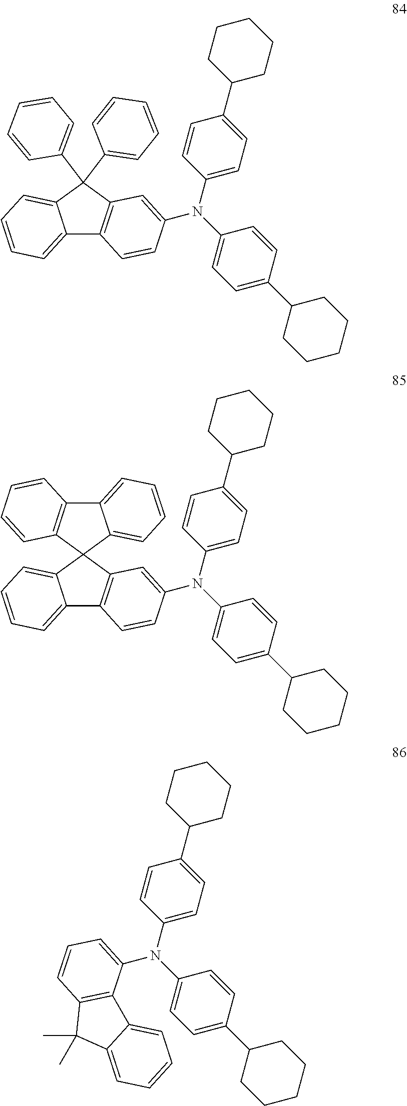 [SMILES: c1ccc(C2(c3ccccc3)c3ccccc3-c3ccc(N(c4ccc(C5CCCCC5)cc4)c4ccc(C5CCCCC5)cc4)cc32)cc1 ; c1ccc2c(c1)-c1ccccc1C21c2ccccc2-c2ccc(N(c3ccc(C4CCCCC4)cc3)c3ccc(C4CCCCC4)cc3)cc21 ; CC1(C)c2ccccc2-c2c(N(c3ccc(C4CCCCC4)cc3)c3ccc(C4CCCCC4)cc3)cccc21]

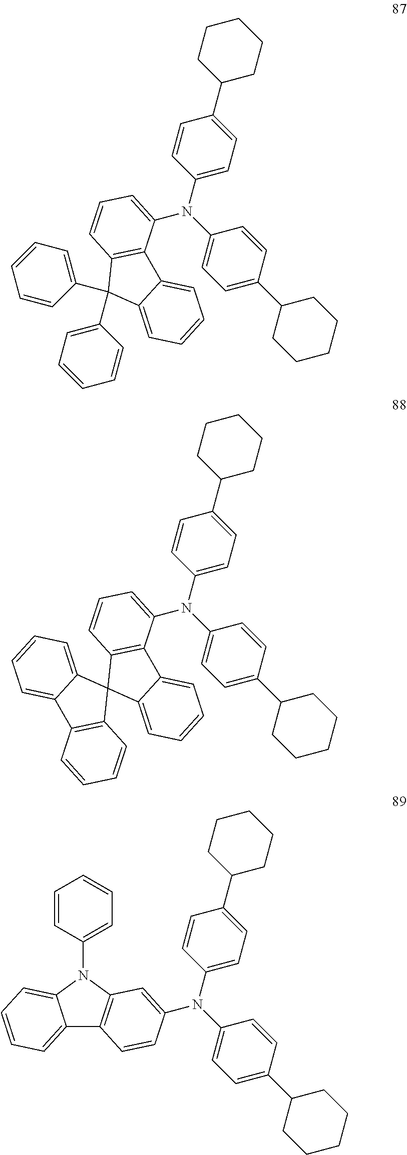 [SMILES: c1ccc(C2(c3ccccc3)c3ccccc3-c3c(N(c4ccc(C5CCCCC5)cc4)c4ccc(C5CCCCC5)cc4)cccc32)cc1 ; c1ccc2c(c1)-c1ccccc1C21c2ccccc2-c2c(N(c3ccc(C4CCCCC4)cc3)c3ccc(C4CCCCC4)cc3)cccc21 ; c1ccc(-n2c3ccccc3c3ccc(N(c4ccc(C5CCCCC5)cc4)c4ccc(C5CCCCC5)cc4)cc32)cc1]

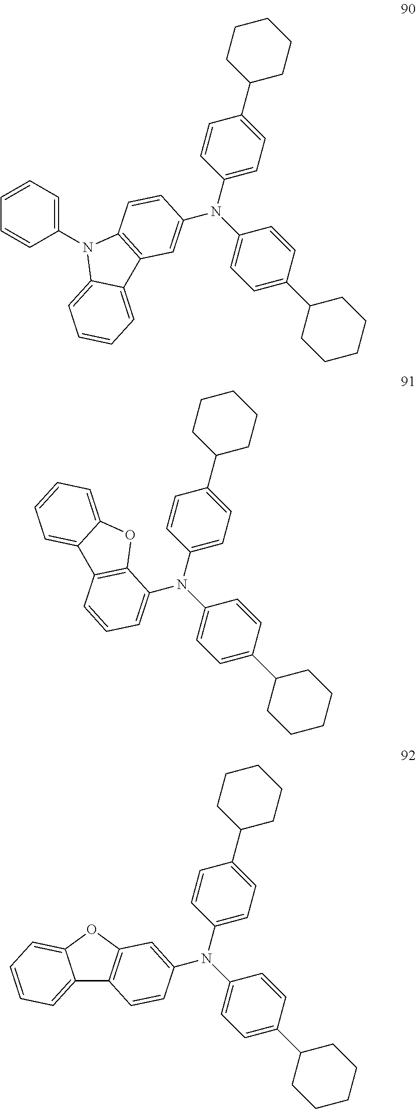

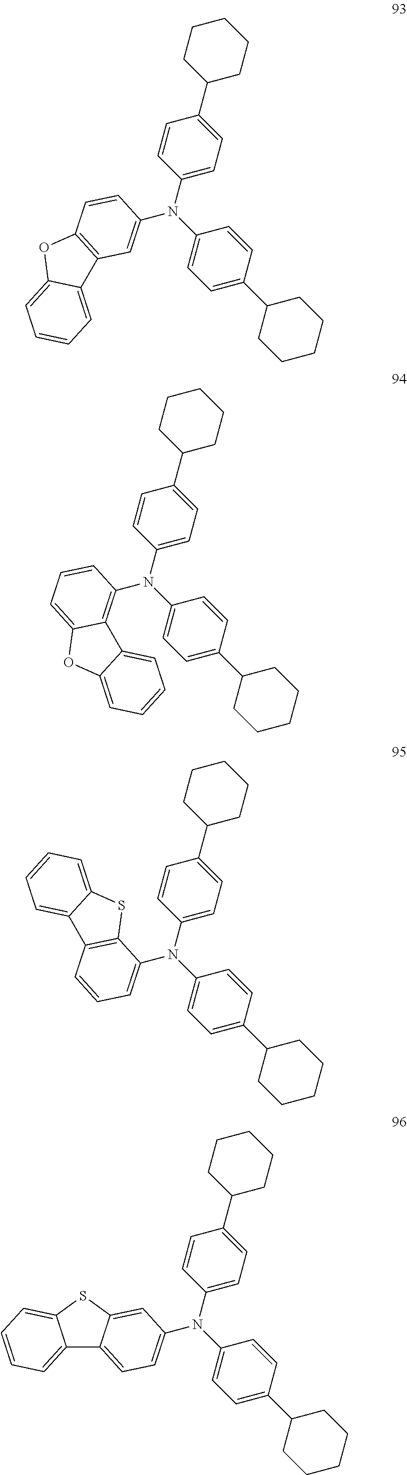

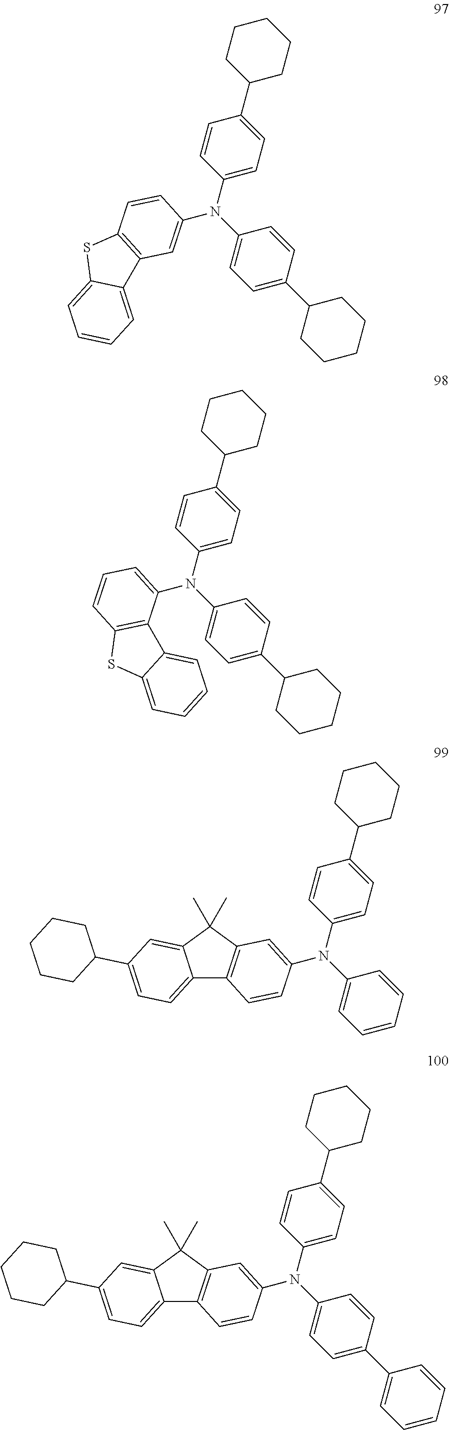 [SMILES: c1ccc2c(c1)sc1ccc(N(c3ccc(C4CCCCC4)cc3)c3ccc(C4CCCCC4)cc3)cc12 ; c1ccc2c(c1)sc1cccc(N(c3ccc(C4CCCCC4)cc3)c3ccc(C4CCCCC4)cc3)c12 ; CC1(C)c2cc(C3CCCCC3)ccc2-c2ccc(N(c3ccccc3)c3ccc(C4CCCCC4)cc3)cc21 ; CC1(C)c2cc(C3CCCCC3)ccc2-c2ccc(N(c3ccc(-c4ccccc4)cc3)c3ccc(C4CCCCC4)cc3)cc21]

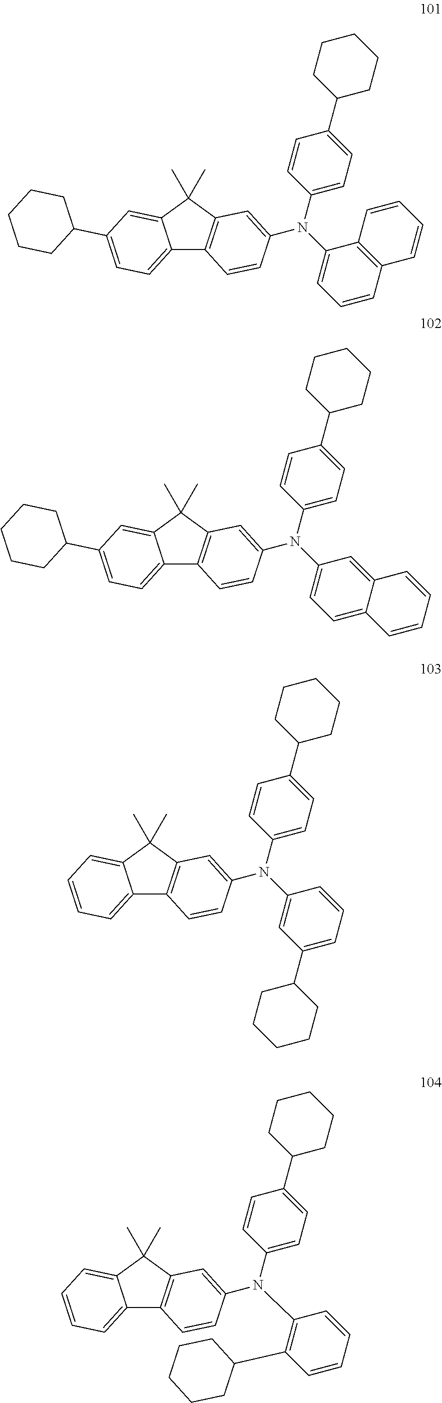

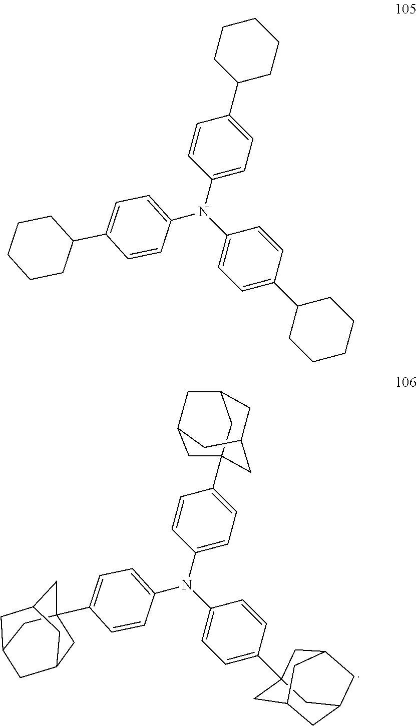 [SMILES: c1cc(N(c2ccc(C3CCCCC3)cc2)c2ccc(C3CCCCC3)cc2)ccc1C1CCCCC1 ; c1cc(C23CC4CC(CC(C4)C2)C3)ccc1N(c1ccc(C23CC4CC(CC(C4)C2)C3)cc1)c1ccc(C23CC4CC(CC(C4)C2)C3)cc1]

18. The light emitting diode of claim 1, wherein the third hole transport layer comprises a compound represented by Formula 2: 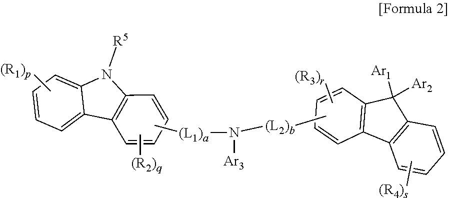
wherein in Formula 2,
Ar₁ and Ar₂ are each independently a hydrogen atom, a deuterium atom, a halogen atom, a substituted or unsubstituted alkyl group of 1 to 30 carbon atoms, a substituted or unsubstituted aryl group of 6 to 30 ring-forming carbon atoms, or a substituted or unsubstituted heteroaryl group of 2 to 30 ring-forming carbon atoms, or combined with an adjacent group to form a ring,
Ar₃ is a substituted or unsubstituted aryl group of 6 to 30 ring-forming carbon atoms, or a substituted or unsubstituted heteroaryl group of 2 to 30 ring-forming carbon atoms,
a and b are each independently 0 or 1,
L₁ and L₂ are each independently a substituted or unsubstituted cycloalkylene group of 3 to 10 ring-forming carbon atoms, a substituted or unsubstituted heterocycloalkylene group of 2 to 10 ring-forming carbon atoms, a substituted or unsubstituted cycloalkenylene group of 3 to 10 ring-forming carbon atoms, a substituted or unsubstituted arylene group of 6 to 30 ring-forming carbon atoms, or a substituted or unsubstituted heteroarylene group of 2 to 60 ring-forming carbon atoms,
p and s are each independently an integer from 0 to 4,
q and r are each independently an integer from 0 to 3, and
R₁ to R₅ are each independently a hydrogen atom, a deuterium atom, a halogen atom, a hydroxyl group, a cyano group, a nitro group, an amino group, a substituted or unsubstituted silyl group, a substituted or unsubstituted oxy group, a substituted or unsubstituted alkyl group of 1 to 60 carbon atoms, a substituted or unsubstituted heterocycloalkyl group of 3 to 60 ring-forming carbon atoms, a substituted or unsubstituted aryl group of 6 to 60 ring-forming carbon atoms, or a substituted or unsubstituted heteroaryl group of 2 to 60 ring-forming carbon atoms.

19. The light emitting diode of claim 1, wherein the third hole transport layer comprises at least one compound selected from Compound Group 2:

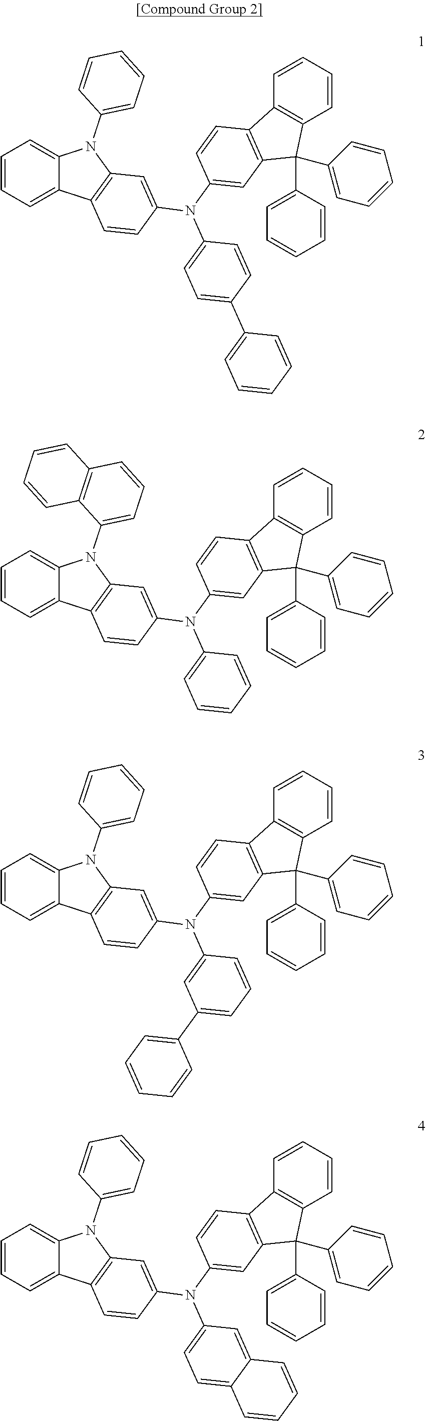

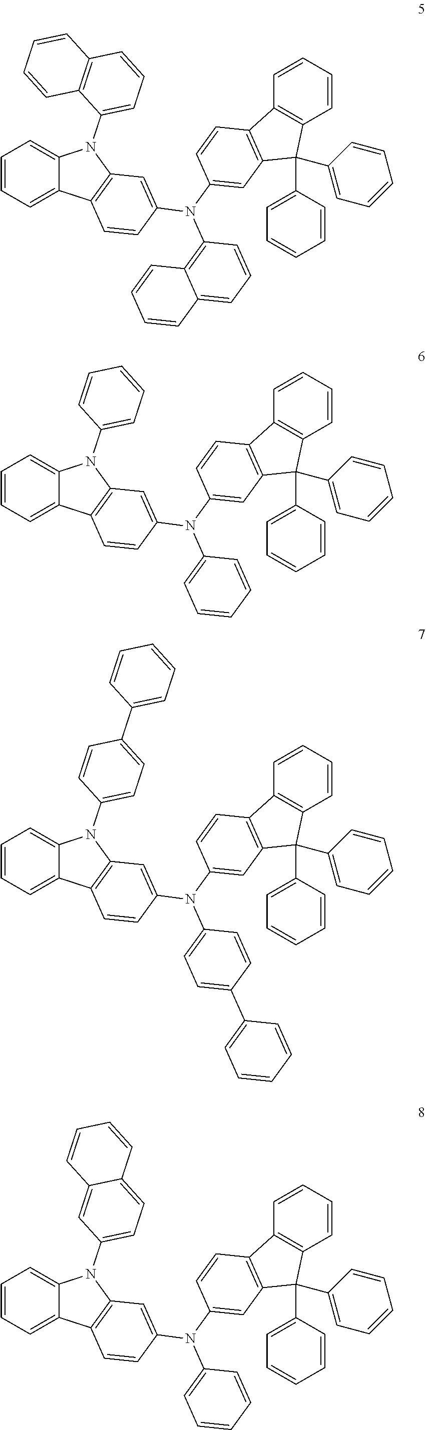 [SMILES: c1ccc(C2(c3ccccc3)c3ccccc3-c3ccc(N(c4ccc5c6ccccc6n(-c6cccc7ccccc67)c5c4)c4cccc5ccccc45)cc32)cc1 ; c1ccc(N(c2ccc3c(c2)C(c2ccccc2)(c2ccccc2)c2ccccc2-3)c2ccc3c4ccccc4n(-c4ccccc4)c3c2)cc1 ; c1ccc(-c2ccc(N(c3ccc4c(c3)C(c3ccccc3)(c3ccccc3)c3ccccc3-4)c3ccc4c5ccccc5n(-c5ccc(-c6ccccc6)cc5)c4c3)cc2)cc1 ; c1ccc(N(c2ccc3c(c2)C(c2ccccc2)(c2ccccc2)c2ccccc2-3)c2ccc3c4ccccc4n(-c4ccc5ccccc5c4)c3c2)cc1]

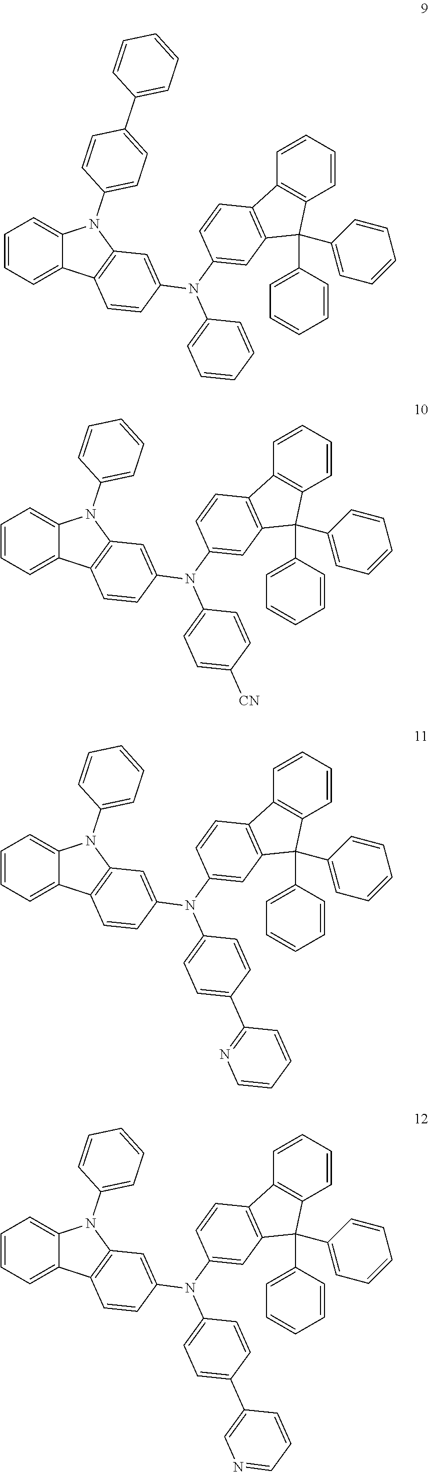 [SMILES: c1ccc(-c2ccc(-n3c4ccccc4c4ccc(N(c5ccccc5)c5ccc6c(c5)C(c5ccccc5)(c5ccccc5)c5ccccc5-6)cc43)cc2)cc1 ; N#Cc1ccc(N(c2ccc3c(c2)C(c2ccccc2)(c2ccccc2)c2ccccc2-3)c2ccc3c4ccccc4n(-c4ccccc4)c3c2)cc1 ; c1ccc(-n2c3ccccc3c3ccc(N(c4ccc(-c5ccccn5)cc4)c4ccc5c(c4)C(c4ccccc4)(c4ccccc4)c4ccccc4-5)cc32)cc1 ; c1ccc(-n2c3ccccc3c3ccc(N(c4ccc(-c5cccnc5)cc4)c4ccc5c(c4)C(c4ccccc4)(c4ccccc4)c4ccccc4-5)cc32)cc1]

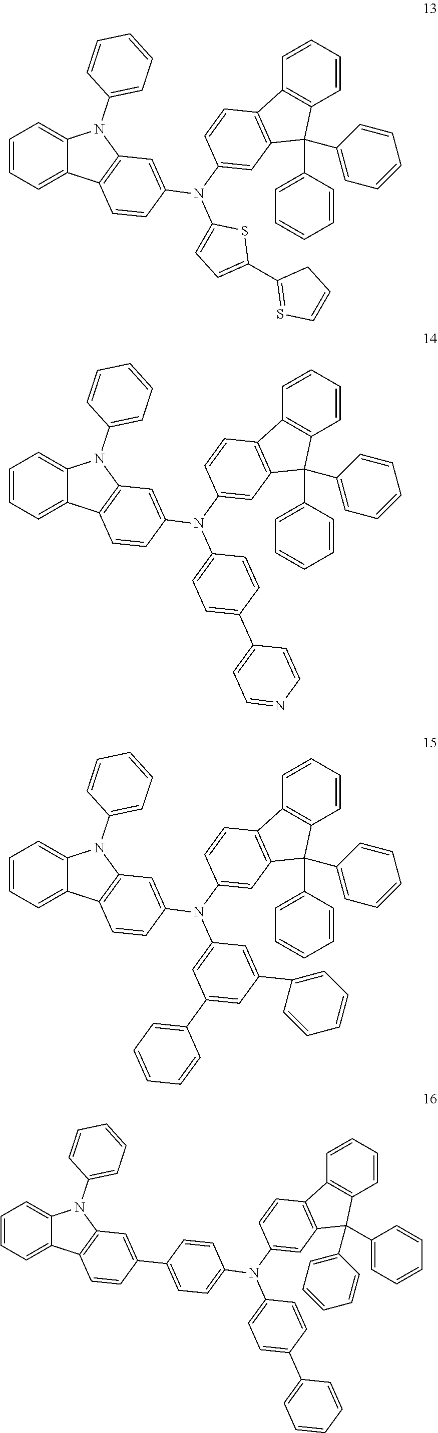

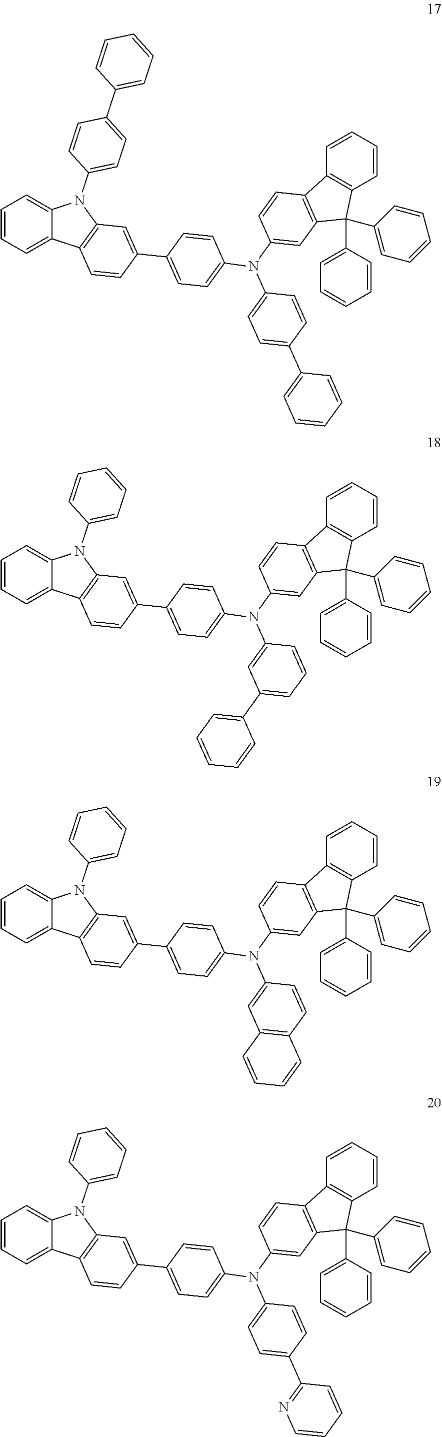 [SMILES: c1ccc(-c2ccc(N(c3ccc(-c4ccc5c6ccccc6n(-c6ccc(-c7ccccc7)cc6)c5c4)cc3)c3ccc4c(c3)C(c3ccccc3)(c3ccccc3)c3ccccc3-4)cc2)cc1 ; c1ccc(-c2cccc(N(c3ccc(-c4ccc5c6ccccc6n(-c6ccccc6)c5c4)cc3)c3ccc4c(c3)C(c3ccccc3)(c3ccccc3)c3ccccc3-4)c2)cc1 ; c1ccc(-n2c3ccccc3c3ccc(-c4ccc(N(c5ccc6c(c5)C(c5ccccc5)(c5ccccc5)c5ccccc5-6)c5ccc6ccccc6c5)cc4)cc32)cc1 ; c1ccc(-n2c3ccccc3c3ccc(-c4ccc(N(c5ccc(-c6ccccn6)cc5)c5ccc6c(c5)C(c5ccccc5)(c5ccccc5)c5ccccc5-6)cc4)cc32)cc1]

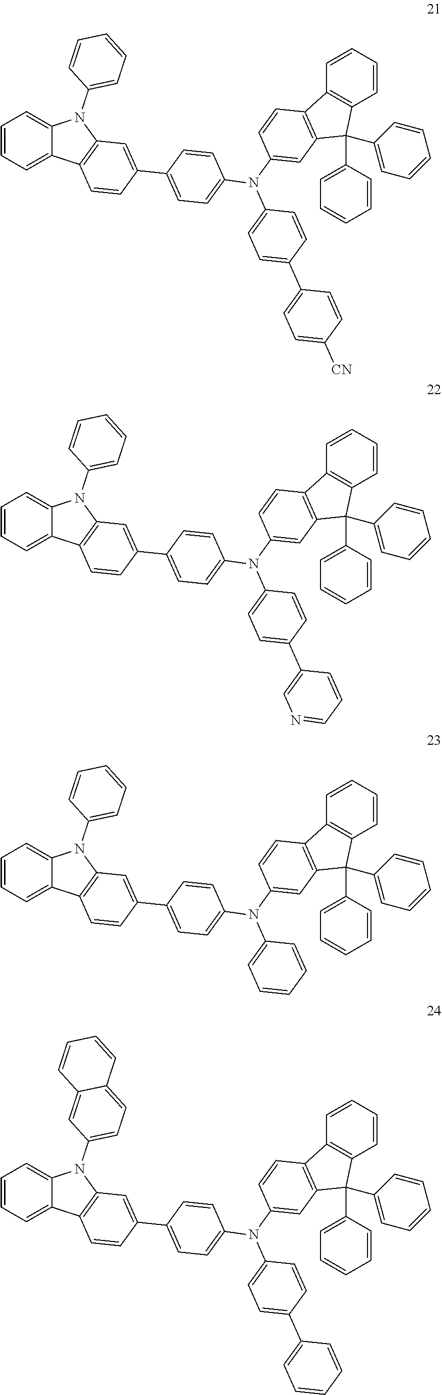 [SMILES: N#Cc1ccc(-c2ccc(N(c3ccc(-c4ccc5c6ccccc6n(-c6ccccc6)c5c4)cc3)c3ccc4c(c3)C(c3ccccc3)(c3ccccc3)c3ccccc3-4)cc2)cc1 ; c1ccc(-n2c3ccccc3c3ccc(-c4ccc(N(c5ccc(-c6cccnc6)cc5)c5ccc6c(c5)C(c5ccccc5)(c5ccccc5)c5ccccc5-6)cc4)cc32)cc1 ; c1ccc(N(c2ccc(-c3ccc4c5ccccc5n(-c5ccccc5)c4c3)cc2)c2ccc3c(c2)C(c2ccccc2)(c2ccccc2)c2ccccc2-3)cc1 ; c1ccc(-c2ccc(N(c3ccc(-c4ccc5c6ccccc6n(-c6ccc7ccccc7c6)c5c4)cc3)c3ccc4c(c3)C(c3ccccc3)(c3ccccc3)c3ccccc3-4)cc2)cc1]

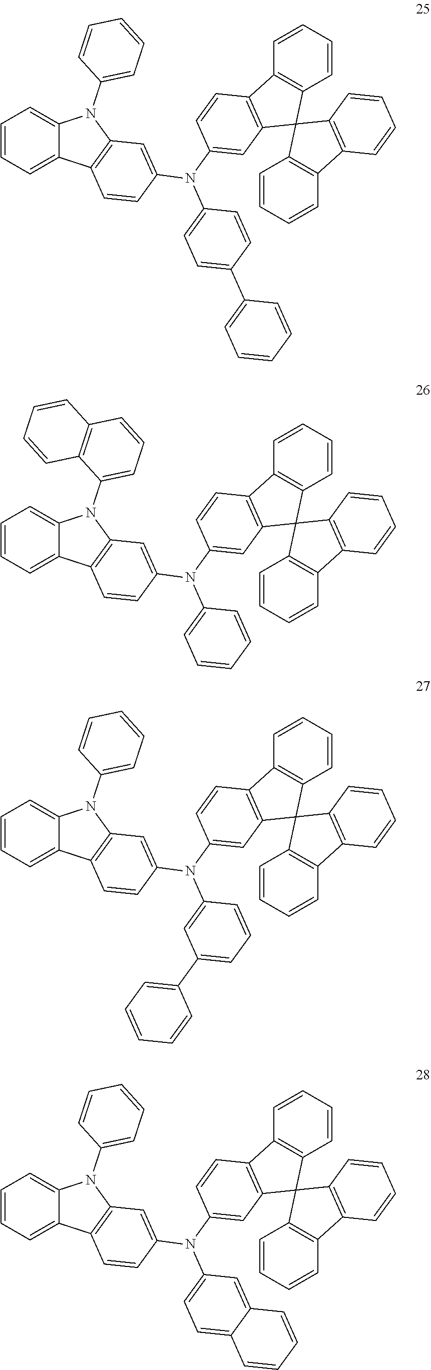 [SMILES: c1ccc(-c2ccc(N(c3ccc4c(c3)C3(c5ccccc5-c5ccccc53)c3ccccc3-4)c3ccc4c5ccccc5n(-c5ccccc5)c4c3)cc2)cc1 ; c1ccc(N(c2ccc3c(c2)C2(c4ccccc4-c4ccccc42)c2ccccc2-3)c2ccc3c4ccccc4n(-c4cccc5ccccc45)c3c2)cc1 ; c1ccc(-c2cccc(N(c3ccc4c(c3)C3(c5ccccc5-c5ccccc53)c3ccccc3-4)c3ccc4c5ccccc5n(-c5ccccc5)c4c3)c2)cc1 ; c1ccc(-n2c3ccccc3c3ccc(N(c4ccc5c(c4)C4(c6ccccc6-c6ccccc64)c4ccccc4-5)c4ccc5ccccc5c4)cc32)cc1]

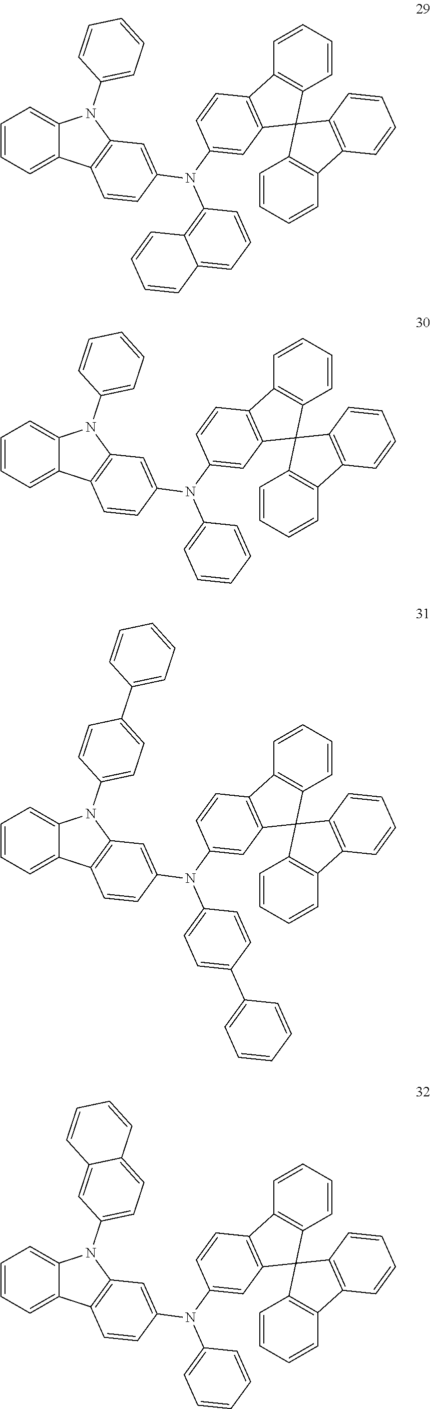 [SMILES: c1ccc(-n2c3ccccc3c3ccc(N(c4ccc5c(c4)C4(c6ccccc6-c6ccccc64)c4ccccc4-5)c4cccc5ccccc45)cc32)cc1 ; c1ccc(N(c2ccc3c(c2)C2(c4ccccc4-c4ccccc42)c2ccccc2-3)c2ccc3c4ccccc4n(-c4ccccc4)c3c2)cc1 ; c1ccc(-c2ccc(N(c3ccc4c(c3)C3(c5ccccc5-c5ccccc53)c3ccccc3-4)c3ccc4c5ccccc5n(-c5ccc(-c6ccccc6)cc5)c4c3)cc2)cc1 ; c1ccc(N(c2ccc3c(c2)C2(c4ccccc4-c4ccccc42)c2ccccc2-3)c2ccc3c4ccccc4n(-c4ccc5ccccc5c4)c3c2)cc1]

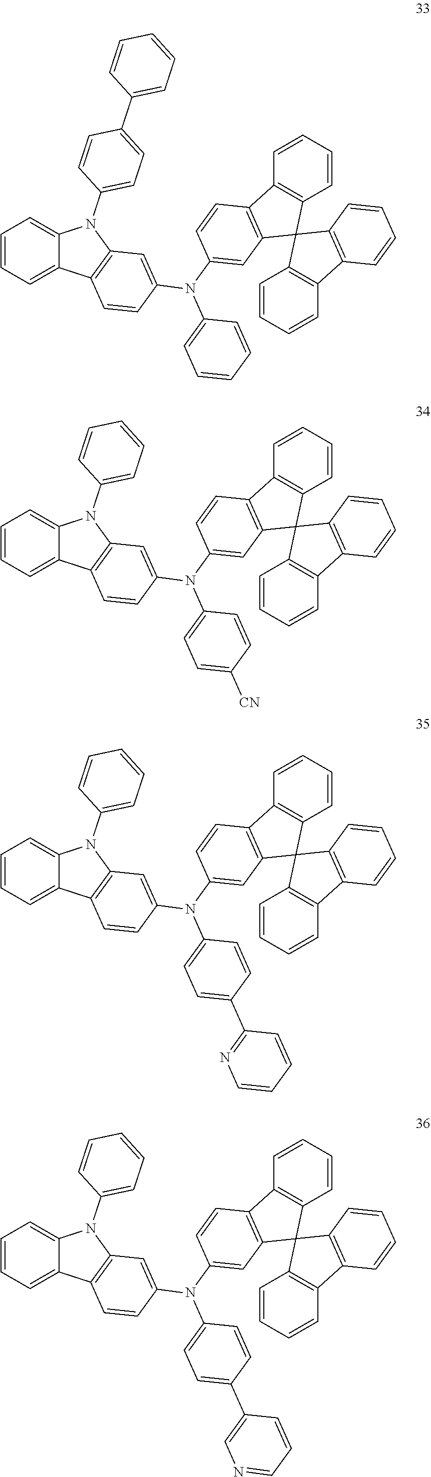 [SMILES: c1ccc(-c2ccc(-n3c4ccccc4c4ccc(N(c5ccccc5)c5ccc6c(c5)C5(c7ccccc7-c7ccccc75)c5ccccc5-6)cc43)cc2)cc1 ; N#Cc1ccc(N(c2ccc3c(c2)C2(c4ccccc4-c4ccccc42)c2ccccc2-3)c2ccc3c4ccccc4n(-c4ccccc4)c3c2)cc1 ; c1ccc(-n2c3ccccc3c3ccc(N(c4ccc(-c5ccccn5)cc4)c4ccc5c(c4)C4(c6ccccc6-c6ccccc64)c4ccccc4-5)cc32)cc1 ; c1ccc(-n2c3ccccc3c3ccc(N(c4ccc(-c5cccnc5)cc4)c4ccc5c(c4)C4(c6ccccc6-c6ccccc64)c4ccccc4-5)cc32)cc1]

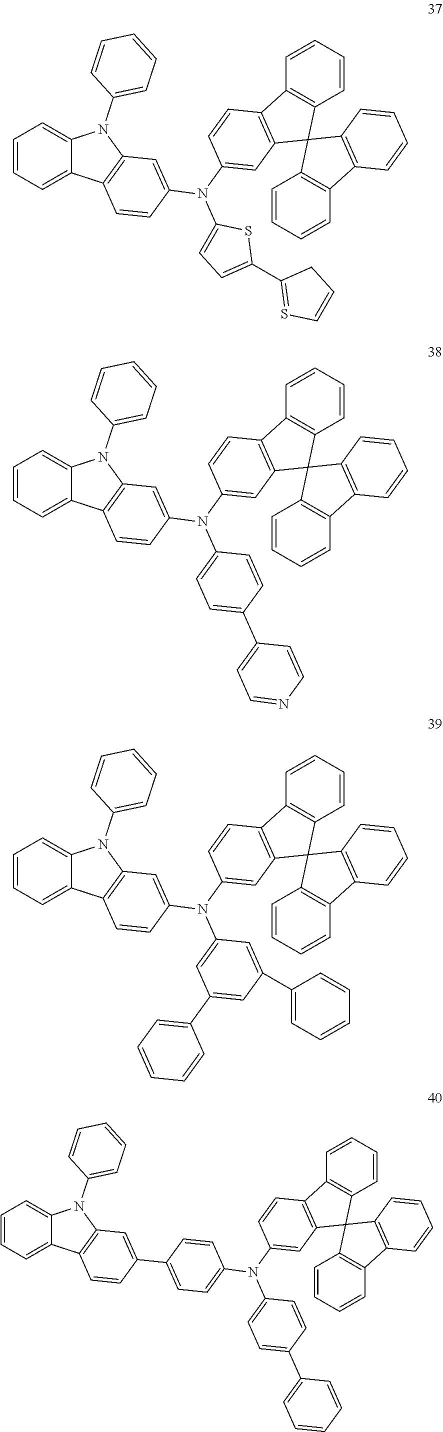

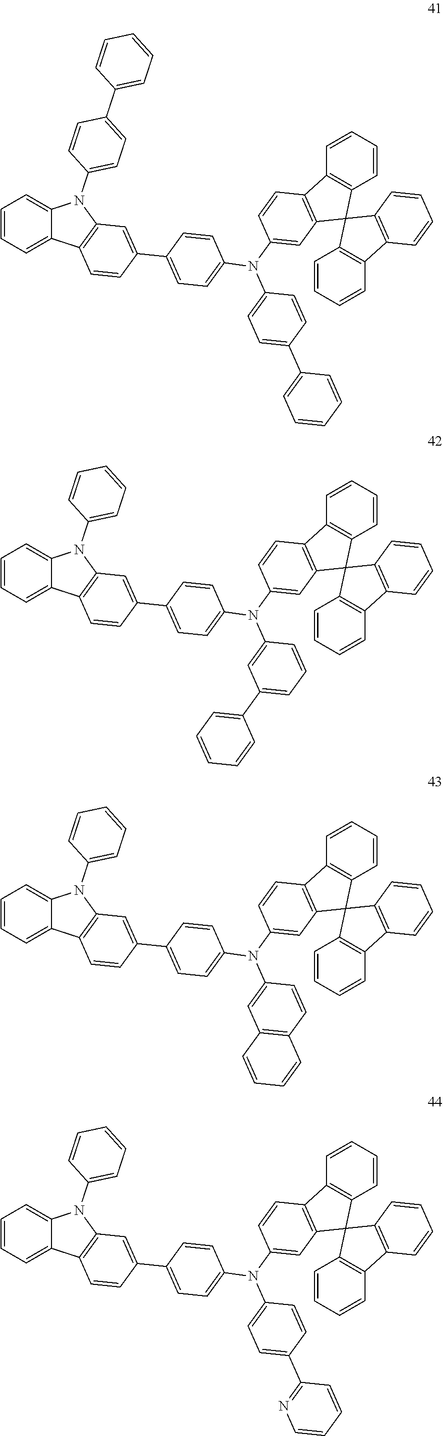 [SMILES: c1ccc(-c2ccc(N(c3ccc(-c4ccc5c6ccccc6n(-c6ccc(-c7ccccc7)cc6)c5c4)cc3)c3ccc4c(c3)C3(c5ccccc5-c5ccccc53)c3ccccc3-4)cc2)cc1 ; c1ccc(-c2cccc(N(c3ccc(-c4ccc5c6ccccc6n(-c6ccccc6)c5c4)cc3)c3ccc4c(c3)C3(c5ccccc5-c5ccccc53)c3ccccc3-4)c2)cc1 ; c1ccc(-n2c3ccccc3c3ccc(-c4ccc(N(c5ccc6c(c5)C5(c7ccccc7-c7ccccc75)c5ccccc5-6)c5ccc6ccccc6c5)cc4)cc32)cc1 ; c1ccc(-n2c3ccccc3c3ccc(-c4ccc(N(c5ccc(-c6ccccn6)cc5)c5ccc6c(c5)C5(c7ccccc7-c7ccccc75)c5ccccc5-6)cc4)cc32)cc1]

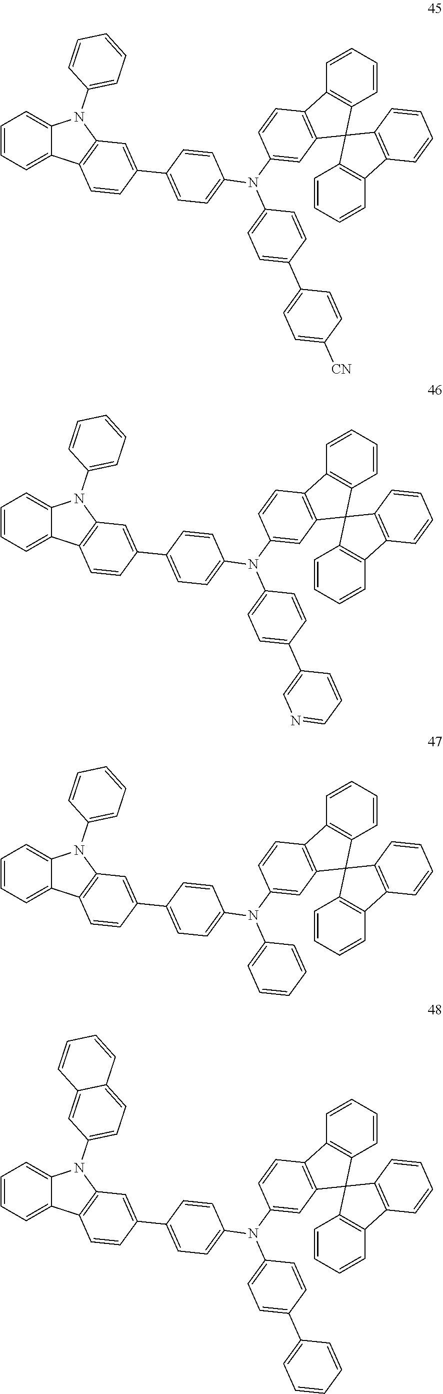 [SMILES: N#Cc1ccc(-c2ccc(N(c3ccc(-c4ccc5c6ccccc6n(-c6ccccc6)c5c4)cc3)c3ccc4c(c3)C3(c5ccccc5-c5ccccc53)c3ccccc3-4)cc2)cc1 ; c1ccc(-n2c3ccccc3c3ccc(-c4ccc(N(c5ccc(-c6cccnc6)cc5)c5ccc6c(c5)C5(c7ccccc7-c7ccccc75)c5ccccc5-6)cc4)cc32)cc1 ; c1ccc(N(c2ccc(-c3ccc4c5ccccc5n(-c5ccccc5)c4c3)cc2)c2ccc3c(c2)C2(c4ccccc4-c4ccccc42)c2ccccc2-3)cc1 ; c1ccc(-c2ccc(N(c3ccc(-c4ccc5c6ccccc6n(-c6ccc7ccccc7c6)c5c4)cc3)c3ccc4c(c3)C3(c5ccccc5-c5ccccc53)c3ccccc3-4)cc2)cc1]

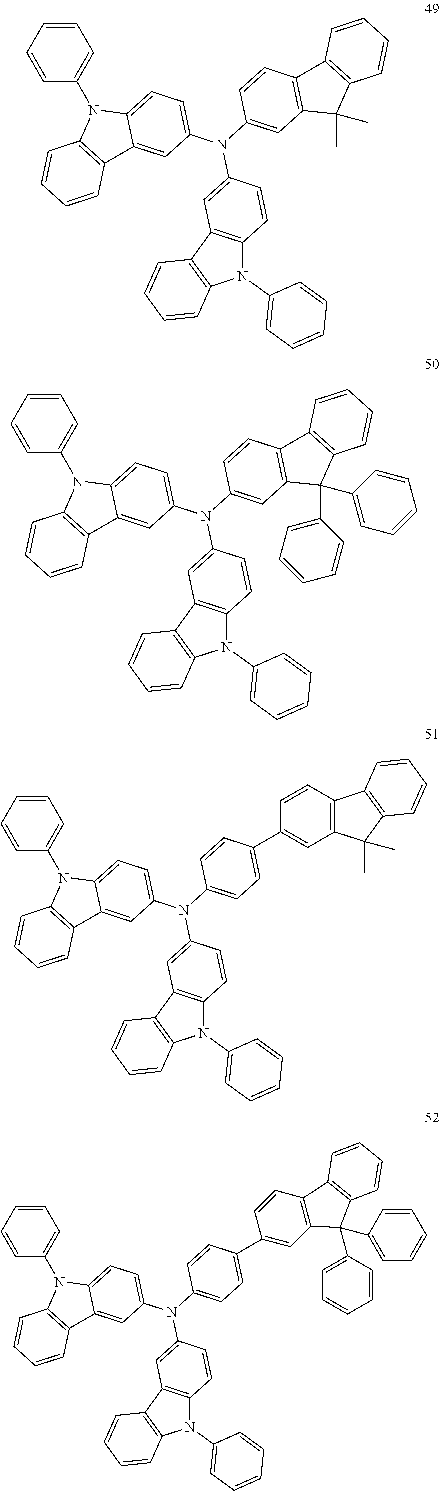 [SMILES: CC1(C)c2ccccc2-c2ccc(N(c3ccc4c(c3)c3ccccc3n4-c3ccccc3)c3ccc4c(c3)c3ccccc3n4-c3ccccc3)cc21 ; c1ccc(-n2c3ccccc3c3cc(N(c4ccc5c(c4)C(c4ccccc4)(c4ccccc4)c4ccccc4-5)c4ccc5c(c4)c4ccccc4n5-c4ccccc4)ccc32)cc1 ; CC1(C)c2ccccc2-c2ccc(-c3ccc(N(c4ccc5c(c4)c4ccccc4n5-c4ccccc4)c4ccc5c(c4)c4ccccc4n5-c4ccccc4)cc3)cc21 ; c1ccc(-n2c3ccccc3c3cc(N(c4ccc(-c5ccc6c(c5)C(c5ccccc5)(c5ccccc5)c5ccccc5-6)cc4)c4ccc5c(c4)c4ccccc4n5-c4ccccc4)ccc32)cc1]

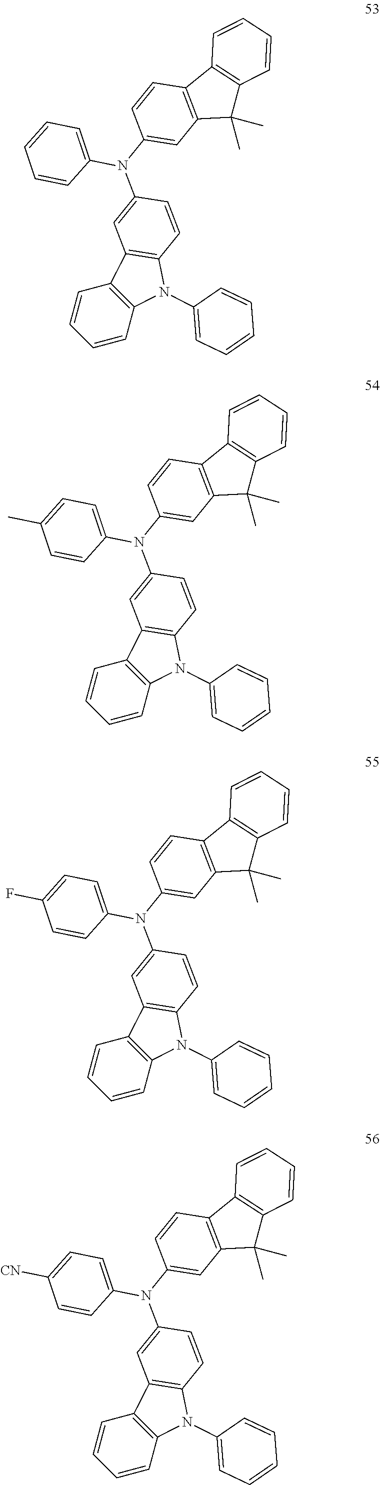

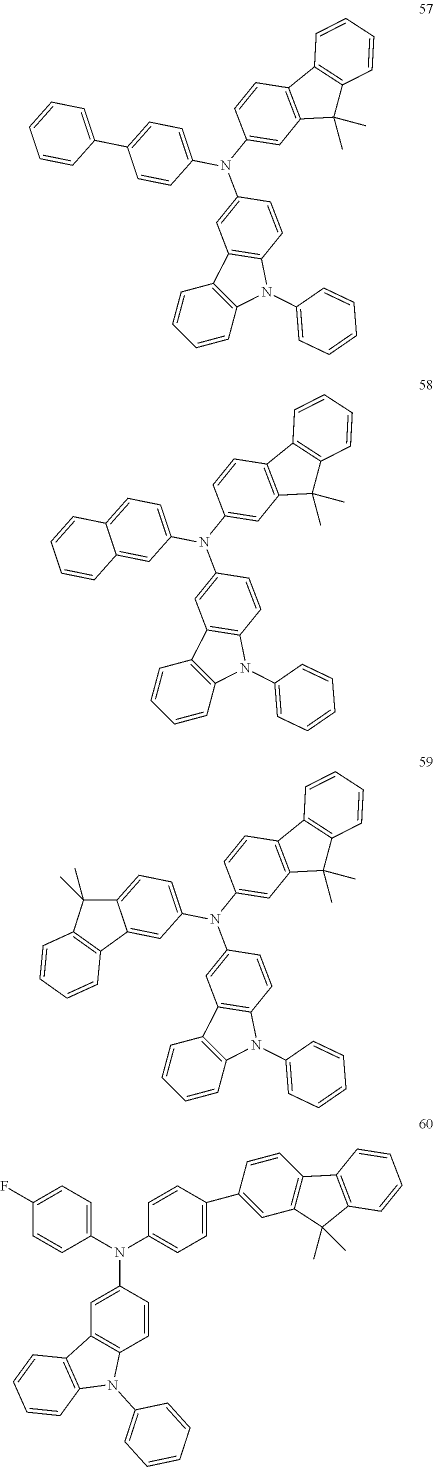

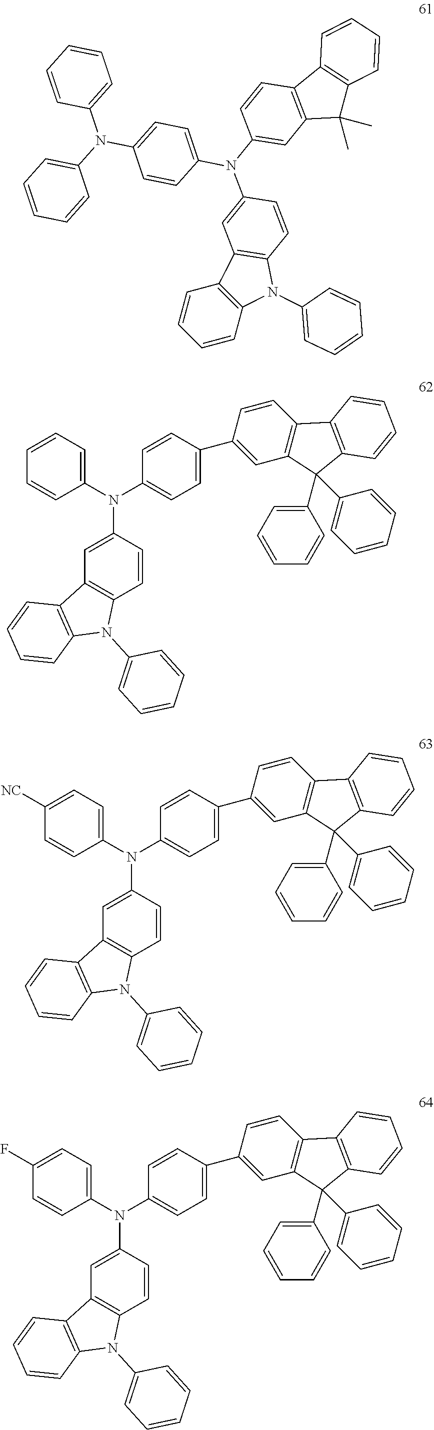

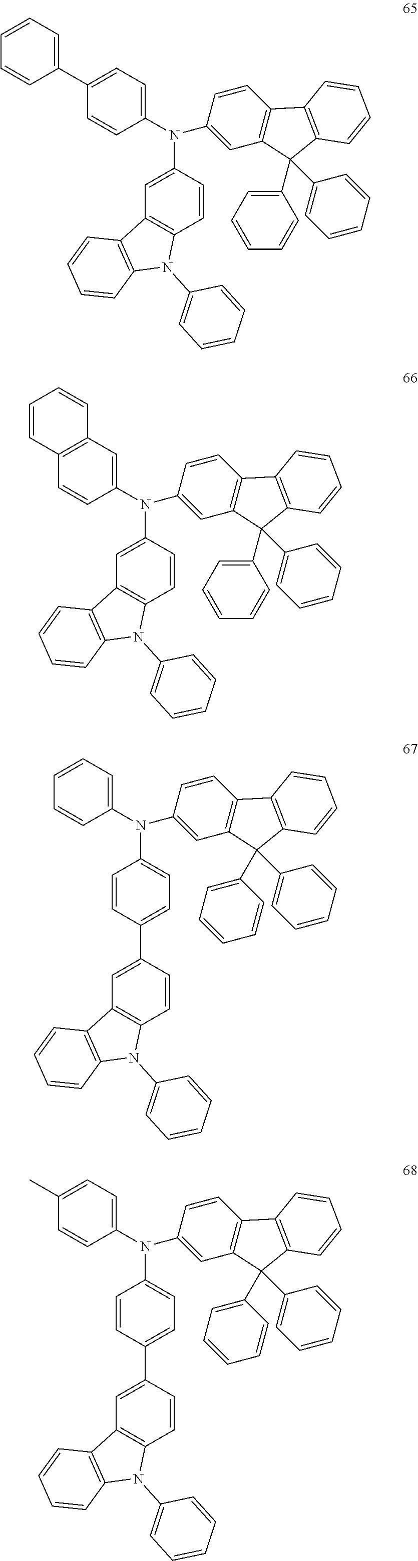 [SMILES: c1ccc(-c2ccc(N(c3ccc4c(c3)C(c3ccccc3)(c3ccccc3)c3ccccc3-4)c3ccc4c(c3)c3ccccc3n4-c3ccccc3)cc2)cc1 ; c1ccc(-n2c3ccccc3c3cc(N(c4ccc5c(c4)C(c4ccccc4)(c4ccccc4)c4ccccc4-5)c4ccc5ccccc5c4)ccc32)cc1 ; c1ccc(N(c2ccc(-c3ccc4c(c3)c3ccccc3n4-c3ccccc3)cc2)c2ccc3c(c2)C(c2ccccc2)(c2ccccc2)c2ccccc2-3)cc1 ; Cc1ccc(N(c2ccc(-c3ccc4c(c3)c3ccccc3n4-c3ccccc3)cc2)c2ccc3c(c2)C(c2ccccc2)(c2ccccc2)c2ccccc2-3)cc1]

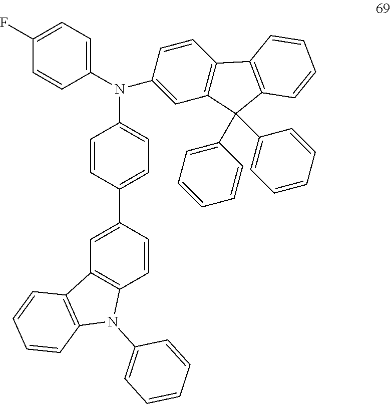 [SMILES: Fc1ccc(N(c2ccc(-c3ccc4c(c3)c3ccccc3n4-c3ccccc3)cc2)c2ccc3c(c2)C(c2ccccc2)(c2ccccc2)c2ccccc2-3)cc1]

20. The light emitting diode of claim 1, wherein the hole transport region further comprises:
a fourth hole transport layer disposed between the first hole transport layer and the third hole transport layer, the fourth hole transport layer having a refractive index greater than the first refractive index and less than the third refractive index; and
a fifth hole transport layer disposed between the second hole transport layer and the third hole transport layer, the fifth hole transport layer having a refractive index greater than the second refractive index and less than the third refractive index.

21. The light emitting diode of claim 20, wherein
the first hole transport layer and the second hole transport layer each independently comprises an amine compound represented by Formula 1,
the third hole transport layer comprises a compound represented by Formula 2, and
the fourth hole transport layer and the fifth hole transport layer each independently comprises an amine compound represented by Formula 1 and a compound represented by Formula 2: 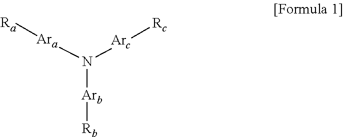
wherein in Formula 1,
Arₐ to Ar_{c} are each independently a substituted or unsubstituted aryl group of 6 to 30 ring-forming carbon atoms, or a substituted or unsubstituted heteroaryl group of 3 to 30 ring-forming carbon atoms,
at least two of Rₐ to R_{c} are each independently an adamantyl group or a cyclohexyl group, and
the remainder of Rₐ to R_{c} is a hydrogen atom, a deuterium atom, a halogen atom, a cyano group, a substituted or unsubstituted oxy group, a substituted or unsubstituted thio group, a substituted or unsubstituted amine group, or a substituted or unsubstituted alkyl group of 1 to 20 carbon atoms, 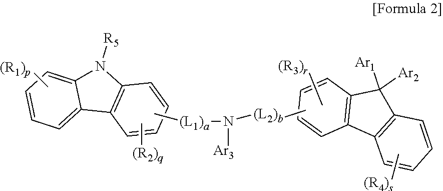
wherein in Formula 2,
Ar₁ and Ar₂ are each independently a hydrogen atom, a deuterium atom, a halogen atom, a substituted or unsubstituted alkyl group of 1 to 30 carbon atoms, a substituted or unsubstituted aryl group of 6 to 30 ring-forming carbon atoms, or a substituted or unsubstituted heteroaryl group of 2 to 30 ring-forming carbon atoms, or combined with an adjacent group to form a ring,
Ar₃ is a substituted or unsubstituted aryl group of 6 to 30 ring-forming carbon atoms, or a substituted or unsubstituted heteroaryl group of 2 to 30 ring-forming carbon atoms,
a and b are each independently 0 or 1,
L₁ and L₂ are each independently a substituted or unsubstituted cycloalkylene group of 3 to 10 ring-forming carbon atoms, a substituted or unsubstituted heterocycloalkylene group of 2 to 10 ring-forming carbon atoms, a substituted or unsubstituted cycloalkenylene group of 3 to 10 ring-forming carbon atoms, a substituted or unsubstituted arylene group of 6 to 30 ring-forming carbon atoms, or a substituted or unsubstituted heteroarylene group of 2 to 60 ring-forming carbon atoms,
p and s are each independently an integer from 0 to 4,
q and r are each independently an integer from 0 to 3, and
R₁ to R₅ are each independently a hydrogen atom, a deuterium atom, a halogen atom, a hydroxyl group, a cyano group, a nitro group, an amino group, a substituted or unsubstituted silyl group, a substituted or unsubstituted oxy group, a substituted or unsubstituted alkyl group of 1 to 60 carbon atoms, a substituted or unsubstituted heterocycloalkyl group of 3 to 60 ring-forming carbon atoms, a substituted or unsubstituted aryl group of 6 to 60 ring-forming carbon atoms, or a substituted or unsubstituted heteroaryl group of 2 to 60 ring-forming carbon atoms.

22. The light emitting diode of claim 20, wherein the first to fifth hole transport layers each have a thickness in a range of about 100 Å to about 1,000 Å.

23. A display device comprising:
a plurality of light emitting diodes, each of the plurality of light emitting diodes including:
a first electrode;
a hole transport region disposed on the first electrode;
an emission layer disposed on the hole transport region;
an electron transport region disposed on the emission layer; and
a second electrode disposed on the electron transport region,
wherein the hole transport region of at least one of the plurality of light emitting diodes comprises:
a first hole transport layer disposed adjacent to the first electrode, the first hole transport layer having a first refractive index;
a second hole transport layer disposed adjacent to the emission layer, the second hole transport layer having a second refractive index; and
a third hole transport layer disposed between the first hole transport layer and the second hole transport layer, the third hole transport layer having a third refractive index which is greater than each of the first refractive index and the second refractive index.

24. The display device of claim 23, wherein
a difference between the third refractive index and the first refractive index is greater than about 0.1, and
a difference between the third refractive index and the second refractive index is greater than about 0.1.

25. The display device of claim 23, wherein
the first electrode is a reflective electrode, and
the second electrode is a transmissive electrode or a transflective electrode.

26. The display device of claim 23, wherein
the first hole transport layer and the second hole transport layer each independently comprises an amine compound represented by Formula 1, and
the third hole transport layer comprises a compound represented by Formula 2: 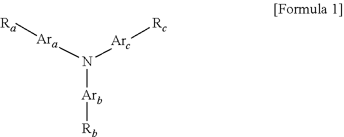
wherein in Formula 1,
Arₐ to Ar_{c} are each independently a substituted or unsubstituted aryl group of 6 to 30 ring-forming carbon atoms, or a substituted or unsubstituted heteroaryl group of 3 to 30 ring-forming carbon atoms,
at least two of Rₐ to R_{c} are each independently an adamantyl group or a cyclohexyl group, and
the remainder of Rₐ to R_{c} is a hydrogen atom, a deuterium atom, a halogen atom, a cyano group, a substituted or unsubstituted oxy group, a substituted or unsubstituted thio group, a substituted or unsubstituted amine group, or a substituted or unsubstituted alkyl group of 1 to 20 carbon atoms, 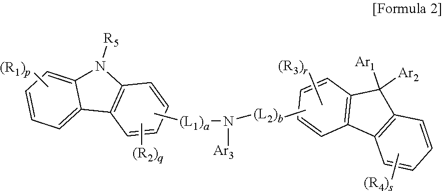
wherein in Formula 2,
Ar₁ and Ar₂ are each independently a hydrogen atom, a deuterium atom, a halogen atom, a substituted or unsubstituted alkyl group of 1 to 30 carbon atoms, a substituted or unsubstituted aryl group of 6 to 30 ring-forming carbon atoms, or a substituted or unsubstituted heteroaryl group of 2 to 30 ring-forming carbon atoms, or combined with an adjacent group to form a ring,
Ar₃ is a substituted or unsubstituted aryl group of 6 to 30 ring-forming carbon atoms, or a substituted or unsubstituted heteroaryl group of 2 to 30 ring-forming carbon atoms,
a and b are each independently 0 or 1,
L₁ and L₂ are each independently a substituted or unsubstituted cycloalkylene group of 3 to 10 ring-forming carbon atoms, a substituted or unsubstituted heterocycloalkylene group of 2 to 10 ring-forming carbon atoms, a substituted or unsubstituted cycloalkenylene group of 3 to 10 ring-forming carbon atoms, a substituted or unsubstituted arylene group of 6 to 30 ring-forming carbon atoms, or a substituted or unsubstituted heteroarylene group of 2 to 60 ring-forming carbon atoms,
p and s are each independently an integer from 0 to 4,
q and r are each independently an integer from 0 to 3, and
R₁ to R₅ are each independently a hydrogen atom, a deuterium atom, a halogen atom, a hydroxyl group, a cyano group, a nitro group, an amino group, a substituted or unsubstituted silyl group, a substituted or unsubstituted oxy group, a substituted or unsubstituted alkyl group of 1 to 60 carbon atoms, a substituted or unsubstituted heterocycloalkyl group of 3 to 60 ring-forming carbon atoms, a substituted or unsubstituted aryl group of 6 to 60 ring-forming carbon atoms, or a substituted or unsubstituted heteroaryl group of 2 to 60 ring-forming carbon atoms.

27. A light emitting diode, comprising:
a first electrode;
a second electrode oppositely disposed to the first electrode; and
a plurality of light emitting units disposed between the first electrode and the second electrode, wherein
each of the light emitting units comprises a hole transport region, an emission layer, and an electron transport region stacked in order, and
the hole transport region of at least one of the light emitting units comprises an amine compound represented by Formula 1: 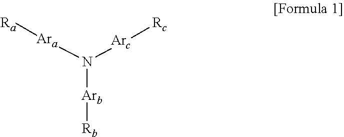
wherein in Formula 1,
Arₐ to Ar_{c} are each independently a substituted or unsubstituted aryl group of 6 to 30 ring-forming carbon atoms, or a substituted or unsubstituted heteroaryl group of 3 to 30 ring-forming carbon atoms,
at least two of Rₐ to R_{c} are each independently an adamantyl group or a cyclohexyl group, and
the remainder of Rₐ to R_{c} is a hydrogen atom, a deuterium atom, a halogen atom, a cyano group, a substituted or unsubstituted oxy group, a substituted or unsubstituted thio group, a substituted or unsubstituted amine group, or a substituted or unsubstituted alkyl group of 1 to 20 carbon atoms.

28. The light emitting diode of claim 27, wherein the hole transport region of a light emitting unit adjacent to the second electrode comprises the amine compound.

29. The light emitting diode of claim 27, wherein the plurality of light emitting units comprise at least one blue light emitting unit that emits blue light.

30. The light emitting diode of claim 27, further comprising a charge generating layer disposed between the plurality of light emitting units.

31. The light emitting diode of claim 30, wherein the charge generating layer comprises:
a p-type charge generating layer comprising a p-dopant; and
an n-type charge generating layer comprising an n-dopant.

32. The light emitting diode of claim 27, further comprising a capping layer disposed on the second electrode, wherein
the capping layer has a refractive index equal to or greater than about 1.6.

33. The light emitting diode of claim 27, wherein Arₐ to Ar_{c} are each independently a substituted or unsubstituted phenyl group, a substituted or unsubstituted naphthyl group, a substituted or unsubstituted biphenyl group, a substituted or unsubstituted terphenyl group, a substituted or unsubstituted fluorenyl group, a substituted or unsubstituted carbazole group, a substituted or unsubstituted dibenzofuranyl group, or a substituted or unsubstituted dibenzothiophenyl group.

34. The light emitting diode of claim 27, wherein the amine compound is selected from Compound Group 1:

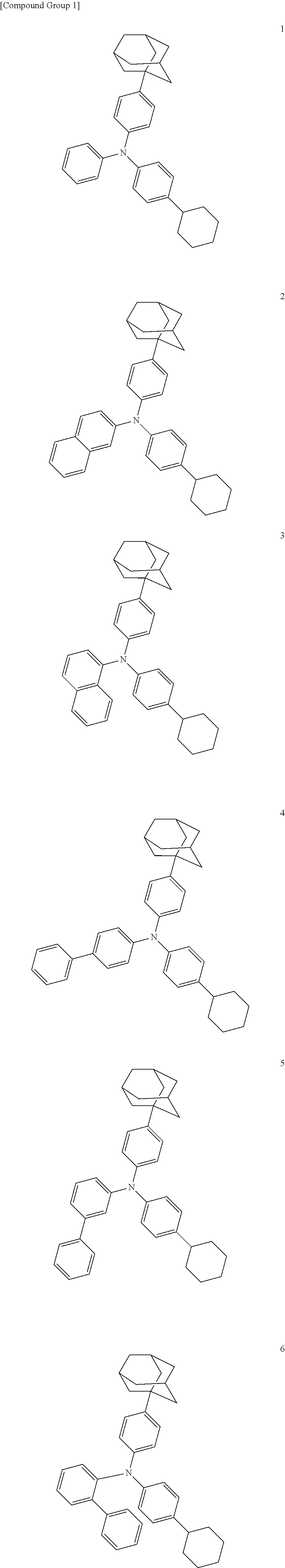 [SMILES: c1ccc(N(c2ccc(C3CCCCC3)cc2)c2ccc(C34CC5CC(CC(C5)C3)C4)cc2)cc1 ; c1ccc2cc(N(c3ccc(C4CCCCC4)cc3)c3ccc(C45CC6CC(CC(C6)C4)C5)cc3)ccc2c1 ; c1ccc2c(N(c3ccc(C4CCCCC4)cc3)c3ccc(C45CC6CC(CC(C6)C4)C5)cc3)cccc2c1 ; c1ccc(-c2ccc(N(c3ccc(C4CCCCC4)cc3)c3ccc(C45CC6CC(CC(C6)C4)C5)cc3)cc2)cc1 ; c1ccc(-c2cccc(N(c3ccc(C4CCCCC4)cc3)c3ccc(C45CC6CC(CC(C6)C4)C5)cc3)c2)cc1 ; c1ccc(-c2ccccc2N(c2ccc(C3CCCCC3)cc2)c2ccc(C34CC5CC(CC(C5)C3)C4)cc2)cc1]

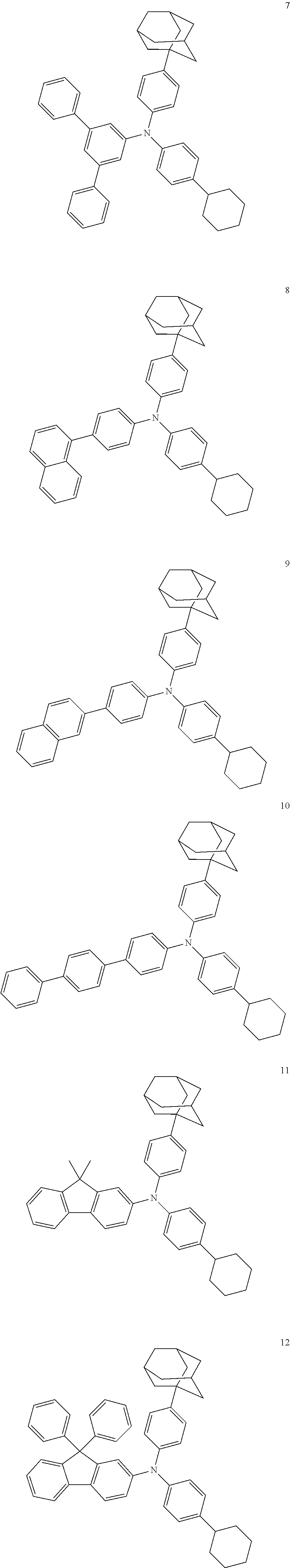

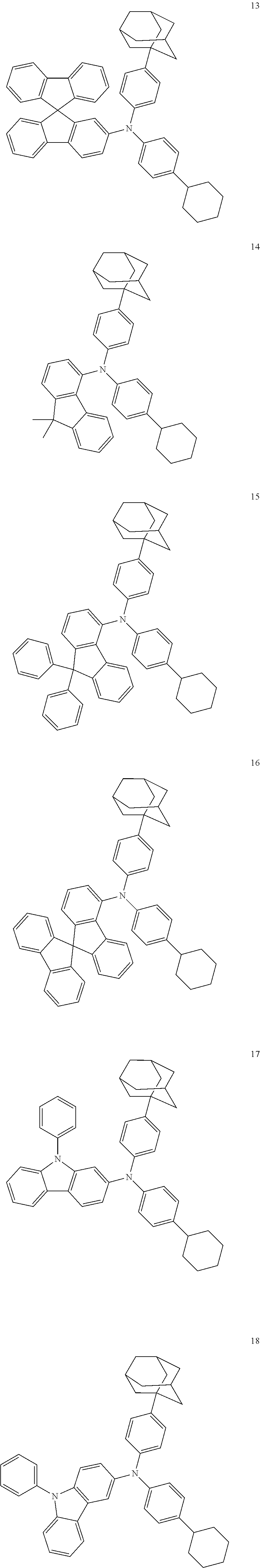

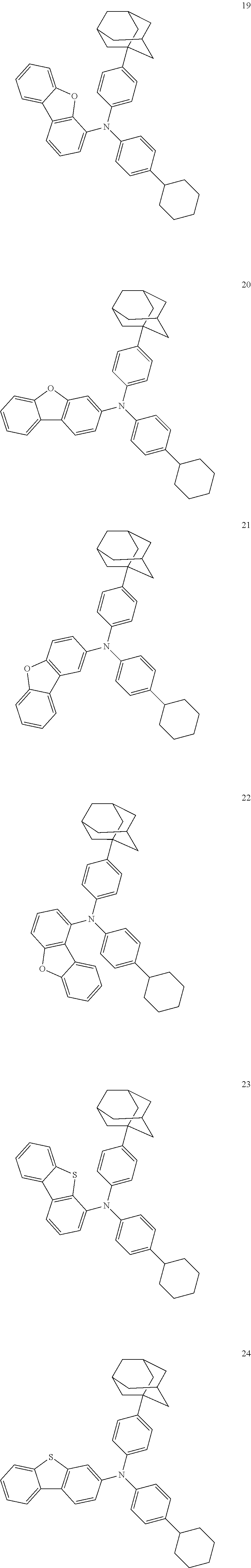 [SMILES: c1ccc2c(c1)oc1c(N(c3ccc(C4CCCCC4)cc3)c3ccc(C45CC6CC(CC(C6)C4)C5)cc3)cccc12 ; c1ccc2c(c1)oc1cc(N(c3ccc(C4CCCCC4)cc3)c3ccc(C45CC6CC(CC(C6)C4)C5)cc3)ccc12 ; c1ccc2c(c1)oc1ccc(N(c3ccc(C4CCCCC4)cc3)c3ccc(C45CC6CC(CC(C6)C4)C5)cc3)cc12 ; c1ccc2c(c1)oc1cccc(N(c3ccc(C4CCCCC4)cc3)c3ccc(C45CC6CC(CC(C6)C4)C5)cc3)c12 ; c1ccc2c(c1)sc1c(N(c3ccc(C4CCCCC4)cc3)c3ccc(C45CC6CC(CC(C6)C4)C5)cc3)cccc12 ; c1ccc2c(c1)sc1cc(N(c3ccc(C4CCCCC4)cc3)c3ccc(C45CC6CC(CC(C6)C4)C5)cc3)ccc12]

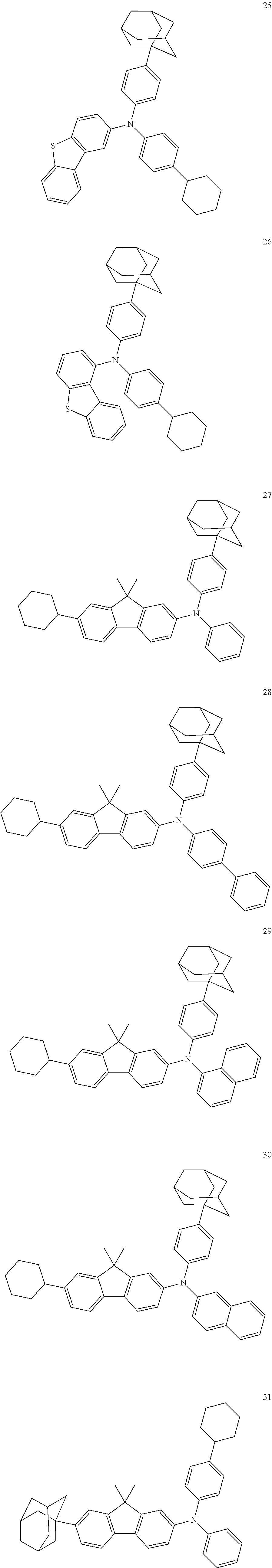

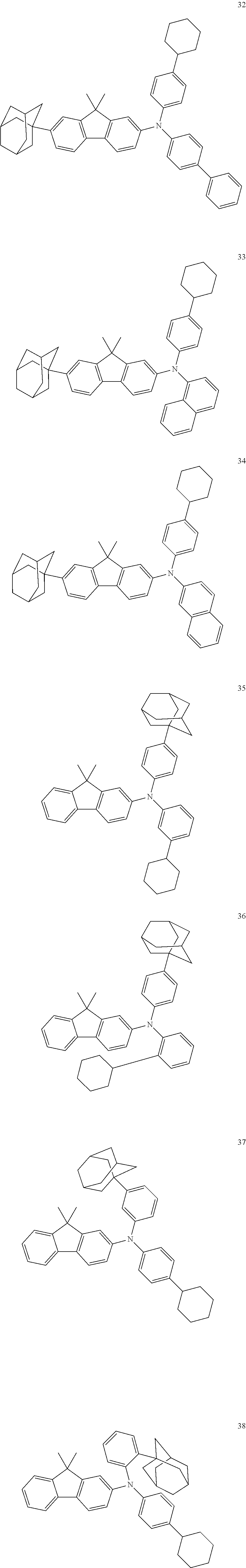

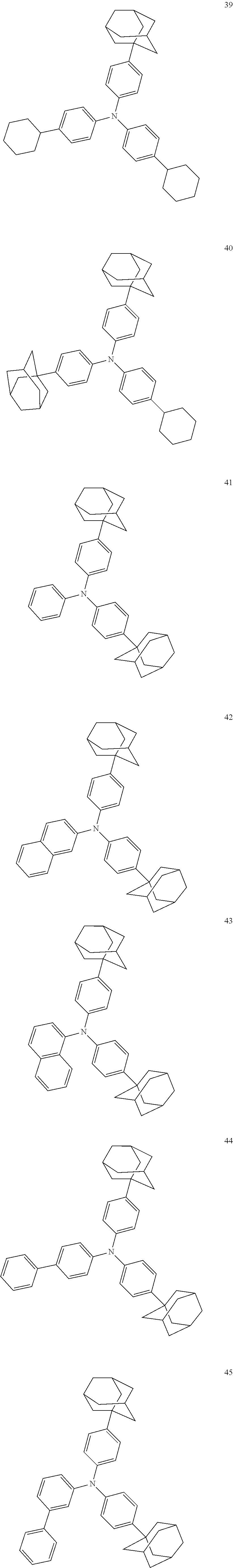

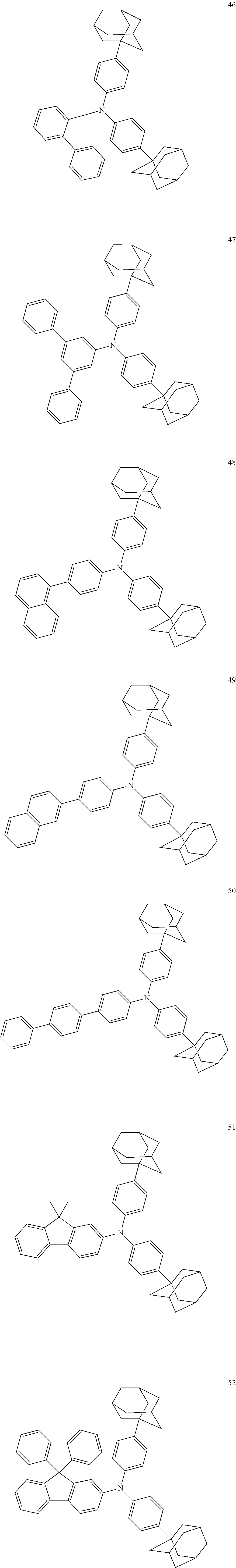

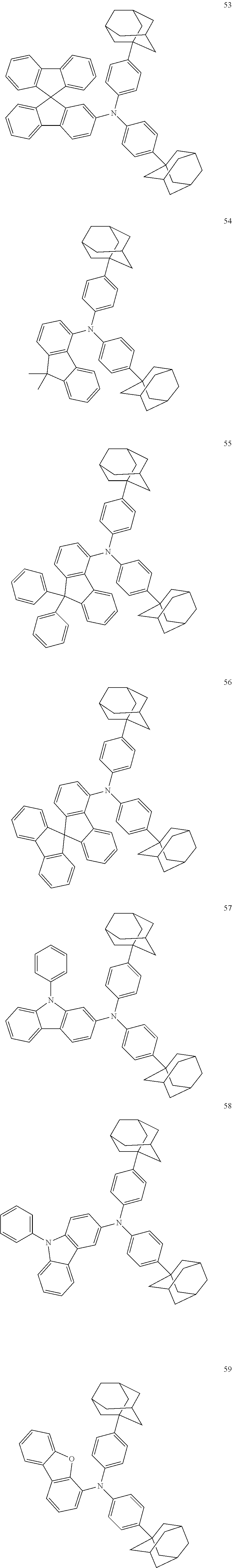

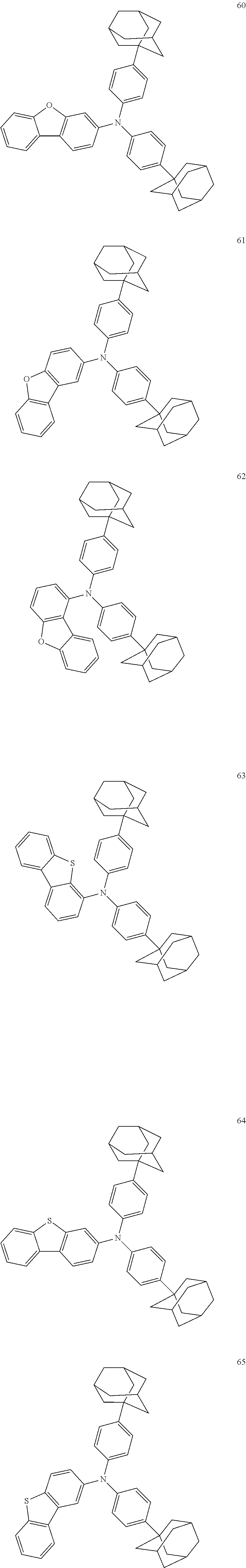

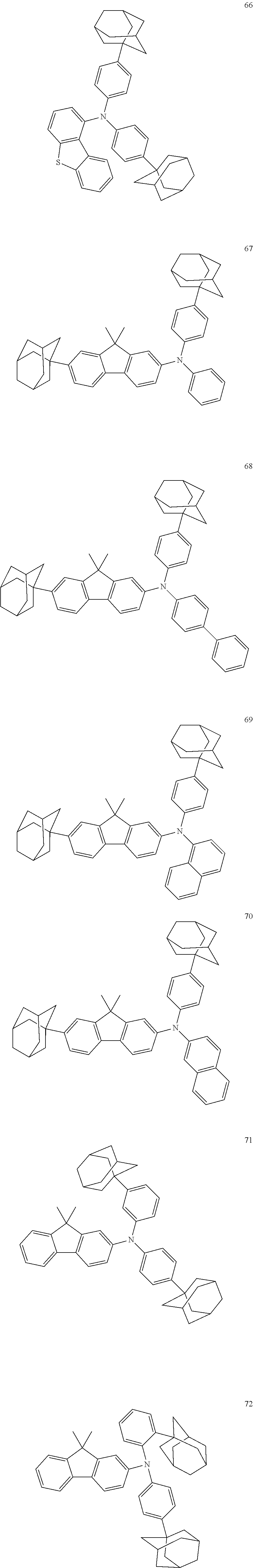

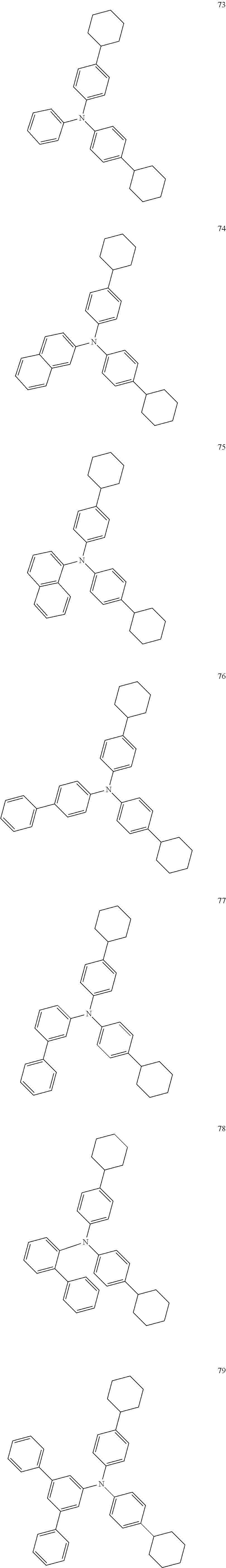

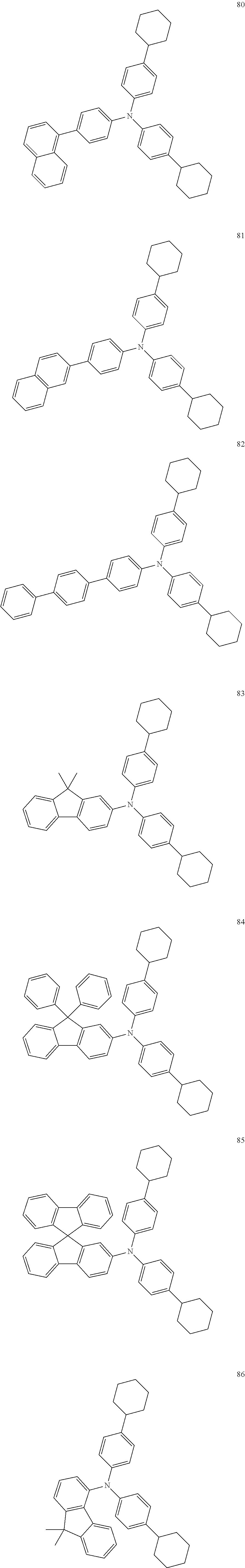

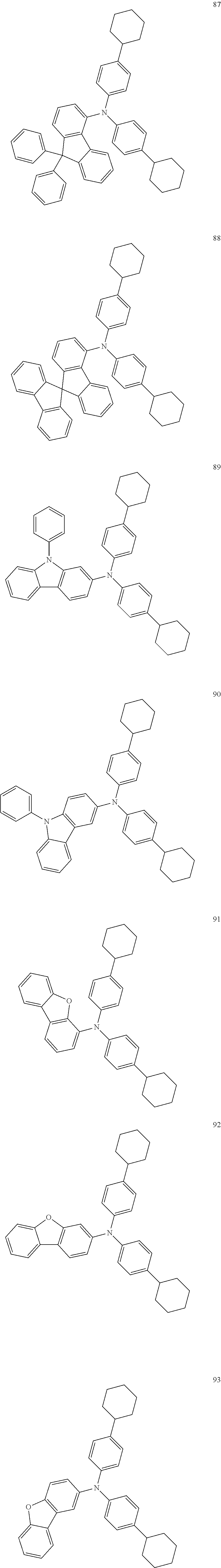

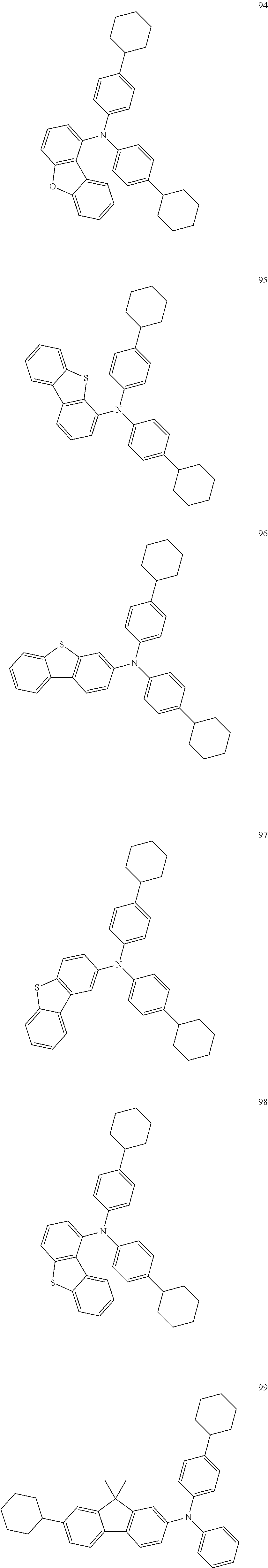 [SMILES: c1ccc2c(c1)oc1cccc(N(c3ccc(C4CCCCC4)cc3)c3ccc(C4CCCCC4)cc3)c12 ; c1ccc2c(c1)sc1c(N(c3ccc(C4CCCCC4)cc3)c3ccc(C4CCCCC4)cc3)cccc12 ; c1ccc2c(c1)sc1cc(N(c3ccc(C4CCCCC4)cc3)c3ccc(C4CCCCC4)cc3)ccc12 ; c1ccc2c(c1)sc1ccc(N(c3ccc(C4CCCCC4)cc3)c3ccc(C4CCCCC4)cc3)cc12 ; c1ccc2c(c1)sc1cccc(N(c3ccc(C4CCCCC4)cc3)c3ccc(C4CCCCC4)cc3)c12 ; CC1(C)c2cc(C3CCCCC3)ccc2-c2ccc(N(c3ccccc3)c3ccc(C4CCCCC4)cc3)cc21]

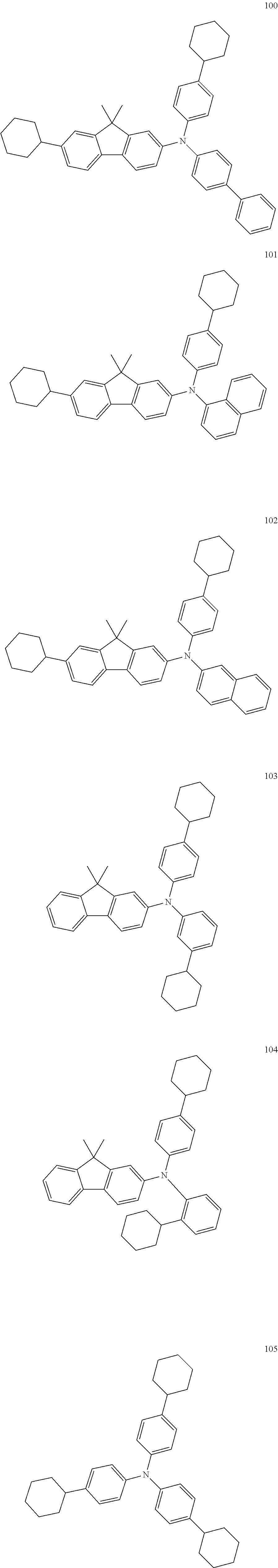 [SMILES: CC1(C)c2cc(C3CCCCC3)ccc2-c2ccc(N(c3ccc(-c4ccccc4)cc3)c3ccc(C4CCCCC4)cc3)cc21 ; CC1(C)c2cc(C3CCCCC3)ccc2-c2ccc(N(c3ccc(C4CCCCC4)cc3)c3cccc4ccccc34)cc21 ; CC1(C)c2cc(C3CCCCC3)ccc2-c2ccc(N(c3ccc(C4CCCCC4)cc3)c3ccc4ccccc4c3)cc21 ; CC1(C)c2ccccc2-c2ccc(N(c3ccc(C4CCCCC4)cc3)c3cccc(C4CCCCC4)c3)cc21 ; CC1(C)c2ccccc2-c2ccc(N(c3ccc(C4CCCCC4)cc3)c3ccccc3C3CCCCC3)cc21 ; c1cc(N(c2ccc(C3CCCCC3)cc2)c2ccc(C3CCCCC3)cc2)ccc1C1CCCCC1]

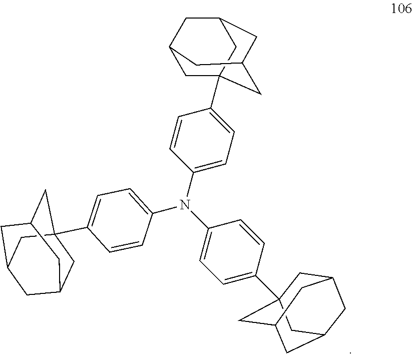 [SMILES: c1cc(C23CC4CC(CC(C4)C2)C3)ccc1N(c1ccc(C23CC4CC(CC(C4)C2)C3)cc1)c1ccc(C23CC4CC(CC(C4)C2)C3)cc1]

35. The light emitting diode of claim 27, wherein the hole transport region comprising the amine compound has a refractive index in a range of about 1.30 to about 1.80, with respect to light at a wavelength of about 460 nm.

36. The light emitting diode of claim 27, wherein the hole transport region of at least one of the light emitting units comprising the amine compound comprises:
a first hole transport layer having a first refractive index;
a second hole transport layer disposed adjacent to the emission layer and having a second refractive index; and
a third hole transport layer disposed between the first hole transport layer and the second hole transport layer, and having a third refractive index greater than each of the first refractive index and the second refractive index.

37. The light emitting diode of claim 36, wherein the first hole transport layer and the second hole transport layer each includes the amine compound, each independently represented by Formula 1.

38. The light emitting diode of claim 36, wherein the third hole transport layer comprises a compound represented by Formula 2: 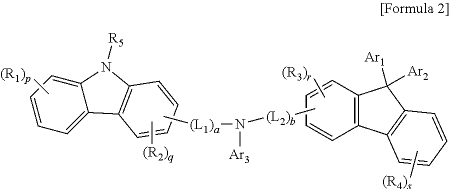
wherein in Formula 2,
Ar₁ and Ar₂ are each independently a hydrogen atom, a deuterium atom, a halogen atom, a substituted or unsubstituted alkyl group of 1 to 30 carbon atoms, a substituted or unsubstituted aryl group of 6 to 30 ring-forming carbon atoms, or a substituted or unsubstituted heteroaryl group of 2 to 30 ring-forming carbon atoms, or combined with an adjacent group to form a ring,
Ar₃ is a substituted or unsubstituted aryl group of 6 to 30 ring-forming carbon atoms, or a substituted or unsubstituted heteroaryl group of 2 to 30 ring-forming carbon atoms,
a and b are each independently 0 or 1,
L₁ and L₂ are each independently a substituted or unsubstituted cycloalkylene group of 3 to 10 ring-forming carbon atoms, a substituted or unsubstituted heterocycloalkylene group of 2 to 10 ring-forming carbon atoms, a substituted or unsubstituted cycloalkenylene group of 3 to 10 ring-forming carbon atoms, a substituted or unsubstituted arylene group of 6 to 30 ring-forming carbon atoms, or a substituted or unsubstituted heteroarylene group of 2 to 60 ring-forming carbon atoms,
p and s are each independently an integer from 0 to 4,
q and r are each independently an integer from 0 to 3, and
R₁ to R₅ are each independently a hydrogen atom, a deuterium atom, a halogen atom, a hydroxyl group, a cyano group, a nitro group, an amino group, a substituted or unsubstituted silyl group, a substituted or unsubstituted oxy group, a substituted or unsubstituted alkyl group of 1 to 60 carbon atoms, a substituted or unsubstituted heterocycloalkyl group of 3 to 60 ring-forming carbon atoms, a substituted or unsubstituted aryl group of 6 to 60 ring-forming carbon atoms, or a substituted or unsubstituted heteroaryl group of 2 to 60 ring-forming carbon atoms.

39. The light emitting diode of claim 36, wherein the third hole transport layer comprises at least one compound selected from Compound Group 2:

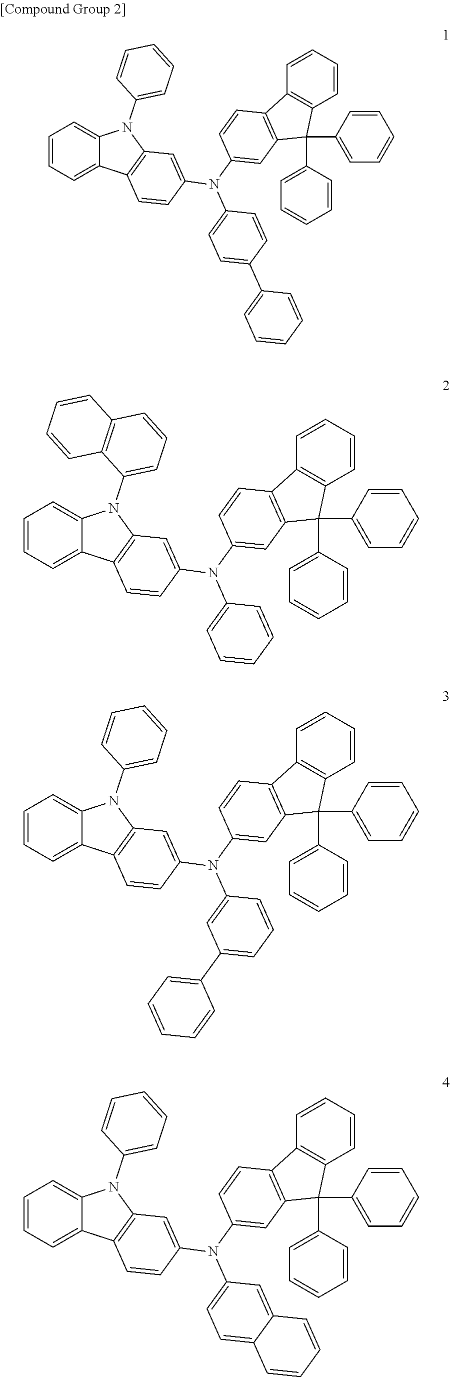 [SMILES: c1ccc(-c2ccc(N(c3ccc4c(c3)C(c3ccccc3)(c3ccccc3)c3ccccc3-4)c3ccc4c5ccccc5n(-c5ccccc5)c4c3)cc2)cc1 ; c1ccc(N(c2ccc3c(c2)C(c2ccccc2)(c2ccccc2)c2ccccc2-3)c2ccc3c4ccccc4n(-c4cccc5ccccc45)c3c2)cc1 ; c1ccc(-c2cccc(N(c3ccc4c(c3)C(c3ccccc3)(c3ccccc3)c3ccccc3-4)c3ccc4c5ccccc5n(-c5ccccc5)c4c3)c2)cc1 ; c1ccc(-n2c3ccccc3c3ccc(N(c4ccc5c(c4)C(c4ccccc4)(c4ccccc4)c4ccccc4-5)c4ccc5ccccc5c4)cc32)cc1]

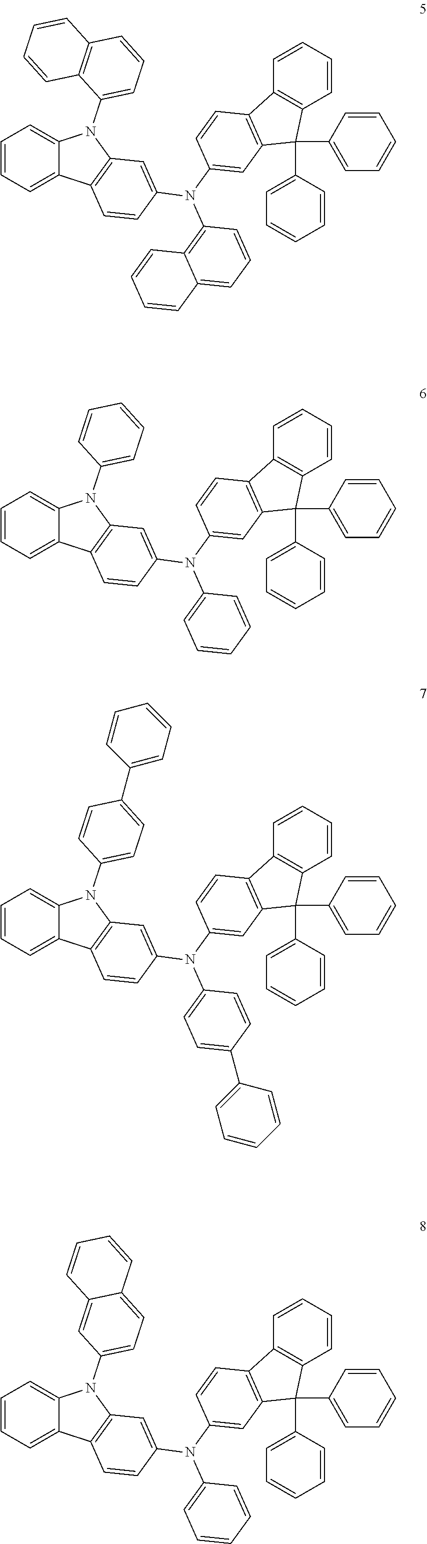 [SMILES: c1ccc(C2(c3ccccc3)c3ccccc3-c3ccc(N(c4ccc5c6ccccc6n(-c6cccc7ccccc67)c5c4)c4cccc5ccccc45)cc32)cc1 ; c1ccc(N(c2ccc3c(c2)C(c2ccccc2)(c2ccccc2)c2ccccc2-3)c2ccc3c4ccccc4n(-c4ccccc4)c3c2)cc1 ; c1ccc(-c2ccc(N(c3ccc4c(c3)C(c3ccccc3)(c3ccccc3)c3ccccc3-4)c3ccc4c5ccccc5n(-c5ccc(-c6ccccc6)cc5)c4c3)cc2)cc1 ; c1ccc(N(c2ccc3c(c2)C(c2ccccc2)(c2ccccc2)c2ccccc2-3)c2ccc3c4ccccc4n(-c4ccc5ccccc5c4)c3c2)cc1]

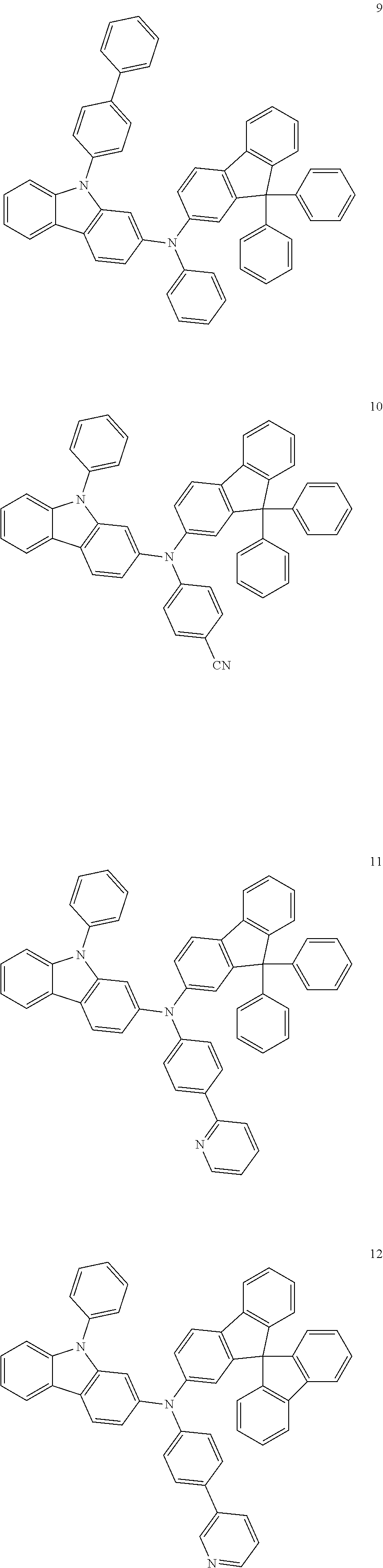 [SMILES: c1ccc(-c2ccc(-n3c4ccccc4c4ccc(N(c5ccccc5)c5ccc6c(c5)C(c5ccccc5)(c5ccccc5)c5ccccc5-6)cc43)cc2)cc1 ; N#Cc1ccc(N(c2ccc3c(c2)C(c2ccccc2)(c2ccccc2)c2ccccc2-3)c2ccc3c4ccccc4n(-c4ccccc4)c3c2)cc1 ; c1ccc(-n2c3ccccc3c3ccc(N(c4ccc(-c5ccccn5)cc4)c4ccc5c(c4)C(c4ccccc4)(c4ccccc4)c4ccccc4-5)cc32)cc1 ; c1ccc(-n2c3ccccc3c3ccc(N(c4ccc(-c5cccnc5)cc4)c4ccc5c(c4)C4(c6ccccc6-c6ccccc64)c4ccccc4-5)cc32)cc1]

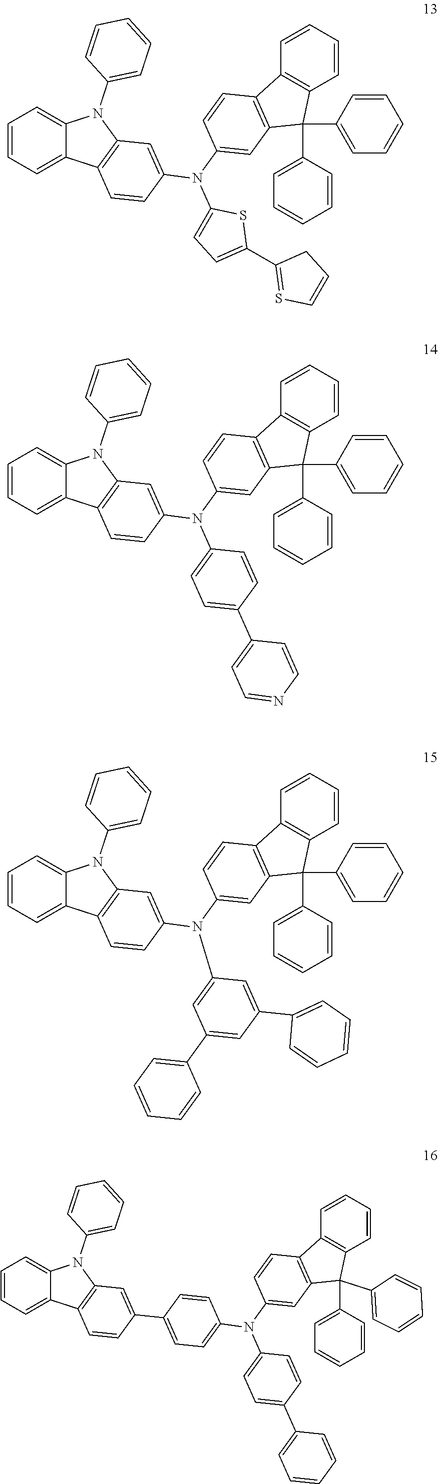

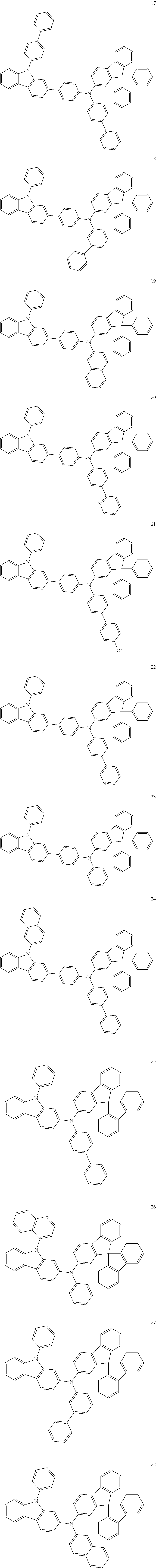

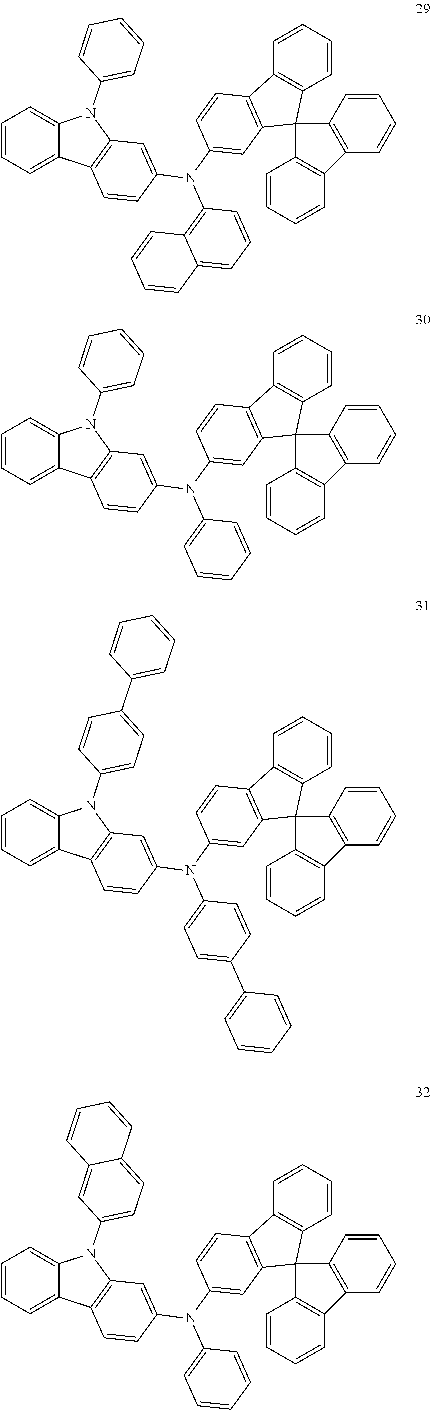

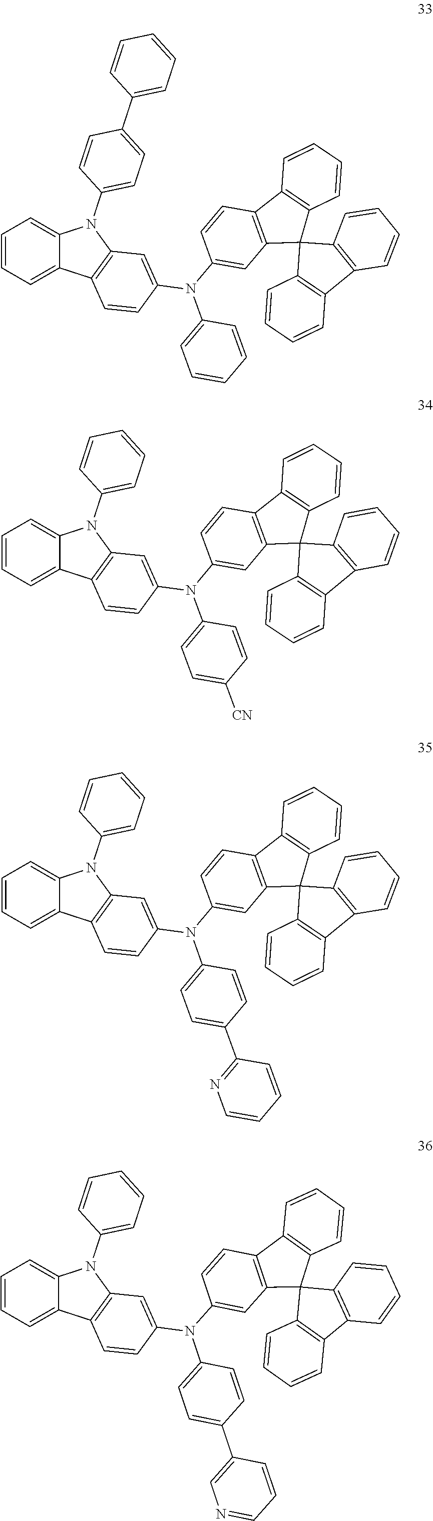 [SMILES: c1ccc(-c2ccc(-n3c4ccccc4c4ccc(N(c5ccccc5)c5ccc6c(c5)C5(c7ccccc7-c7ccccc75)c5ccccc5-6)cc43)cc2)cc1 ; N#Cc1ccc(N(c2ccc3c(c2)C2(c4ccccc4-c4ccccc42)c2ccccc2-3)c2ccc3c4ccccc4n(-c4ccccc4)c3c2)cc1 ; c1ccc(-n2c3ccccc3c3ccc(N(c4ccc(-c5ccccn5)cc4)c4ccc5c(c4)C4(c6ccccc6-c6ccccc64)c4ccccc4-5)cc32)cc1 ; c1ccc(-n2c3ccccc3c3ccc(N(c4ccc(-c5cccnc5)cc4)c4ccc5c(c4)C4(c6ccccc6-c6ccccc64)c4ccccc4-5)cc32)cc1]

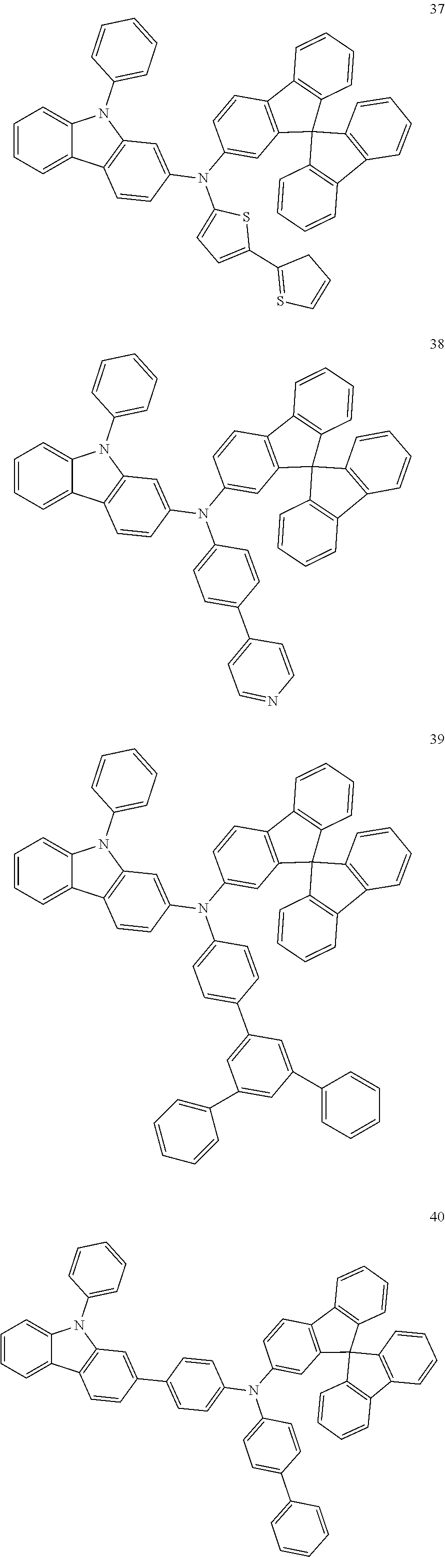

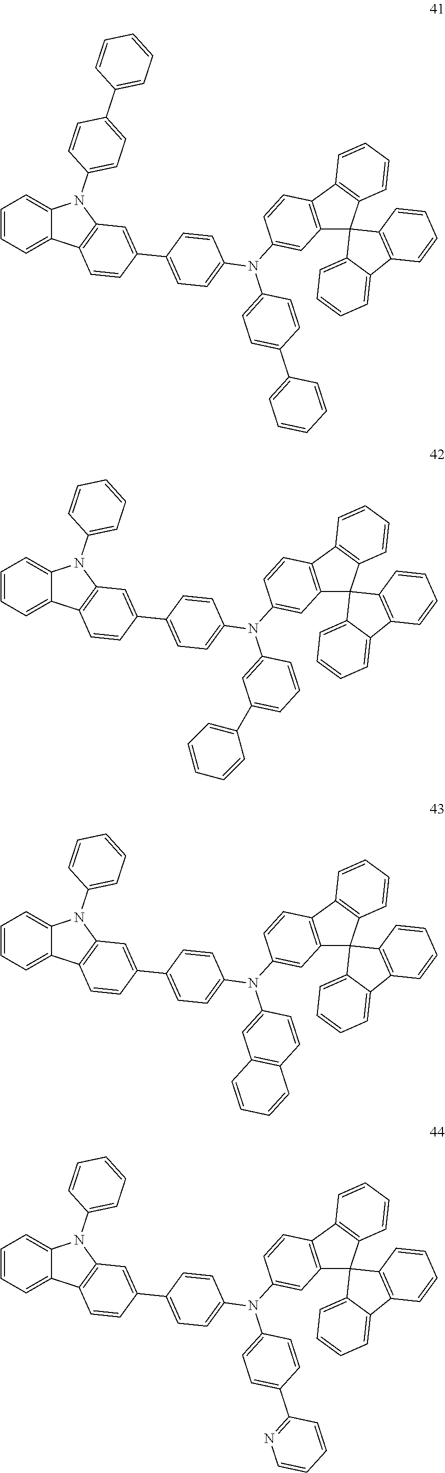 [SMILES: c1ccc(-c2ccc(N(c3ccc(-c4ccc5c6ccccc6n(-c6ccc(-c7ccccc7)cc6)c5c4)cc3)c3ccc4c(c3)C3(c5ccccc5-c5ccccc53)c3ccccc3-4)cc2)cc1 ; c1ccc(-c2cccc(N(c3ccc(-c4ccc5c6ccccc6n(-c6ccccc6)c5c4)cc3)c3ccc4c(c3)C3(c5ccccc5-c5ccccc53)c3ccccc3-4)c2)cc1 ; c1ccc(-n2c3ccccc3c3ccc(-c4ccc(N(c5ccc6c(c5)C5(c7ccccc7-c7ccccc75)c5ccccc5-6)c5ccc6ccccc6c5)cc4)cc32)cc1 ; c1ccc(-n2c3ccccc3c3ccc(-c4ccc(N(c5ccc(-c6ccccn6)cc5)c5ccc6c(c5)C5(c7ccccc7-c7ccccc75)c5ccccc5-6)cc4)cc32)cc1]

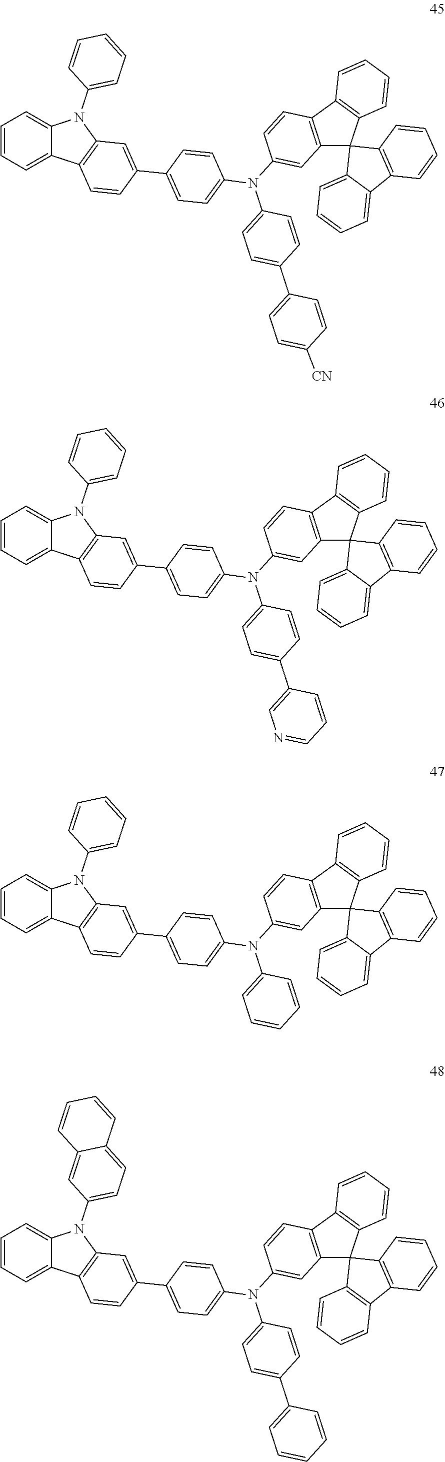 [SMILES: N#Cc1ccc(-c2ccc(N(c3ccc(-c4ccc5c6ccccc6n(-c6ccccc6)c5c4)cc3)c3ccc4c(c3)C3(c5ccccc5-c5ccccc53)c3ccccc3-4)cc2)cc1 ; c1ccc(-n2c3ccccc3c3ccc(-c4ccc(N(c5ccc(-c6cccnc6)cc5)c5ccc6c(c5)C5(c7ccccc7-c7ccccc75)c5ccccc5-6)cc4)cc32)cc1 ; c1ccc(N(c2ccc(-c3ccc4c5ccccc5n(-c5ccccc5)c4c3)cc2)c2ccc3c(c2)C2(c4ccccc4-c4ccccc42)c2ccccc2-3)cc1 ; c1ccc(-c2ccc(N(c3ccc(-c4ccc5c6ccccc6n(-c6ccc7ccccc7c6)c5c4)cc3)c3ccc4c(c3)C3(c5ccccc5-c5ccccc53)c3ccccc3-4)cc2)cc1]

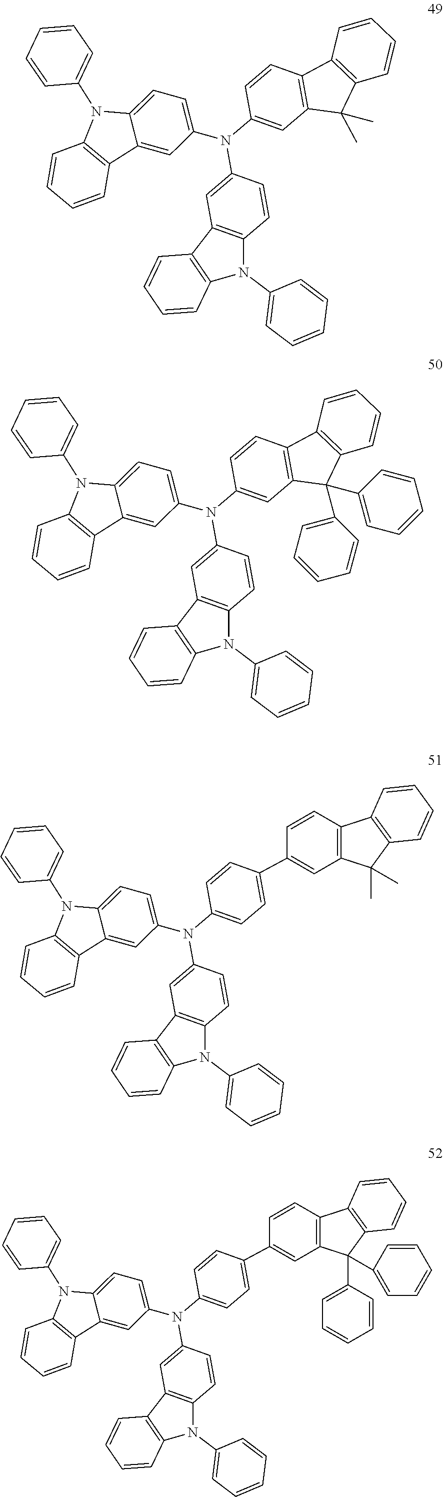 [SMILES: CC1(C)c2ccccc2-c2ccc(N(c3ccc4c(c3)c3ccccc3n4-c3ccccc3)c3ccc4c(c3)c3ccccc3n4-c3ccccc3)cc21 ; c1ccc(-n2c3ccccc3c3cc(N(c4ccc5c(c4)C(c4ccccc4)(c4ccccc4)c4ccccc4-5)c4ccc5c(c4)c4ccccc4n5-c4ccccc4)ccc32)cc1 ; CC1(C)c2ccccc2-c2ccc(-c3ccc(N(c4ccc5c(c4)c4ccccc4n5-c4ccccc4)c4ccc5c(c4)c4ccccc4n5-c4ccccc4)cc3)cc21 ; c1ccc(-n2c3ccccc3c3cc(N(c4ccc(-c5ccc6c(c5)C(c5ccccc5)(c5ccccc5)c5ccccc5-6)cc4)c4ccc5c(c4)c4ccccc4n5-c4ccccc4)ccc32)cc1]

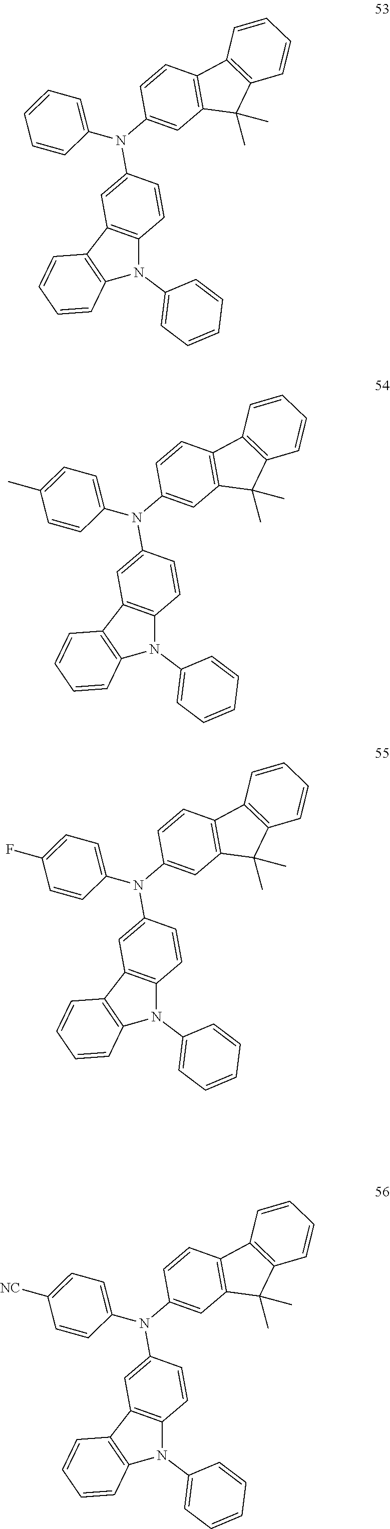 [SMILES: CC1(C)c2ccccc2-c2ccc(N(c3ccccc3)c3ccc4c(c3)c3ccccc3n4-c3ccccc3)cc21 ; Cc1ccc(N(c2ccc3c(c2)C(C)(C)c2ccccc2-3)c2ccc3c(c2)c2ccccc2n3-c2ccccc2)cc1 ; CC1(C)c2ccccc2-c2ccc(N(c3ccc(F)cc3)c3ccc4c(c3)c3ccccc3n4-c3ccccc3)cc21 ; CC1(C)c2ccccc2-c2ccc(N(c3ccc(C#N)cc3)c3ccc4c(c3)c3ccccc3n4-c3ccccc3)cc21]

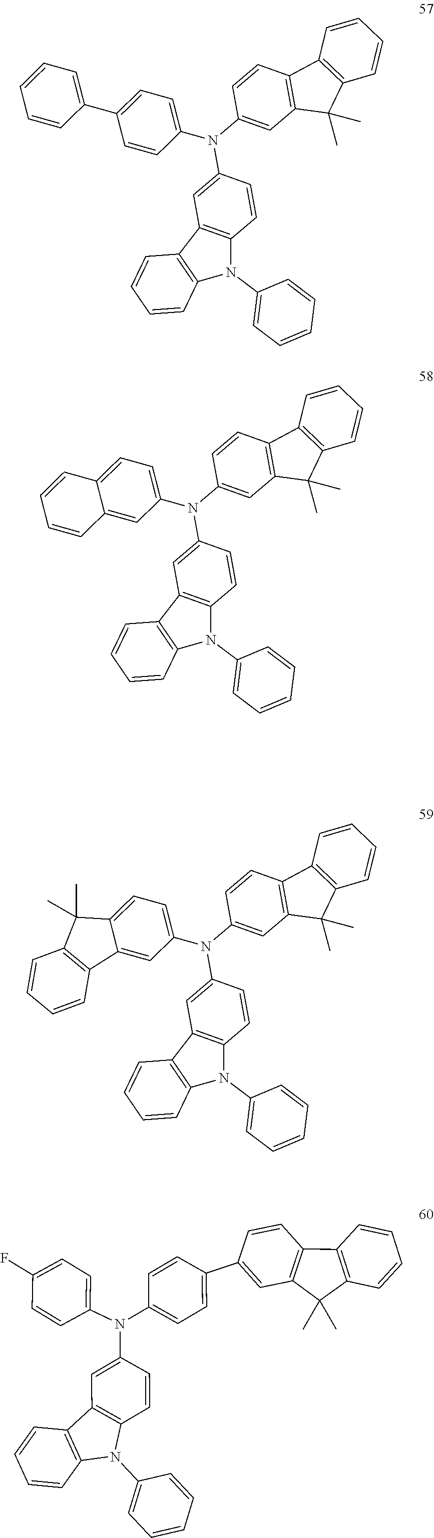 [SMILES: CC1(C)c2ccccc2-c2ccc(N(c3ccc(-c4ccccc4)cc3)c3ccc4c(c3)c3ccccc3n4-c3ccccc3)cc21 ; CC1(C)c2ccccc2-c2ccc(N(c3ccc4ccccc4c3)c3ccc4c(c3)c3ccccc3n4-c3ccccc3)cc21 ; CC1(C)c2ccccc2-c2cc(N(c3ccc4c(c3)C(C)(C)c3ccccc3-4)c3ccc4c(c3)c3ccccc3n4-c3ccccc3)ccc21 ; CC1(C)c2ccccc2-c2ccc(-c3ccc(N(c4ccc(F)cc4)c4ccc5c(c4)c4ccccc4n5-c4ccccc4)cc3)cc21]

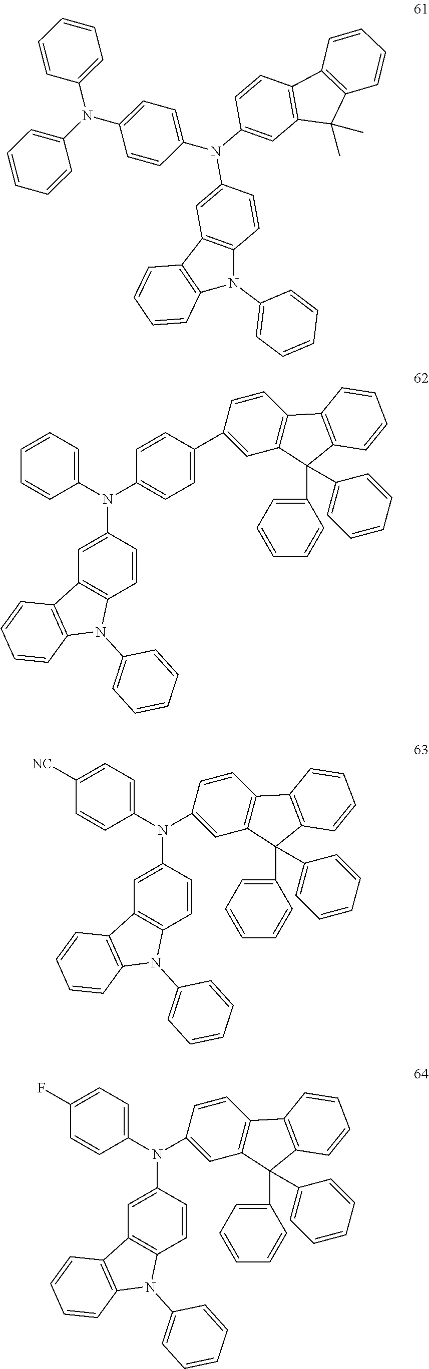

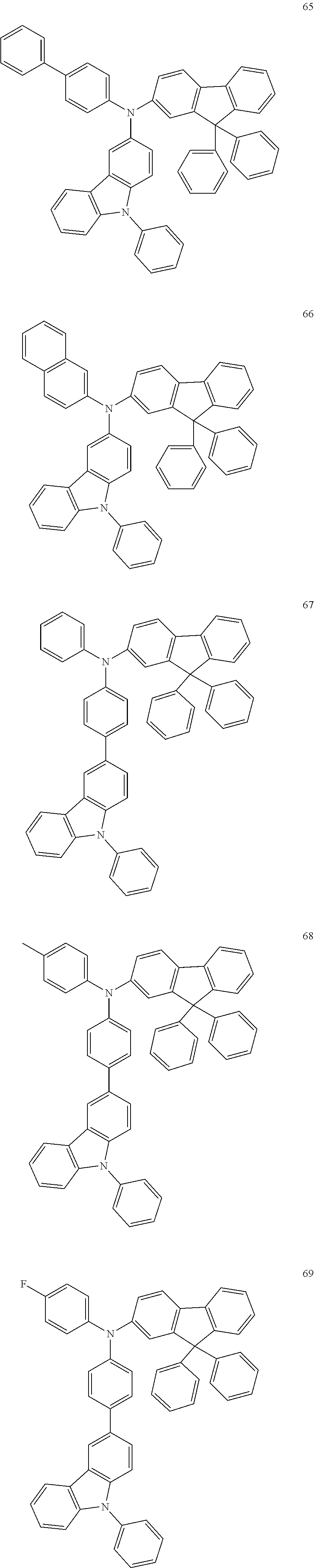 [SMILES: c1ccc(-c2ccc(N(c3ccc4c(c3)C(c3ccccc3)(c3ccccc3)c3ccccc3-4)c3ccc4c(c3)c3ccccc3n4-c3ccccc3)cc2)cc1 ; c1ccc(-n2c3ccccc3c3cc(N(c4ccc5c(c4)C(c4ccccc4)(c4ccccc4)c4ccccc4-5)c4ccc5ccccc5c4)ccc32)cc1 ; c1ccc(N(c2ccc(-c3ccc4c(c3)c3ccccc3n4-c3ccccc3)cc2)c2ccc3c(c2)C(c2ccccc2)(c2ccccc2)c2ccccc2-3)cc1 ; Cc1ccc(N(c2ccc(-c3ccc4c(c3)c3ccccc3n4-c3ccccc3)cc2)c2ccc3c(c2)C(c2ccccc2)(c2ccccc2)c2ccccc2-3)cc1 ; Fc1ccc(N(c2ccc(-c3ccc4c(c3)c3ccccc3n4-c3ccccc3)cc2)c2ccc3c(c2)C(c2ccccc2)(c2ccccc2)c2ccccc2-3)cc1]

40. A light emitting diode, comprising:
a first electrode;
first to fourth light emitting units, each comprising a hole transport region, an emission layer, and an electron transport region, disposed in order on the first electrode;
a charge generating layer disposed between the first to fourth light emitting units; and
a second electrode disposed on the fourth light emitting unit, wherein
the fourth light emitting unit emits light having a longer wavelength than the first to third light emitting units, and
the hole transport region of the fourth light emitting unit comprises an amine compound represented by Formula 1: 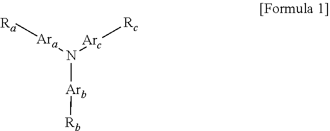
wherein in Formula 1,
Arₐ to Ar_{c} are each independently a substituted or unsubstituted aryl group of 6 to 30 ring-forming carbon atoms, or a substituted or unsubstituted heteroaryl group of 3 to 30 ring-forming carbon atoms,
at least two of Rₐ to R_{c} are each independently an adamantyl group or a cyclohexyl group, and
the remainder of Rₐ to R_{c} is a hydrogen atom, a deuterium atom, a halogen atom, a cyano group, a substituted or unsubstituted oxy group, a substituted or unsubstituted thio group, a substituted or unsubstituted amine group, or a substituted or unsubstituted alkyl group of 1 to 20 carbon atoms.

41. The light emitting diode of claim 40, wherein the hole transport region of at least one of the first to third light emitting units comprises the amine compound.

42. The light emitting diode of claim 41, wherein the hole transport region comprising the amine compound has a refractive index in a range of about 1.30 to about 1.80, with respect to light at a wavelength of about 460 nm.

43. The light emitting diode of claim 40, wherein
each of the first light emitting unit, the second light emitting unit, and the third light emitting unit emits light having a first wavelength in a range of about 430 nm to about 470 nm, and
the fourth light emitting unit emits light having a second wavelength in a range of about 520 nm to about 600 nm.

44. The light emitting diode of claim 43, wherein each of the first light emitting unit, the second light emitting unit, and the third light emitting unit comprises:
a first sub-emission layer comprising a first host, and a first dopant emitting the light having a first wavelength; and
a second sub-emission layer comprising a second host that is different from the first host, and a second dopant emitting the light having a first wavelength.

45. The light emitting diode of claim 40, wherein Arₐ to Ar_{c} are each independently a substituted or unsubstituted phenyl group, a substituted or unsubstituted naphthyl group, a substituted or unsubstituted biphenyl group, a substituted or unsubstituted terphenyl group, a substituted or unsubstituted fluorenyl group, a substituted or unsubstituted carbazole group, a substituted or unsubstituted dibenzofuranyl group, or a substituted or unsubstituted dibenzothiophenyl group.

46. The light emitting diode of claim 40, wherein the amine compound is represented by any one among compounds in the following Compound Group 1:

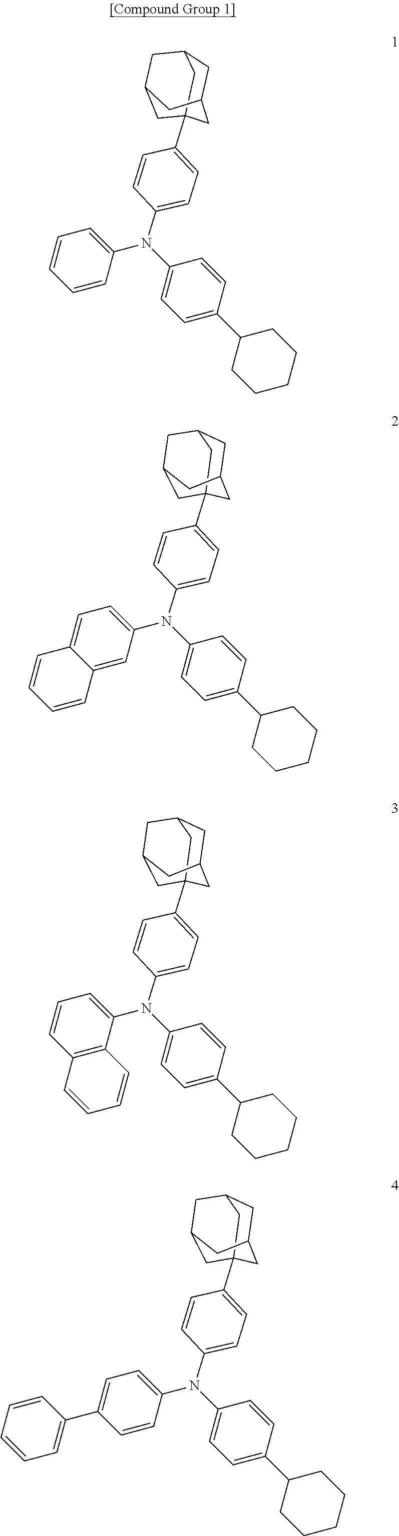 [SMILES: c1ccc(N(c2ccc(C3CCCCC3)cc2)c2ccc(C34CC5CC(CC(C5)C3)C4)cc2)cc1 ; c1ccc2cc(N(c3ccc(C4CCCCC4)cc3)c3ccc(C45CC6CC(CC(C6)C4)C5)cc3)ccc2c1 ; c1ccc2c(N(c3ccc(C4CCCCC4)cc3)c3ccc(C45CC6CC(CC(C6)C4)C5)cc3)cccc2c1 ; c1ccc(-c2ccc(N(c3ccc(C4CCCCC4)cc3)c3ccc(C45CC6CC(CC(C6)C4)C5)cc3)cc2)cc1]

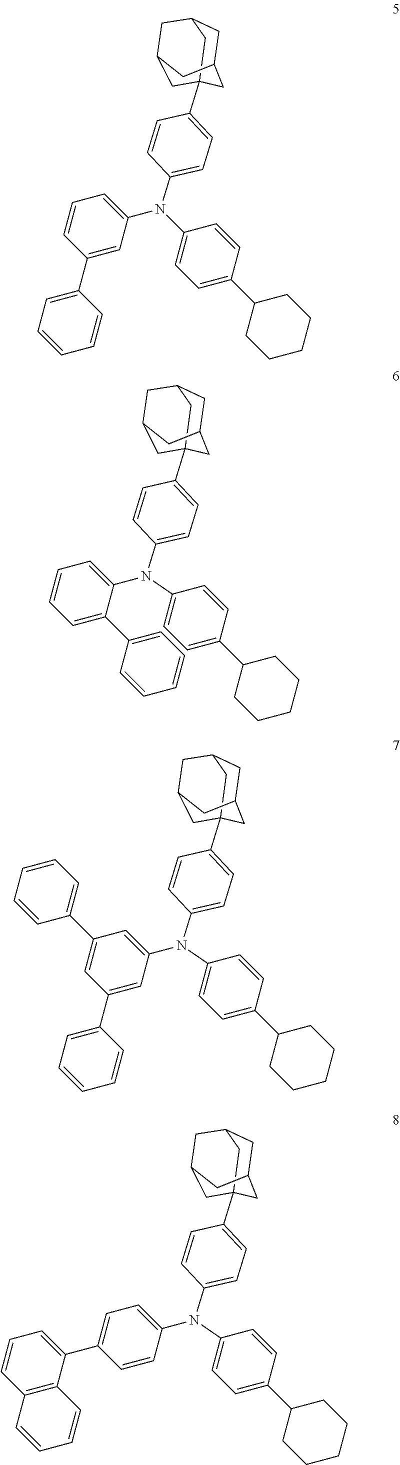 [SMILES: c1ccc(-c2cccc(N(c3ccc(C4CCCCC4)cc3)c3ccc(C45CC6CC(CC(C6)C4)C5)cc3)c2)cc1 ; c1ccc(-c2ccccc2N(c2ccc(C3CCCCC3)cc2)c2ccc(C34CC5CC(CC(C5)C3)C4)cc2)cc1 ; c1ccc(-c2cc(-c3ccccc3)cc(N(c3ccc(C4CCCCC4)cc3)c3ccc(C45CC6CC(CC(C6)C4)C5)cc3)c2)cc1 ; c1ccc2c(-c3ccc(N(c4ccc(C5CCCCC5)cc4)c4ccc(C56CC7CC(CC(C7)C5)C6)cc4)cc3)cccc2c1]

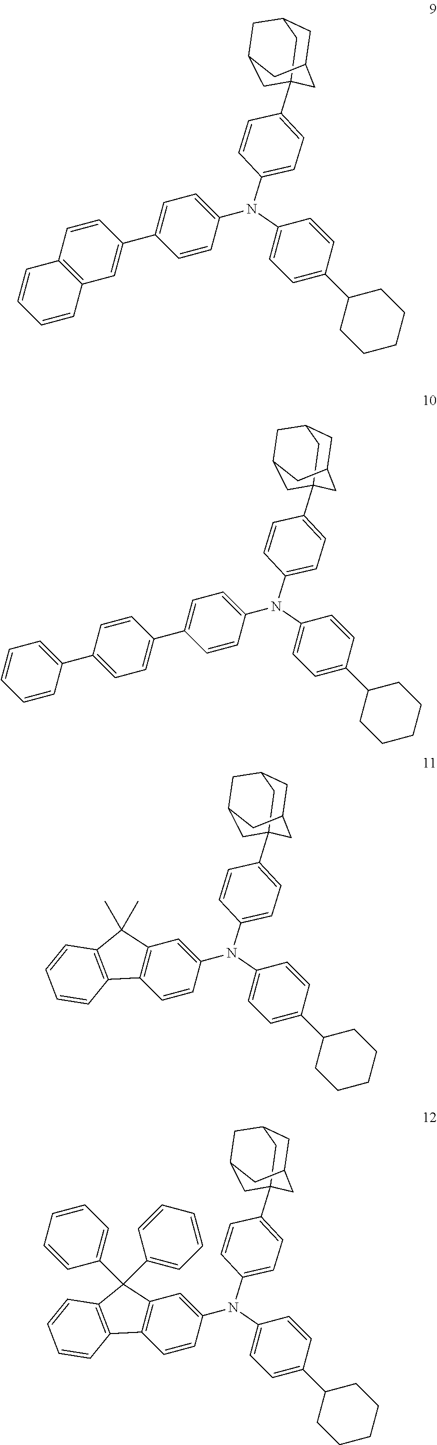 [SMILES: c1ccc2cc(-c3ccc(N(c4ccc(C5CCCCC5)cc4)c4ccc(C56CC7CC(CC(C7)C5)C6)cc4)cc3)ccc2c1 ; c1ccc(-c2ccc(-c3ccc(N(c4ccc(C5CCCCC5)cc4)c4ccc(C56CC7CC(CC(C7)C5)C6)cc4)cc3)cc2)cc1 ; CC1(C)c2ccccc2-c2ccc(N(c3ccc(C4CCCCC4)cc3)c3ccc(C45CC6CC(CC(C6)C4)C5)cc3)cc21 ; c1ccc(C2(c3ccccc3)c3ccccc3-c3ccc(N(c4ccc(C5CCCCC5)cc4)c4ccc(C56CC7CC(CC(C7)C5)C6)cc4)cc32)cc1]

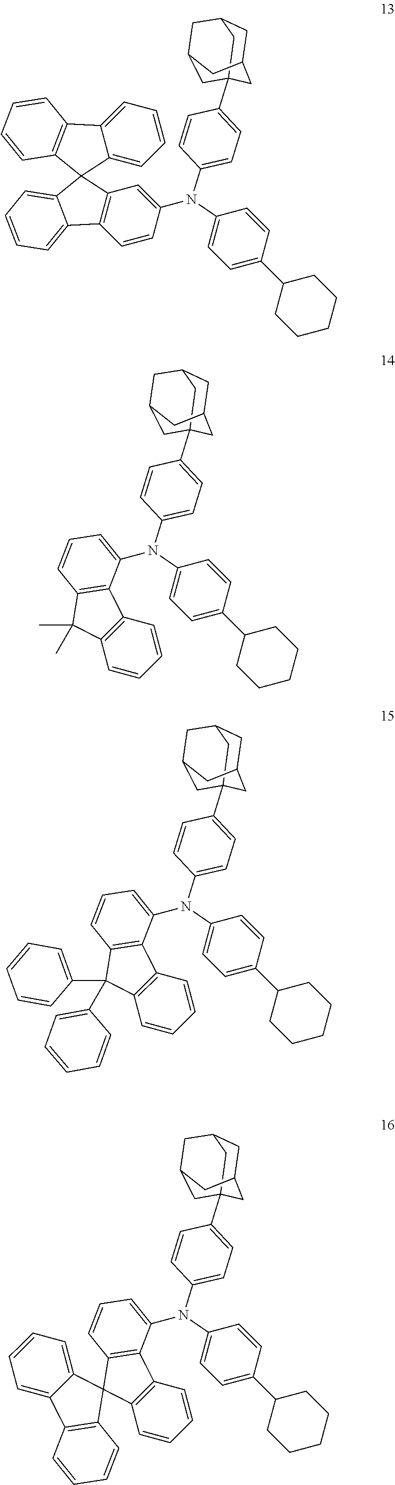 [SMILES: c1ccc2c(c1)-c1ccccc1C21c2ccccc2-c2ccc(N(c3ccc(C4CCCCC4)cc3)c3ccc(C45CC6CC(CC(C6)C4)C5)cc3)cc21 ; CC1(C)c2ccccc2-c2c(N(c3ccc(C4CCCCC4)cc3)c3ccc(C45CC6CC(CC(C6)C4)C5)cc3)cccc21 ; c1ccc(C2(c3ccccc3)c3ccccc3-c3c(N(c4ccc(C5CCCCC5)cc4)c4ccc(C56CC7CC(CC(C7)C5)C6)cc4)cccc32)cc1 ; c1ccc2c(c1)-c1ccccc1C21c2ccccc2-c2c(N(c3ccc(C4CCCCC4)cc3)c3ccc(C45CC6CC(CC(C6)C4)C5)cc3)cccc21]

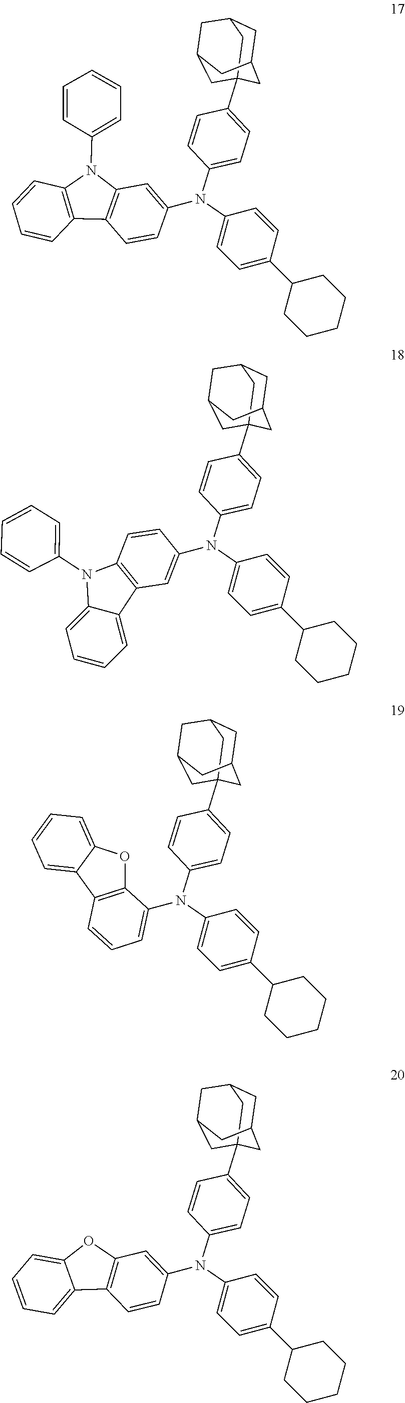 [SMILES: c1ccc(-n2c3ccccc3c3ccc(N(c4ccc(C5CCCCC5)cc4)c4ccc(C56CC7CC(CC(C7)C5)C6)cc4)cc32)cc1 ; c1ccc(-n2c3ccccc3c3cc(N(c4ccc(C5CCCCC5)cc4)c4ccc(C56CC7CC(CC(C7)C5)C6)cc4)ccc32)cc1 ; c1ccc2c(c1)oc1c(N(c3ccc(C4CCCCC4)cc3)c3ccc(C45CC6CC(CC(C6)C4)C5)cc3)cccc12 ; c1ccc2c(c1)oc1cc(N(c3ccc(C4CCCCC4)cc3)c3ccc(C45CC6CC(CC(C6)C4)C5)cc3)ccc12]

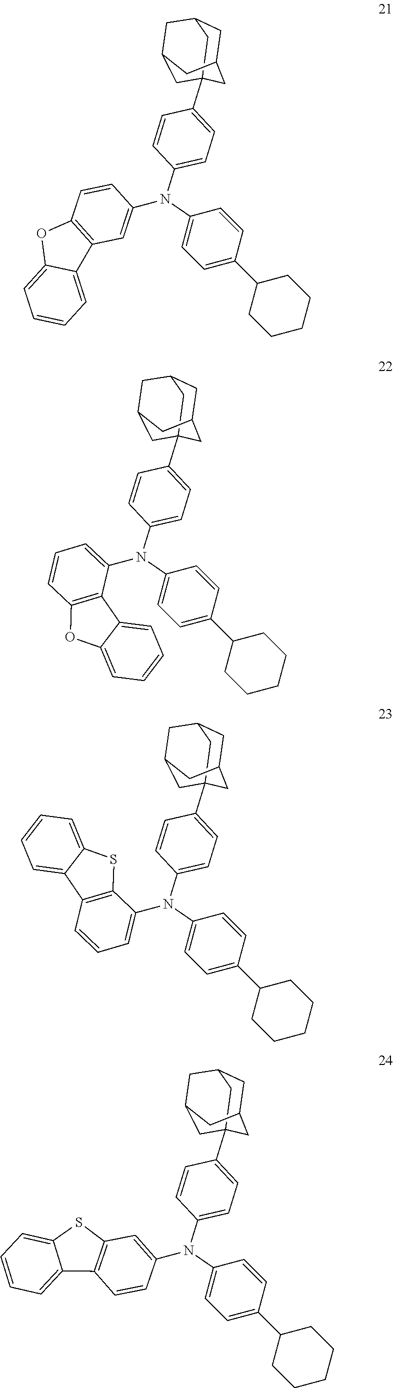 [SMILES: c1ccc2c(c1)oc1ccc(N(c3ccc(C4CCCCC4)cc3)c3ccc(C45CC6CC(CC(C6)C4)C5)cc3)cc12 ; c1ccc2c(c1)oc1cccc(N(c3ccc(C4CCCCC4)cc3)c3ccc(C45CC6CC(CC(C6)C4)C5)cc3)c12 ; c1ccc2c(c1)sc1c(N(c3ccc(C4CCCCC4)cc3)c3ccc(C45CC6CC(CC(C6)C4)C5)cc3)cccc12 ; c1ccc2c(c1)sc1cc(N(c3ccc(C4CCCCC4)cc3)c3ccc(C45CC6CC(CC(C6)C4)C5)cc3)ccc12]

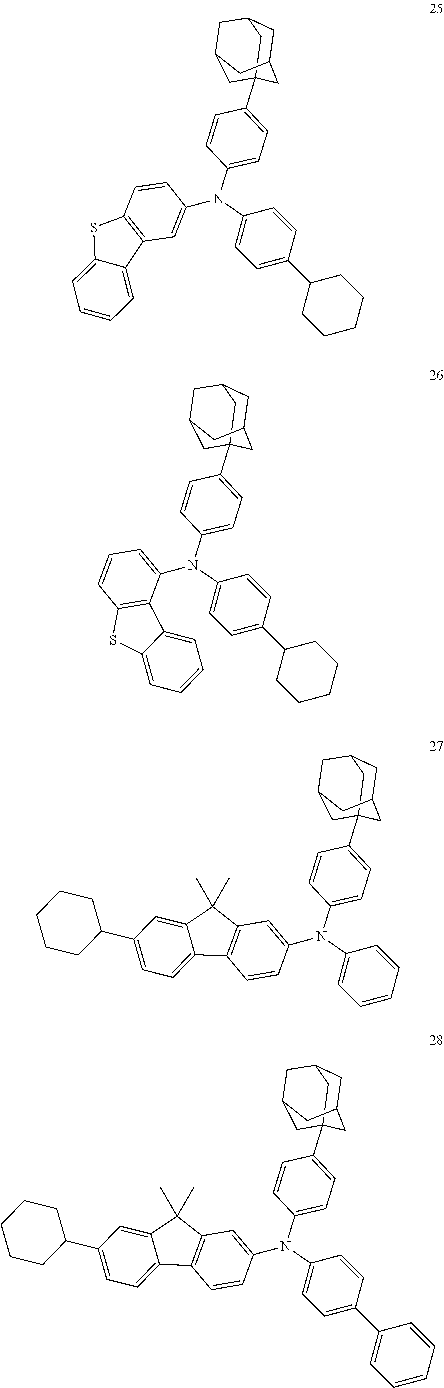 [SMILES: c1ccc2c(c1)sc1ccc(N(c3ccc(C4CCCCC4)cc3)c3ccc(C45CC6CC(CC(C6)C4)C5)cc3)cc12 ; c1ccc2c(c1)sc1cccc(N(c3ccc(C4CCCCC4)cc3)c3ccc(C45CC6CC(CC(C6)C4)C5)cc3)c12 ; CC1(C)c2cc(C3CCCCC3)ccc2-c2ccc(N(c3ccccc3)c3ccc(C45CC6CC(CC(C6)C4)C5)cc3)cc21 ; CC1(C)c2cc(C3CCCCC3)ccc2-c2ccc(N(c3ccc(-c4ccccc4)cc3)c3ccc(C45CC6CC(CC(C6)C4)C5)cc3)cc21]

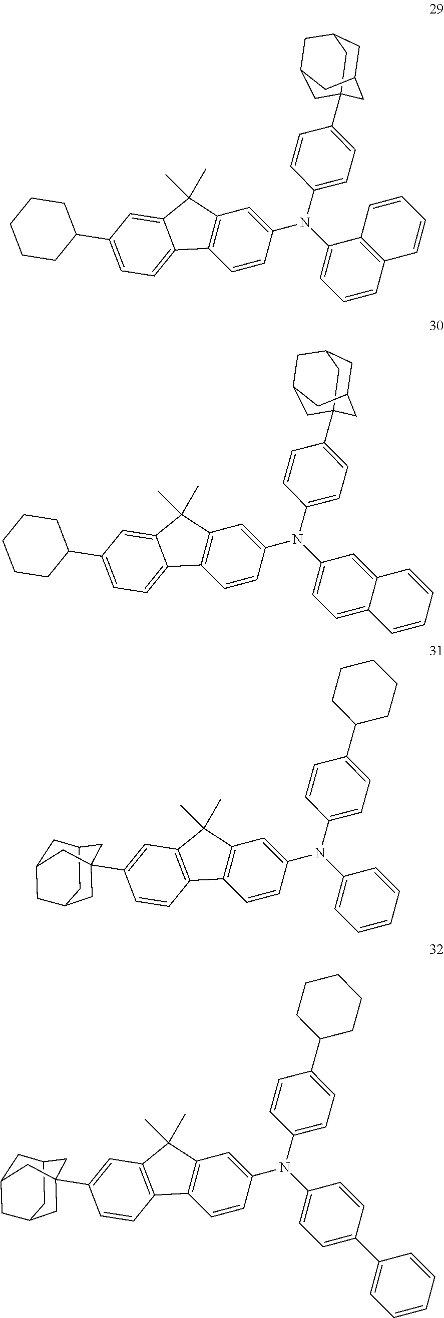

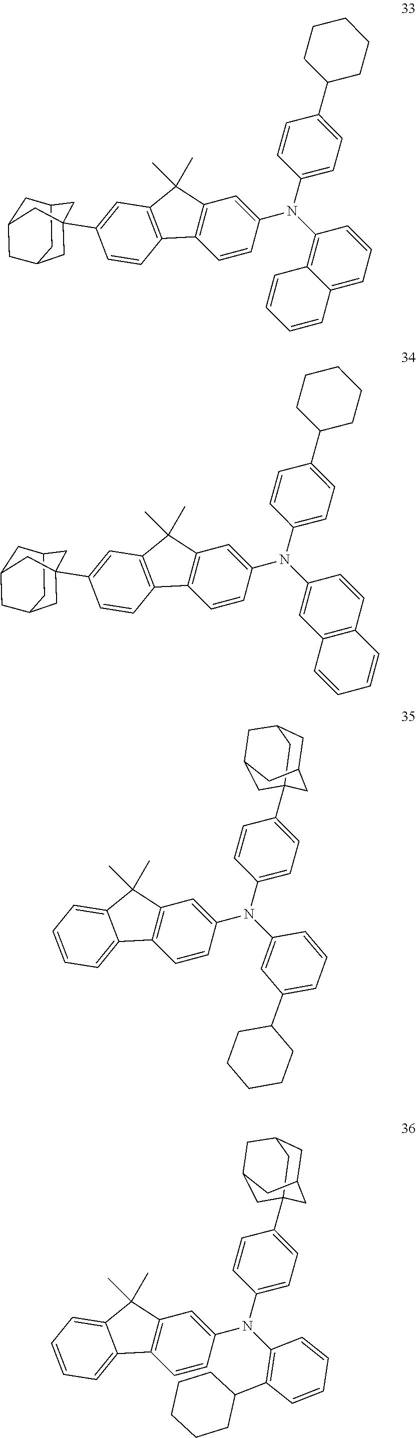 [SMILES: CC1(C)c2cc(N(c3ccc(C4CCCCC4)cc3)c3cccc4ccccc34)ccc2-c2ccc(C34CC5CC(CC(C5)C3)C4)cc21 ; CC1(C)c2cc(N(c3ccc(C4CCCCC4)cc3)c3ccc4ccccc4c3)ccc2-c2ccc(C34CC5CC(CC(C5)C3)C4)cc21 ; CC1(C)c2ccccc2-c2ccc(N(c3ccc(C45CC6CC(CC(C6)C4)C5)cc3)c3cccc(C4CCCCC4)c3)cc21 ; CC1(C)c2ccccc2-c2ccc(N(c3ccc(C45CC6CC(CC(C6)C4)C5)cc3)c3ccccc3C3CCCCC3)cc21]

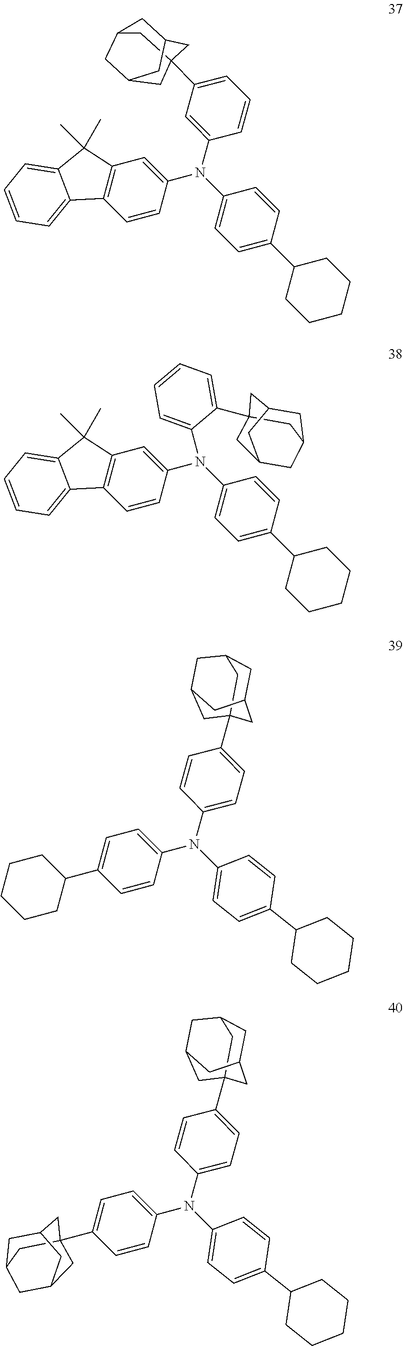 [SMILES: CC1(C)c2ccccc2-c2ccc(N(c3ccc(C4CCCCC4)cc3)c3cccc(C45CC6CC(CC(C6)C4)C5)c3)cc21 ; CC1(C)c2ccccc2-c2ccc(N(c3ccc(C4CCCCC4)cc3)c3ccccc3C34CC5CC(CC(C5)C3)C4)cc21 ; c1cc(N(c2ccc(C3CCCCC3)cc2)c2ccc(C34CC5CC(CC(C5)C3)C4)cc2)ccc1C1CCCCC1 ; c1cc(N(c2ccc(C34CC5CC(CC(C5)C3)C4)cc2)c2ccc(C34CC5CC(CC(C5)C3)C4)cc2)ccc1C1CCCCC1]

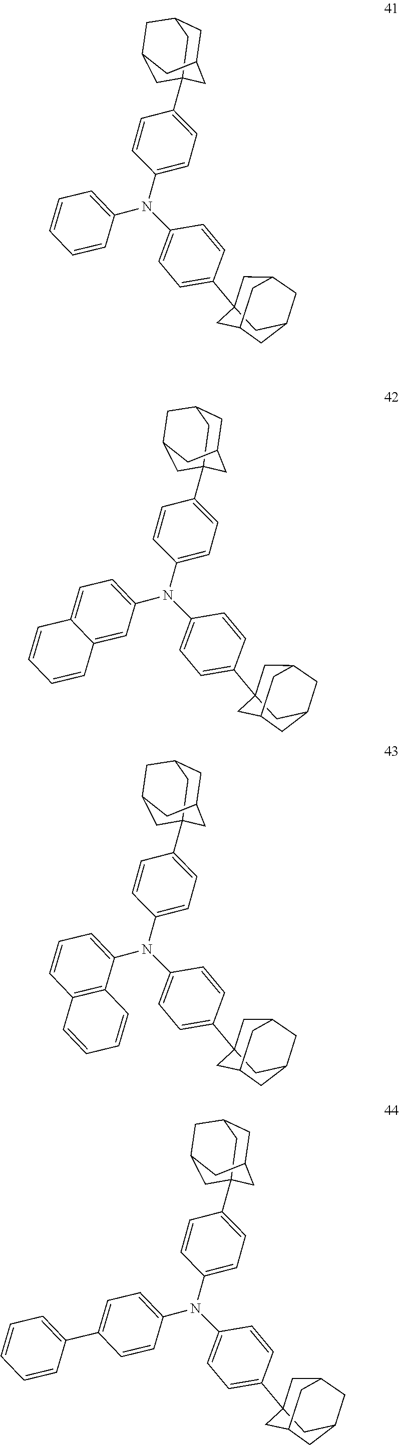 [SMILES: c1ccc(N(c2ccc(C34CC5CC(CC(C5)C3)C4)cc2)c2ccc(C34CC5CC(CC(C5)C3)C4)cc2)cc1 ; c1ccc2cc(N(c3ccc(C45CC6CC(CC(C6)C4)C5)cc3)c3ccc(C45CC6CC(CC(C6)C4)C5)cc3)ccc2c1 ; c1ccc2c(N(c3ccc(C45CC6CC(CC(C6)C4)C5)cc3)c3ccc(C45CC6CC(CC(C6)C4)C5)cc3)cccc2c1 ; c1ccc(-c2ccc(N(c3ccc(C45CC6CC(CC(C6)C4)C5)cc3)c3ccc(C45CC6CC(CC(C6)C4)C5)cc3)cc2)cc1]

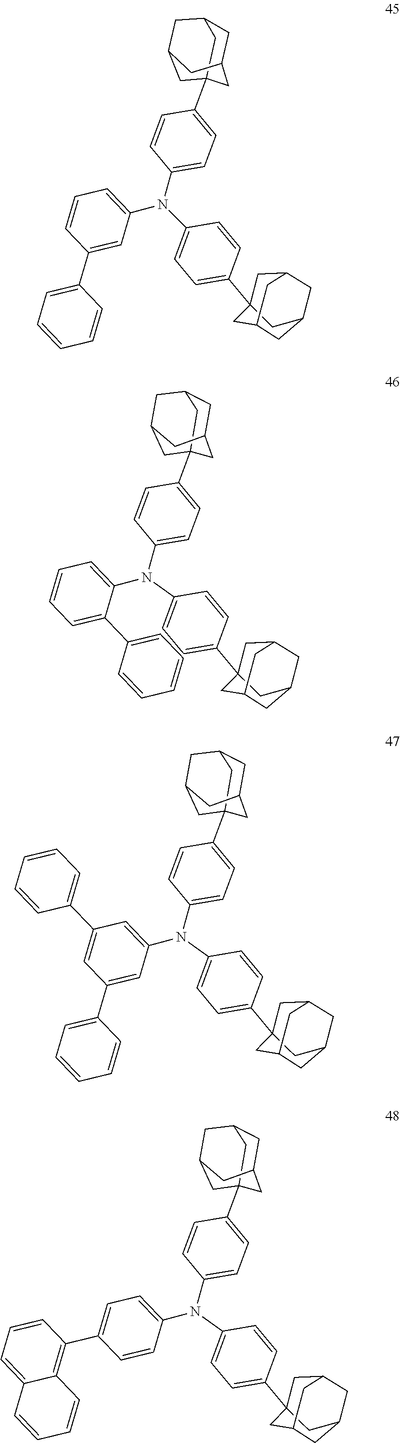 [SMILES: c1ccc(-c2cccc(N(c3ccc(C45CC6CC(CC(C6)C4)C5)cc3)c3ccc(C45CC6CC(CC(C6)C4)C5)cc3)c2)cc1 ; c1ccc(-c2ccccc2N(c2ccc(C34CC5CC(CC(C5)C3)C4)cc2)c2ccc(C34CC5CC(CC(C5)C3)C4)cc2)cc1 ; c1ccc(-c2cc(-c3ccccc3)cc(N(c3ccc(C45CC6CC(CC(C6)C4)C5)cc3)c3ccc(C45CC6CC(CC(C6)C4)C5)cc3)c2)cc1 ; c1ccc2c(-c3ccc(N(c4ccc(C56CC7CC(CC(C7)C5)C6)cc4)c4ccc(C56CC7CC(CC(C7)C5)C6)cc4)cc3)cccc2c1]

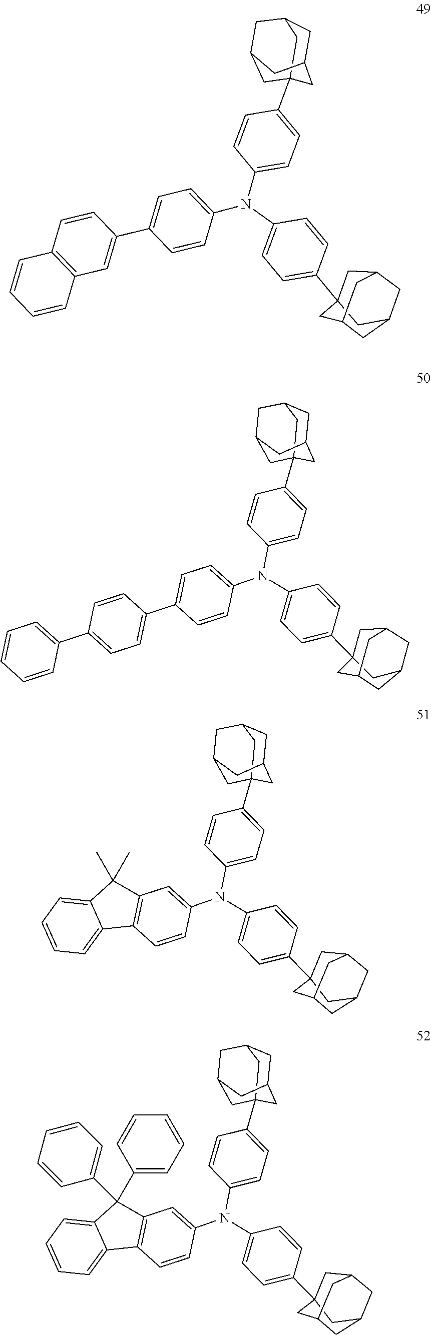 [SMILES: c1ccc2cc(-c3ccc(N(c4ccc(C56CC7CC(CC(C7)C5)C6)cc4)c4ccc(C56CC7CC(CC(C7)C5)C6)cc4)cc3)ccc2c1 ; c1ccc(-c2ccc(-c3ccc(N(c4ccc(C56CC7CC(CC(C7)C5)C6)cc4)c4ccc(C56CC7CC(CC(C7)C5)C6)cc4)cc3)cc2)cc1 ; CC1(C)c2ccccc2-c2ccc(N(c3ccc(C45CC6CC(CC(C6)C4)C5)cc3)c3ccc(C45CC6CC(CC(C6)C4)C5)cc3)cc21 ; c1ccc(C2(c3ccccc3)c3ccccc3-c3ccc(N(c4ccc(C56CC7CC(CC(C7)C5)C6)cc4)c4ccc(C56CC7CC(CC(C7)C5)C6)cc4)cc32)cc1]

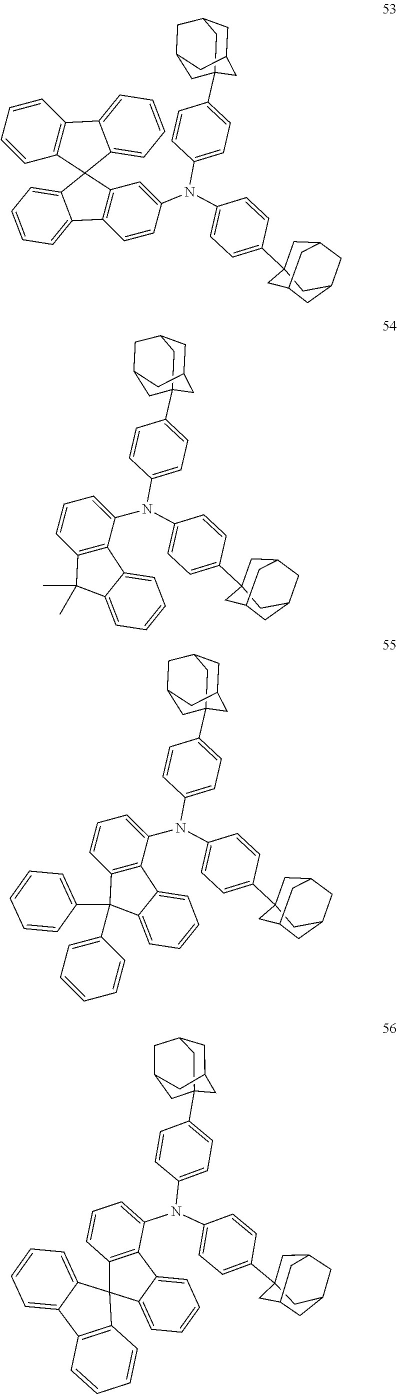

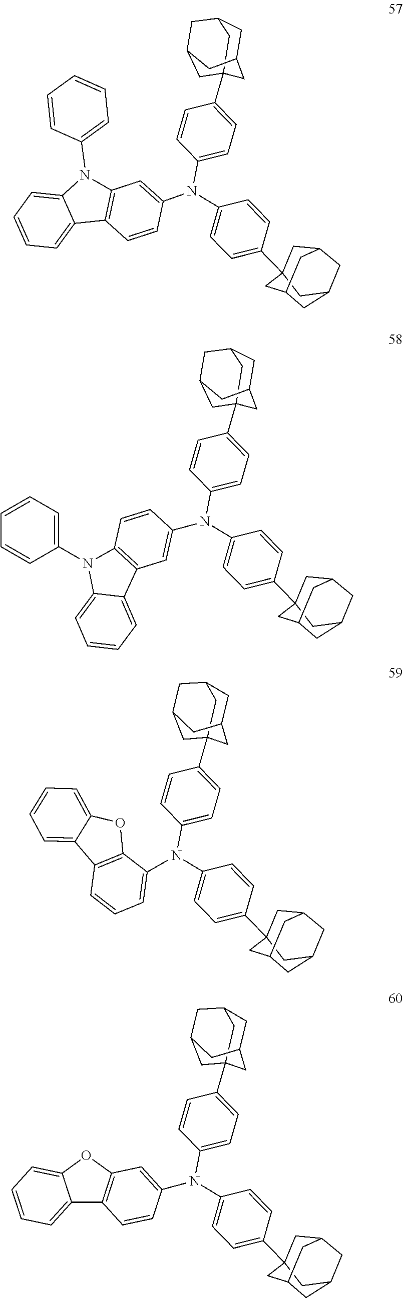 [SMILES: c1ccc(-n2c3ccccc3c3ccc(N(c4ccc(C56CC7CC(CC(C7)C5)C6)cc4)c4ccc(C56CC7CC(CC(C7)C5)C6)cc4)cc32)cc1 ; c1ccc(-n2c3ccccc3c3cc(N(c4ccc(C56CC7CC(CC(C7)C5)C6)cc4)c4ccc(C56CC7CC(CC(C7)C5)C6)cc4)ccc32)cc1 ; c1ccc2c(c1)oc1c(N(c3ccc(C45CC6CC(CC(C6)C4)C5)cc3)c3ccc(C45CC6CC(CC(C6)C4)C5)cc3)cccc12 ; c1ccc2c(c1)oc1cc(N(c3ccc(C45CC6CC(CC(C6)C4)C5)cc3)c3ccc(C45CC6CC(CC(C6)C4)C5)cc3)ccc12]

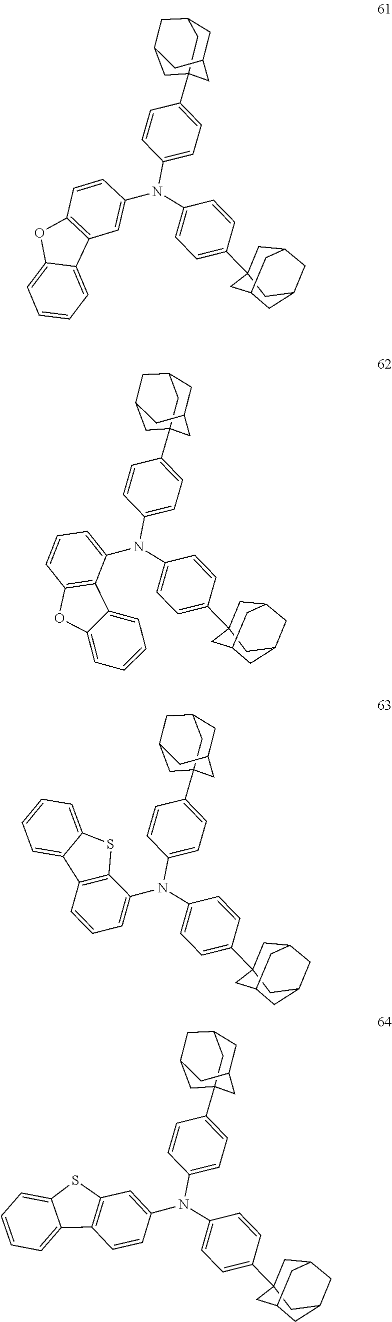 [SMILES: c1ccc2c(c1)oc1ccc(N(c3ccc(C45CC6CC(CC(C6)C4)C5)cc3)c3ccc(C45CC6CC(CC(C6)C4)C5)cc3)cc12 ; c1ccc2c(c1)oc1cccc(N(c3ccc(C45CC6CC(CC(C6)C4)C5)cc3)c3ccc(C45CC6CC(CC(C6)C4)C5)cc3)c12 ; c1ccc2c(c1)sc1c(N(c3ccc(C45CC6CC(CC(C6)C4)C5)cc3)c3ccc(C45CC6CC(CC(C6)C4)C5)cc3)cccc12 ; c1ccc2c(c1)sc1cc(N(c3ccc(C45CC6CC(CC(C6)C4)C5)cc3)c3ccc(C45CC6CC(CC(C6)C4)C5)cc3)ccc12]

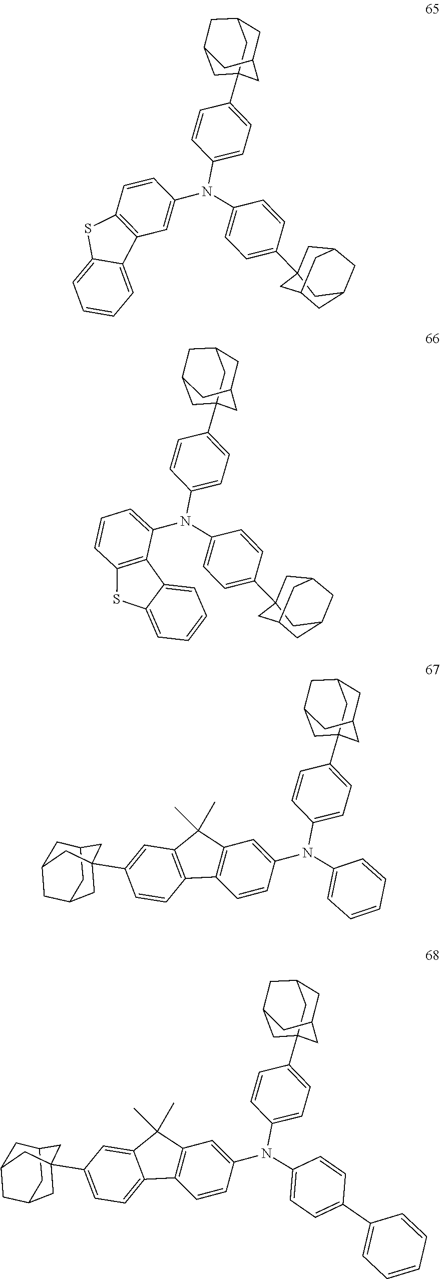 [SMILES: c1ccc2c(c1)sc1ccc(N(c3ccc(C45CC6CC(CC(C6)C4)C5)cc3)c3ccc(C45CC6CC(CC(C6)C4)C5)cc3)cc12 ; c1ccc2c(c1)sc1cccc(N(c3ccc(C45CC6CC(CC(C6)C4)C5)cc3)c3ccc(C45CC6CC(CC(C6)C4)C5)cc3)c12 ; CC1(C)c2cc(N(c3ccccc3)c3ccc(C45CC6CC(CC(C6)C4)C5)cc3)ccc2-c2ccc(C34CC5CC(CC(C5)C3)C4)cc21 ; CC1(C)c2cc(N(c3ccc(-c4ccccc4)cc3)c3ccc(C45CC6CC(CC(C6)C4)C5)cc3)ccc2-c2ccc(C34CC5CC(CC(C5)C3)C4)cc21]

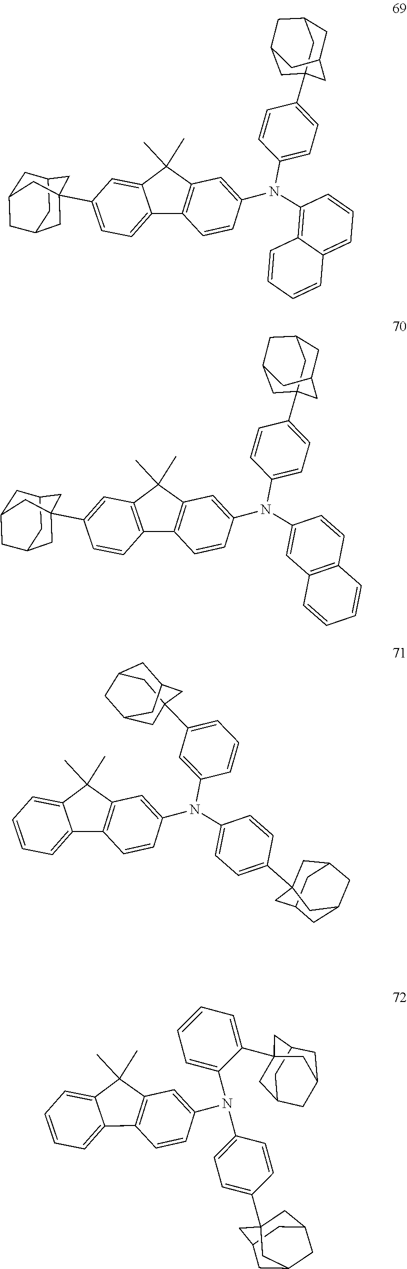

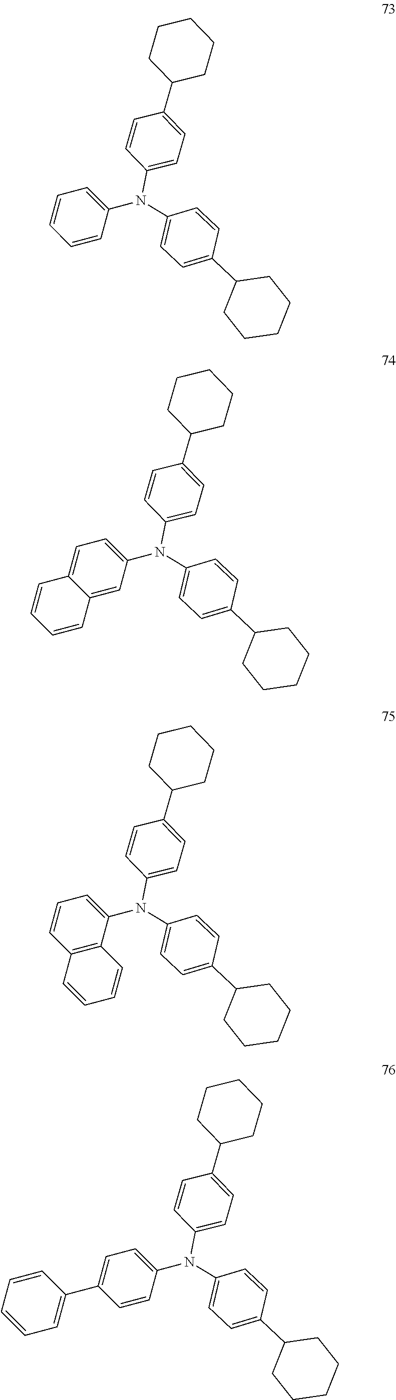

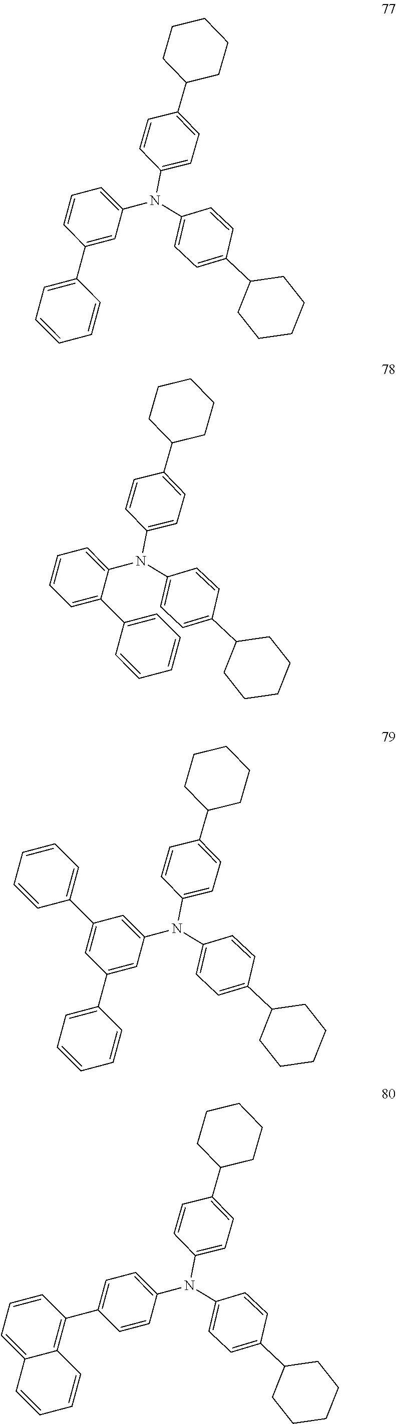 [SMILES: c1ccc(-c2cccc(N(c3ccc(C4CCCCC4)cc3)c3ccc(C4CCCCC4)cc3)c2)cc1 ; c1ccc(-c2ccccc2N(c2ccc(C3CCCCC3)cc2)c2ccc(C3CCCCC3)cc2)cc1 ; c1ccc(-c2cc(-c3ccccc3)cc(N(c3ccc(C4CCCCC4)cc3)c3ccc(C4CCCCC4)cc3)c2)cc1 ; c1ccc2c(-c3ccc(N(c4ccc(C5CCCCC5)cc4)c4ccc(C5CCCCC5)cc4)cc3)cccc2c1]

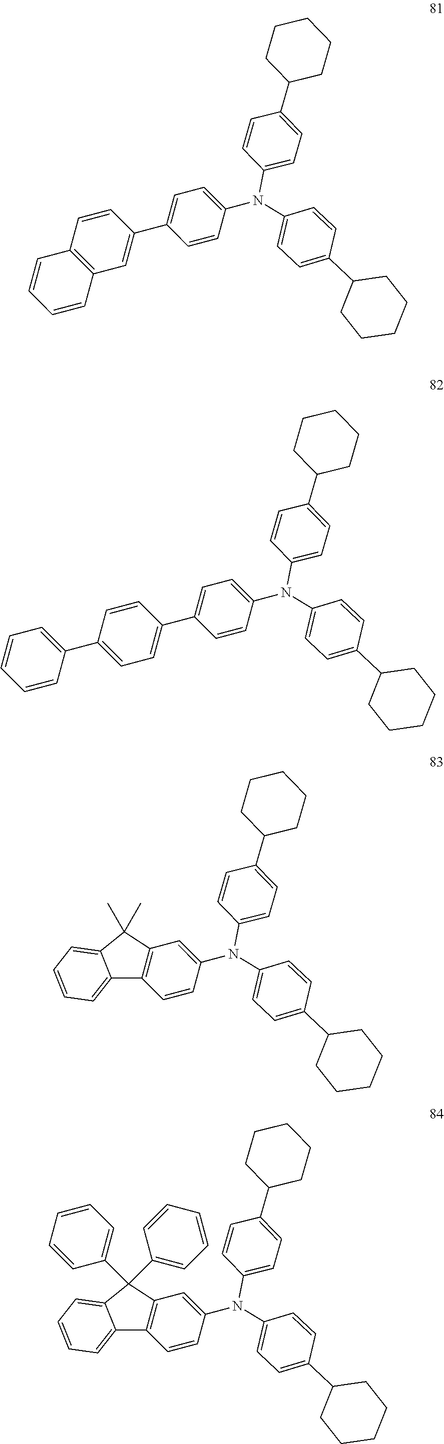 [SMILES: c1ccc2cc(-c3ccc(N(c4ccc(C5CCCCC5)cc4)c4ccc(C5CCCCC5)cc4)cc3)ccc2c1 ; c1ccc(-c2ccc(-c3ccc(N(c4ccc(C5CCCCC5)cc4)c4ccc(C5CCCCC5)cc4)cc3)cc2)cc1 ; CC1(C)c2ccccc2-c2ccc(N(c3ccc(C4CCCCC4)cc3)c3ccc(C4CCCCC4)cc3)cc21 ; c1ccc(C2(c3ccccc3)c3ccccc3-c3ccc(N(c4ccc(C5CCCCC5)cc4)c4ccc(C5CCCCC5)cc4)cc32)cc1]

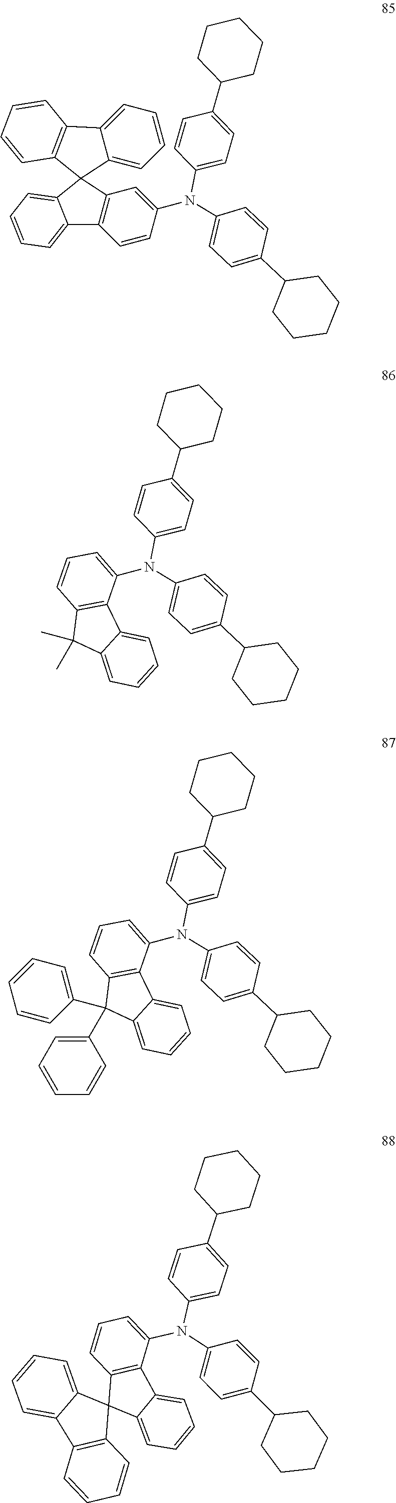 [SMILES: c1ccc2c(c1)-c1ccccc1C21c2ccccc2-c2ccc(N(c3ccc(C4CCCCC4)cc3)c3ccc(C4CCCCC4)cc3)cc21 ; CC1(C)c2ccccc2-c2c(N(c3ccc(C4CCCCC4)cc3)c3ccc(C4CCCCC4)cc3)cccc21 ; c1ccc(C2(c3ccccc3)c3ccccc3-c3c(N(c4ccc(C5CCCCC5)cc4)c4ccc(C5CCCCC5)cc4)cccc32)cc1 ; c1ccc2c(c1)-c1ccccc1C21c2ccccc2-c2c(N(c3ccc(C4CCCCC4)cc3)c3ccc(C4CCCCC4)cc3)cccc21]

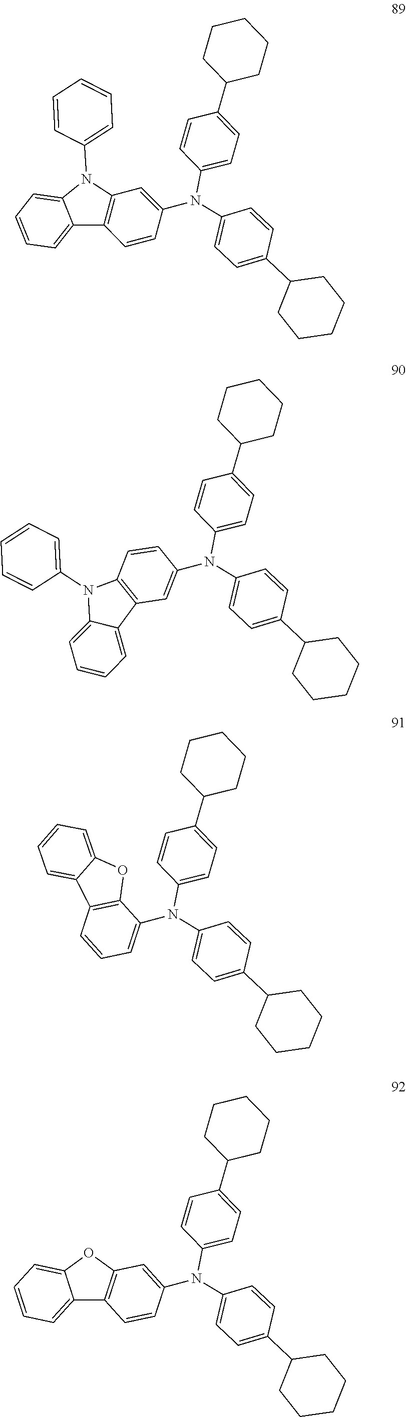 [SMILES: c1ccc(-n2c3ccccc3c3ccc(N(c4ccc(C5CCCCC5)cc4)c4ccc(C5CCCCC5)cc4)cc32)cc1 ; c1ccc(-n2c3ccccc3c3cc(N(c4ccc(C5CCCCC5)cc4)c4ccc(C5CCCCC5)cc4)ccc32)cc1 ; c1ccc2c(c1)oc1c(N(c3ccc(C4CCCCC4)cc3)c3ccc(C4CCCCC4)cc3)cccc12 ; c1ccc2c(c1)oc1cc(N(c3ccc(C4CCCCC4)cc3)c3ccc(C4CCCCC4)cc3)ccc12]

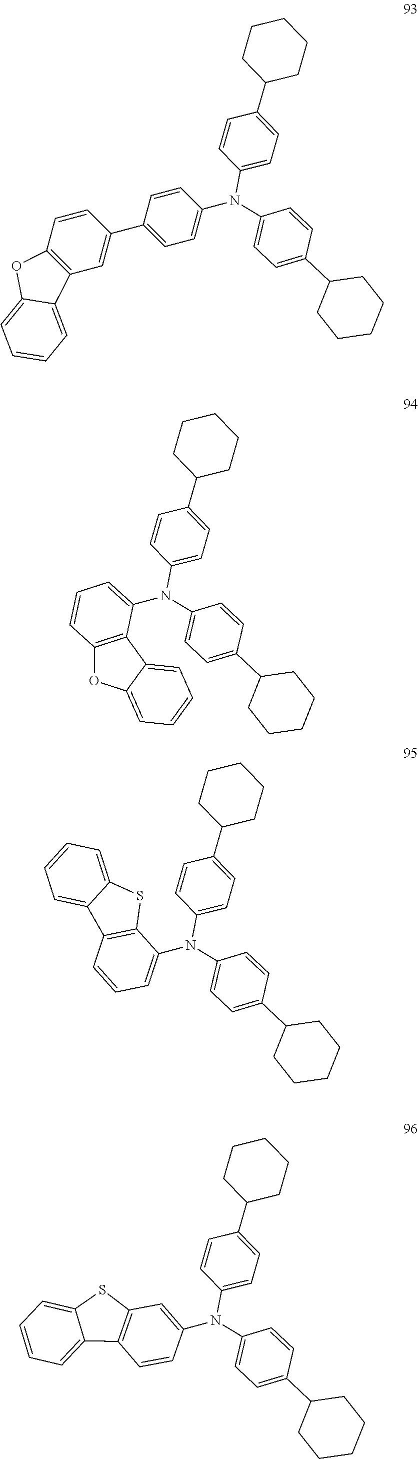

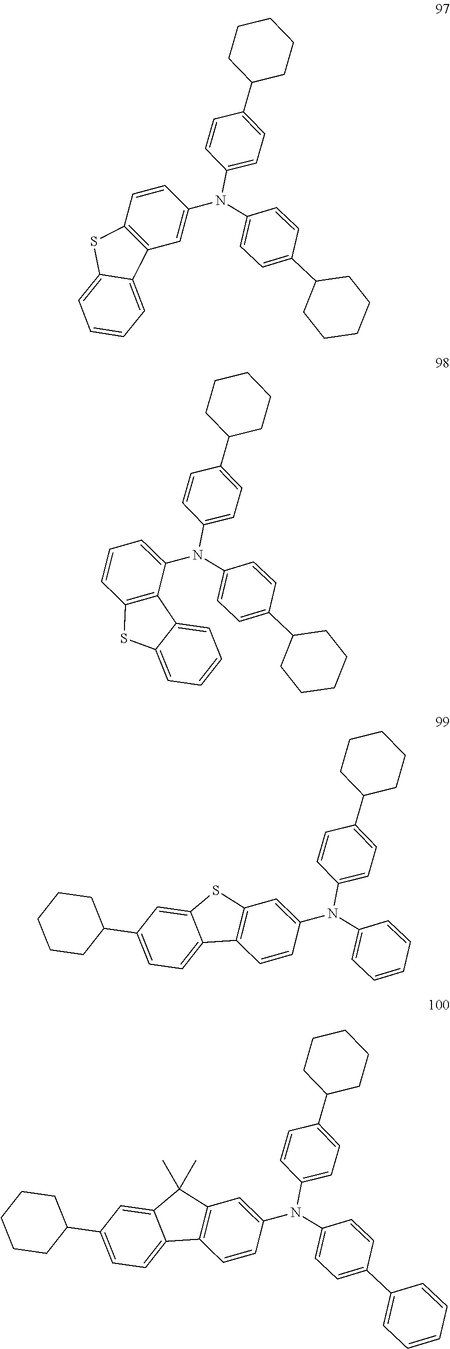 [SMILES: c1ccc2c(c1)sc1ccc(N(c3ccc(C4CCCCC4)cc3)c3ccc(C4CCCCC4)cc3)cc12 ; c1ccc2c(c1)sc1cccc(N(c3ccc(C4CCCCC4)cc3)c3ccc(C4CCCCC4)cc3)c12 ; c1ccc(N(c2ccc(C3CCCCC3)cc2)c2ccc3c(c2)sc2cc(C4CCCCC4)ccc23)cc1 ; CC1(C)c2cc(C3CCCCC3)ccc2-c2ccc(N(c3ccc(-c4ccccc4)cc3)c3ccc(C4CCCCC4)cc3)cc21]

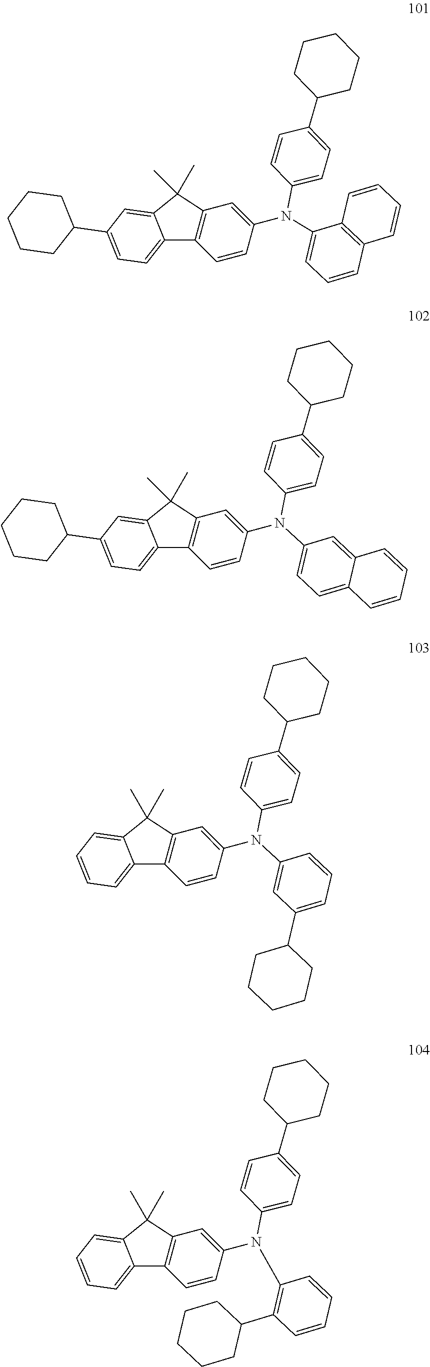 [SMILES: CC1(C)c2cc(C3CCCCC3)ccc2-c2ccc(N(c3ccc(C4CCCCC4)cc3)c3cccc4ccccc34)cc21 ; CC1(C)c2cc(C3CCCCC3)ccc2-c2ccc(N(c3ccc(C4CCCCC4)cc3)c3ccc4ccccc4c3)cc21 ; CC1(C)c2ccccc2-c2ccc(N(c3ccc(C4CCCCC4)cc3)c3cccc(C4CCCCC4)c3)cc21 ; CC1(C)c2ccccc2-c2ccc(N(c3ccc(C4CCCCC4)cc3)c3ccccc3C3CCCCC3)cc21]

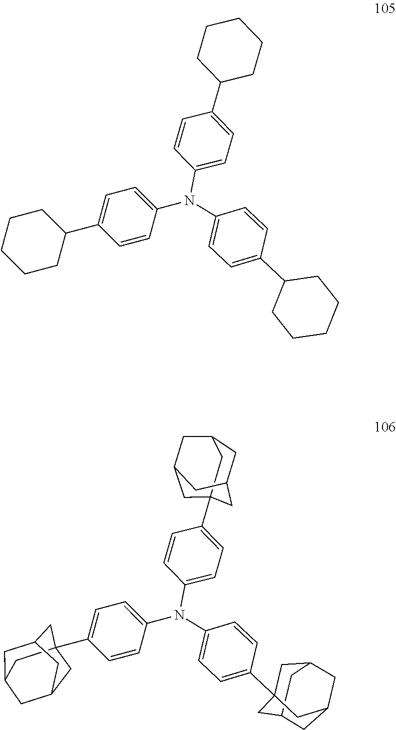 [SMILES: c1cc(N(c2ccc(C3CCCCC3)cc2)c2ccc(C3CCCCC3)cc2)ccc1C1CCCCC1 ; c1cc(C23CC4CC(CC(C4)C2)C3)ccc1N(c1ccc(C23CC4CC(CC(C4)C2)C3)cc1)c1ccc(C23CC4CC(CC(C4)C2)C3)cc1]

47. A display device, divided into:
a first luminous area emitting light with a first wavelength;
a second luminous area emitting light with a second wavelength that is different from the first wavelength; and
a third luminous area emitting light with a third wavelength that is different from the first wavelength and the second wavelength, wherein
the display device comprises:
a display device layer disposed on a base substrate and comprising a light emitting diode; and
a light controlling layer disposed on the display device layer and comprising a quantum dot,
the light emitting diode comprises:
a first electrode;
a second electrode oppositely disposed to the first electrode; and
a plurality of light emitting units disposed between the first electrode and the second electrode,
each of the light emitting units comprises a hole transport region, an emission layer and an electron transport region stacked in order, and
the hole transport region of at least one of the light emitting units comprises an amine compound represented by Formula 1: 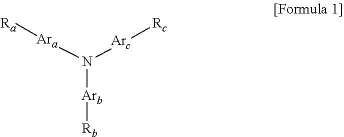
wherein in Formula 1,
Arₐ to Ar_{c} are each independently a substituted or unsubstituted aryl group of 6 to 30 ring-forming carbon atoms, or a substituted or unsubstituted heteroaryl group of 3 to 30 ring-forming carbon atoms,
at least two of Rₐ to R_{c} are each independently an adamantyl group or a cyclohexyl group, and
the remainder of Rₐ to R_{c} is a hydrogen atom, a deuterium atom, a halogen atom, a cyano group, a substituted or unsubstituted oxy group, a substituted or unsubstituted thio group, a substituted or unsubstituted amine group, or a substituted or unsubstituted alkyl group of 1 to 20 carbon atoms.

48. The display device of claim 47, wherein the hole transport region of a light emitting unit adjacent to the second electrode comprises the amine compound.

49. The display device of claim 47, wherein the plurality of light emitting units comprise at least one blue light emitting unit that emits blue light.

50. The display device of claim 47, further comprising a charge generating layer disposed between the plurality of light emitting units.

51. The display device of claim 47, wherein Arₐ to Ar_{c} are each independently a substituted or unsubstituted phenyl group, a substituted or unsubstituted naphthyl group, a substituted or unsubstituted biphenyl group, a substituted or unsubstituted terphenyl group, a substituted or unsubstituted fluorenyl group, a substituted or unsubstituted carbazole group, a substituted or unsubstituted dibenzofuranyl group, or a substituted or unsubstituted dibenzothiophenyl group.

52. The display device of claim 47, wherein the amine compound is selected from Compound Group 1:

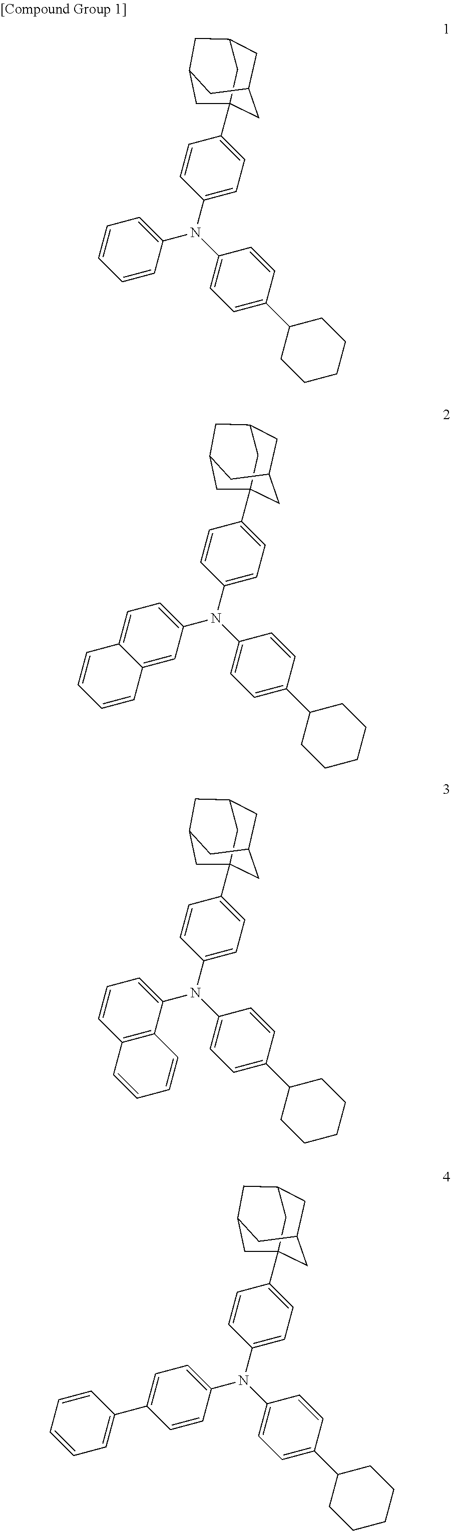 [SMILES: c1ccc(N(c2ccc(C3CCCCC3)cc2)c2ccc(C34CC5CC(CC(C5)C3)C4)cc2)cc1 ; c1ccc2cc(N(c3ccc(C4CCCCC4)cc3)c3ccc(C45CC6CC(CC(C6)C4)C5)cc3)ccc2c1 ; c1ccc2c(N(c3ccc(C4CCCCC4)cc3)c3ccc(C45CC6CC(CC(C6)C4)C5)cc3)cccc2c1 ; c1ccc(-c2ccc(N(c3ccc(C4CCCCC4)cc3)c3ccc(C45CC6CC(CC(C6)C4)C5)cc3)cc2)cc1]

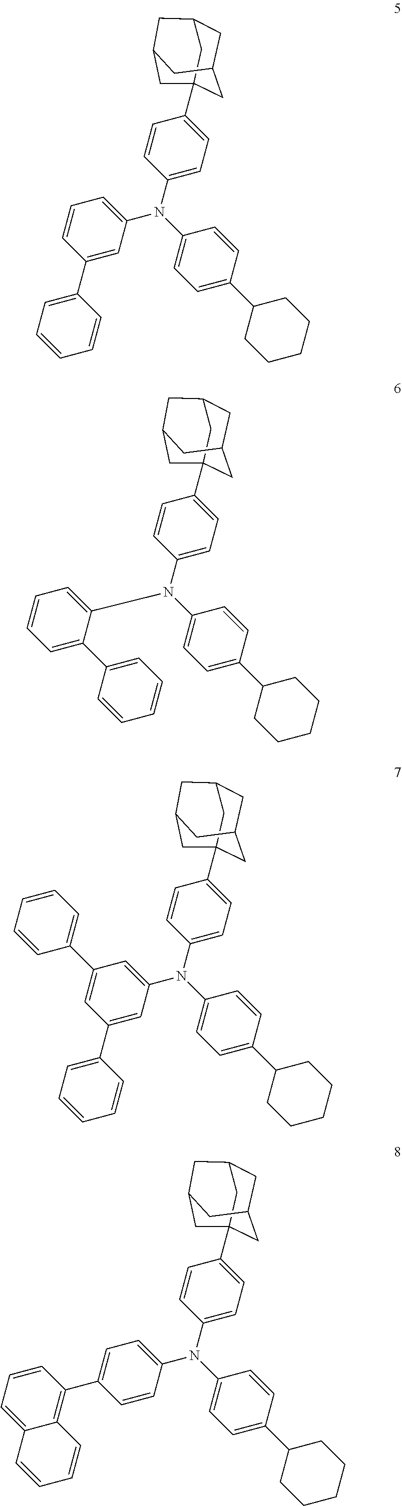 [SMILES: c1ccc(-c2cccc(N(c3ccc(C4CCCCC4)cc3)c3ccc(C45CC6CC(CC(C6)C4)C5)cc3)c2)cc1 ; c1ccc(-c2ccccc2N(c2ccc(C3CCCCC3)cc2)c2ccc(C34CC5CC(CC(C5)C3)C4)cc2)cc1 ; c1ccc(-c2cc(-c3ccccc3)cc(N(c3ccc(C4CCCCC4)cc3)c3ccc(C45CC6CC(CC(C6)C4)C5)cc3)c2)cc1 ; c1ccc2c(-c3ccc(N(c4ccc(C5CCCCC5)cc4)c4ccc(C56CC7CC(CC(C7)C5)C6)cc4)cc3)cccc2c1]

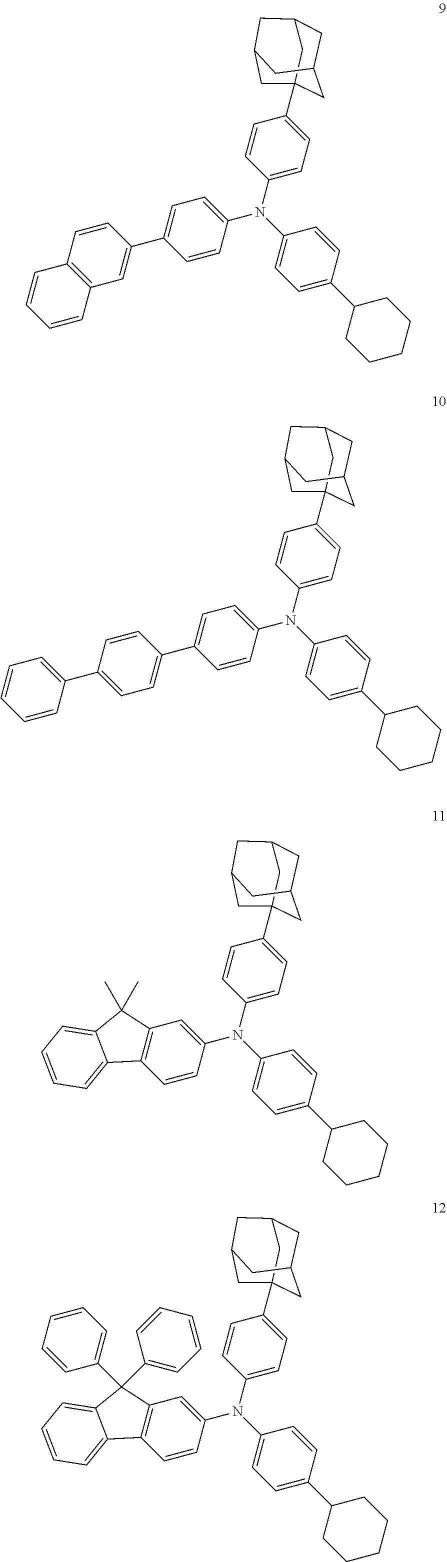 [SMILES: c1ccc2cc(-c3ccc(N(c4ccc(C5CCCCC5)cc4)c4ccc(C56CC7CC(CC(C7)C5)C6)cc4)cc3)ccc2c1 ; c1ccc(-c2ccc(-c3ccc(N(c4ccc(C5CCCCC5)cc4)c4ccc(C56CC7CC(CC(C7)C5)C6)cc4)cc3)cc2)cc1 ; CC1(C)c2ccccc2-c2ccc(N(c3ccc(C4CCCCC4)cc3)c3ccc(C45CC6CC(CC(C6)C4)C5)cc3)cc21 ; c1ccc(C2(c3ccccc3)c3ccccc3-c3ccc(N(c4ccc(C5CCCCC5)cc4)c4ccc(C56CC7CC(CC(C7)C5)C6)cc4)cc32)cc1]

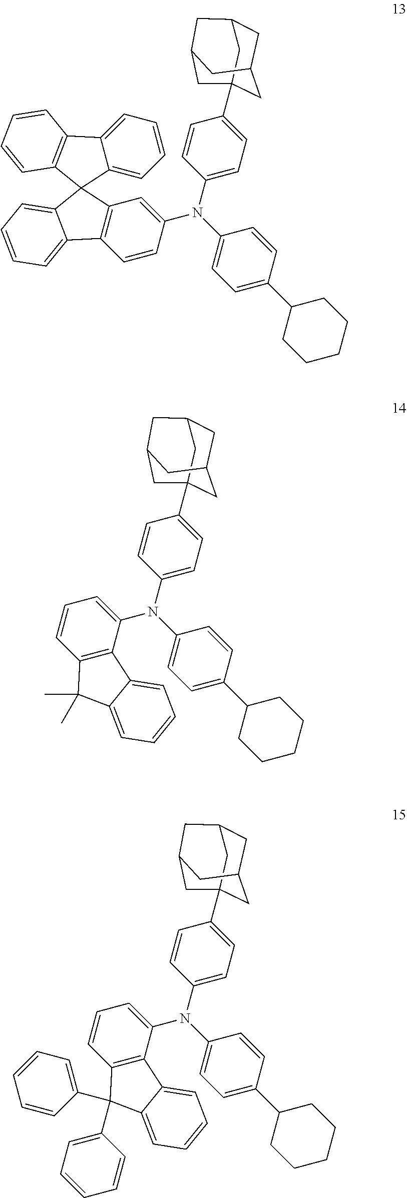 [SMILES: c1ccc2c(c1)-c1ccccc1C21c2ccccc2-c2ccc(N(c3ccc(C4CCCCC4)cc3)c3ccc(C45CC6CC(CC(C6)C4)C5)cc3)cc21 ; CC1(C)c2ccccc2-c2c(N(c3ccc(C4CCCCC4)cc3)c3ccc(C45CC6CC(CC(C6)C4)C5)cc3)cccc21 ; c1ccc(C2(c3ccccc3)c3ccccc3-c3c(N(c4ccc(C5CCCCC5)cc4)c4ccc(C56CC7CC(CC(C7)C5)C6)cc4)cccc32)cc1]

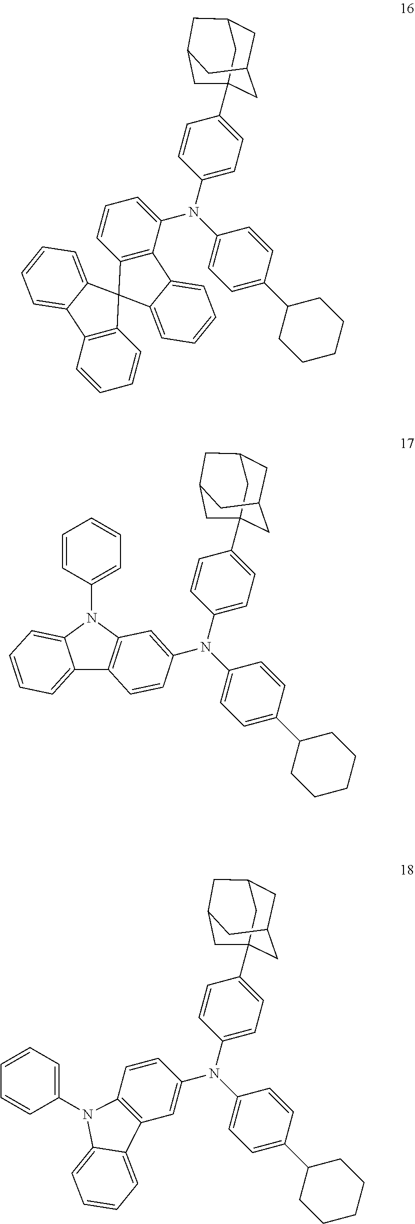 [SMILES: c1ccc2c(c1)-c1ccccc1C21c2ccccc2-c2c(N(c3ccc(C4CCCCC4)cc3)c3ccc(C45CC6CC(CC(C6)C4)C5)cc3)cccc21 ; c1ccc(-n2c3ccccc3c3ccc(N(c4ccc(C5CCCCC5)cc4)c4ccc(C56CC7CC(CC(C7)C5)C6)cc4)cc32)cc1 ; c1ccc(-n2c3ccccc3c3cc(N(c4ccc(C5CCCCC5)cc4)c4ccc(C56CC7CC(CC(C7)C5)C6)cc4)ccc32)cc1]

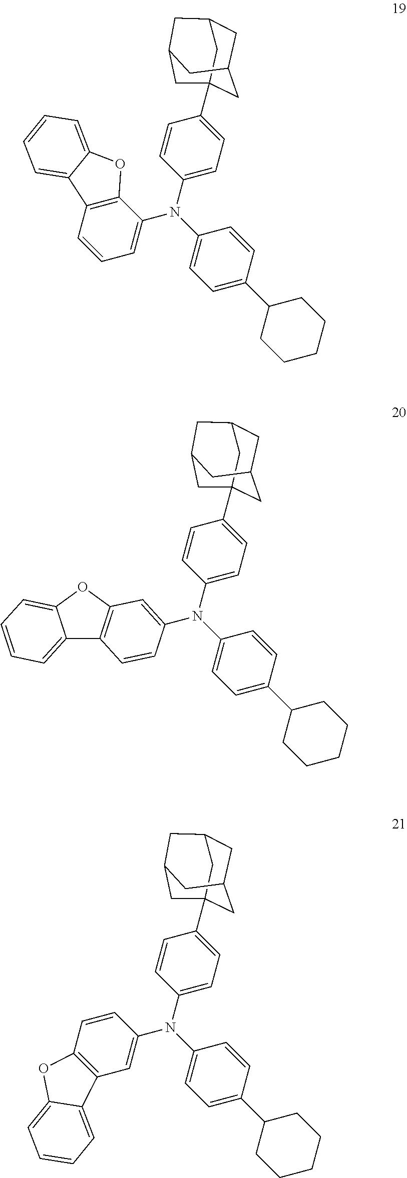 [SMILES: c1ccc2c(c1)oc1c(N(c3ccc(C4CCCCC4)cc3)c3ccc(C45CC6CC(CC(C6)C4)C5)cc3)cccc12 ; c1ccc2c(c1)oc1cc(N(c3ccc(C4CCCCC4)cc3)c3ccc(C45CC6CC(CC(C6)C4)C5)cc3)ccc12 ; c1ccc2c(c1)oc1ccc(N(c3ccc(C4CCCCC4)cc3)c3ccc(C45CC6CC(CC(C6)C4)C5)cc3)cc12]

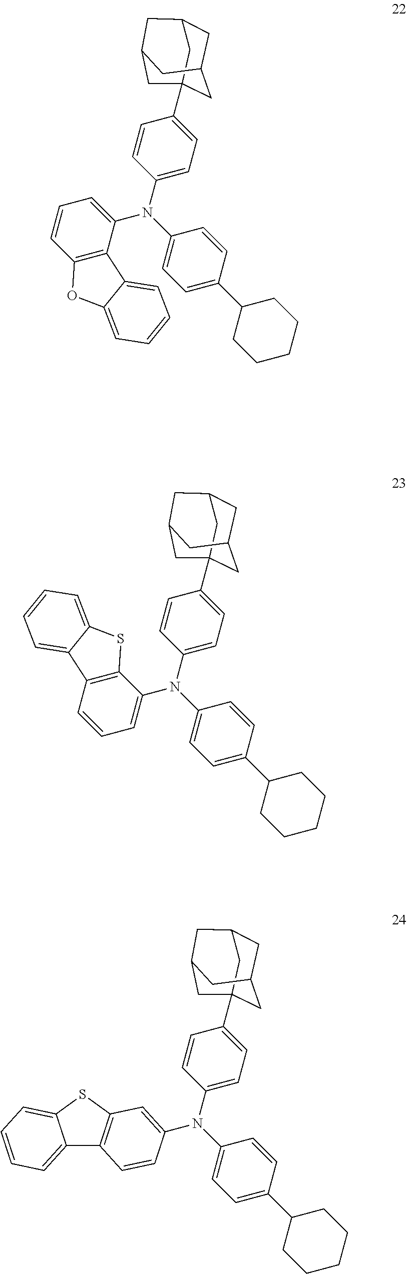 [SMILES: c1ccc2c(c1)oc1cccc(N(c3ccc(C4CCCCC4)cc3)c3ccc(C45CC6CC(CC(C6)C4)C5)cc3)c12 ; c1ccc2c(c1)sc1c(N(c3ccc(C4CCCCC4)cc3)c3ccc(C45CC6CC(CC(C6)C4)C5)cc3)cccc12 ; c1ccc2c(c1)sc1cc(N(c3ccc(C4CCCCC4)cc3)c3ccc(C45CC6CC(CC(C6)C4)C5)cc3)ccc12]

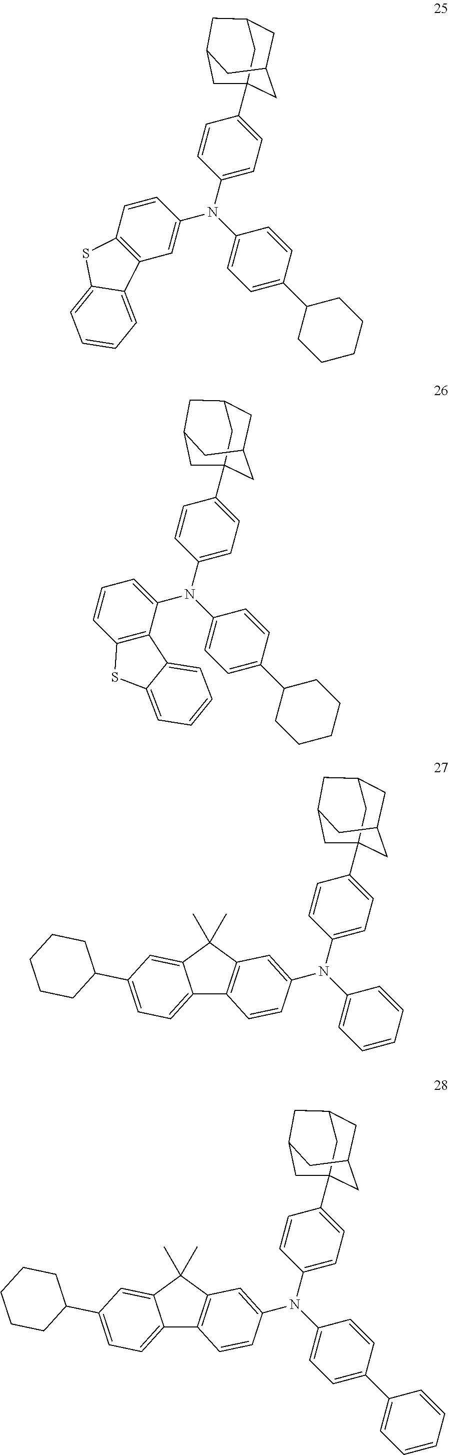

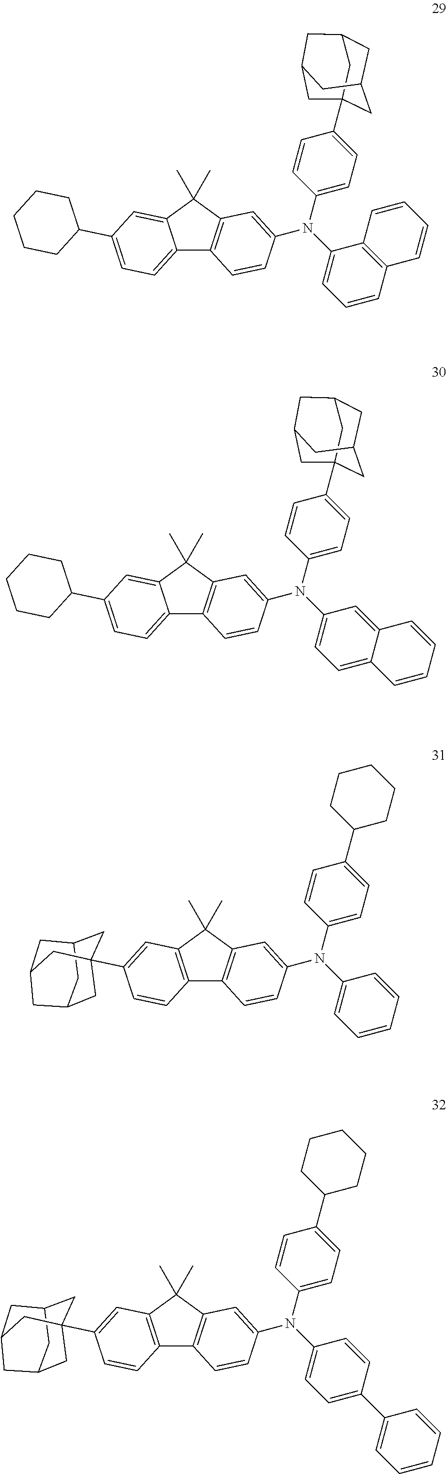 [SMILES: CC1(C)c2cc(C3CCCCC3)ccc2-c2ccc(N(c3ccc(C45CC6CC(CC(C6)C4)C5)cc3)c3cccc4ccccc34)cc21 ; CC1(C)c2cc(C3CCCCC3)ccc2-c2ccc(N(c3ccc(C45CC6CC(CC(C6)C4)C5)cc3)c3ccc4ccccc4c3)cc21 ; CC1(C)c2cc(N(c3ccccc3)c3ccc(C4CCCCC4)cc3)ccc2-c2ccc(C34CC5CC(CC(C5)C3)C4)cc21 ; CC1(C)c2cc(N(c3ccc(-c4ccccc4)cc3)c3ccc(C4CCCCC4)cc3)ccc2-c2ccc(C34CC5CC(CC(C5)C3)C4)cc21]

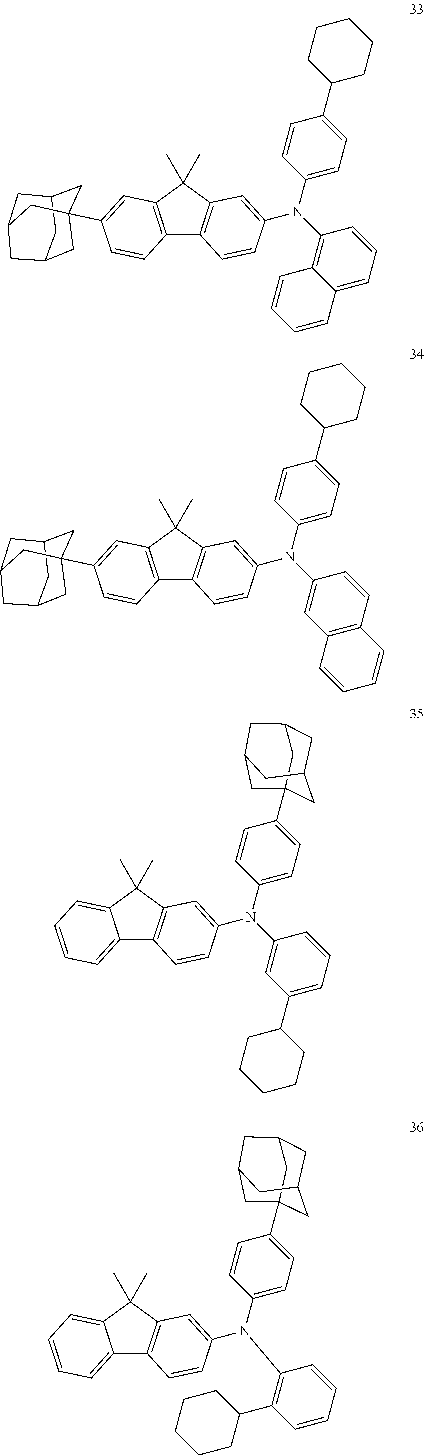

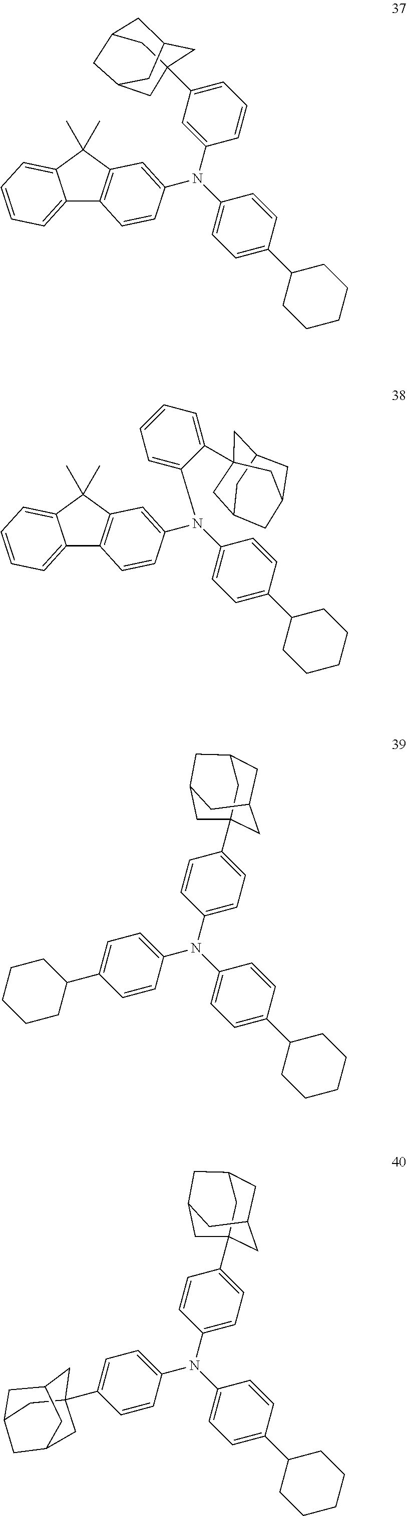 [SMILES: CC1(C)c2ccccc2-c2ccc(N(c3ccc(C4CCCCC4)cc3)c3cccc(C45CC6CC(CC(C6)C4)C5)c3)cc21 ; CC1(C)c2ccccc2-c2ccc(N(c3ccc(C4CCCCC4)cc3)c3ccccc3C34CC5CC(CC(C5)C3)C4)cc21 ; c1cc(N(c2ccc(C3CCCCC3)cc2)c2ccc(C34CC5CC(CC(C5)C3)C4)cc2)ccc1C1CCCCC1 ; c1cc(N(c2ccc(C34CC5CC(CC(C5)C3)C4)cc2)c2ccc(C34CC5CC(CC(C5)C3)C4)cc2)ccc1C1CCCCC1]

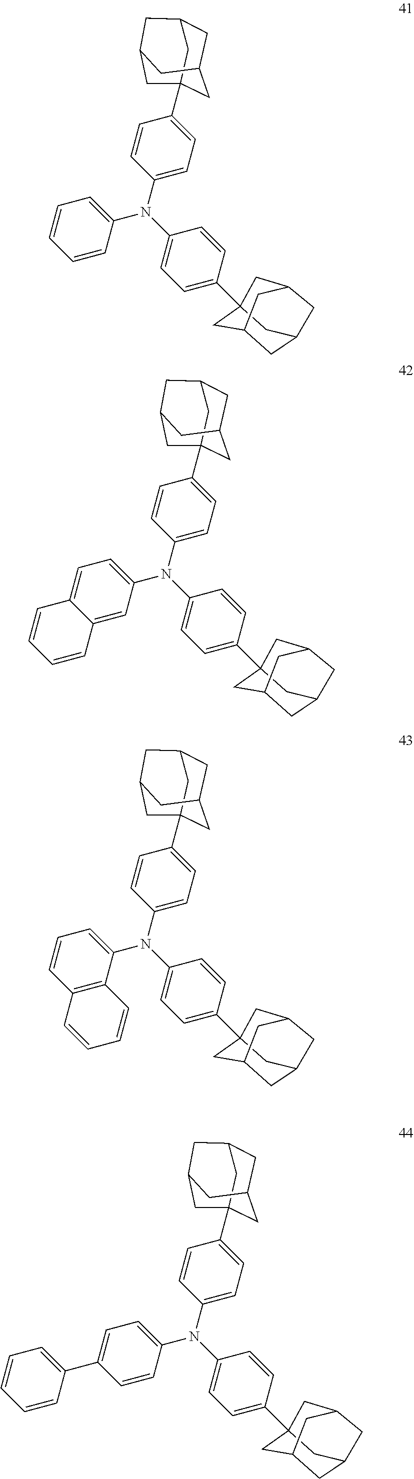 [SMILES: c1ccc(N(c2ccc(C34CC5CC(CC(C5)C3)C4)cc2)c2ccc(C34CC5CC(CC(C5)C3)C4)cc2)cc1 ; c1ccc2cc(N(c3ccc(C45CC6CC(CC(C6)C4)C5)cc3)c3ccc(C45CC6CC(CC(C6)C4)C5)cc3)ccc2c1 ; c1ccc2c(N(c3ccc(C45CC6CC(CC(C6)C4)C5)cc3)c3ccc(C45CC6CC(CC(C6)C4)C5)cc3)cccc2c1 ; c1ccc(-c2ccc(N(c3ccc(C45CC6CC(CC(C6)C4)C5)cc3)c3ccc(C45CC6CC(CC(C6)C4)C5)cc3)cc2)cc1]

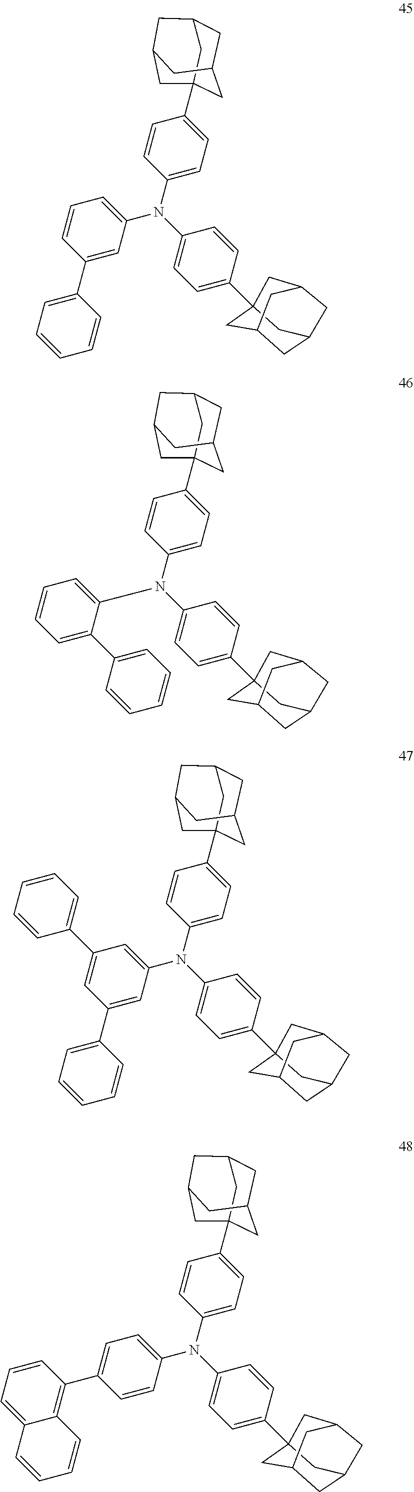 [SMILES: c1ccc(-c2cccc(N(c3ccc(C45CC6CC(CC(C6)C4)C5)cc3)c3ccc(C45CC6CC(CC(C6)C4)C5)cc3)c2)cc1 ; c1ccc(-c2ccccc2N(c2ccc(C34CC5CC(CC(C5)C3)C4)cc2)c2ccc(C34CC5CC(CC(C5)C3)C4)cc2)cc1 ; c1ccc(-c2cc(-c3ccccc3)cc(N(c3ccc(C45CC6CC(CC(C6)C4)C5)cc3)c3ccc(C45CC6CC(CC(C6)C4)C5)cc3)c2)cc1 ; c1ccc2c(-c3ccc(N(c4ccc(C56CC7CC(CC(C7)C5)C6)cc4)c4ccc(C56CC7CC(CC(C7)C5)C6)cc4)cc3)cccc2c1]

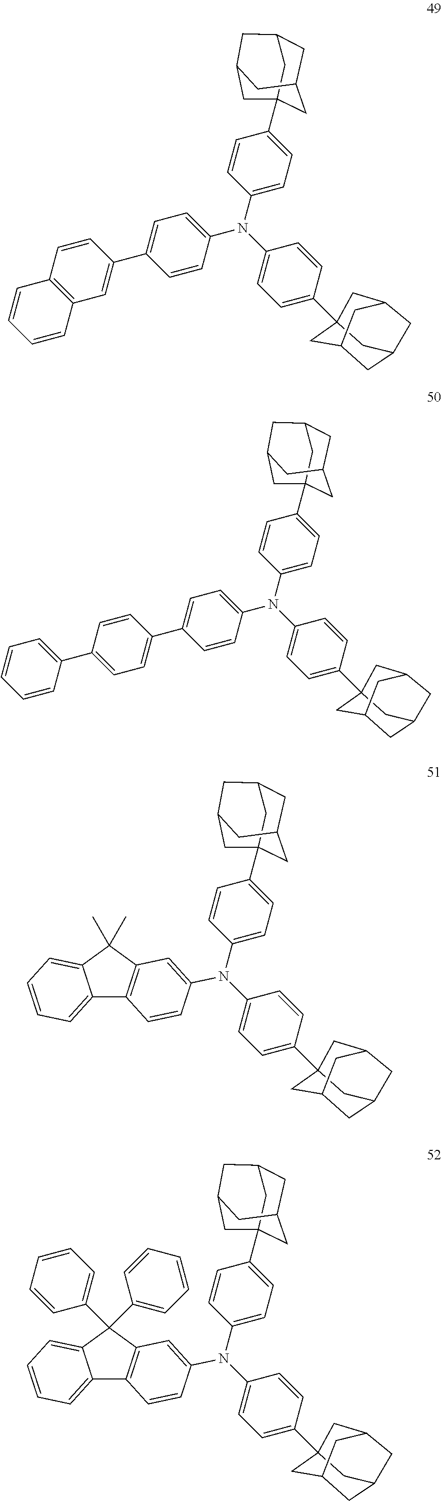 [SMILES: c1ccc2cc(-c3ccc(N(c4ccc(C56CC7CC(CC(C7)C5)C6)cc4)c4ccc(C56CC7CC(CC(C7)C5)C6)cc4)cc3)ccc2c1 ; c1ccc(-c2ccc(-c3ccc(N(c4ccc(C56CC7CC(CC(C7)C5)C6)cc4)c4ccc(C56CC7CC(CC(C7)C5)C6)cc4)cc3)cc2)cc1 ; CC1(C)c2ccccc2-c2ccc(N(c3ccc(C45CC6CC(CC(C6)C4)C5)cc3)c3ccc(C45CC6CC(CC(C6)C4)C5)cc3)cc21 ; c1ccc(C2(c3ccccc3)c3ccccc3-c3ccc(N(c4ccc(C56CC7CC(CC(C7)C5)C6)cc4)c4ccc(C56CC7CC(CC(C7)C5)C6)cc4)cc32)cc1]

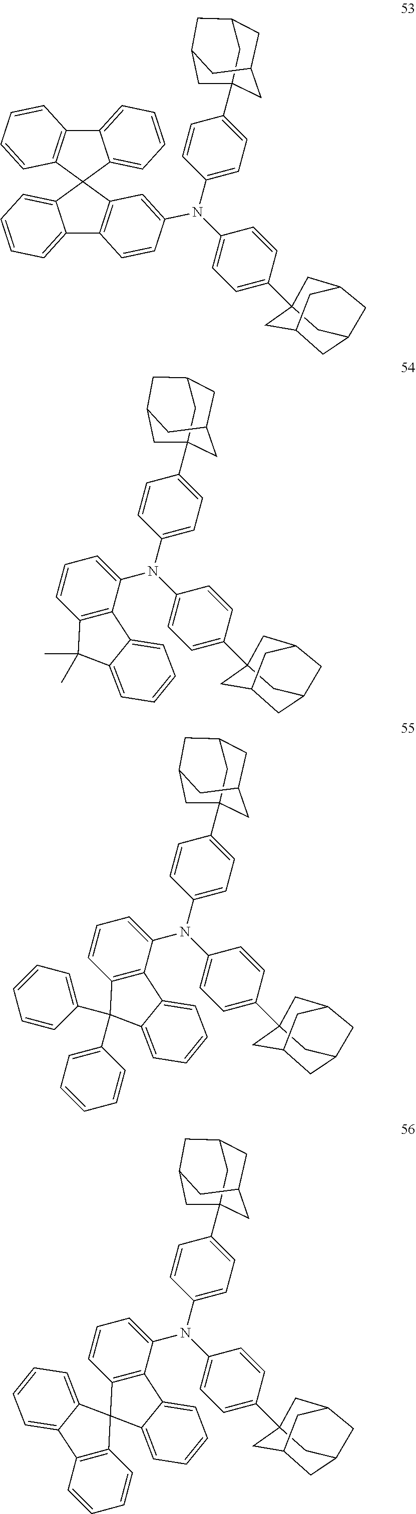 [SMILES: c1ccc2c(c1)-c1ccccc1C21c2ccccc2-c2ccc(N(c3ccc(C45CC6CC(CC(C6)C4)C5)cc3)c3ccc(C45CC6CC(CC(C6)C4)C5)cc3)cc21 ; CC1(C)c2ccccc2-c2c(N(c3ccc(C45CC6CC(CC(C6)C4)C5)cc3)c3ccc(C45CC6CC(CC(C6)C4)C5)cc3)cccc21 ; c1ccc(C2(c3ccccc3)c3ccccc3-c3c(N(c4ccc(C56CC7CC(CC(C7)C5)C6)cc4)c4ccc(C56CC7CC(CC(C7)C5)C6)cc4)cccc32)cc1 ; c1ccc2c(c1)-c1ccccc1C21c2ccccc2-c2c(N(c3ccc(C45CC6CC(CC(C6)C4)C5)cc3)c3ccc(C45CC6CC(CC(C6)C4)C5)cc3)cccc21]

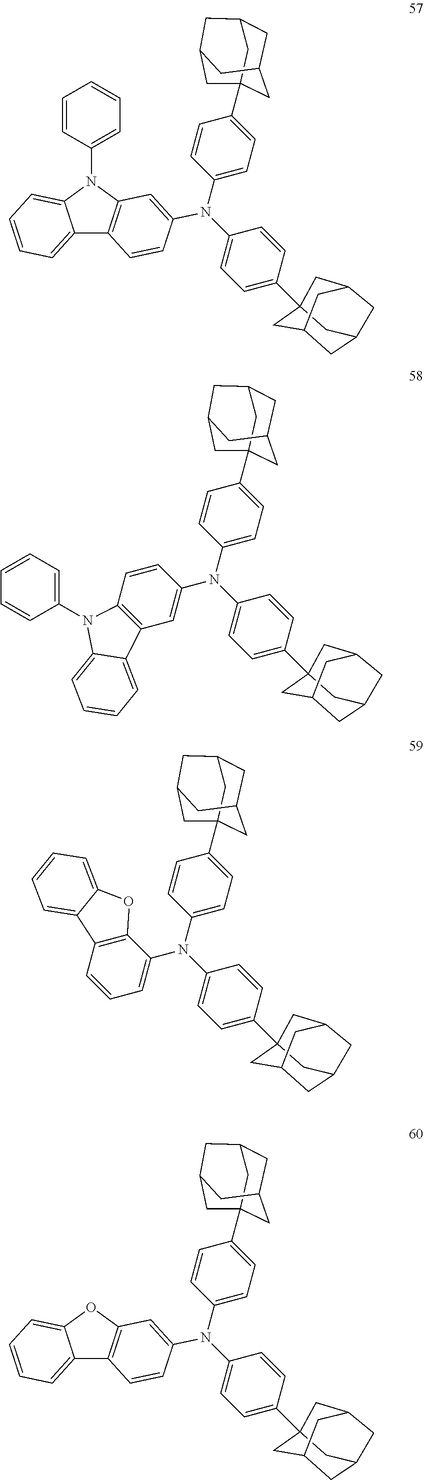 [SMILES: c1ccc(-n2c3ccccc3c3ccc(N(c4ccc(C56CC7CC(CC(C7)C5)C6)cc4)c4ccc(C56CC7CC(CC(C7)C5)C6)cc4)cc32)cc1 ; c1ccc(-n2c3ccccc3c3cc(N(c4ccc(C56CC7CC(CC(C7)C5)C6)cc4)c4ccc(C56CC7CC(CC(C7)C5)C6)cc4)ccc32)cc1 ; c1ccc2c(c1)oc1c(N(c3ccc(C45CC6CC(CC(C6)C4)C5)cc3)c3ccc(C45CC6CC(CC(C6)C4)C5)cc3)cccc12 ; c1ccc2c(c1)oc1cc(N(c3ccc(C45CC6CC(CC(C6)C4)C5)cc3)c3ccc(C45CC6CC(CC(C6)C4)C5)cc3)ccc12]

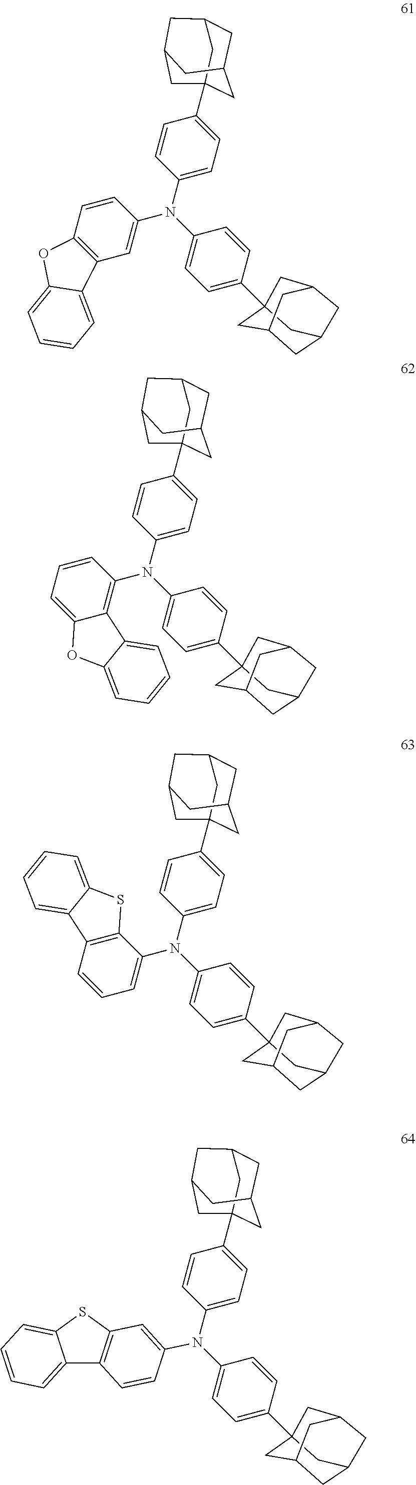 [SMILES: c1ccc2c(c1)oc1ccc(N(c3ccc(C45CC6CC(CC(C6)C4)C5)cc3)c3ccc(C45CC6CC(CC(C6)C4)C5)cc3)cc12 ; c1ccc2c(c1)oc1cccc(N(c3ccc(C45CC6CC(CC(C6)C4)C5)cc3)c3ccc(C45CC6CC(CC(C6)C4)C5)cc3)c12 ; c1ccc2c(c1)sc1c(N(c3ccc(C45CC6CC(CC(C6)C4)C5)cc3)c3ccc(C45CC6CC(CC(C6)C4)C5)cc3)cccc12 ; c1ccc2c(c1)sc1cc(N(c3ccc(C45CC6CC(CC(C6)C4)C5)cc3)c3ccc(C45CC6CC(CC(C6)C4)C5)cc3)ccc12]

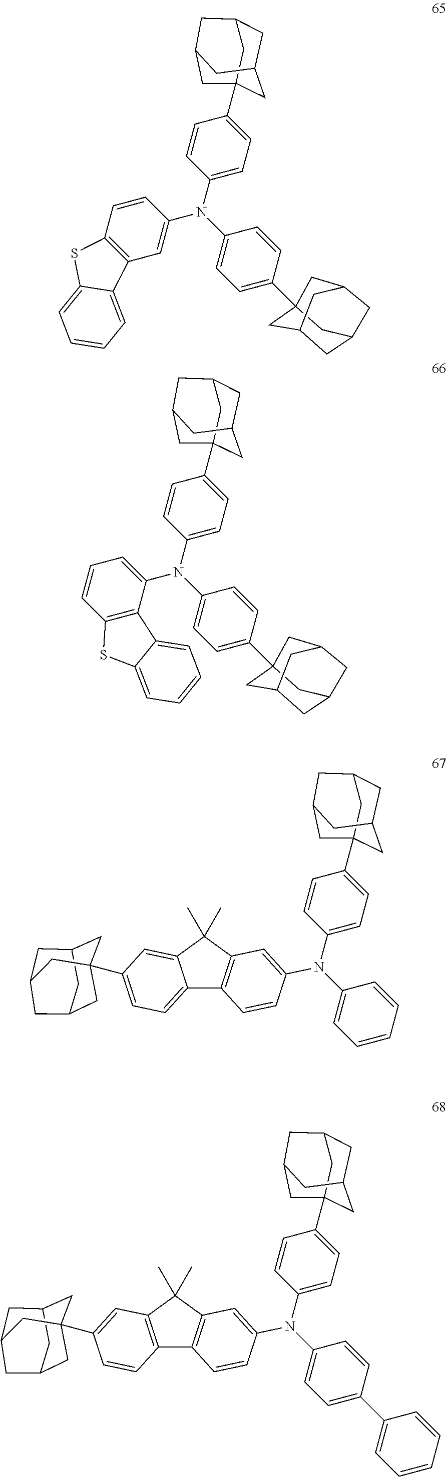 [SMILES: c1ccc2c(c1)sc1ccc(N(c3ccc(C45CC6CC(CC(C6)C4)C5)cc3)c3ccc(C45CC6CC(CC(C6)C4)C5)cc3)cc12 ; c1ccc2c(c1)sc1cccc(N(c3ccc(C45CC6CC(CC(C6)C4)C5)cc3)c3ccc(C45CC6CC(CC(C6)C4)C5)cc3)c12 ; CC1(C)c2cc(N(c3ccccc3)c3ccc(C45CC6CC(CC(C6)C4)C5)cc3)ccc2-c2ccc(C34CC5CC(CC(C5)C3)C4)cc21 ; CC1(C)c2cc(N(c3ccc(-c4ccccc4)cc3)c3ccc(C45CC6CC(CC(C6)C4)C5)cc3)ccc2-c2ccc(C34CC5CC(CC(C5)C3)C4)cc21]

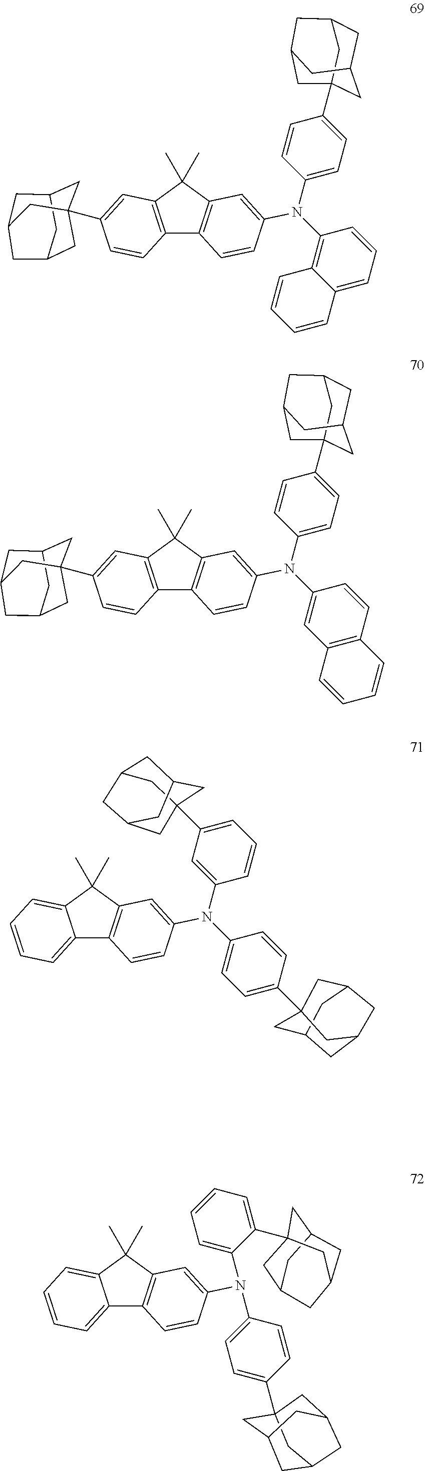

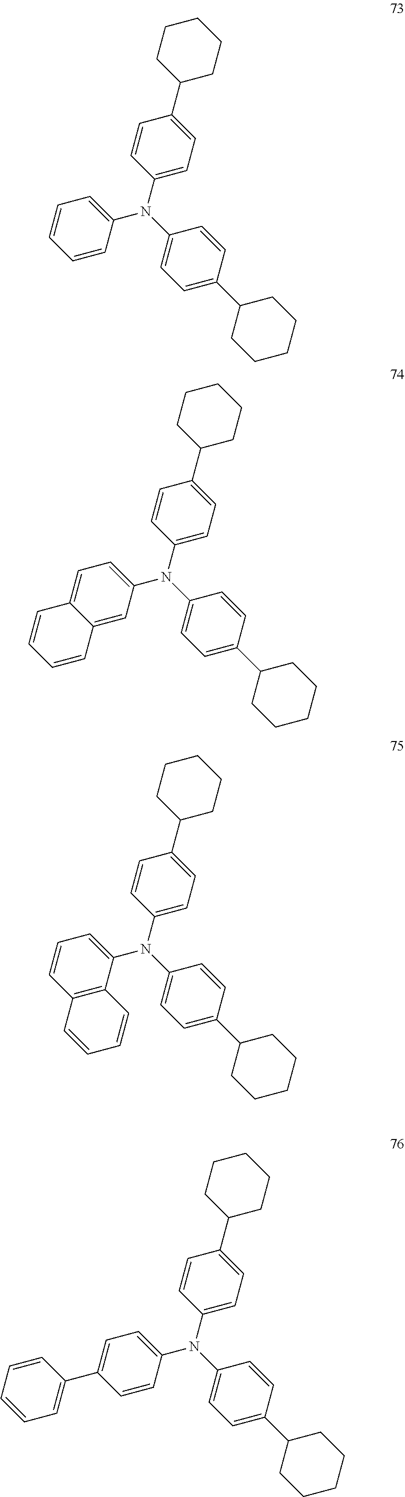

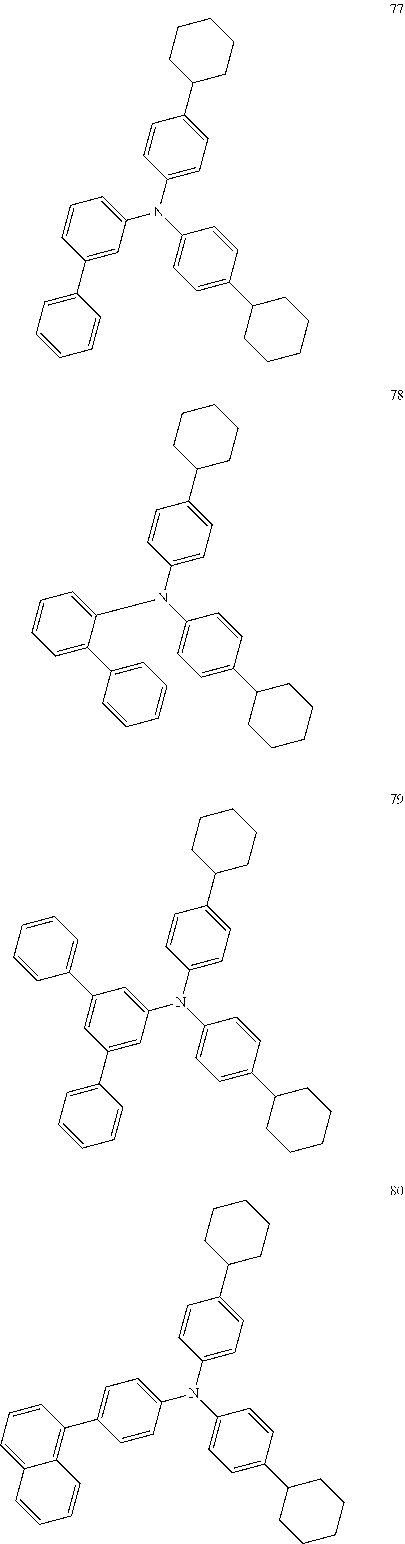 [SMILES: c1ccc(-c2cccc(N(c3ccc(C4CCCCC4)cc3)c3ccc(C4CCCCC4)cc3)c2)cc1 ; c1ccc(-c2ccccc2N(c2ccc(C3CCCCC3)cc2)c2ccc(C3CCCCC3)cc2)cc1 ; c1ccc(-c2cc(-c3ccccc3)cc(N(c3ccc(C4CCCCC4)cc3)c3ccc(C4CCCCC4)cc3)c2)cc1 ; c1ccc2c(-c3ccc(N(c4ccc(C5CCCCC5)cc4)c4ccc(C5CCCCC5)cc4)cc3)cccc2c1]

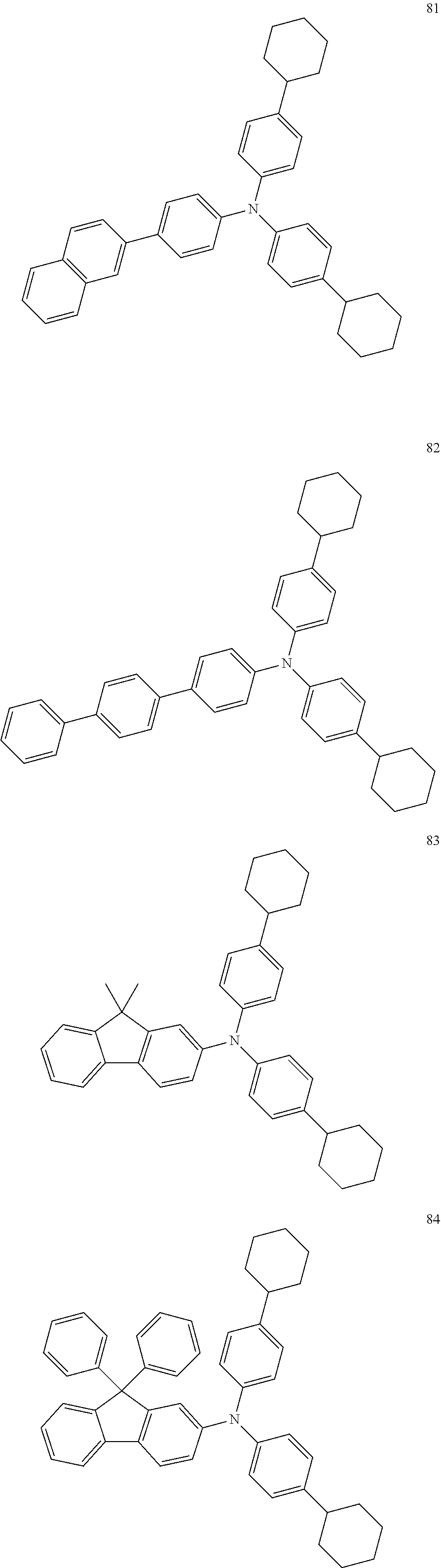 [SMILES: c1ccc2cc(-c3ccc(N(c4ccc(C5CCCCC5)cc4)c4ccc(C5CCCCC5)cc4)cc3)ccc2c1 ; c1ccc(-c2ccc(-c3ccc(N(c4ccc(C5CCCCC5)cc4)c4ccc(C5CCCCC5)cc4)cc3)cc2)cc1 ; CC1(C)c2ccccc2-c2ccc(N(c3ccc(C4CCCCC4)cc3)c3ccc(C4CCCCC4)cc3)cc21 ; c1ccc(C2(c3ccccc3)c3ccccc3-c3ccc(N(c4ccc(C5CCCCC5)cc4)c4ccc(C5CCCCC5)cc4)cc32)cc1]

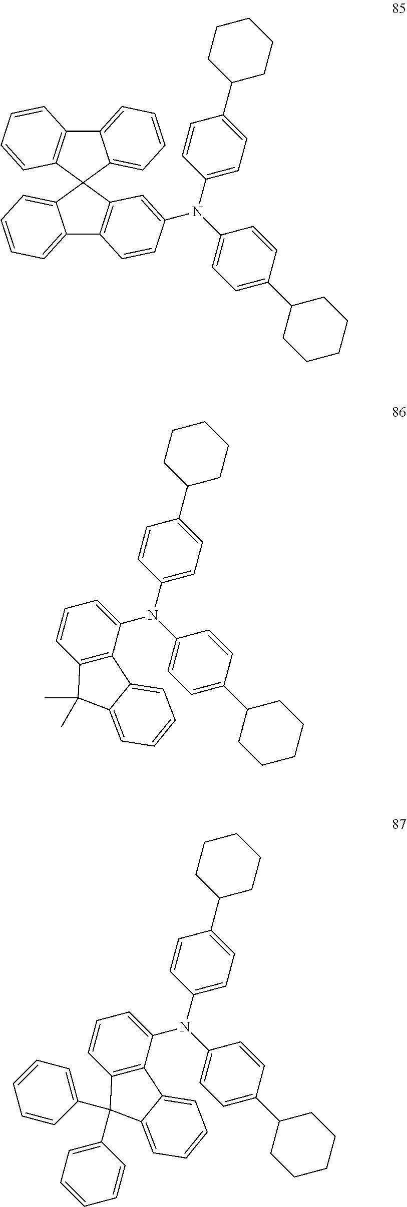 [SMILES: c1ccc2c(c1)-c1ccccc1C21c2ccccc2-c2ccc(N(c3ccc(C4CCCCC4)cc3)c3ccc(C4CCCCC4)cc3)cc21 ; CC1(C)c2ccccc2-c2c(N(c3ccc(C4CCCCC4)cc3)c3ccc(C4CCCCC4)cc3)cccc21 ; c1ccc(C2(c3ccccc3)c3ccccc3-c3c(N(c4ccc(C5CCCCC5)cc4)c4ccc(C5CCCCC5)cc4)cccc32)cc1]

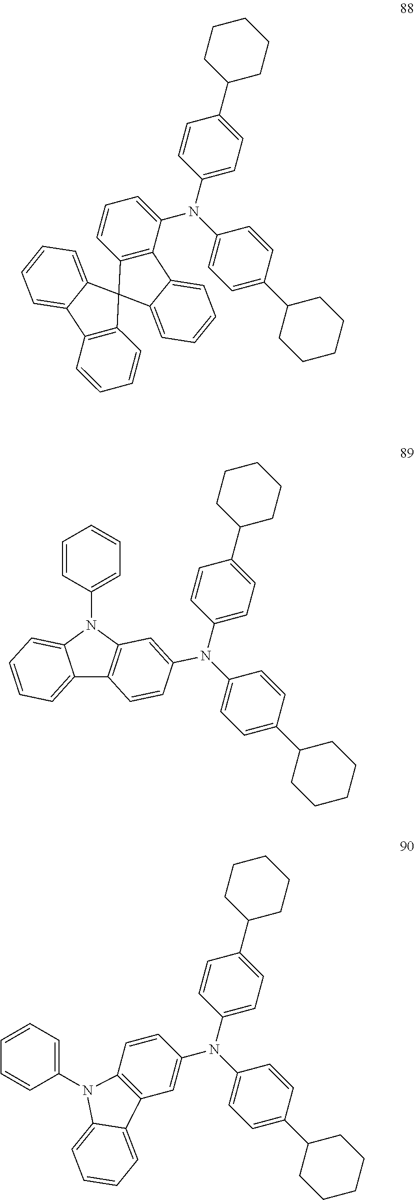 [SMILES: c1ccc2c(c1)-c1ccccc1C21c2ccccc2-c2c(N(c3ccc(C4CCCCC4)cc3)c3ccc(C4CCCCC4)cc3)cccc21 ; c1ccc(-n2c3ccccc3c3ccc(N(c4ccc(C5CCCCC5)cc4)c4ccc(C5CCCCC5)cc4)cc32)cc1 ; c1ccc(-n2c3ccccc3c3cc(N(c4ccc(C5CCCCC5)cc4)c4ccc(C5CCCCC5)cc4)ccc32)cc1]

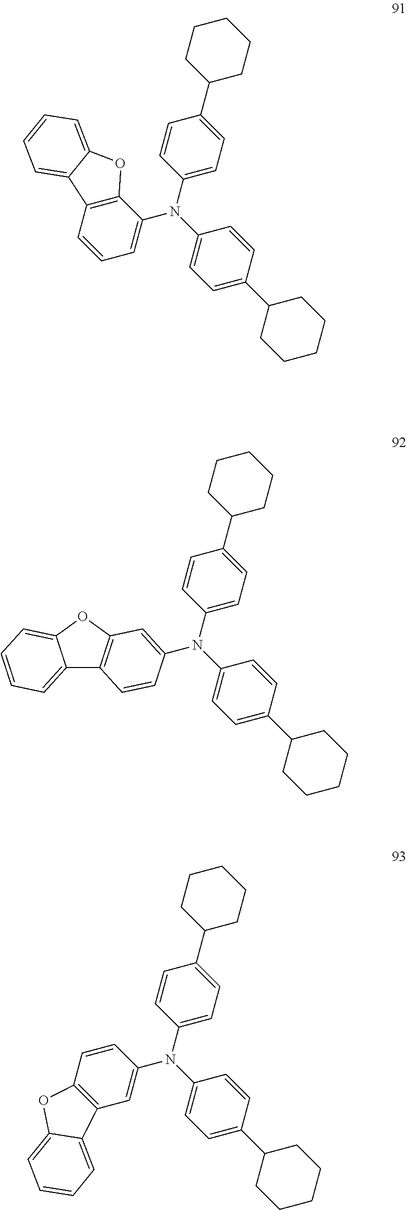 [SMILES: c1ccc2c(c1)oc1c(N(c3ccc(C4CCCCC4)cc3)c3ccc(C4CCCCC4)cc3)cccc12 ; c1ccc2c(c1)oc1cc(N(c3ccc(C4CCCCC4)cc3)c3ccc(C4CCCCC4)cc3)ccc12 ; c1ccc2c(c1)oc1ccc(N(c3ccc(C4CCCCC4)cc3)c3ccc(C4CCCCC4)cc3)cc12]

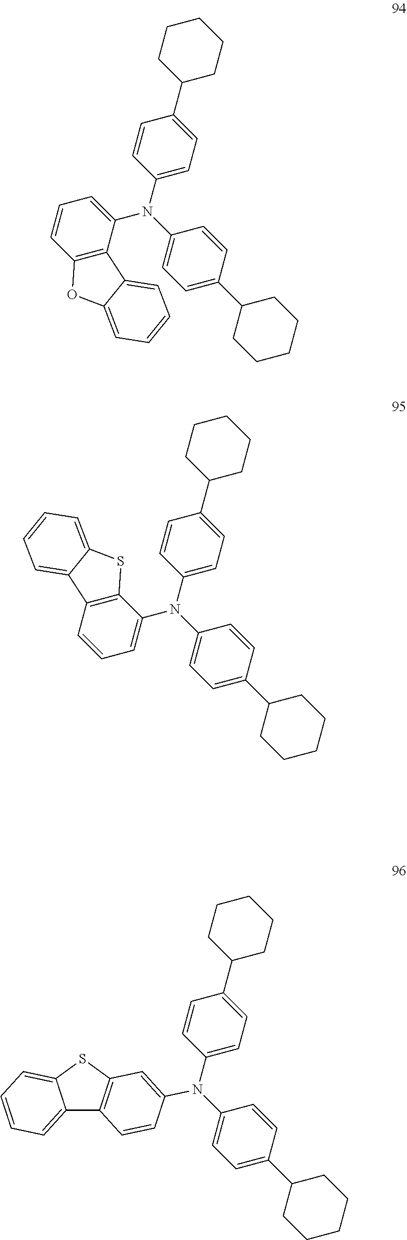 [SMILES: c1ccc2c(c1)oc1cccc(N(c3ccc(C4CCCCC4)cc3)c3ccc(C4CCCCC4)cc3)c12 ; c1ccc2c(c1)sc1c(N(c3ccc(C4CCCCC4)cc3)c3ccc(C4CCCCC4)cc3)cccc12 ; c1ccc2c(c1)sc1cc(N(c3ccc(C4CCCCC4)cc3)c3ccc(C4CCCCC4)cc3)ccc12]

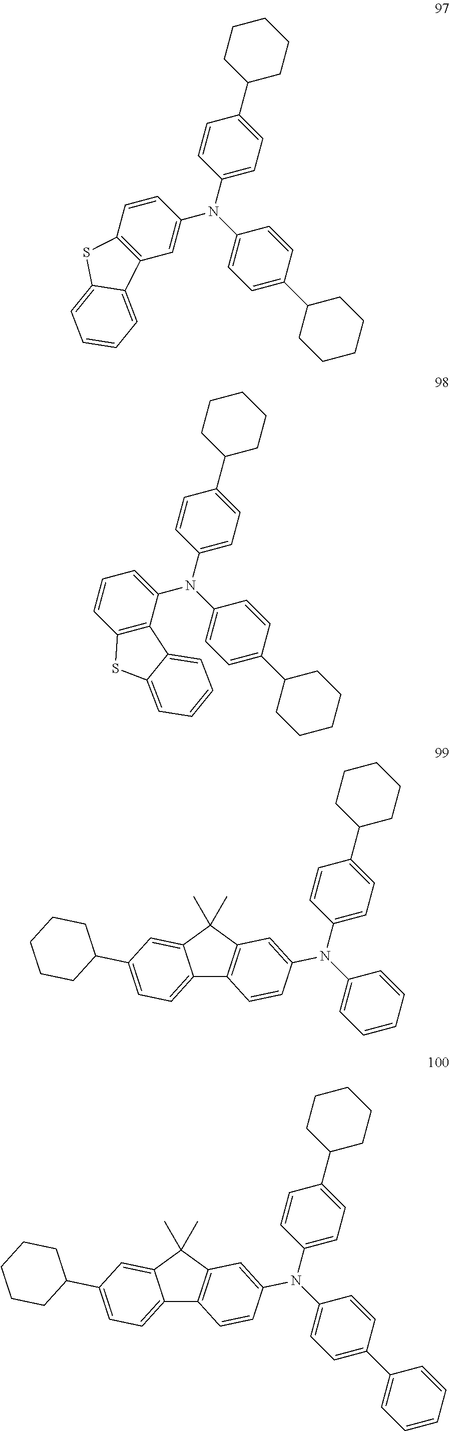

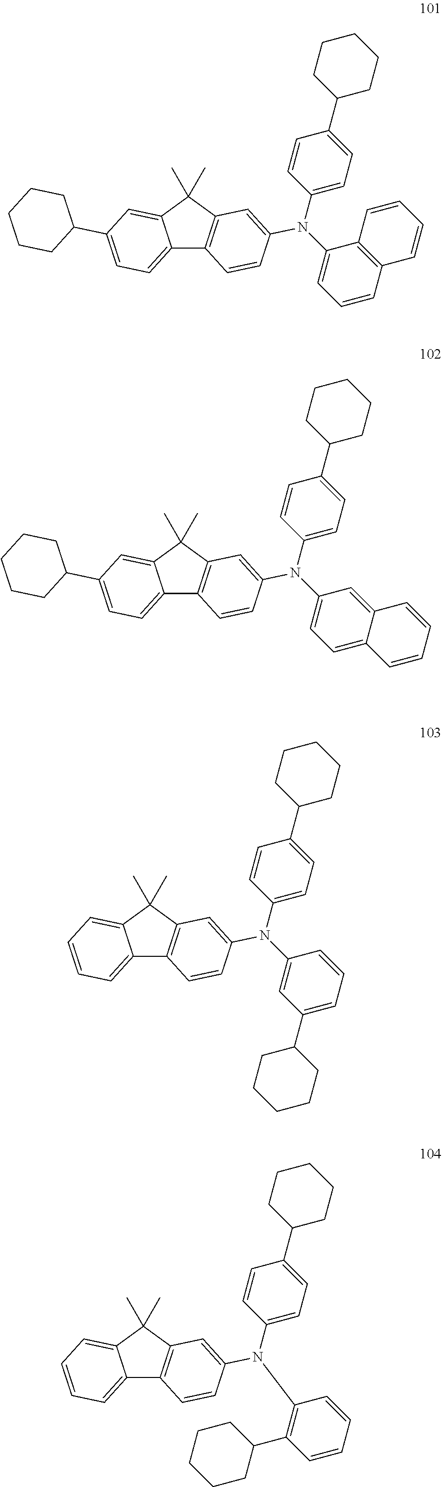 [SMILES: CC1(C)c2cc(C3CCCCC3)ccc2-c2ccc(N(c3ccc(C4CCCCC4)cc3)c3cccc4ccccc34)cc21 ; CC1(C)c2cc(C3CCCCC3)ccc2-c2ccc(N(c3ccc(C4CCCCC4)cc3)c3ccc4ccccc4c3)cc21 ; CC1(C)c2ccccc2-c2ccc(N(c3ccc(C4CCCCC4)cc3)c3cccc(C4CCCCC4)c3)cc21 ; CC1(C)c2ccccc2-c2ccc(N(c3ccc(C4CCCCC4)cc3)c3ccccc3C3CCCCC3)cc21]

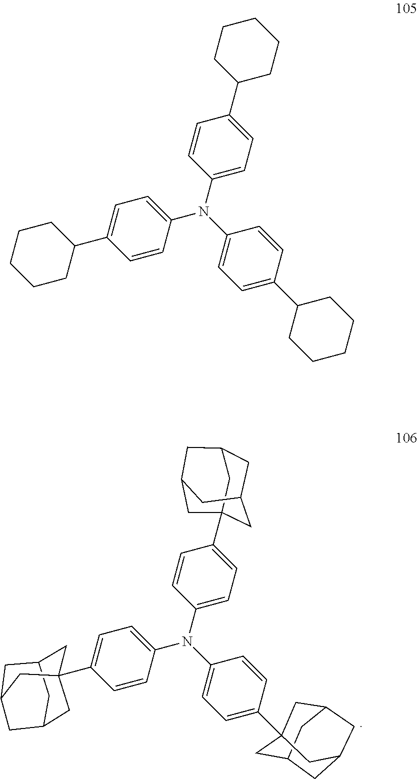 [SMILES: c1cc(N(c2ccc(C3CCCCC3)cc2)c2ccc(C3CCCCC3)cc2)ccc1C1CCCCC1 ; c1cc(C23CC4CC(CC(C4)C2)C3)ccc1N(c1ccc(C23CC4CC(CC(C4)C2)C3)cc1)c1ccc(C23CC4CC(CC(C4)C2)C3)cc1]

53. The display device of claim 47, wherein the hole transport region comprising the amine compound has a refractive index in a range of about 1.30 to about 1.80, with respect to light at a wavelength of about 460 nm.

54. The display device of claim 47, wherein
the plurality of light emitting units comprise first to fourth light emitting units disposed in order,
each of the first light emitting unit, the second light emitting unit, and the third light emitting unit emits light having a first wavelength in a range of about 430 nm to about 470 nm, and
the fourth light emitting unit emits light having a second wavelength in a range of about 520 nm to about 600 nm.
